(19) United States
(12) Patent Application Publication
(10) Pub. No.: US 2023/0020089 A1
(43) Pub. Date: Jan. 19, 2023

(54) SHARED NEOANTIGEN VACCINES

(71) Applicant: Gritstone bio, Inc., Emeryville, CA (US)

(72) Inventors: Andrew Ferguson, Hingham, MA (US); Raphael Rousseau, Los Altos, CA (US); Roman Yelensky, Newton, MA (US); James Xin Sun, Newton, MA (US); Matthew Joseph Davis, Scituate, MA (US); Karin Jooss, Emeryville, CA (US); Amy Rachel Rappaport, Daly City, CA (US); Ciaran Daniel Scallan, San Francisco, CA (US); Leonid Gitlin, Foster City, CA (US); Christine Denise Palmer, Cambridge, MA (US); Jennifer Busby, Burlington, MA (US); Brendan Bulik-Sullivan, Cambridge, MA (US); Mojca Skoberne, Cambridge, MA (US); Wade Blair, Gaithersburg, MD (US)

(21) Appl. No.: 17/736,738

(22) Filed: May 4, 2022

Related U.S. Application Data
(63) Continuation of application No. PCT/US2020/058983, filed on Nov. 4, 2020.
(60) Provisional application No. 63/051,227, filed on Jul. 13, 2020.
(60) Provisional application No. 62/959,798, filed on Jan. 10, 2020.
(60) Provisional application No. 62/946,956, filed on Dec. 11, 2019.
(60) Provisional application No. 62/930,460, filed on Nov. 4, 2019.

(51) Int. Cl. A61K 39/00; A61P 35/00; C12N 15/86; C12N 15/62

(52) U.S. Cl. CPC A61K 39/001154; A61P 35/00; C12N 15/86; C12N 15/625; A61K 2039/5256; A61K 2039/545; C12N 2770/36143; C12N 2770/36134; C12N 2710/10343; C12N 2710/10334

(57) ABSTRACT

Disclosed herein are compositions that include antigen-encoding nucleic acid sequences and/or antigen peptides. Also disclosed are nucleotides, cells, and methods associated with the compositions including their use as vaccines, including vectors and methods for a heterologous prime/boost vaccincation strategy.

### CROSS REFERENCE TO RELATED APPLICATIONS

This application is a continuation of International Application No. PCT/US20/58983, filed Nov. 4, 2020, which claims the benefit of U.S. Provisional Application Nos. 62/930,460 filed Nov. 4, 2019; 62/946,956 filed Dec. 11, 2019; 62/959,798 filed Jan. 10, 2020; and 63/051,227 filed Jul. 13, 2020, each of which is hereby incorporated in its entirety by reference for all purposes.

### SEQUENCE LISTING

The instant application contains a Sequence Listing which has been submitted electronically in ASCII format and is hereby incorporated by reference in its entirety. Said ASCII copy, created on Nov. 4, 2020, is named GSO-082WO_Sequence_Listing.txt and is 6,969,609 bytes in size.

### BACKGROUND

Therapeutic vaccines based on tumor-specific antigens hold great promise as a next-generation of personalized cancer immunotherapy.¹⁻³ For example, cancers with a high mutational burden, such as non-small cell lung cancer (NSCLC) and melanoma, are particularly attractive targets of such therapy given the relatively greater likelihood of neoantigen generation.^{4,5} Early evidence shows that neoantigen-based vaccination can elicit T-cell responses' and that neoantigen targeted cell-therapy can cause tumor regression under certain circumstances in selected patients.⁷

One question for neoantigen vaccine design is which of the many coding mutations present in subject tumors can generate the “best” therapeutic neoantigens, e.g., antigens that can elicit anti-tumor immunity and cause tumor regression.

Initial methods have been proposed incorporating mutation-based analysis using next-generation sequencing, RNA gene expression, and prediction of MHC binding affinity of candidate neoantigen peptides⁸. However, these proposed methods can fail to model the entirety of the epitope generation process, which contains many steps (e.g., TAP transport, proteasomal cleavage, and/or TCR recognition) in addition to gene expression and MHC binding⁹. Consequently, existing methods are likely to suffer from reduced low positive predictive value (PPV).

Indeed, analyses of peptides presented by tumor cells performed by multiple groups have shown that <5% of peptides that are predicted to be presented using gene expression and MHC binding affinity can be found on the tumor surface MHC^{10,11}. This low correlation between binding prediction and MHC presentation was further reinforced by recent observations of the lack of predictive accuracy improvement of binding-restricted neoantigens for checkpoint inhibitor response over the number of mutations alone.¹²

This low positive predictive value (PPV) of existing methods for predicting presentation presents a problem for neoantigen-based vaccine design. If vaccines are designed using predictions with a low PPV, most patients are unlikely to receive a therapeutic neoantigen and fewer still are likely to receive more than one (even assuming all presented peptides are immunogenic). Thus, neoantigen vaccination with current methods is unlikely to succeed in a substantial number of subjects having tumors.

Additionally, previous approaches generated candidate neoantigens using only cis-acting mutations, and largely neglected to consider additional sources of neo-ORFs, including mutations in splicing factors, which occur in multiple tumor types and lead to aberrant splicing of many genes¹³, and mutations that create or remove protease cleavage sites.

Finally, standard approaches to tumor genome and transcriptome analysis can miss somatic mutations that give rise to candidate neoantigens due to suboptimal conditions in library construction, exome and transcriptome capture, sequencing, or data analysis. Likewise, standard tumor analysis approaches can inadvertently promote sequence artifacts or germline polymorphisms as neoantigens, leading to inefficient use of vaccine capacity or auto-immunity risk, respectively.

In addition to the challenges of current neoantigen prediction methods certain challenges also exist with the available vector systems that can be used for neoantigen delivery in humans, many of which are derived from humans. For example, many humans have pre-existing immunity to human viruses as a result of previous natural exposure, and this immunity can be a major obstacle to the use of recombinant human viruses for neoantigen delivery for cancer treatment.

In addition, targeting antigens that are shared among patients with cancer hold great promise as a vaccine strategy, including targeting both neoantigens with a mutation as well as tumor antigens without a mutation (e.g., tumors antigens that are improperly expressed). The challenges with shared antigen vaccine strategies include at least those discussed above.

### SUMMARY

Disclosed herein is a composition for delivery of a self-amplifying alphavirus-based expression system, wherein the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; (ii) optionally, a second promoter nucleotide sequence operably linked to the at least one antigen-encoding nucleic acid sequence; and (iii) optionally, at least one second poly(A) sequence, wherein the second poly(A) sequence is a native poly(A) sequence or an exogenous poly(A) sequence to the alphavirus, and (B) a lipid-nanoparticle (LNP), wherein the LNP encapsulates the self-amplifying alphavirus-based expression system, and wherein the composition comprises at least 10 μg of each of the one or more vectors.

Also disclosed herein is a composition for delivery of a self-amplifying alphavirus-based expression system, wherein the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises the nucleic acid sequence set forth in SEQ ID NO:6, wherein the RNA alphavirus backbone sequence comprises a 26S promoter nucleotide sequence and a poly(A) sequence, wherein the 26S promoter sequence is endogenous to the RNA alphavirus backbone, and wherein the poly(A) sequence is endogenous to the RNA alphavirus backbone; and (b) a cassette integrated between the 26S promoter nucleotide sequence and the poly(A) sequence, wherein the cassette is operably linked to the 26S promoter nucleotide sequence, and wherein the cassette comprises at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and (B) a lipid-nanoparticle (LNP), wherein the LNP encapsulates the self-amplifying alphavirus-based expression system, and wherein the composition comprises at least 30 μg of each of the one or more vectors.

In some aspects, the composition for delivery of the self-amplifying alphavirus-based expression system comprises at least 30 μg of each of the one or more vectors. In some aspects, the composition for delivery of the self-amplifying alphavirus-based expression system comprises at least 100 μg of each of the one or more vectors. In some aspects, the composition for delivery of the self-amplifying alphavirus-based expression system comprises at least 300 μg of each of the one or more vectors. In some aspects, the composition for delivery of the self-amplifying alphavirus-based expression system comprises at least 400 μg, at least 500 μg, at least 600 μg, at least 700 μg, at least 800 μg, at least 900 μg, at least 1000 μg of each of the one or more vectors. In some aspects, the composition for delivery of the self-amplifying alphavirus-based expression system comprises between 10-30 μg, 10-100 μg, 10-300 μg, 30-100 μg, 30-300 μg, or 100-300 μg of each of the one or more vectors. In some aspects, the composition for delivery of the self-amplifying alphavirus-based expression system comprises between 10-500 μg, 10-1000 μg, 30-500 μg, 30-1000 μg, or 500-1000 μg of each of the one or more vectors. In some aspects, the composition for delivery of the self-amplifying alphavirus-based expression system comprises 10 μg, 30 μg, 100 μg, or 300 μg of each of the one or more vectors. In some aspects, the composition for delivery of the self-amplifying alphavirus-based expression system comprises 400 μg, 500 μg, 600 μg, 700 μg, 800 μg, 900 μg, or 1000 μg of each of the one or more vectors. In some aspects, the composition for delivery of the self-amplifying alphavirus-based expression system comprises less than or equal to 300 μg of each of the one or more vectors.

In some aspects, the weight to weight ratio of the LNP to total weight of the one or more vectors is between 10-40 to 1. In some aspects, the weight to weight ratio of the LNP to total weight of the one or more vectors is between 16-32 to 1. In some aspects, the weight to weight ratio of the LNP to total weight of the one or more vectors is about 24 to 1. In some aspects, the weight to weight ratio of the LNP to total weight of the one or more vectors is 24 to 1.

In some aspects, the one or more vectors is at a concentration of 1 mg/mL.

In some aspects, an ordered sequence of each element of the cassette in the composition for delivery of the self-amplifying alphavirus-based expression system is described in the formula, from 5′ to 3′, comprising Pₐ-(L5_{b}-N_{c}-L3_{d})_{X}-(G5ₑ-U_{f})_{Y}-G3_{g} wherein P comprises the second promoter nucleotide sequence, where a=0 or 1, N comprises one of the epitope-encoding nucleic acid sequences, wherein the epitope-encoding nucleic acid sequence comprises an MHC class I epitope-encoding nucleic acid sequence, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding N_{c} is an MHC class I epitope-encoding nucleic acid sequence, and Y=0, 1, or 2, where for each Y the corresponding U_{f} is an MHC class II epitope-encoding nucleic acid sequence. In some aspects, for each X the corresponding N_{c} is a distinct MHC class I epitope-encoding nucleic acid sequence. In some aspects, for each Y the corresponding U_{f} is a distinct MHC class II epitope-encoding nucleic acid sequence. In some aspects, wherein a=0, b=1, d=1, e=1, g=1, h=1, X=20, Y=2, the at least one promoter nucleotide sequence is a single 26S promoter nucleotide sequence provided by the RNA alphavirus backbone, the at least one polyadenylation poly(A) sequence is a poly(A) sequence of at least 100 consecutive A nucleotides (SEQ ID NO: 29358) provided by the RNA alphavirus backbone, the cassette is integrated between the 26S promoter nucleotide sequence and the poly(A) sequence, wherein the cassette is operably linked to the 26S promoter nucleotide sequence and the poly(A) sequence, each N encodes a MHC class I epitope 7-15 amino acids in length, L5 is a native 5′ linker sequence that encodes a native N-terminal amino acid sequence of the MHC I epitope, and wherein the 5′ linker sequence encodes a peptide that is at least 3 amino acids in length, L3 is a native 3′ linker sequence that encodes a native C-terminal amino acid sequence of the MHC I epitope, and wherein the 3′ linker sequence encodes a peptide that is at least 3 amino acids in length, U is each of a PADRE class II sequence and a Tetanus toxoid MHC class II sequence, the RNA alphavirus backbone is the sequence set forth in SEQ ID NO:6, and each of the MHC class I epitope-encoding nucleic acid sequences encodes a polypeptide that is between 13 and 25 amino acids in length.

In some aspects, the LNP comprises a lipid selected from the group consisting of: an ionizable amino lipid, a phosphatidylcholine, cholesterol, a PEG-based coat lipid, or a combination thereof. In some aspects, the LNP comprises an ionizable amino lipid, a phosphatidylcholine, cholesterol, and a PEG-based coat lipid. In some aspects, the ionizable amino lipids comprise MC3-like (dilinoleylmethyl-4-dimethylaminobutyrate) molecules. In some aspects, the LNP-encapsulated expression system has a diameter of about 100 nm.

In some aspects, the composition for delivery of the self-amplifying alphavirus-based expression system is formulated for intramuscular (IM), intradermal (ID), subcutaneous (SC), or intravenous (IV) administration. In some aspects, the composition for delivery of the self-amplifying alphavirus-based expression system is formulated for intramuscular (IM) administration.

In some aspects, the cassette is integrated between the at least one promoter nucleotide sequence and the at least one poly(A) sequence. In some aspects, the at least one promoter nucleotide sequence is operably linked to the cassette.

In some aspects, the one or more vectors comprise one or more +-stranded RNA vectors. In some aspects, the one or more +-stranded RNA vectors comprise a 5′ 7-methylguanosine (m7g) cap. In some aspects, the one or more +-stranded RNA vectors are produced by in vitro transcription. In some aspects, the one or more vectors are self-amplifying within a mammalian cell. In some aspects, the RNA alphavirus backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the RNA alphavirus backbone comprises at least one nucleotide sequence of a Venezuelan equine encephalitis virus. In some aspects, the RNA alphavirus backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the RNA alphavirus backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the RNA alphavirus backbone does not encode structural virion proteins capsid, E2 and E1. In some aspects, the cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11175. In some aspects, the RNA alphavirus backbone comprises the sequence set forth in SEQ ID NO:6 or SEQ ID NO:7. In some aspects, the cassette is inserted at position 7544 to replace the deletion between base pairs 7544 and 11175 as set forth in the sequence of SEQ ID NO:3 or SEQ ID NO:5. In some aspects, the insertion of the cassette provides for transcription of a polycistronic RNA comprising the nsP1-4 genes and the at least one nucleic acid sequence, wherein the nsP1-4 genes and the at least one nucleic acid sequence are in separate open reading frames.

In some aspects, the at least one promoter nucleotide sequence is the native 26S promoter nucleotide sequence encoded by the RNA alphavirus backbone. In some aspects, the at least one promoter nucleotide sequence is an exogenous RNA promoter. In some aspects, the second promoter nucleotide sequence is a 26S promoter nucleotide sequence. In some aspects, the second promoter nucleotide sequence comprises multiple 26S promoter nucleotide sequences, wherein each 26S promoter nucleotide sequence provides for transcription of one or more of the separate open reading frames.

In some aspects, the one or more vectors are each at least 300 nt in size. In some aspects, the one or more vectors are each at least 1 kb in size. In some aspects, the one or more vectors are each 2 kb in size. In some aspects, the one or more vectors are each less than 5 kb in size.

In some aspects, the at least one antigen-encoding nucleic acid sequence comprises two or more antigen-encoding nucleic acid sequences. In some aspects, each antigen-encoding nucleic acid sequence is linked directly to one another. In some aspects, each antigen-encoding nucleic acid sequence is linked to a distinct antigen-encoding nucleic acid sequence with a nucleic acid sequence encoding a linker. In some aspects, the linker links two epitope-encoding nucleic acid sequences or an epitope-encoding nucleic acid sequence to an MHC class II epitope-encoding nucleic acid sequence. In some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II epitope-encoding nucleic acid sequences or an MHC class II sequence to an epitope-encoding nucleic acid sequence. In some aspects, the linker comprises the sequence GPGPG (SEQ ID NO: 56).

In some aspects, the antigen-encoding nucleic acid sequences is linked, operably or directly, to a separate or contiguous sequence that enhances the expression, stability, cell trafficking, processing and presentation, and/or immunogenicity of the antigen-encoding nucleic acid sequence. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, the at least one antigen-encoding nucleic acid sequence comprises at least 2-10, 2, 3, 4, 5, 6, 7, 8, 9, or 10 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode epitope sequences or portions thereof that are presented by MHC class I on a cell surface. In some aspects, the MHC class I epitopes are presented by MHC class I on the tumor cell surface.

In some aspects, the epitope-encoding nucleic acid sequences comprises at least one MHC class I epitope-encoding nucleic acid sequence, and wherein each antigen-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded epitope sequence distinct from the corresponding peptide sequence encoded by a wild-type nucleic acid sequence. In some aspects, the epitope-encoding nucleic acid sequence comprises an MHC class II epitope-encoding nucleic acid sequence and wherein each antigen-encoding nucleic acid sequence encodes a polypeptide sequence that is 12-20, 12, 13, 14, 15, 16, 17, 18, 19, 20, or 20-40 amino acids in length. In some aspects, the epitope-encoding nucleic acid sequences comprises an MHC class II epitope-encoding nucleic acid sequence, wherein the at least one MHC class II epitope-encoding nucleic acid sequence is present, and wherein the at least one MHC class II epitope-encoding nucleic acid sequence comprises at least one universal MHC class II epitope-encoding nucleic acid sequence, optionally wherein the at least one universal sequence comprises at least one of Tetanus toxoid and PADRE.

In some aspects, the at least one promoter nucleotide sequence or the second promoter nucleotide sequence is inducible. In some aspects, the at least one promoter nucleotide sequence or the second promoter nucleotide sequence is non-inducible.

In some aspects, the at least one poly(A) sequence comprises a poly(A) sequence native to the alphavirus. In some aspects, the at least one poly(A) sequence comprises a poly(A) sequence exogenous to the alphavirus. In some aspects, the at least one poly(A) sequence is operably linked to at least one of the at least one nucleic acid sequences. In some aspects, the at least one poly(A) sequence is at least 20, at least 30, at least 40, at least 50, at least 60, at least 70, at least 80, or at least 90 consecutive A nucleotides (SEQ ID NO: 29361). In some aspects, the at least one poly(A) sequence is at least 100 consecutive A nucleotides (SEQ ID NO: 29358).

In some aspects, the epitope-encoding nucleic acid sequence comprises a MHC class I epitope-encoding nucleic acid sequence, and wherein the MHC class I epitope-encoding nucleic acid sequence is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the MHC class I epitope-encoding nucleic acid sequence. In some aspects, each of the MHC class I epitope-encoding nucleic acid sequences is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the at least 20 MHC class I epitope-encoding nucleic acid sequences. In some aspects, a number of the set of selected epitopes is 2-20. In some aspects, the presentation model represents dependence between: (a) presence of a pair of a particular one of the MHC alleles and a particular amino acid at a particular position of a peptide sequence; and (b) likelihood of presentation on the tumor cell surface, by the particular one of the MHC alleles of the pair, of such a peptide sequence comprising the particular amino acid at the particular position. In some aspects, selecting the set of selected epitopes comprises selecting epitopes that have an increased likelihood of being presented on the tumor cell surface relative to unselected epitopes based on the presentation model. In some aspects, selecting the set of selected epitopes comprises selecting epitopes that have an increased likelihood of being capable of inducing a tumor-specific immune response in the subject relative to unselected epitopes based on the presentation model. In some aspects, selecting the set of selected epitopes comprises selecting epitopes that have an increased likelihood of being capable of being presented to naïve T cells by professional antigen presenting cells (APCs) relative to unselected epitopes based on the presentation model, optionally wherein the APC is a dendritic cell (DC). In some aspects, selecting the set of selected epitopes comprises selecting epitopes that have a decreased likelihood of being subject to inhibition via central or peripheral tolerance relative to unselected epitopes based on the presentation model. In some aspects, selecting the set of selected epitopes comprises selecting epitopes that have a decreased likelihood of being capable of inducing an autoimmune response to normal tissue in the subject relative to unselected epitopes based on the presentation model. In some aspects, exome or transcriptome nucleotide sequencing data is obtained by performing sequencing on the tumor tissue. In some aspects, the sequencing is next generation sequencing (NGS) or any massively parallel sequencing approach.

Also disclosed herein is a composition for delivery of a chimpanzee adenovirus (ChAdV)-based expression system, wherein the composition for delivery of the ChAdV-based expression system comprises: the ChAdV-based expression system, wherein the ChAdV-based expression system comprises a viral particle comprising a ChAdV vector, wherein the ChAdV vector comprises: (a) a ChAdV backbone, wherein the ChAdV backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and wherein the cassette is operably linked to the at least one promoter nucleotide sequence and the at least one poly(A) sequence, and wherein the composition comprises 1×10¹² or less of the viral particles.

Also disclosed herein is a composition for delivery of a ChAdV-based expression system, wherein the composition for delivery of the ChAdV-based expression system comprises: the ChAdV-based expression system, wherein the ChAdV-based expression system comprises a viral particle comprising a ChAdV vector, wherein the ChAdV vector comprises: (a) a ChAdV backbone, wherein the ChAdV backbone comprises: (i) a modified ChAdV68 sequence comprising at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion and (2) nucleotides 27,125 to 31,825 of the sequence shown in SEQ ID NO:1 corresponding to an E3 deletion; and optionally lacks (3) nucleotides 34,916 to 35,642 of the sequence shown in SEQ ID NO:1 corresponding to a partial E4 deletion; (ii) a CMV promoter nucleotide sequence; and (iii) an SV40 polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and wherein the cassette is inserted within the E1 deletion and the cassette is operably linked to the CMV promoter nucleotide sequence and the SV40 poly(A) sequence, and wherein the composition comprises 1×10¹² or less of the viral particles.

In some aspects, the composition for delivery of the ChAdV-based expression system comprises 3×10¹¹ or less of the viral particles. In some aspects, the composition for delivery of the ChAdV-based expression system comprises at least 1×10¹¹ of the viral particles. In some aspects, the composition for delivery of the ChAdV-based expression system comprises between 1×10¹¹ and 1×10¹², between 3×10¹¹ and 1×10¹², or between 1×10¹¹ and 3×10¹¹ of the viral particles. In some aspects, the composition for delivery of the ChAdV-based expression system comprises 1×10¹¹, 3×10¹¹, or 1×10¹² of the viral particles.

In some aspects, the viral particles are at a concentration of at 5×10¹¹ vp/mL.

In some aspects, the epitope-encoding nucleic acid sequence encodes an epitope known or suspected to be presented by MHC class I on a surface of a cell, optionally wherein the surface of the cell is a tumor cell surface or an infected cell surface, and optionally wherein the cell is a subject's cell. In some aspects, the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer, or wherein the cell is an infected cell selected from the group consisting of: a pathogen infected cell, a virally infected cell, a bacterially infected cell, an fungally infected cell, and a parasitically infected cell. In some aspects, the virally infected cell is an HIV infected cell.

In some aspects, an ordered sequence of each element of the cassette in the composition for delivery of the ChAdV-based expression system is described in the formula, from 5′ to 3′, comprising Pₐ-(L5_{b}-N_{c}-L3_{d})_{X}-(G5ₑ-U_{f})_{Y}-G3_{g} wherein P comprises the at least one promoter sequence operably linked to at least one of the at least one antigen-encoding nucleic acid sequences, where a=1, N comprises one of the epitope-encoding nucleic acid sequences, wherein the epitope-encoding nucleic acid sequence comprises an MHC class I epitope-encoding nucleic acid sequence, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding N_{c} is an MHC class I epitope-encoding nucleic acid sequence, and Y=0, 1, or 2, where for each Y the corresponding U_{f} is an MHC class II epitope-encoding nucleic acid sequence. In some aspects, for each X the corresponding N_{c} is a distinct MHC class I epitope-encoding nucleic acid sequence. In some aspects, for each Y the corresponding U_{f} is a distinct MHC class II epitope-encoding nucleic acid sequence. In some aspects, b=1, d=1, e=1, g=1, h=1, X=10, Y=2, P is a CMV promoter sequence, each N encodes a MHC class I epitope 7-15 amino acids in length, L5 is a native 5′ linker sequence that encodes a native N-terminal amino acid sequence of the MHC I epitope, and wherein the 5′ linker sequence encodes a peptide that is at least 3 amino acids in length, L3 is a native 3′ linker sequence that encodes a native C-terminal amino acid sequence of the MHC I epitope, and wherein the 3′ linker sequence encodes a peptide that is at least 3 amino acids in length, U is each of a PADRE class II sequence and a Tetanus toxoid MHC class II sequence, the ChAdV vector comprises a modified ChAdV68 sequence comprising the sequence of SEQ ID NO:1 with an E1 (nt 577 to 3403) deletion and an E3 (nt 27,125-31,825) deletion and the neoantigen cassette is inserted within the E1 deletion, and each of the MHC class I antigen-encoding nucleic acid sequences encodes a polypeptide that is 25 amino acids in length.

In some aspects, the composition for delivery of the ChAdV-based expression system is formulated for intramuscular (IM), intradermal (ID), subcutaneous (SC), or intravenous (IV) administration. In some aspects, the composition for delivery of the ChAdV-based expression system is formulated for intramuscular (IM) administration.

In some aspects, the cassette is integrated between the at least one promoter nucleotide sequence and the at least one poly(A) sequence. In some aspects, the at least one promoter nucleotide sequence is operably linked to the cassette.

In some aspects, the ChAdV backbone comprises a ChAdV68 vector backbone. In some aspects, the ChAdV68 vector backbone comprises the sequence set forth in SEQ ID NO:1. In some aspects, the ChAdV68 vector backbone comprises a functional deletion in at least one gene selected from the group consisting of an adenovirus E1A, E1B, E2A, E2B, E3, L1, L2, L3, L4, and L5 gene with reference to a ChAdV68 genome or with reference to the sequence shown in SEQ ID NO:1, optionally wherein the adenoviral backbone or modified ChAdV68 sequence is fully deleted or functionally deleted in: (1) E1A and E1B; or (2) E1A, E1B, and E3 with reference to the adenovirus genome or with reference to the sequence shown in SEQ ID NO:1, optionally wherein the E1 gene is functionally deleted through an E1 deletion of at least nucleotides 577 to 3403 with reference to the sequence shown in SEQ ID NO:1 and optionally wherein the E3 gene is functionally deleted through an E3 deletion of at least nucleotides 27,125 to 31,825 with reference to the sequence shown in SEQ ID NO:1. In some aspects, the ChAdV68 vector backbone comprises one or more genes or regulatory sequences with reference to a ChAdV68 genome or with reference to the sequence shown in SEQ ID NO:1, optionally wherein the one or more genes or regulatory sequences are selected from the group consisting of the chimpanzee adenovirus inverted terminal repeat (ITR), E1A, E1B, E2A, E2B, E3, E4, L1, L2, L3, L4, and L5 genes. In some aspects, the ChAdV68 vector backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion and (2) nucleotides 27,125 to 31,825 of the sequence shown in SEQ ID NO:1 corresponding to an E3 deletion. In some aspects, the ChAdV68 vector backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) nucleotides 34,916 to 35,642 of the sequence shown in SEQ ID NO:1 corresponding to a partial E4 deletion; optionally wherein the antigen cassette is inserted within the E1 deletion.

In some aspects, the cassette is inserted in the ChAdV backbone at the E1 region, E3 region, and/or any deleted AdV region that allows incorporation of the cassette. In some aspects, the ChAdV backbone is generated from one of a first generation, a second generation, or a helper-dependent adenoviral vector.

In some aspects, the at least one promoter nucleotide sequence is selected from the group consisting of: a CMV, a SV40, an EF-1, a RSV, a PGK, a HSA, a MCK, and a EBV promoter sequence. In some aspects, the at least one promoter nucleotide sequence is a CMV promoter sequence.

In some aspects, at least one of the epitope-encoding nucleic acid sequences encodes an epitope that, when expressed and translated, is capable of being presented by MHC class I on a cell of a subject. In some aspects, at least one of the epitope-encoding nucleic acid sequences encodes an epitope that, when expressed and translated, is capable of being presented by MHC class II on a cell of a subject.

In some aspects, the at least one antigen-encoding nucleic acid sequence comprises two or more antigen-encoding nucleic acid sequences. In some aspects, each antigen-encoding nucleic acid sequence is linked directly to one another. In some aspects, each antigen-encoding nucleic acid sequence is linked to a distinct antigen-encoding nucleic acid sequence with a nucleic acid sequence encoding a linker. In some aspects, the linker links two MHC class I epitope-encoding nucleic acid sequences or an MHC class I epitope-encoding nucleic acid sequence to an MHC class II epitope-encoding nucleic acid sequence. In some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II epitope-encoding nucleic acid sequences or an MHC class II sequence to an MHC class I epitope-encoding nucleic acid sequence. In some aspects, the linker comprises the sequence GPGPG (SEQ ID NO: 56).

In some aspects, the antigen-encoding nucleic acid sequences is linked, operably or directly, to a separate or contiguous sequence that enhances the expression, stability, cell trafficking, processing and presentation, and/or immunogenicity of the antigen-encoding nucleic acid sequence. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, the epitope-encoding nucleic acid sequence comprises at least one alteration that makes the encoded epitope have increased binding affinity to its corresponding MHC allele relative to the translated, corresponding wild-type nucleic acid sequence. In some aspects, the epitope-encoding nucleic acid sequence comprises at least one alteration that makes the encoded epitope have increased binding stability to its corresponding MHC allele relative to the translated, corresponding wild-type nucleic acid sequence. In some aspects, the epitope-encoding nucleic acid sequence comprises at least one alteration that makes the encoded epitope have an increased likelihood of presentation on its corresponding MHC allele relative to the translated, corresponding wild-type nucleic acid sequence. In some aspects, the at least one alteration comprises a point mutation, a frameshift mutation, a non-frameshift mutation, a deletion mutation, an insertion mutation, a splice variant, a genomic rearrangement, or a proteasome-generated spliced antigen. In some aspects, the epitope-encoding nucleic acid sequence encodes an epitope known or suspected to be expressed in a subject known or suspected to have cancer. In some aspects, the cancer is a solid tumor. In some aspects, the cancer is selected from the group consisting of: MSS-CRC, NSCLC, and PDA. In some aspects, the cancer is selected from the group consisting of: lung cancer, melanoma, breast cancer, ovarian cancer, prostate cancer, kidney cancer, gastric cancer, colon cancer, testicular cancer, head and neck cancer, pancreatic cancer, bladder cancer, brain cancer, B-cell lymphoma, acute myelogenous leukemia, adult acute lymphoblastic leukemia, chronic myelogenous leukemia, chronic lymphocytic leukemia, T cell lymphocytic leukemia, non-small cell lung cancer, and small cell lung cancer.

In some aspects, the at least one antigen-encoding nucleic acid sequence comprises at least 2-10, 2, 3, 4, 5, 6, 7, 8, 9, or 10 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode epitope sequences or portions thereof that are presented by MHC class I on a cell surface. In some aspects, at least two of the MHC class I epitopes are presented by MHC class I on the tumor cell surface. In some aspects, the epitope-encoding nucleic acid sequences comprises at least one MHC class I epitope-encoding nucleic acid sequence, and wherein each antigen-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded epitope sequence distinct from the corresponding peptide sequence encoded by a wild-type nucleic acid sequence. In some aspects, the epitope-encoding nucleic acid sequence comprises an MHC class II epitope-encoding nucleic acid sequence and wherein each antigen-encoding nucleic acid sequence encodes a polypeptide sequence that is 12-20, 12, 13, 14, 15, 16, 17, 18, 19, 20, or 20-40 amino acids in length. In some aspects, the epitope-encoding nucleic acid sequences comprises an MHC class II epitope-encoding nucleic acid sequence, wherein the at least one MHC class II epitope-encoding nucleic acid sequence is present, and wherein the at least one MHC class II epitope-encoding nucleic acid sequence comprises at least one universal MHC class II epitope-encoding nucleic acid sequence, optionally wherein the at least one universal sequence comprises at least one of Tetanus toxoid and PADRE.

In some aspects, the at least one promoter nucleotide sequence is inducible. In some aspects, the at least one promoter nucleotide sequence is non-inducible.

In some aspects, the at least one poly(A) sequence comprises a Bovine Growth Hormone (BGH) SV40 polyA sequence. In some aspects, the at least one poly(A) sequence is at least 20, at least 30, at least 40, at least 50, at least 60, at least 70, at least 80, or at least 90 consecutive A nucleotides (SEQ ID NO: 29361). In some aspects, the at least one poly(A) sequence is at least 100 consecutive A nucleotides (SEQ ID NO: 29358).

In some aspects, the cassette further comprises at least one of: an intron sequence, a woodchuck hepatitis virus posttranscriptional regulatory element (WPRE) sequence, an internal ribosome entry sequence (IRES) sequence, a nucleotide sequence encoding a 2A self cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In some aspects, the cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

In some aspects, the epitope-encoding nucleic acid sequence comprises a MHC class I epitope-encoding nucleic acid sequence, and wherein the MHC class I epitope-encoding nucleic acid sequence is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the MHC class I epitope-encoding nucleic acid sequence. In some aspects, each of the MHC class I epitope-encoding nucleic acid sequences is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the at least 20 MHC class I epitope-encoding nucleic acid sequences. In some aspects, a number of the set of selected epitopes is 2-20. In some aspects, the presentation model represents dependence between: (a) presence of a pair of a particular one of the MHC alleles and a particular amino acid at a particular position of a peptide sequence; and

(b) likelihood of presentation on the tumor cell surface, by the particular one of the MHC alleles of the pair, of such a peptide sequence comprising the particular amino acid at the particular position. In some aspects, selecting the set of selected epitopes comprises selecting epitopes that have an increased likelihood of being presented on the tumor cell surface relative to unselected epitopes based on the presentation model. In some aspects, selecting the set of selected epitopes comprises selecting epitopes that have an increased likelihood of being capable of inducing a tumor-specific immune response in the subject relative to unselected epitopes based on the presentation model. In some aspects, selecting the set of selected epitopes comprises selecting epitopes that have an increased likelihood of being capable of being presented to naïve T cells by professional antigen presenting cells (APCs) relative to unselected epitopes based on the presentation model, optionally wherein the APC is a dendritic cell (DC). In some aspects, selecting the set of selected epitopes comprises selecting epitopes that have a decreased likelihood of being subject to inhibition via central or peripheral tolerance relative to unselected epitopes based on the presentation model. In some aspects, selecting the set of selected epitopes comprises selecting epitopes that have a decreased likelihood of being capable of inducing an autoimmune response to normal tissue in the subject relative to unselected epitopes based on the presentation model. In some aspects, exome or transcriptome nucleotide sequencing data is obtained by performing sequencing on the tumor tissue. In some aspects, the sequencing is next generation sequencing (NGS) or any massively parallel sequencing approach.

In some aspects, the cassette comprises junctional epitope sequences formed by adjacent sequences in the cassette. In some aspects, at least one or each junctional epitope sequence has an affinity of greater than 500 nM for MHC. In some aspects, each junctional epitope sequence is non-self. In some aspects, the cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the cassette. In some aspects, the prediction is based on presentation likelihoods generated by inputting sequences of the non-therapeutic epitopes into a presentation model. In some aspects, an order of the antigen-encoding nucleic acid sequences in the cassette is determined by a series of steps comprising: (a) generating a set of candidate cassette sequences corresponding to different orders of the antigen-encoding nucleic acid sequences; (b) determining, for each candidate cassette sequence, a presentation score based on presentation of non-therapeutic epitopes in the candidate cassette sequence; and (c) selecting a candidate cassette sequence associated with a presentation score below a predetermined threshold as the cassette sequence for a vaccine.

In some aspects, the composition for delivery of the ChAdV-based expression system is formulated in a pharmaceutical composition comprising a pharmaceutically acceptable carrier.

In some aspects, one or more of the epitope-encoding nucleic acid sequences are derived from a tumor of a subject. In some aspects, each of the epitope-encoding nucleic acid sequences are derived from a tumor of a subject. In some aspects, one or more of the epitope-encoding nucleic acid sequences are not derived from a tumor of a subject. In some aspects, each of the epitope-encoding nucleic acid sequences are not derived from a tumor of a subject. In some aspects, the epitope-encoding nucleic acid sequence comprises an epitope selected from the group consisting of SEQ ID NO: 57-29,357 and SEQ ID NO: 29,512-29,519. In some aspects, the at least one antigen-encoding nucleic acid sequence comprises at least 10, 11, 12, 13, 14, 15, 16, 17, 18, 19, or 20 tumor-specific MHC class I antigen-encoding nucleic acid sequences linearly linked to each other, comprising: (A) a KRAS_G12C MHC class I epitope encoding nucleic acid sequence, wherein the KRAS_G12C MHC class I epitope encoding nucleic acid sequence encodes a MHC class I epitope selected from the group consisting of SEQ ID NO: 14,954; 19,848; and 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; and 19,863, and (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, wherein each of the tumor-specific MHC class I antigen-encoding nucleic acid sequences comprises a class I epitope encoding nucleic acid sequence, optionally wherein each MHC I epitope encoding nucleic acid sequence encodes a MHC class I epitope selected from the group consisting of SEQ ID NO: 57-29,357 and SEQ ID NO: 29,512-29,519.

In some aspects, the at least one antigen-encoding nucleic acid sequence comprises at least 20 tumor-specific MHC class I antigen-encoding nucleic acid sequences linearly linked to each other, comprising: (A) a KRAS_G12A MHC class I epitope encoding nucleic acid sequence, (B) a KRAS_G12C MHC class I epitope encoding nucleic acid sequence, wherein the KRAS_G12C MHC class I epitope encoding nucleic acid sequence encodes a MHC class I epitope selected from the group consisting of SEQ ID NO: 14,954; 19,848; and 19,850, (C) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, and (D) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, (E) a KRAS_G13D MHC class I epitope encoding nucleic acid sequence, (F) a KRAS_Q61K MHC class I epitope encoding nucleic acid sequence, (G) a TP53_R249M MHC class I epitope encoding nucleic acid sequence, (H) a CTNNB1_S45P MHC class I epitope encoding nucleic acid sequence, (I) a CTNNB1_S45F MHC class I epitope encoding nucleic acid sequence, (J) a ERBB2_Y772_A775dup MHC class I epitope encoding nucleic acid sequence, (K) a KRAS_Q61R MHC class I epitope encoding nucleic acid sequence, (L) a CTNNB1_T41A MHC class I epitope encoding nucleic acid sequence, (M) a TP53_K132N MHC class I epitope encoding nucleic acid sequence, (N) a KRAS_Q61L MHC class I epitope encoding nucleic acid sequence, (O) a TP53_R213L MHC class I epitope encoding nucleic acid sequence, (P) a BRAF_G466V MHC class I epitope encoding nucleic acid sequence, (Q) a KRAS_Q61H MHC class I epitope encoding nucleic acid sequence, (R) a CTNNB1_S37F MHC class I epitope encoding nucleic acid sequence, (S) a TP53_S127Y MHC class I epitope encoding nucleic acid sequence, (T) a TP53_K132E MHC class I epitope encoding nucleic acid sequence, and (U) a KRAS_G12C MHC class I epitope encoding nucleic acid sequence.

In some aspects, the at least one antigen-encoding nucleic acid sequence comprises: (A) a KRAS_G12C MHC class I epitope encoding nucleic acid sequence, (B) a KRAS_G12D MHC class I epitope encoding nucleic acid sequence, (C) a KRAS_G12V MHC class I epitope encoding nucleic acid sequence, D) a KRAS Q61H MHC class I epitope encoding nucleic acid sequence, or combinations thereof. In some aspects, the at least one antigen-encoding nucleic acid sequence comprises at least each of: (A) a KRAS_G12C MHC class I epitope encoding nucleic acid sequence, (B) a KRAS_G12D MHC class I epitope encoding nucleic acid sequence, and (C) a KRAS_G12V MHC class I epitope encoding nucleic acid sequence. In some aspects, the at least one antigen-encoding nucleic acid sequence comprises at least each of: (A) a KRAS_G12C MHC class I epitope encoding nucleic acid sequence, (B) a KRAS_G12D MHC class I epitope encoding nucleic acid sequence, (C) a KRAS_G12V MHC class I epitope encoding nucleic acid sequence, and (D) a KRAS Q61H MHC class I epitope encoding nucleic acid sequence. In some aspects, the at least one antigen-encoding nucleic acid sequence comprises at least each of: (A) a KRAS_G12C MHC class I epitope encoding nucleic acid sequence, wherein the KRAS_G12C MHC class I epitope encoding nucleic acid sequence encodes a MHC class I epitope comprising the sequence of SEQ ID NO: 14,954; 19,848; 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, and (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974. In some aspects, the at least one antigen-encoding nucleic acid sequence comprises at least each of: (A) a KRAS_G12C MHC class I epitope encoding nucleic acid sequence, wherein the KRAS_G12C MHC class I epitope encoding nucleic acid sequence encodes a MHC class I epitope comprising the sequence of SEQ ID NO: 14,954; 19,848; 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, and (D) a KRAS Q61H MHC class I epitope encoding nucleic acid sequence, wherein the KRAS_Q61H MHC class I epitope encoding nucleic acid sequence encodes a MHC class I epitope selected from the group consisting of SEQ ID NO: 14,409.

In some aspects, the at least one antigen-encoding nucleic acid sequence comprises: (A) a TP53_R213L MHC class I epitope encoding nucleic acid sequence, (B) a TP53_S127Y MHC class I epitope encoding nucleic acid sequence, (C) a TP53_R249M MHC class I epitope encoding nucleic acid sequence, or combinations thereof. In some aspects, the at least one antigen-encoding nucleic acid sequence comprises at least each of: (A) a TP53_R213L MHC class I epitope encoding nucleic acid sequence, (B) a TP53_S127Y MHC class I epitope encoding nucleic acid sequence, and (C) a TP53_R249M MHC class I epitope encoding nucleic acid sequence. In some aspects, the TP53_R213L MHC class I epitope encoding nucleic acid sequence encodes a MHC class I epitope selected from the group consisting of SEQ ID NO: 29,519.

Also provided for herein is a kit comprising any of the compositions for delivery of the ChAdV-based expression system described herein, and instructions for use.

Also disclosed herein is a method for stimulating an immune response in a subject, the method comprising administering to the subject a composition for delivery of a self-amplifying alphavirus-based expression system and administering to the subject a composition for delivery of a chimpanzee adenovirus (ChAdV)-based expression system, and wherein either: a. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles, b. wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors, or c. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles and wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors.

In some aspects, the composition for delivery of the ChAdV-based expression system is administered as a priming dose and the composition for delivery of the self-amplifying alphavirus-based expression system is administered as one or more boosting doses. In some aspects, the priming dose is administered on day 1 and the one or more boosting doses are administered every 4 weeks (Q4W) following the priming dose. In some aspects, the one or more boosting doses are administered every 4 weeks for a time period. In some aspects, the time period is the first 6 months following the priming dose. In some aspects, one or more additional boosting doses are administered at a second interval following the time period. In some aspects, the second interval is every 3 months. In some aspects, two or more boosting doses are administered. In some aspects, 1, 2, 3, 4, 5, 6, 7, or 8 boosting doses are administered.

In some aspects, the composition for delivery of the ChAdV-based expression system is administered intramuscularly (IM), intradermally (ID), subcutaneously (SC), or intravenously (IV). In some aspects, the composition for delivery of the ChAdV-based expression system is administered (IM). In some aspects, the IM administration is administered at separate injection sites. In some aspects, the separate injection sites are in opposing deltoid muscles. In some aspects, the separate injection sites are in gluteus or rectus femoris sites on each side.

In some aspects, the composition for delivery of the self-amplifying alphavirus-based expression system is administered intramuscularly (IM), intradermally (ID), subcutaneously (SC), or intravenously (IV). In some aspects, the composition for delivery of the self-amplifying alphavirus-based expression system is administered (IM). In some aspects, the IM administration is administered at separate injection sites. In some aspects, the separate injection sites are in opposing deltoid muscles. In some aspects, the separate injection sites are in gluteus or rectus femoris sites on each side. In some aspects, the injection site of the one or more boosting doses is as close as possible to the injection site of the priming dose.

In some aspects, the method further comprises determining or having determined the HLA-haplotype of the subject.

In some aspects, the method further comprises administering nivolumab. In some aspects, nivolumab is administered as an intravenous (IV) infusion. In some aspects, nivolumab is administered at a dose of 480 mg. In some aspects, nivolumab is administered on day 1. In some aspects, nivolumab is on administered day 1 and administered every 4 weeks (Q4W) following the priming dose. In some aspects, nivolumab is on administered on the same day as the priming dose or on the same day as the one or more boosting doses. In some aspects, nivolumab is formulated in solution at 10 mg/mL.

In some aspects, the method further comprises administering ipilimumab. In some aspects, ipilimumab is administered an intravenous (IV) infusion. In some aspects, ipilimumab is administered subcutaneously (SC). In some aspects, the SC administration is injected proximally (within ˜2 cm) to one or more of the priming dose injection site or the one or more boosting dose injection sites. In some aspects, the SC administration is administered as 4 separate injections or administered as 6 separate injections. In some aspects, ipilimumab is administered at a dose of 30 mg. In some aspects, ipilimumab is administered on day 1. In some aspects, ipilimumab is on administered day 1 and administered every 4 weeks (Q4W) following the priming dose. In some aspects, ipilimumab is on administered on the same day as the priming dose or on the same day as the one or more boosting doses. In some aspects, ipilimumab is formulated in solution at 5 mg/mL.

In some aspects, the composition for delivery of the self-amplifying alphavirus-based expression system comprises: (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; (ii) optionally, a second promoter nucleotide sequence operably linked to the at least one antigen-encoding nucleic acid sequence; and (iii) optionally, at least one second poly(A) sequence, wherein the second poly(A) sequence is a native poly(A) sequence or an exogenous poly(A) sequence to the alphavirus, and (B) a lipid-nanoparticle (LNP), wherein the LNP encapsulates the self-amplifying alphavirus-based expression system.

In some aspects, the composition for delivery of the self-amplifying alphavirus-based expression system comprises, (A) the self-amplifying alphavirus-based expression system, wherein the self-amplifying alphavirus-based expression system comprises one or more vectors, wherein the one or more vectors comprises: (a) an RNA alphavirus backbone, wherein the RNA alphavirus backbone comprises the nucleic acid sequence set forth in SEQ ID NO:6, wherein the RNA alphavirus backbone sequence comprises a 26S promoter nucleotide sequence and a poly(A) sequence, wherein the 26S promoter sequence is endogenous to the RNA alphavirus backbone, and wherein the poly(A) sequence is endogenous to the RNA alphavirus backbone; and (b) a cassette integrated between the 26S promoter nucleotide sequence and the poly(A) sequence, wherein the cassette is operably linked to the 26S promoter nucleotide sequence, and wherein the cassette comprises at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and (B) a lipid-nanoparticle (LNP), wherein the LNP encapsulates the self-amplifying alphavirus-based expression system.

In some aspects, the composition for delivery of the self-amplifying alphavirus-based expression system comprises at least 30 g of each of the one or more vectors. In some aspects, the composition for delivery of the self-amplifying alphavirus-based expression system comprises at least 100 μg of each of the one or more vectors. In some aspects, the composition for delivery of the self-amplifying alphavirus-based expression system comprises at least 300 μg of each of the one or more vectors. In some aspects, the composition for delivery of the self-amplifying alphavirus-based expression system comprises at least 400 μg, at least 500 μg, at least 600 μg, at least 700 μg, at least 800 μg, at least 900 μg, at least 1000 μg of each of the one or more vectors. In some aspects, the composition for delivery of the self-amplifying alphavirus-based expression system comprises between 10-30 g, 10-100 g, 10-300 g, 30-100 g, 30-300 μg, or 100-300 μg of each of the one or more vectors. In some aspects, the composition for delivery of the self-amplifying alphavirus-based expression system comprises between 10-500 μg, 10-1000 μg, 30-500 μg, 30-1000 μg, or 500-1000 μg of each of the one or more vectors. In some aspects, the composition for delivery of the self-amplifying alphavirus-based expression system comprises 400 μg, 500 μg, 600 μg, 700 μg, 800 μg, 900 μg, or 1000 μg of each of the one or more vectors In some aspects, the composition for delivery of the self-amplifying alphavirus-based expression system comprises 10 μg, 30 μg, 100 μg, or 300 μg of each of the one or more vectors. In some aspects, the composition for delivery of the self-amplifying alphavirus-based expression system comprises less than or equal to 300 μg of each of the one or more vectors.

In some aspects, the weight to weight ratio of the LNP to total weight of the one or more vectors is between 10-40 to 1. In some aspects, the weight to weight ratio of the LNP to total weight of the one or more vectors is between 16-32 to 1. In some aspects, the weight to weight ratio of the LNP to total weight of the one or more vectors is about 24 to 1. In some aspects, the weight to weight ratio of the LNP to total weight of the one or more vectors is 24 to 1.

In some aspects, the one or more vectors is at a concentration of 1 mg/mL.

In some aspects, an ordered sequence of each element of the cassette in the composition for delivery of the self-amplifying alphavirus-based expression system is described in the formula, from 5′ to 3′, comprising Pₐ-(L5_{b}-N_{c}-L3_{d})_{X}-(G5ₑ-U_{f})_{Y}-G3_{g} wherein P comprises the second promoter nucleotide sequence, where a=0 or 1, N comprises one of the epitope-encoding nucleic acid sequences, wherein the epitope-encoding nucleic acid sequence comprises an MHC class I epitope-encoding nucleic acid sequence, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding N_{c} is an MHC class I epitope-encoding nucleic acid sequence, and Y=0, 1, or 2, where for each Y the corresponding U_{f} is an MHC class II epitope-encoding nucleic acid sequence. In some aspects, for each X the corresponding N_{c} is a distinct MHC class I epitope-encoding nucleic acid sequence. In some aspects, for each Y the corresponding U_{f} is a distinct MHC class II epitope-encoding nucleic acid sequence. In some aspects, a=0, b=1, d=1, e=1, g=1, h=1, X=20, Y=2, the at least one promoter nucleotide sequence is a single 26S promoter nucleotide sequence provided by the RNA alphavirus backbone, the at least one polyadenylation poly(A) sequence is a poly(A) sequence of at least 100 consecutive A nucleotides (SEQ ID NO: 29358) provided by the RNA alphavirus backbone, the cassette is integrated between the 26S promoter nucleotide sequence and the poly(A) sequence, wherein the cassette is operably linked to the 26S promoter nucleotide sequence and the poly(A) sequence, each N encodes a MHC class I epitope 7-15 amino acids in length, L5 is a native 5′ linker sequence that encodes a native N-terminal amino acid sequence of the MHC I epitope, and wherein the 5′ linker sequence encodes a peptide that is at least 3 amino acids in length, L3 is a native 3′ linker sequence that encodes a native C-terminal amino acid sequence of the MHC I epitope, and wherein the 3′ linker sequence encodes a peptide that is at least 3 amino acids in length, U is each of a PADRE class II sequence and a Tetanus toxoid MHC class II sequence, the RNA alphavirus backbone is the sequence set forth in SEQ ID NO:6, and each of the MHC class I epitope-encoding nucleic acid sequences encodes a polypeptide that is between 13 and 25 amino acids in length.

In some aspects, the LNP comprises a lipid selected from the group consisting of: an ionizable amino lipid, a phosphatidylcholine, cholesterol, a PEG-based coat lipid, or a combination thereof. In some aspects, the LNP comprises an ionizable amino lipid, a phosphatidylcholine, cholesterol, and a PEG-based coat lipid. In some aspects, the ionizable amino lipids comprise MC3-like (dilinoleylmethyl-4-dimethylaminobutyrate) molecules. In some aspects, the LNP-encapsulated expression system has a diameter of about 100 nm.

In some aspects, the cassette is integrated between the at least one promoter nucleotide sequence and the at least one poly(A) sequence.

In some aspects, the at least one promoter nucleotide sequence is operably linked to the cassette.

In some aspects, the one or more vectors comprise one or more +-stranded RNA vectors. In some aspects, the one or more +-stranded RNA vectors comprise a 5′ 7-methylguanosine (m7g) cap. In some aspects, the one or more +-stranded RNA vectors are produced by in vitro transcription. In some aspects, the one or more vectors are self-amplifying within a mammalian cell. In some aspects, the RNA alphavirus backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the RNA alphavirus backbone comprises at least one nucleotide sequence of a Venezuelan equine encephalitis virus. In some aspects, the RNA alphavirus backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the RNA alphavirus backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the RNA alphavirus backbone does not encode structural virion proteins capsid, E2 and E1. In some aspects, the cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11175. In some aspects, the RNA alphavirus backbone comprises the sequence set forth in SEQ ID NO:6 or SEQ ID NO:7. In some aspects, the cassette is inserted at position 7544 to replace the deletion between base pairs 7544 and 11175 as set forth in the sequence of SEQ ID NO:3 or SEQ ID NO:5. In some aspects, the insertion of the cassette provides for transcription of a polycistronic RNA comprising the nsP1-4 genes and the at least one nucleic acid sequence, wherein the nsP1-4 genes and the at least one nucleic acid sequence are in separate open reading frames.

In some aspects, the at least one promoter nucleotide sequence is the native 26S promoter nucleotide sequence encoded by the RNA alphavirus backbone. In some aspects, the at least one promoter nucleotide sequence is an exogenous RNA promoter. In some aspects, the second promoter nucleotide sequence is a 26S promoter nucleotide sequence. In some aspects, the second promoter nucleotide sequence comprises multiple 26S promoter nucleotide sequences, wherein each 26S promoter nucleotide sequence provides for transcription of one or more of the separate open reading frames.

In some aspects, the one or more vectors are each at least 300 nt in size. In some aspects, the one or more vectors are each at least 1 kb in size. In some aspects, the one or more vectors are each 2 kb in size. In some aspects, the one or more vectors are each less than 5 kb in size.

In some aspects, the at least one antigen-encoding nucleic acid sequence comprises two or more antigen-encoding nucleic acid sequences. In some aspects, each antigen-encoding nucleic acid sequence is linked directly to one another. In some aspects, each antigen-encoding nucleic acid sequence is linked to a distinct antigen-encoding nucleic acid sequence with a nucleic acid sequence encoding a linker. In some aspects, the linker links two MHC class I epitope-encoding nucleic acid sequences or an MHC class I epitope-encoding nucleic acid sequence to an MHC class II epitope-encoding nucleic acid sequence. In some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II epitope-encoding nucleic acid sequences or an MHC class II sequence to an MHC class I epitope-encoding nucleic acid sequence. In some aspects, the linker comprises the sequence GPGPG (SEQ ID NO: 56).

In some aspects, the antigen-encoding nucleic acid sequences is linked, operably or directly, to a separate or contiguous sequence that enhances the expression, stability, cell trafficking, processing and presentation, and/or immunogenicity of the antigen-encoding nucleic acid sequence. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, the at least one antigen-encoding nucleic acid sequence comprises at least 2-10, 2, 3, 4, 5, 6, 7, 8, 9, or 10 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 orup to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode epitope sequences or portions thereof that are presented by MHC class I on a cell surface. In some aspects, at least two of the MHC class I epitopes are presented by MHC class I on the tumor cell surface.

In some aspects, the epitope-encoding nucleic acid sequences comprises at least one MHC class I epitope-encoding nucleic acid sequence, and wherein each antigen-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded epitope sequence distinct from the corresponding peptide sequence encoded by a wild-type nucleic acid sequence. In some aspects, the epitope-encoding nucleic acid sequence comprises an MHC class II epitope-encoding nucleic acid sequence and wherein each antigen-encoding nucleic acid sequence encodes a polypeptide sequence that is 12-20, 12, 13, 14, 15, 16, 17, 18, 19, 20, or 20-40 amino acids in length. In some aspects, the epitope-encoding nucleic acid sequences comprises an MHC class II epitope-encoding nucleic acid sequence, wherein the at least one MHC class II epitope-encoding nucleic acid sequence is present, and wherein the at least one MHC class II epitope-encoding nucleic acid sequence comprises at least one universal MHC class II epitope-encoding nucleic acid sequence, optionally wherein the at least one universal sequence comprises at least one of Tetanus toxoid and PADRE.

In some aspects, the at least one promoter nucleotide sequence or the second promoter nucleotide sequence is inducible. In some aspects, the at least one promoter nucleotide sequence or the second promoter nucleotide sequence is non-inducible. In some aspects, the at least one poly(A) sequence comprises a poly(A) sequence native to the alphavirus. In some aspects, the at least In some aspects, the at least one poly(A) sequence is operably linked to at least one of the at least one nucleic acid sequences. In some aspects, the at least one poly(A) sequence is at least 20, at least 30, at least 40, at least 50, at least 60, at least 70, at least 80, or at least 90 consecutive A nucleotides (SEQ ID NO: 29361). In some aspects, the at least one poly(A) sequence is at least 100 consecutive A nucleotides (SEQ ID NO: 29358).

In some aspects, the epitope-encoding nucleic acid sequence comprises a MHC class I epitope-encoding nucleic acid sequence, and wherein the MHC class I epitope-encoding nucleic acid sequence is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the MHC class I epitope-encoding nucleic acid sequence. In some aspects, each of the MHC class I epitope-encoding nucleic acid sequences is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the at least 20 MHC class I epitope-encoding nucleic acid sequences. In some aspects, a number of the set of selected epitopes is 2-20. In some aspects, the presentation model represents dependence between: (a) presence of a pair of a particular one of the MHC alleles and a particular amino acid at a particular position of a peptide sequence; and (b) likelihood of presentation on the tumor cell surface, by the particular one of the MHC alleles of the pair, of such a peptide sequence comprising the particular amino acid at the particular position. In some aspects, selecting the set of selected epitopes comprises selecting epitopes that have an increased likelihood of being presented on the tumor cell surface relative to unselected epitopes based on the presentation model. In some aspects, selecting the set of selected epitopes comprises selecting epitopes that have an increased likelihood of being capable of inducing a tumor-specific immune response in the subject relative to unselected epitopes based on the presentation model. In some aspects, selecting the set of selected epitopes comprises selecting epitopes that have an increased likelihood of being capable of being presented to naïve T cells by professional antigen presenting cells (APCs) relative to unselected epitopes based on the presentation model, optionally wherein the APC is a dendritic cell (DC). In some aspects, selecting the set of selected epitopes comprises selecting epitopes that have a decreased likelihood of being subject to inhibition via central or peripheral tolerance relative to unselected epitopes based on the presentation model. In some aspects, selecting the set of selected epitopes comprises selecting epitopes that have a decreased likelihood of being capable of inducing an autoimmune response to normal tissue in the subject relative to unselected epitopes based on the presentation model. In some aspects, exome or transcriptome nucleotide sequencing data is obtained by performing sequencing on the tumor tissue. In some aspects, the sequencing is next generation sequencing (NGS) or any massively parallel sequencing approach.

In some aspects, the ChAdV vector comprises: (a) a ChAdV backbone, wherein the ChAdV backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and wherein the cassette is operably linked to the at least one promoter nucleotide sequence and the at least one poly(A) sequence.

In some aspects, the ChAdV vector comprises: (a) a ChAdV backbone, wherein the ChAdV backbone comprises: (i) a modified ChAdV68 sequence comprising at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion and (2) nucleotides 27,125 to 31,825 of the sequence shown in SEQ ID NO:1 corresponding to an E3 deletion; and optionally lacks (3) nucleotides 34,916 to 35,642 of the sequence shown in SEQ ID NO:1 corresponding to a partial E4 deletion; (ii) a CMV promoter nucleotide sequence; and (iii) an SV40 polyadenylation (poly(A)) sequence; and (b) a cassette, wherein the cassette comprises: (i) at least one antigen-encoding nucleic acid sequence comprising: a. an epitope-encoding nucleic acid sequence, optionally comprising at least one alteration that makes the encoded epitope sequence distinct from the corresponding peptide sequence encoded by a wild-type nucleic acid sequence, b. optionally a 5′ linker sequence, and c. optionally a 3′ linker sequence; and wherein the cassette is inserted within the E1 deletion and the cassette is operably linked to the CMV promoter nucleotide sequence and the SV40 poly(A) sequence.

In some aspects, the composition for delivery of the ChAdV-based expression system comprises 3×10¹¹ or less of the viral particles. In some aspects, the composition for delivery of the ChAdV-based expression system comprises at least 1×10¹¹ of the viral particles. In some aspects, the composition for delivery of the ChAdV-based expression system comprises between 1×10¹¹ and 1×10¹², between 3×10¹¹ and 1×10¹², or between 1×10¹¹ and 3×10¹¹ of the viral particles. In some aspects, the composition for delivery of the ChAdV-based expression system comprises 1×10¹¹, 3×10¹¹, or 1×10¹² of the viral particles. In some aspects, the viral particles are at a concentration of at 5×10¹¹ vp/mL.

In some aspects, the epitope-encoding nucleic acid sequence encodes an epitope known or suspected to be presented by MHC class I on a surface of a cell, optionally wherein the surface of the cell is a tumor cell surface or an infected cell surface, and optionally wherein the cell is the subject's cell. In some aspects, the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer, or wherein the cell is an infected cell selected from the group consisting of: a pathogen infected cell, a virally infected cell, a bacterially infected cell, an fungally infected cell, and a parasitically infected cell. In some aspects, the virally infected cell is an HIV infected cell.

In some aspects, an ordered sequence of each element of the cassette in the composition for delivery of the ChAdV-based expression system is described in the formula, from 5′ to 3′, comprising Pₐ-(L5_{b}-N_{c}-L3_{d})_{X}-(G5ₑ-U_{f})_{Y}-G3_{g} wherein P comprises the at least one promoter sequence operably linked to at least one of the at least one antigen-encoding nucleic acid sequences, where a=1, N comprises one of the epitope-encoding nucleic acid sequences, wherein the epitope-encoding nucleic acid sequence comprises an MHC class I epitope-encoding nucleic acid sequence, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding No is an MHC class I epitope-encoding nucleic acid sequence, and Y=0, 1, or 2, where for each Y the corresponding U_{f} is an MHC class II epitope-encoding nucleic acid sequence. In some aspects, for each X the corresponding No is a distinct MHC class I epitope-encoding nucleic acid sequence. In some aspects, for each Y the corresponding U_{f} is a distinct MHC class II epitope-encoding nucleic acid sequence. In some aspects, b=1, d=1, e=1, g=1, h=1, X=10, Y=2, P is a CMV promoter sequence, each N encodes a MHC class I epitope 7-15 amino acids in length, L5 is a native 5′ linker sequence that encodes a native N-terminal amino acid sequence of the MHC I epitope, and wherein the 5′ linker sequence encodes a peptide that is at least 3 amino acids in length, L3 is a native 3′ linker sequence that encodes a native C-terminal amino acid sequence of the MHC I epitope, and wherein the 3′ linker sequence encodes a peptide that is at least 3 amino acids in length, U is each of a PADRE class II sequence and a Tetanus toxoid MHC class II sequence, the ChAdV vector comprises a modified ChAdV68 sequence comprising the sequence of SEQ ID NO:1 with an E1 (nt 577 to 3403) deletion and an E3 (nt 27,125-31,825) deletion and the neoantigen cassette is inserted within the E1 deletion, and each of the MHC class I antigen-encoding nucleic acid sequences encodes a polypeptide that is 25 amino acids in length.

In some aspects, the cassette is integrated between the at least one promoter nucleotide sequence and the at least one poly(A) sequence. In some aspects, the at least one promoter nucleotide sequence is operably linked to the cassette.

In some aspects, the ChAdV backbone comprises a ChAdV68 vector backbone. In some aspects, the ChAdV68 vector backbone comprises the sequence set forth in SEQ ID NO: 1. In some aspects, the ChAdV68 vector backbone comprises a functional deletion in at least one gene selected from the group consisting of an adenovirus E1A, E1B, E2A, E2B, E3, L1, L2, L3, L4, and L5 gene with reference to a ChAdV68 genome or with reference to the sequence shown in SEQ ID NO:1, optionally wherein the adenoviral backbone or modified ChAdV68 sequence is fully deleted or functionally deleted in: (1) E1A and E1B; or (2) E1A, E1B, and E3 with reference to the adenovirus genome or with reference to the sequence shown in SEQ ID NO:1, optionally wherein the E1 gene is functionally deleted through an E1 deletion of at least nucleotides 577 to 3403 with reference to the sequence shown in SEQ ID NO:1 and optionally wherein the E3 gene is functionally deleted through an E3 deletion of at least nucleotides 27,125 to 31,825 with reference to the sequence shown in SEQ ID NO:1. In some aspects, the ChAdV68 vector backbone comprises one or more genes or regulatory sequences with reference to a ChAdV68 genome or with reference to the sequence shown in SEQ ID NO: 1, optionally wherein the one or more genes or regulatory sequences are selected from the group consisting of the chimpanzee adenovirus inverted terminal repeat (ITR), E1A, E1B, E2A, E2B, E3, E4, L1, L2, L3, L4, and L5 genes. In some aspects, the ChAdV68 vector backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion and (2) nucleotides 27,125 to 31,825 of the sequence shown in SEQ ID NO:1 corresponding to an E3 deletion. In some aspects, the ChAdV68 vector backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) nucleotides 34,916 to 35,642 of the sequence shown in SEQ ID NO:1 corresponding to a partial E4 deletion; optionally wherein the antigen cassette is inserted within the E1 deletion. In some aspects, the cassette is inserted in the ChAdV backbone at the E1 region, E3 region, and/or any deleted AdV region that allows incorporation of the cassette. In some aspects, the ChAdV backbone is generated from one of a first generation, a second generation, or a helper-dependent adenoviral vector.

In some aspects, the at least one promoter nucleotide sequence is selected from the group consisting of: a CMV, a SV40, an EF-1, a RSV, a PGK, a HSA, a MCK, and a EBV promoter sequence. In some aspects, the at least one promoter nucleotide sequence is a CMV promoter sequence.

In some aspects, at least one of the epitope-encoding nucleic acid sequences encodes an epitope that, when expressed and translated, is capable of being presented by MHC class I on a cell of the subject. In some aspects, at least one of the epitope-encoding nucleic acid sequences encodes an epitope that, when expressed and translated, is capable of being presented by MHC class II on a cell of the subject.

In some aspects, the at least one antigen-encoding nucleic acid sequence comprises two or more antigen-encoding nucleic acid sequences. In some aspects, each antigen-encoding nucleic acid sequence is linked directly to one another.

In some aspects, each antigen-encoding nucleic acid sequence is linked to a distinct antigen-encoding nucleic acid sequence with a nucleic acid sequence encoding a linker. In some aspects, the linker links two MHC class I epitope-encoding nucleic acid sequences or an MHC class I epitope-encoding nucleic acid sequence to an MHC class II epitope-encoding nucleic acid sequence. In some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II epitope-encoding nucleic acid sequences or an MHC class II sequence to an MHC class I epitope-encoding nucleic acid sequence. In some aspects, the linker comprises the sequence GPGPG (SEQ ID NO: 56).

In some aspects, the antigen-encoding nucleic acid sequences is linked, operably or directly, to a separate or contiguous sequence that enhances the expression, stability, cell trafficking, processing and presentation, and/or immunogenicity of the antigen-encoding nucleic acid sequence. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, the epitope-encoding nucleic acid sequence comprises at least one alteration that makes the encoded epitope have increased binding affinity to its corresponding MHC allele relative to the translated, corresponding wild-type nucleic acid sequence. In some aspects, the epitope-encoding nucleic acid sequence comprises at least one alteration that makes the encoded epitope have increased binding stability to its corresponding MHC allele relative to the translated, corresponding wild-type nucleic acid sequence. In some aspects, the epitope-encoding nucleic acid sequence comprises at least one alteration that makes the encoded epitope have an increased likelihood of presentation on its corresponding MHC allele relative to the translated, corresponding wild-type nucleic acid sequence. In some aspects, the at least one alteration comprises a point mutation, a frameshift mutation, a non-frameshift mutation, a deletion mutation, an insertion mutation, a splice variant, a genomic rearrangement, or a proteasome-generated spliced antigen.

In some aspects, the epitope-encoding nucleic acid sequence encodes an epitope known or suspected to be expressed in the subject known or suspected to have cancer. In some aspects, the cancer comprises a solid tumor. In some aspects, the cancer is selected from the group consisting of: microsatellite stable-colorectal cancer (MSS-CRC), non-small cell lung cancer (NSCLC), pancreatic ductal adenocarcinoma (PDA), and gastroesophageal adenocarcinoma (GEA). In some aspects, the cancer is selected from the group consisting of: lung cancer, melanoma, breast cancer, ovarian cancer, prostate cancer, kidney cancer, gastric cancer, colon cancer, testicular cancer, head and neck cancer, pancreatic cancer, bladder cancer, brain cancer, B-cell lymphoma, acute myelogenous leukemia, adult acute lymphoblastic leukemia, chronic myelogenous leukemia, chronic lymphocytic leukemia, T cell lymphocytic leukemia, non-small cell lung cancer, and small cell lung cancer.

In some aspects, the at least one antigen-encoding nucleic acid sequence comprises at least 2-10, 2, 3, 4, 5, 6, 7, 8, 9, or 10 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences, optionally wherein each antigen-encoding nucleic acid sequence encodes a distinct antigen-encoding nucleic acid sequence. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode epitope sequences or portions thereof that are presented by MHC class I on a cell surface. In some aspects, at least two of the MHC class I epitopes are presented by MHC class I on the tumor cell surface.

In some aspects, the epitope-encoding nucleic acid sequences comprises at least one MHC class I epitope-encoding nucleic acid sequence, and wherein each antigen-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded epitope sequence distinct from the corresponding peptide sequence encoded by a wild-type nucleic acid sequence. In some aspects, the epitope-encoding nucleic acid sequence comprises an MHC class II epitope-encoding nucleic acid sequence and wherein each antigen-encoding nucleic acid sequence encodes a polypeptide sequence that is 12-20, 12, 13, 14, 15, 16, 17, 18, 19, 20, or 20-40 amino acids in length. In some aspects, the epitope-encoding nucleic acid sequences comprises an MHC class II epitope-encoding nucleic acid sequence, wherein the at least one MHC class II epitope-encoding nucleic acid sequence is present, and wherein the at least one MHC class II epitope-encoding nucleic acid sequence comprises at least one universal MHC class II epitope-encoding nucleic acid sequence, optionally wherein the at least one universal sequence comprises at least one of Tetanus toxoid and PADRE.

In some aspects, the at least one promoter nucleotide sequence is inducible. In some aspects, wherein the at least one promoter nucleotide sequence is non-inducible. In some aspects, the at least one poly(A) sequence comprises a Bovine Growth Hormone (BGH) SV40 polyA sequence. In some aspects, the at least one poly(A) sequence is at least 20, at least 30, at least 40, at least 50, at least 60, at least 70, at least 80, or at least 90 consecutive A nucleotides (SEQ ID NO: 29361). In some aspects, the at least one poly(A) sequence is at least 100 consecutive A nucleotides (SEQ ID NO: 29358).

In some aspects, the cassette further comprises at least one of: an intron sequence, a woodchuck hepatitis virus posttranscriptional regulatory element (WPRE) sequence, an internal ribosome entry sequence (IRES) sequence, a nucleotide sequence encoding a 2A self cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In some aspects, the cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

In some aspects, the epitope-encoding nucleic acid sequence comprises a MHC class I epitope-encoding nucleic acid sequence, and wherein the MHC class I epitope-encoding nucleic acid sequence is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the MHC class I epitope-encoding nucleic acid sequence. In some aspects, each of the MHC class I epitope-encoding nucleic acid sequences is selected by performing the steps of: (a) obtaining at least one of exome, transcriptome, or whole genome tumor nucleotide sequencing data from the tumor, wherein the tumor nucleotide sequencing data is used to obtain data representing peptide sequences of each of a set of epitopes; (b) inputting the peptide sequence of each epitope into a presentation model to generate a set of numerical likelihoods that each of the epitopes is presented by one or more of the MHC alleles on the tumor cell surface of the tumor, the set of numerical likelihoods having been identified at least based on received mass spectrometry data; and (c) selecting a subset of the set of epitopes based on the set of numerical likelihoods to generate a set of selected epitopes which are used to generate the at least 20 MHC class I epitope-encoding nucleic acid sequences. In some aspects, a number of the set of selected epitopes is 2-20. In some aspects, the presentation model represents dependence between: (a) presence of a pair of a particular one of the MHC alleles and a particular amino acid at a particular position of a peptide sequence; and (b) likelihood of presentation on the tumor cell surface, by the particular one of the MHC alleles of the pair, of such a peptide sequence comprising the particular amino acid at the particular position. In some aspects, selecting the set of selected epitopes comprises selecting epitopes that have an increased likelihood of being presented on the tumor cell surface relative to unselected epitopes based on the presentation model. In some aspects, selecting the set of selected epitopes comprises selecting epitopes that have an increased likelihood of being capable of inducing a tumor-specific immune response in the subject relative to unselected epitopes based on the presentation model. In some aspects, selecting the set of selected epitopes comprises selecting epitopes that have an increased likelihood of being capable of being presented to naïve T cells by professional antigen presenting cells (APCs) relative to unselected epitopes based on the presentation model, optionally wherein the APC is a dendritic cell (DC). In some aspects, selecting the set of selected epitopes comprises selecting epitopes that have a decreased likelihood of being subject to inhibition via central or peripheral tolerance relative to unselected epitopes based on the presentation model. In some aspects, selecting the set of selected epitopes comprises selecting epitopes that have a decreased likelihood of being capable of inducing an autoimmune response to normal tissue in the subject relative to unselected epitopes based on the presentation model. In some aspects, exome or transcriptome nucleotide sequencing data is obtained by performing sequencing on the tumor tissue. In some aspects, the sequencing is next generation sequencing (NGS) or any massively parallel sequencing approach.

In some aspects, the cassette comprises junctional epitope sequences formed by adjacent sequences in the cassette. In some aspects, at least one or each junctional epitope sequence has an affinity of greater than 500 nM for MHC. In some aspects, each junctional epitope sequence is non-self.

In some aspects, the cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the cassette. In some aspects, the prediction is based on presentation likelihoods generated by inputting sequences of the non-therapeutic epitopes into a presentation model. In some aspects, an order of the antigen-encoding nucleic acid sequences in the cassette is determined by a series of steps comprising: (a) generating a set of candidate cassette sequences corresponding to different orders of the antigen-encoding nucleic acid sequences; (b) determining, for each candidate cassette sequence, a presentation score based on presentation of non-therapeutic epitopes in the candidate cassette sequence; and (c) selecting a candidate cassette sequence associated with a presentation score below a predetermined threshold as the cassette sequence for a vaccine.

In some aspects, the composition for delivery of the ChAdV-based expression system is formulated in a pharmaceutical composition comprising a pharmaceutically acceptable carrier.

In some aspects, one or more of the epitope-encoding nucleic acid sequences are derived from a tumor of the subject. In some aspects, each of the epitope-encoding nucleic acid sequences are derived from a tumor of the subject. In some aspects, one or more of the epitope-encoding nucleic acid sequences are not derived from a tumor of the subject. In some aspects, each of the epitope-encoding nucleic acid sequences are not derived from a tumor of the subject. In some aspects, the epitope-encoding nucleic acid sequence comprises an epitope selected from the group consisting of SEQ ID NO: 57-29,357 and SEQ ID NO: 29,512-29,519. In some aspects, the at least one antigen-encoding nucleic acid sequence comprises at least 10, 11, 12, 13, 14, 15, 16, 17, 18, 19, or 20 tumor-specific MHC class I antigen-encoding nucleic acid sequences linearly linked to each other, comprising: (A) a KRAS_G12C MHC class I epitope encoding nucleic acid sequence, wherein the KRAS_G12C MHC class I epitope encoding nucleic acid sequence encodes a MHC class I epitope selected from the group consisting of SEQ ID NO: 14,954; 19,848; and 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, and (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, wherein each of the tumor-specific MHC class I antigen-encoding nucleic acid sequences comprises a class I epitope encoding nucleic acid sequence, optionally wherein each MHC I epitope encoding nucleic acid sequence encodes a MHC class I epitope selected from the group consisting of SEQ ID NO: 57-29,357 and SEQ ID NO: 29,512-29,519. In some aspects, the at least one antigen-encoding nucleic acid sequence comprises at least at least 20 tumor-specific MHC class I antigen-encoding nucleic acid sequences linearly linked to each other, comprising: (A) a KRAS_G12A MHC class I epitope encoding nucleic acid sequence, (B) a KRAS_G12C MHC class I epitope encoding nucleic acid sequence, wherein the KRAS_G12C MHC class I epitope encoding nucleic acid sequence encodes a MHC class I epitope selected from the group consisting of SEQ ID NO: 14,954; 19,848; and 19,850, (C) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, and (D) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, (E) a KRAS_G13D MHC class I epitope encoding nucleic acid sequence, (F) a KRAS_Q61K MHC class I epitope encoding nucleic acid sequence, (G) a TP53_R249M MHC class I epitope encoding nucleic acid sequence, (H) a CTNNB1_S45P MHC class I epitope encoding nucleic acid sequence, (I) a CTNNB1_S45F MHC class I epitope encoding nucleic acid sequence, (J) a ERBB2_Y772_A775dup MHC class I epitope encoding nucleic acid sequence, (K) a KRAS_Q61R MHC class I epitope encoding nucleic acid sequence, (L) a CTNNB1_T41A MHC class I epitope encoding nucleic acid sequence, (M) a TP53_K132N MHC class I epitope encoding nucleic acid sequence, (N) a KRAS_Q61L MHC class I epitope encoding nucleic acid sequence, (O) a TP53_R213L MHC class I epitope encoding nucleic acid sequence, (P) a BRAF_G466V MHC class I epitope encoding nucleic acid sequence, (Q) a KRAS_Q61H MHC class I epitope encoding nucleic acid sequence, (R) a CTNNB1_S37F MHC class I epitope encoding nucleic acid sequence, (S) a TP53_S127Y MHC class I epitope encoding nucleic acid sequence, (T) a TP53_K132E MHC class I epitope encoding nucleic acid sequence, and (U) a KRAS_G12C MHC class I epitope encoding nucleic acid sequence.

In some aspects, the at least one antigen-encoding nucleic acid sequence comprises: (A) a KRAS_G12C MHC class I epitope encoding nucleic acid sequence, (B) a KRAS_G12D MHC class I epitope encoding nucleic acid sequence, (C) a KRAS_G12V MHC class I epitope encoding nucleic acid sequence, D) a KRAS Q61H MHC class I epitope encoding nucleic acid sequence, or combinations thereof. In some aspects, the at least one antigen-encoding nucleic acid sequence comprises at least each of: (A) a KRAS_G12C MHC class I epitope encoding nucleic acid sequence, (B) a KRAS_G12D MHC class I epitope encoding nucleic acid sequence, and (C) a KRAS_G12V MHC class I epitope encoding nucleic acid sequence. In some aspects, the at least one antigen-encoding nucleic acid sequence comprises at least each of: (A) a KRAS_G12C MHC class I epitope encoding nucleic acid sequence, (B) a KRAS_G12D MHC class I epitope encoding nucleic acid sequence, (C) a KRAS_G12V MHC class I epitope encoding nucleic acid sequence, and (D) a KRAS Q61H MHC class I epitope encoding nucleic acid sequence. In some aspects, the at least one antigen-encoding nucleic acid sequence comprises at least each of: (A) a KRAS_G12C MHC class I epitope encoding nucleic acid sequence, wherein the KRAS_G12C MHC class I epitope encoding nucleic acid sequence encodes a MHC class I epitope comprising the sequence of SEQ ID NO: 14,954; 19,848; 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, and (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974. In some aspects, the at least one antigen-encoding nucleic acid sequence comprises at least each of: (A) a KRAS_G12C MHC class I epitope encoding nucleic acid sequence, wherein the KRAS_G12C MHC class I epitope encoding nucleic acid sequence encodes a MHC class I epitope comprising the sequence of SEQ ID NO: 14,954; 19,848; 19,850, (B) a KRAS_G12D MHC class I epitope encoding nucleic acid sequence, wherein the KRAS_G12D MHC class I epitope encoding nucleic acid sequence encodes a MHC class I epitope selected from the group consisting of SEQ ID NO: 19,749; 19,865; 19,863, (C) a KRAS_G12V MHC class I epitope encoding nucleic acid sequence, wherein the KRAS_G12V MHC class I epitope encoding nucleic acid sequence encodes a MHC class I epitope selected from the group consisting of SEQ ID NO: 19,976; 19,779; 11,495; and 19,974, and (D) a KRAS Q61H MHC class I epitope encoding nucleic acid sequence, wherein the KRAS_Q61H MHC class I epitope encoding nucleic acid sequence encodes a MHC class I epitope selected from the group consisting of SEQ ID NO: 14,409.

In some aspects, the at least one antigen-encoding nucleic acid sequence comprises: (A) a TP53_R213L MHC class I epitope encoding nucleic acid sequence, (B) a TP53_S127Y MHC class I epitope encoding nucleic acid sequence, (C) a TP53_R249M MHC class I epitope encoding nucleic acid sequence, or combinations thereof. In some aspects, the at least one antigen-encoding nucleic acid sequence comprises at least each of: (A) a TP53_R213L MHC class I epitope encoding nucleic acid sequence, (B) a TP53_S127Y MHC class I epitope encoding nucleic acid sequence, and (C) a TP53_R249M MHC class I epitope encoding nucleic acid sequence. In some aspects, the TP53_R213L MHC class I epitope encoding nucleic acid sequence encodes a MHC class I epitope selected from the group consisting of SEQ ID NO: 29,519.

In some aspects, the cassette of the composition for delivery of the ChAdV-based expression system is identical to the cassette of the composition for delivery of the self-amplifying alphavirus-based expression system. In some aspects, the cassette of the composition for delivery of the ChAdV-based expression system is different from the cassette of the composition for delivery of the self-amplifying alphavirus-based expression system.

In some aspects, stimulating the immune response comprises eliciting a cytotoxic T lymphocyte response to at least one of the one or more antigens. In some aspects, stimulating the immune response comprises a reduction in a tumor of the subject. In some aspects, the reduction is at least a 5%, at least a 10%, at least a 15%, at least a 20%, at least a 25%, at least a 30%, at least a 35%, at least a 40%, at least a 45%, at least a 50%, at least a 55%, at least a 60%, at least a 65%, at least a 70%, at least a 75%, at least a 80%, at least a 85%, at least a 90%, or at least a 95% reduction. In some aspects, the reduction is at least a 15% reduction. In some aspects, the reduction is at least a 20% reduction.

In some aspects, stimulating the immune response comprises stabilization of a tumor of the subject. In some aspects, stimulating the immune response comprises ameliorating a disease of the subject. In some aspects, ameliorating the disease comprises a complete response (CR), a partial response (PR), or a stable disease (SD).

In some aspects, the method further comprises administering one or more immune modulators. In some aspects, the one or more immune modulators are administered before, concurrently with, or after administration of any of the above compositions or pharmaceutical compositions. In some aspects, the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the anti-CTLA4 antibody is Ipilimumab. In some aspects, the anti-PD-1 is Nivolumab. In some aspects, the one or more immune modulators is administered intravenously (IV), intramuscularly (IM), intradermally (ID), or subcutaneously (SC). In some aspects, the subcutaneous administration is near the site of the composition or pharmaceutical composition administration or in close proximity to one or more vector or composition draining lymph nodes.

In some aspects, at least one of the one or more immune modulators is Ipilimumab. In some aspects, the Ipilimumab is administered subcutaneously (SC). In some aspects, the subcutaneous administration is proximal to a draining lymph node of the administration site of the self-amplifying alphavirus-based expression system or the composition for delivery of the ChAdV-based expression system. In some aspects, the Ipilimumab is administered at a dose of 30 mg. In some aspects, the dose of 30 mg is administered as four separate doses. In some aspects, at least one of the one or more immune modulators is Nivolumab. In some aspects, the Nivolumab is administered intravenously (IV). In some aspects, the Nivolumab is administered at a dose of 480 mg. In some aspects, the one or more immune modulators is each of Ipilimumab and Nivolumab. In some aspects, the Ipilimumab modulator is administered subcutaneously (SC) and wherein the Nivolumab modulator is administered intravenously (IV). In some aspects, the one or more immune modulators are administered concurrently with each administration of the self-amplifying alphavirus-based expression system or the composition for delivery of the ChAdV-based expression system.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG.) 1 illustrates development of an in vitro T cell activation assay. Schematic of the assay in which the delivery of a vaccine cassette to antigen presenting cells, leads to expression, processing and MHC-restricted presentation of distinct peptide antigens. Reporter T cells engineered with T cell receptors that match the specific peptide-MHC combination become activated resulting in luciferase expression.

FIG. 2A illustrates evaluation of linker sequences in short cassettes and shows five class I MHC restricted epitopes (epitopes 1 through 5) concatenated in the same position relative to each other followed by two universal class II MHC epitopes (MHC-II). Various iterations were generated using different linkers. In some cases the T cell epitopes are directly linked to each other. In others, the T cell epitopes are flanked on one or both sides by its natural sequence. In other iterations, the T cell epitopes are linked by the non-natural sequences AAY, RR, and DPP.

FIG. 2B illustrates evaluation of linker sequences in short cassettes and shows sequence information on the T cell epitopes embedded in the short cassettes. Figure discloses SEQ ID NOS 29365-29366, 29369, 29368, 29367, and 29494-29495, respectively, in order of appearance.

FIG. 3 illustrates evaluation of cellular targeting sequences added to model vaccine cassettes. The targeting cassettes extend the short cassette designs with ubiquitin (Ub), signal peptides (SP) and/or transmembrane (TM) domains, feature next to the five marker human T cell epitopes (epitopes 1 through 5) also two mouse T cell epitopes SIINFEKL (SEQ ID NO: 29362) (SII) and SPSYAYHQF (SEQ ID NO: 29363) (A5), and use either the non natural linker AAY- or natural linkers flanking the T cell epitopes on both sides (25mer).

FIG. 4 illustrates in vivo evaluation of linker sequences in short cassettes. A) Experimental design of the in vivo evaluation of vaccine cassettes using HLA-A2 transgenic mice.

FIG. 5A illustrates in vivo evaluation of the impact of epitope position in long 21-mer cassettes and shows the design of long cassettes entails five marker class I epitopes (epitopes 1 through 5) contained in their 25-mer natural sequence (linker=natural flanking sequences), spaced with additional well-known T cell class I epitopes (epitopes 6 through 21) contained in their 25-mer natural sequence, and two universal class II epitopes (MHC-II0, with only the relative position of the class I epitopes varied.

FIG. 5B illustrates in vivo evaluation of the impact of epitope position in long 21-mer cassettes and shows the sequence information on the T cell epitopes used. Figure discloses SEQ ID NOS 29365-29366, 29369, 29368, 29367, 29496-29498, 29370, and 29499-29510, respectively, in order of appearance.

FIG. 6A illustrates final cassette design for preclinical IND-enabling studies and shows the design of the final cassettes comprises 20 MHC I epitopes contained in their 25-mer natural sequence (linker=natural flanking sequences), composed of 6 non-human primate (NHP) epitopes, 5 human epitopes, 9 murine epitopes, as well as 2 universal MHC class II epitopes.

FIG. 6B illustrates final cassette design for preclinical IND-enabling studies and shows the sequence information for the T cell epitopes used that are presented on class I MHC of non-human primate, mouse and human origin, as well as sequences of 2 universal MHC class II epitopes PADRE and Tetanus toxoid. Figure discloses SEQ ID NOS 29426-29431, 29362-29363, 29456, 29511, 29460-29462, 29458-29459, 29367-29369, 29365-29366, 29494, and 29512, respectively, in order of appearance.

FIG. 7A illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using light microscopy (40× magnification).

FIG. 7B illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using fluorescent microscopy at 40× magnification.

FIG. 7C illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using fluorescent microscopy at 100× magnification.

FIG. 8A illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using light microscopy (40× magnification)

FIG. 8B illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using fluorescent microscopy at 40× magnification.

FIG. 8C illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using fluorescent microscopy at 100× magnification.

FIG. 9 illustrates the viral particle production scheme.

FIG. 10 illustrates the alphavirus derived VEE self-replicating RNA (srRNA) vector.

FIG. 11 illustrates in vivo reporter expression after inoculation of C57BL/6J mice with VEE-Luciferase srRNA. Shown are representative images of luciferase signal following immunization of C57BL/6J mice with VEE-Luciferase srRNA (10 ug per mouse, bilateral intramuscular injection, MC3 encapsulated) at various timepoints.

FIG. 12A illustrates T-cell responses measured 14 days after immunization with VEE srRNA formulated with MC3 LNP in B16-OVA tumor bearing mice. B16-OVA tumor bearing C57BL/6J mice were injected with 10 ug of VEE-Luciferase srRNA (control), VEE-UbAAY srRNA (Vax), VEE-Luciferase srRNA and anti-CTLA-4 (aCTLA-4) or VEE-UbAAY srRNA and anti-CTLA-4 (Vax+aCTLA-4). In addition, all mice were treated with anti-PD1 mAb starting at day 7. Each group consisted of 8 mice. Mice were sacrificed and spleens and lymph nodes were collected 14 days after immunization. SIINFEKL (SEQ ID NO: 29362)-specific T-cell responses were assessed by IFN-gamma ELISpot and are reported as spot-forming cells (SFC) per 106 splenocytes. Lines represent medians.

FIG. 12B illustrates T-cell responses measured 14 days after immunization with VEE srRNA formulated with MC3 LNP in B16-OVA tumor bearing mice. B16-OVA tumor bearing C57BL/6J mice were injected with 10 ug of VEE-Luciferase srRNA (control), VEE-UbAAY srRNA (Vax), VEE-Luciferase srRNA and anti-CTLA-4 (aCTLA-4) or VEE-UbAAY srRNA and anti-CTLA-4 (Vax+aCTLA-4). In addition, all mice were treated with anti-PD1 mAb starting at day 7. Each group consisted of 8 mice. Mice were sacrificed and spleens and lymph nodes were collected 14 days after immunization. SIINFEKL (SEQ ID NO: 29362)-specific T-cell responses were assessed by MHCI-pentamer staining, reported as pentamer positive cells as a percent of CD8 positive cells. Lines represent medians.

FIG. 13A illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by IFN-gamma ELISpot. Mice were sacrificed and spleens and lymph nodes collected at 14 days post immunization with adenovirus.

FIG. 13B illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by IFN-gamma ELISpot. Mice were sacrificed and spleens and lymph nodes collected at 14 days post immunization with adenovirus and 14 days post boost with srRNA (day 28 after prime).

FIG. 13C illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by MHC class I pentamer staining. Mice were sacrificed and spleens and lymph nodes collected at 14 days post immunization with adenovirus.

FIG. 13D illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by MHC class I pentamer staining. Mice were sacrificed and spleens and lymph nodes collected at 14 days post immunization with adenovirus and 14 days post boost with srRNA (day 28 after prime).

FIG. 14A illustrates antigen-specific T-cell responses following heterologous prime/boost in CT26 (Balb/c) tumor bearing mice. Mice were immunized with Ad5-GFP and boosted 15 days after the adenovirus prime with VEE-Luciferase srRNA formulated with MC3 LNP (Control) or primed with Ad5-UbAAY and boosted with VEE-UbAAY srRNA (Vax). Both the Control and Vax groups were also treated with an IgG control mAb. A separate group was administered the Ad5-GFP/VEE-Luciferase srRNA prime/boost in combination with anti-PD-1 (aPD1), while a fourth group received the Ad5-UbAAY/VEE-UbAAY srRNA prime/boost in combination with an anti-PD-1 mAb (Vax+aPD1). T-cell responses to the AH1 peptide were measured using IFN-gamma ELISpot. Mice were sacrificed and spleens and lymph nodes collected at 12 days post immunization with adenovirus.

FIG. 14B illustrates antigen-specific T-cell responses following heterologous prime/boost in CT26 (Balb/c) tumor bearing mice. Mice were immunized with Ad5-GFP and boosted 15 days after the adenovirus prime with VEE-Luciferase srRNA formulated with MC3 LNP (Control) or primed with Ad5-UbAAY and boosted with VEE-UbAAY srRNA (Vax). Both the Control and Vax groups were also treated with an IgG control mAb. A separate group was administered the Ad5-GFP/VEE-Luciferase srRNA prime/boost in combination with anti-PD-1 (aPD1), while a fourth group received the Ad5-UbAAY/VEE-UbAAY srRNA prime/boost in combination with an anti-PD-1 mAb (Vax+aPD1). T-cell responses to the AH1 peptide were measured using IFN-gamma ELISpot. Mice were sacrificed and spleens and lymph nodes collected at 12 days post immunization with adenovirus and 6 days post boost with srRNA (day 21 after prime).

FIG. 15 illustrates ChAdV68 eliciting T-Cell responses to mouse tumor antigens in mice. Mice were immunized with ChAdV68.5WTnt.MAG25mer, and T-cell responses to the MHC class I epitope SIINFEKL (SEQ ID NO: 29362) (OVA) were measured in C57BL/6J female mice and the MHC class I epitope AH1-A5 measured in Balb/c mice. Mean spot forming cells (SFCs) per 10⁶ splenocytes measured in ELISpot assays presented. Error bars represent standard deviation.

FIG. 16 illustrates cellular immune responses in a CT26 tumor model following a single immunization with either ChAdV6, ChAdV+anti-PD-1, srRNA, srRNA+anti-PD-1, or anti-PD-1 alone. Antigen-specific IFN-gamma production was measured in splenocytes for 6 mice from each group using ELISpot. Results are presented as spot forming cells (SFC) per 10⁶ splenocytes. Median for each group indicated by horizontal line. P values determined using the Dunnett's multiple comparison test; ***P<0.0001, **P<0.001, *P<0.05. ChAdV=ChAdV68.5WTnt.MAG25mer; srRNA=VEE-MAG25mer srRNA.

FIG. 17 illustrates CD8 T-Cell responses in a CT26 tumor model following a single immunization with either ChAdV6, ChAdV+anti-PD-1, srRNA, srRNA+anti-PD-1, or anti-PD-1 alone. Antigen-specific IFN-gamma production in CD8 T cells measured using ICS and results presented as antigen-specific CD8 T cells as a percentage of total CD8 T cells. Median for each group indicated by horizontal line. P values determined using the Dunnett's multiple comparison test; ***P<0.0001, **P<0.001, *P<0.05. ChAdV=ChAdV68.5WTnt.MAG25mer; srRNA=VEE-MAG25mer srRNA.

FIG. 18 illustrates tumor growth in a CT26 tumor model following immunization with a ChAdV/srRNA heterologous prime/boost, a srRNA/ChAdV heterologous prime/boost, or a srRNA/srRNA homologous primer/boost. Also illustrated in a comparison of the prime/boost immunizations with or without administration of anti-PD1 during prime and boost. Tumor volumes measured twice per week and mean tumor volumes presented for the first 21 days of the study. 22-28 mice per group at study initiation. Error bars represent standard error of the mean (SEM). P values determined using the Dunnett's test; ***P<0.0001, **P<0.001, *P<0.05. ChAdV=ChAdV68.5WTnt.MAG25mer; srRNA=VEE-MAG25mer srRNA.

FIG. 19 illustrates survival in a CT26 tumor model following immunization with a ChAdV/srRNA heterologous prime/boost, a srRNA/ChAdV heterologous prime/boost, or a srRNA/srRNA homologous primer/boost. Also illustrated in a comparison of the prime/boost immunizations with or without administration of anti-PD1 during prime and boost. P values determined using the log-rank test; ***P<0.0001, **P<0.001, *P<0.01. ChAdV=ChAdV68.5WTnt.MAG25mer; srRNA=VEE-MAG25mer srRNA.

FIG. 20 illustrates antigen-specific cellular immune responses measured using ELISpot. Antigen-specific IFN-gamma production to six different mamu A01 restricted epitopes was measured in PBMCs for the VEE-MAG25mer srRNA-LNP1 (30 μg) (FIG. 20A), VEE-MAG25mer srRNA-LNP1 (100 μg) (FIG. 20B), or VEE-MAG25mer srRNA-LNP2 (100 μg) (FIG. 20C) homologous prime/boost or the ChAdV68.5WTnt.MAG25mer/VEE-MAG25mer srRNA heterologous prime/boost group (FIG. 20D) using ELISpot 1, 2, 3, 4, 5, 6, 8, 9, or 10 weeks after the first boost immunization (6 rhesus macaques per group). Results are presented as mean spot forming cells (SFC) per 10⁶ PBMCs for each epitope in a stacked bar graph format. Values for each animal were normalized to the levels at pre-bleed (week 0).

FIG. 21 shows antigen-specific cellular immune response measured using ELISpot. Antigen-specific IFN-gamma production to six different mamu A01 restricted epitopes was measured in PBMCs after immunization with the ChAdV68.5WTnt.MAG25mer/VEE-MAG25mer srRNA heterologous prime/boost regimen using ELISpot prior to immunization and 4, 5, 6, 7, 8, 10, 11, 12, 13, 14, 15, 16, 17, 18, 19, 20, 21, 22, 23 or 24 weeks after the initial immunization. Results are presented as mean spot forming cells (SFC) per 10⁶ PBMCs for each epitope (6 rhesus macaques per group) in a stacked bar graph format.

FIG. 22 shows antigen-specific cellular immune response measured using ELISpot. Antigen-specific IFN-gamma production to six different mamu A01 restricted epitopes was measured in PBMCs after immunization with the VEE-MAG25mer srRNA LNP2 homologous prime/boost regimen using ELISpot prior to immunization and 4, 5, 6, 7, 8, 10, 11, 12, 13, 14, or 15 weeks after the initial immunization. Results are presented as mean spot forming cells (SFC) per 10⁶ PBMCs for each epitope (6 rhesus macaques per group) in a stacked bar graph format.

FIG. 23 shows antigen-specific cellular immune response measured using ELISpot. Antigen-specific IFN-gamma production to six different mamu A01 restricted epitopes was measured in PBMCs after immunization with the VEE-MAG25mer srRNA LNP1 homologous prime/boost regimen using ELISpot prior to immunization and 4, 5, 6, 7, 8, 10, 11, 12, 13, 14, or 15 weeks after the initial immunization. Results are presented as mean spot forming cells (SFC) per 10⁶ PBMCs for each epitope (6 rhesus macaques per group) in a stacked bar graph format.

FIG. 24A and FIG. 24B show example peptide spectrums generated from Promega's dynamic range standard. Figure discloses SEQ ID NO: 29364.

FIG. 25 shows the correlation between EDGE score and the probability of detection of candidate shared neoantigen peptides by targeted MS.

FIG. 26 shows expanded TILs from a patient stained with mutated peptide HLA-A*11:01 tetramers. The flow cytometry gating strategy on CD8+ cells (left panel) and the staining of CD8+ cells by KRAS-G12V/HLA-A*11:01 tetramer (right panel) are shown.

FIG. 27 illustrates the general TCR sequencing strategy and workflow.

FIG. 28 shows the TCR sequencing strategy for a representative example using a KRAS-G12V/HLA-A*11:01 tetramer.

FIG. 29 illustrates the general organization of the model epitopes from the various species for large antigen cassettes that had either 30 (L), 40 (XL) or 50 (XXL) epitopes.

FIG. 30 shows ChAd vectors express long cassettes as indicated by the above Western blot using an anti-class II (PADRE) antibody that recognizes a sequence common to all cassettes. HEK293 cells were infected with chAd68 vectors expressing large cassettes (chAd68-50XXL, chAd68-40XL & chAd68-30L) of variable size. Infections were set up at a MOI of 0.2. Twenty-four hours post infection MG132 a proteasome inhibitor was added to a set of the infected wells (indicated by the plus sign). Another set of virus treated wells were not treated with MG132 (indicated by minus sign). Uninfected HEK293 cells (293F) were used as a negative control. Forty-eight hours post infection cell pellets were harvested and analyzed by SDS/PAGE electrophoresis, and immunoblotting using a rabbit anti-Class II PADRE antibody. A HRP anti-rabbit antibody and ECL chemiluminescent substrate was used for detection.

FIG. 31 shows CD8+ immune responses in chAd68 large cassette immunized mice, detected against AH1 (top) and SIINFEKL (SEQ ID NO: 29362) (bottom) by ICS. Data is presented as IFNg+ cells against the model epitope as % of total CD8 cells

FIG. 32 shows CD8+ responses to LD-AH1+(top) and Kb-SIINFEKL+(bottom) Tetramers post chAd68 large cassette vaccination. Data is presented as % of total CD8 cells reactive against the model Tetramer peptide complex. *p<0.05, **p<0.01 by ANOVA with Tukey's test. All p-values compared to MAG 20-antigen cassette.

FIG. 33 shows CD8+ immune responses in alphavirus large cassette treated mice, detected against AH1 (top) and SIINFEKL (SEQ ID NO: 29362) (bottom) by ICS. Data is presented as IFNg+ cells against the model epitope as % of total CD8 cells. *p<0.05, **p<0.01, ***p<0.001 by ANOVA with Tukey's test. All p-values compared to MAG 20-antigen cassette.

FIG. 34 illustrates the vaccination strategy used to evaluate immunogenicity of the antigen-cassette containing vectors in rhesus macaques. Triangles indicate chAd68 vaccination (1e12 vp/animal) at weeks 0 & 32. Circles represent alphavirus vaccination at weeks 0, 4, 12, 20, 28 & 32. Squares represent administration of an anti-CTLA4 antibody.

FIG. 35 shows a time course of CD8+ anti-epitope responses in Rhesus Macaques dosed with chAd-MAG alone (Group 4). Mean SFC/1e6 splenocytes is shown.

FIG. 36 shows a time course of CD8+ anti-epitope responses in Rhesus Macaques dosed with chAd-MAG plus anti-CTLA4 antibody (Ipilimumab) delivered IV. (Group 5). Mean SFC/1e6 splenocytes is shown.

FIG. 37 shows a time course of CD8+ anti-epitope responses in Rhesus Macaques dosed with chAd-MAG plus anti-CTLA4 antibody (Ipilimumab) delivered SC (Group 6). Mean SFC/1e6 splenocytes is shown.

FIG. 38 shows antigen-specific memory responses generated by ChAdV68/samRNA vaccine protocol measured by ELISpot. Results are presented as individual dot plots, with each dot representing a single animal. Pre-immunization baseline (left panel) and memory response at 18 months post-prime (right panel) are shown.

FIG. 39 shows memory cell phenotyping of antigen-specific CD8+ T-cells by flow cytometry using combinatorial tetramer staining and CD45RA/CCR7 co-staining.

FIG. 40 shows the distribution of memory cell types within the sum of the four Mamu-A*01 tetramer+CD8+ T-cell populations at study month 18. Memory cells were characterized as follows: CD45RA+CCR7+=naïve, CD45RA+CCR7-=effector (Teff), CD45RA-CCR7+=central memory (Tcm), CD45RA-CCR7-=effector memory (Tem).

FIG. 41 shows frequency of CD8+ T cells recognizing the CT26 tumor antigen AH1 in CT26 tumor-bearing mice. P values determined using the one-way ANOVA with Tukey's multiple comparisons test; **P<0.001, *P<0.05. ChAdV=ChAdV68.5WTnt.MAG25mer; aCTLA4=anti-CTLA4 antibody, clone 9D9.

FIG. 42 shows titration of DOX administration in regulating expression of a representative neoantigen under a Tet-On system in multiple K562-HLA cell-lines.

FIG. 43 shows a representative KRAS G12V peptide VVGAVGVGK observed by mass-spectrometry in a HLA-A*11:01 expressing K562 cell line. Top panels shows detection was DOX dependent (left column no DOX; right panel DOX added), and bottom panels show detection of the heavy peptide control standard was equivalent.

FIG. 44A-F shows antigen/HLA prevalence (the frequency of antigen (A) in a given population multiplied by the frequency of an HLA allele (B) in the given population) plotted for each of the mutations shown in various cancer subsets. The total Antigen/HLA prevalence of all shared neoantigens combined is indicated (see percentage next to given cancer). Also indicated are the number of distinct HLAs predicted to present the given neoantigen (see number above bar for each specific mutation). FIG. 44A shows Pancreatic, AML, Hepatocellular (left, middle, right panels, respectively). FIG. 44B shows Melanoma, Rectal Adeno, Uterine Endometrial (left, middle, right panels, respectively). FIG. 44C shows Colon Adeno, Myelodysplastic, Lung Adeno (left, middle, right panels, respectively). FIG. 44D shows Esophageal Adeno, Bladder, Lung Squamous (left, middle, right panels, respectively). FIG. 44E shows Thyroid, Small Cell Lung, Serous Ovarian (left, middle, right panels, respectively). FIG. 44F shows Gallbladder, Breast [lobular], Breast [ductal] (left, middle, right panels, respectively).

FIG. 45A illustrates a Phase 1 study designed to assess the dose, safety and tolerability, immunogenicity, and early clinical activity of a personalized neoantigen cancer vaccine (“GRANITE”) administered in combination with immune checkpoint blockade in patients with advanced cancer. The heterologous prime/boost vaccine regimen involves (1) a ChAdV that is used as a prime vaccination [GRT-C901] and (2) a SAM formulated in a LNP that is used for boost vaccinations [GRT-R902] following GRT-C901. Abbreviations: IM=intramuscular; IV=intravenous; vp=viral particles; MSS-CRC=microsatellite stable colorectal cancer; NSCLC=non-small cell lung cancer; PDA=pancreatic ductal adenocarcinoma; SC=subcutaneous; GEA=gastroesophageal adenocarcinoma

FIG. 45B illustrates a Phase 1 study designed to assess the dose, safety and tolerability, immunogenicity, and early clinical activity of a shared neoantigen cancer vaccine (“SLATE”) administered in combination with immune checkpoint blockade in patients with advanced cancer. The heterologous prime/boost vaccine regimen involves (1) a ChAdV that is used as a prime vaccination [GRT-C903] and (2) a SAM formulated in a LNP that is used for boost vaccinations [GRT-R904] following GRT-C903. Abbreviations: IM=intramuscular; IV=intravenous; vp=viral particles; MSS-CRC=microsatellite stable colorectal cancer; NSCLC=non-small cell lung cancer; PDA=pancreatic ductal adenocarcinoma; SC=subcutaneous

FIG. 46A illustrates a Phase 2 study designed to assess the dose, safety and tolerability, immunogenicity, and early clinical activity of a personalized neoantigen cancer vaccine (“GRANITE”) administered in combination with immune checkpoint blockade in patients with advanced cancer for tumor-specific expansion cohorts.

FIG. 46B illustrates a Phase 2 study designed to assess the dose, safety and tolerability, immunogenicity, and early clinical activity of a shared neoantigen cancer vaccine (“SLATE”) administered in combination with immune checkpoint blockade in patients with advanced cancer for tumor-specific expansion cohorts.

FIG. 47 illustrates aspects of the 1) HLA Screening Stage and the 2) Study Treatment Stage of patient selection.

FIG. 48 illustrates an improved version of the dose selection design, referred to as mTPI-2. DLT=dose-limiting toxicity; E=Escalate to the next higher dose level; S=Stay at the current dose level; D=De-escalate to the next lower dose level; DU=Dose unacceptable due to toxicity; Target toxicity rate=30%; Sample size=24

FIG. 49 illustrates the Phase 1 dosing schedule GRT-C903, GRT-R904, Nivolumab, and Ipilimumab. Abbreviations: Bx=biopsy; DLT=dose limiting toxicity; a: DLT period for Phase 1 only; b: ipilimumab not administered in Phase 1 Dose Level 1

FIG. 50 shows evaluation of the presence of T cell precursors in the naïve T cell repertoire of healthy donors as assessed by tetramer-staining using flow-cytometry.

FIG. 51A illustrates the treatment history and schedule for 10 GRANITE patients enrolled in the study.

FIG. 51B shows a ELISpot CD8 T cell response timecourse for GRANITE patients G1, G2, and G3. Shown are responses to vehicle (left column at each time point) and a peptide pool of neoepitopes encoded by each GRANITE patient respective antigen cassette (right column at each time point). Dashed line represents the limit of detection (LOD).

FIG. 51C shows a ELISpot CD8 T cell response timecourse for GRANITE patients G4, G6, G7, and G8. Shown are responses to vehicle (left column at each time point) and a peptide pool of neoepitopes encoded by each GRANITE patient respective antigen cassette (right column at each time point). Dashed line represents the limit of detection (LOD). * Poor cellular recovery

FIG. 51D illustrates the treatment summary and indications for 19 SLATE patients enrolled in the study.

FIG. 52 illustrates the GRANITE treatment schedule and shows the accompanying ELISpot CD8 T cell response for GRANITE patient G1. Shown are responses to vehicle (left column at each time point) and a peptide pool of 40 neoepitopes encoded by GRANITE patient G1's antigen cassette (right column at each time point).

FIG. 53 illustrates the GRANITE treatment schedule and shows the accompanying ELISpot CD8 T cell response for GRANITE patient G2. Shown are responses to vehicle and a peptide pool of 40 neoepitopes encoded by GRANITE patient G2's antigen cassette.

FIG. 54A shows cytokine secretion (IL-2 and granzyme B) by CD8 T cells following peptide stimulation a peptide pool of 40 neoepitopes encoded by GRANITE patient G1's and GRANITE patient G2's antigen cassette, respectively, for T cells isolated at Day 0 and Day 35.

FIG. 54B shows IFN-gamma production by CD8 T cells following peptide stimulation by 40 neoepitopes split into separate pools (4 pools of 10) for neoepitopes encoded by the antigen cassette for GRANITE patients G1, G2, G3, G4, G7, and G8, respectively.

FIG. 54C shows the 12 of the 20 vaccine-encoded neoantigens (left panel) and fraction of the 20 neoantigens (right panel) encoded by GRANITE patient G2's antigen cassette that resulted in a T cell response as assessed by peptide stimulation for each of the 40 neoepitopes separately (multiple peptides of the 40 were derived from the same encoded neoantigen).

FIG. 55A shows the ELISpot CD8 T cell response for GRANITE patient G1 for T cells before and after the heterologous prime/boost vaccine strategy. Shown are responses to a peptide pool of 40 neoepitopes (“CD8 pool”) and the 40 neoepitopes split into separate pools (4 pools of 10) encoded by GRANITE patient G1's antigen cassette. Pool 4 was not tested.

FIG. 55B shows the ELISpot CD8 T cell response for GRANITE patient #2 for T cells before and after the heterologous prime/boost vaccine strategy. Shown are responses to a peptide pool of 40 neoepitopes (“CD8 pool”) and 40 neoepitopes split into separate pools (4 pools of 10) encoded by GRANITE patient G2's antigen cassette.

FIG. 55C shows the general sequencing workflow for TCR β sequencing of the expanded CD8 T cells.

FIG. 55D shows the expansion profile of 27 TCR-βs from PBMCs stimulated by IVS for GRANITE patient G3 following treatment as determined by the percent proportion of productive T cells.

FIG. 55E shows the expansion profile of TCR-βs from tumor-infiltrating T cells for GRANITE patient G3 following treatment as determined by the percent proportion of productive T cells.

FIG. 56 illustrates the GRANITE treatment schedule and shows the accompanying ELISpot CD8 T cell response for GRANITE patient G3. Shown are responses to vehicle (left column at each time point) and a peptide pool of 40 neoepitopes encoded by GRANITE patient G3's antigen cassette (right column at each time point).

FIG. 57 illustrates the planned SLATE treatment schedule (bottom panel) and shows the accompanying summary of ELISpot CD8 T cell response for each of 4 SLATE patients enrolled in the study (top panel).

FIG. 58A illustrates the SLATE treatment schedule and shows the accompanying ELISpot CD8 T cell response for SLATE patient S2. Shown are responses to vehicle (left column at each time point) and a peptide pool of 40 KRAS G12C epitopes (right column at each time point).

FIG. 58B shows ELISpot CD8 T cell response for SLATE patient S4. Shown are responses to vehicle (left column at each time point), a peptide pool of 40 KRAS G12C epitopes (middle column at each time point), or single peptide ILDTAGHEEY (right column at each time point).

FIG. 58C shows CD8 T cell responses to peptide stimulation using G12C, Q61H, or G12V peptide pools with data shown for Week 4 for S4 and S11; Week 8 for S7, S9, and S15; Week 12 for S2; and Week 20 for S3.

FIG. 58D illustrates the SLATE treatment schedule and shows the accompanying ELISpot CD8 T cell response for SLATE patient S3. Shown are responses to vehicle (left column at each time point) and a peptide pool of 40 KRAS G12C epitopes (right column at each time point).

FIG. 58E shows ELISpot CD8 T cell response for SLATE patients S2, S3, S9, S11, and S13. Shown are responses to vehicle (left column at each time point) and a peptide pools featuring TP53 mutations R213L, S127Y, and R249M (right column at each time point).

FIG. 59A shows radiological lung CT scans for GRANITE patient G3 at baseline (left panels), week 8 (second column panels), and week 16 (third column panels), and week 24 (right panels). Arrows and boxes highlight lesions.

FIG. 59B shows a first series of radiological lung CT scans for GRANITE patient G8 at baseline (left panels), week 8 (middle panels), and week 16 (right panels). Arrows highlight lesions.

FIG. 59C shows a second series of radiological lung CT scans for GRANITE patient G8 at baseline (left panels), week 8 (middle panels), and week 16 (right panels). Arrows highlight lesions.

FIG. 59D shows a series of radiological liver CT scans for GRANITE patient G8 at baseline (left panels), week 8 (middle panels), and week 16 (right panels). Arrows highlight lesions.

FIG. 60A shows radiological CT scans for SLATE patient S2 at baseline (left panels), week 8 (second column panels), and week 16 (third column panels), and week 24 (right panels). *Sum of longest diameters of two target lesions

FIG. 60B shows quantification of tumor-infiltrating CD8 T cells.

FIG. 61 shows a schematic of an antigen cassette featuring either single or multiple (4×) iterations of epitopes and representative data following immunization with the cassettes.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21;354(6310):354-358. Exemplary shared neoantigens are shown in Table A and in the AACR GENIE Results (SEQ ID NO:10,755-29,357); corresponding HLA allele(s) for each antigen are also shown. Such shared neoantigens are useful for inducing an immune response in a subject via administration. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISpot” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or energizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, and smoking history.

The term “antigen-encoding nucleic acid sequences derived from a tumor” refers to nucleic acid sequences directly extracted from the tumor, e.g. via RT-PCR; or sequence data obtained by sequencing the tumor and then synthesizing the nucleic acid sequences using the sequencing data, e.g., via various synthetic or PCR-based methods known in the art.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a 26S promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, or other 26S subgenomic promoter sequence, a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethylenegly col (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between.

The term “pharmaceutically effective amount” is an amount of a vaccine component (such as a peptide, engineered vector, and/or adjuvant) that is effective in a route of administration to provide a cell with sufficient levels of protein, protein expression, and/or cell-signaling activity (e.g., adjuvant-mediated activation) to provide a vaccinal benefit, i.e., some measurable level of immunity.

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Methods of Identifying Antigens

Methods for identifying shared antigens (e.g., neoantigens) include identifying antigens from a tumor of a subject that are likely to be presented on the cell surface of the tumor or immune cells, including professional antigen presenting cells such as dendritic cells, and/or are likely to be immunogenic. As an example, one such method may comprise the steps of: obtaining at least one of exome, transcriptome or whole genome tumor nucleotide sequencing and/or expression data from the tumor cell of the subject, wherein the tumor nucleotide sequencing and/or expression data is used to obtain data representing peptide sequences of each of a set of antigens (e.g., in the case of neoantigens wherein the peptide sequence of each neoantigen comprises at least one alteration that makes it distinct from the corresponding wild-type peptide sequence or in cases of shared antigens without a mutation where peptides are derived from any polypeptide known to or have been found to have altered expression in a tumor cell or cancerous tissue in comparison to a normal cell or tissue); inputting the peptide sequence of each antigen into one or more presentation models to generate a set of numerical likelihoods that each of the antigens is presented by one or more MHC alleles on the tumor cell surface of the tumor cell of the subject or cells present in the tumor, the set of numerical likelihoods having been identified at least based on received mass spectrometry data; and selecting a subset of the set of antigens based on the set of numerical likelihoods to generate a set of selected antigens.

The presentation model can comprise a statistical regression or a machine learning (e.g., deep learning) model trained on a set of reference data (also referred to as a training data set) comprising a set of corresponding labels, wherein the set of reference data is obtained from each of a plurality of distinct subjects where optionally some subjects can have a tumor, and wherein the set of reference data comprises at least one of: data representing exome nucleotide sequences from tumor tissue, data representing exome nucleotide sequences from normal tissue, data representing transcriptome nucleotide sequences from tumor tissue, data representing proteome sequences from tumor tissue, and data representing MHC peptidome sequences from tumor tissue, and data representing MHC peptidome sequences from normal tissue. The reference data can further comprise mass spectrometry data, sequencing data, RNA sequencing data, expression profiling data, and proteomics data for single-allele cell lines engineered to express a predetermined MHC allele that are subsequently exposed to synthetic protein, normal and tumor human cell lines, and fresh and frozen primary samples, and T cell assays (e.g., ELISpot). In certain aspects, the set of reference data includes each form of reference data.

The presentation model can comprise a set of features derived at least in part from the set of reference data, and wherein the set of features comprises at least one of allele dependent-features and allele-independent features. In certain aspects each feature is included.

Methods for identifying shared antigens also include generating an output for constructing a personalized cancer vaccine by identifying one or more antigens from one or more tumor cells of a subject that are likely to be presented on a surface of the tumor cells. As an example, one such method may comprise the steps of: obtaining at least one of exome, transcriptome, or whole genome nucleotide sequencing and/or expression data from the tumor cells and normal cells of the subject, wherein the nucleotide sequencing and/or expression data is used to obtain data representing peptide sequences of each of a set of antigens identified by comparing the nucleotide sequencing and/or expression data from the tumor cells and the nucleotide sequencing and/or expression data from the normal cells (e.g., in the case of neoantigens wherein the peptide sequence of each neoantigen comprises at least one alteration that makes it distinct from the corresponding wild-type peptide sequence or in cases of shared antigens without a mutation where peptides are derived from any polypeptide known to or have been found to have altered expression in a tumor cell or cancerous tissue in comparison to a normal cell or tissue), peptide sequence identified from the normal cells of the subject; encoding the peptide sequences of each of the antigens into a corresponding numerical vector, each numerical vector including information regarding a plurality of amino acids that make up the peptide sequence and a set of positions of the amino acids in the peptide sequence; inputting the numerical vectors, using a computer processor, into a deep learning presentation model to generate a set of presentation likelihoods for the set of antigens, each presentation likelihood in the set representing the likelihood that a corresponding antigen is presented by one or more class II MHC alleles on the surface of the tumor cells of the subject, the deep learning presentation model; selecting a subset of the set of antigens based on the set of presentation likelihoods to generate a set of selected antigens; and generating the output for constructing the personalized cancer vaccine based on the set of selected antigens.

Specific methods for identifying antigens, including neoantigens, are known to those skilled in the art, for example the methods described in more detail in international patent application publications WO/2017/106638, WO/2018/195357, and WO/2018/208856, each herein incorporated by reference, in their entirety, for all purposes.

A method of treating a subject having a tumor is disclosed herein, comprising performing the steps of any of the antigen identification methods described herein, and further comprising obtaining a tumor vaccine comprising the set of selected antigens, and administering the tumor vaccine to the subject.

A method disclosed herein can also include identifying one or more T cells that are antigen-specific for at least one of the antigens in the subset. In some embodiments, the identification comprises co-culturing the one or more T cells with one or more of the antigens in the subset under conditions that expand the one or more antigen-specific T cells. In further embodiments, the identification comprises contacting the one or more T cells with a tetramer comprising one or more of the antigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected antigen in the subset.

Also disclosed herein is a methods for manufacturing a tumor vaccine, comprising the steps of: obtaining at least one of exome, transcriptome or whole genome tumor nucleotide sequencing and/or expression data from the tumor cell of the subject, wherein the tumor nucleotide sequencing and/or expression data is used to obtain data representing peptide sequences of each of a set of antigens (e.g., in the case of neoantigens wherein the peptide sequence of each neoantigen comprises at least one alteration that makes it distinct from the corresponding wild-type peptide sequence or in cases of shared antigens without a mutation where peptides are derived from any polypeptide known to or have been found to have altered expression in a tumor cell or cancerous tissue in comparison to a normal cell or tissue); inputting the peptide sequence of each antigen into one or more presentation models to generate a set of numerical likelihoods that each of the antigens is presented by one or more MHC alleles on the tumor cell surface of the tumor cell of the subject, the set of numerical likelihoods having been identified at least based on received mass spectrometry data; and selecting a subset of the set of antigens based on the set of numerical likelihoods to generate a set of selected antigens; and producing or having produced a tumor vaccine comprising the set of selected antigens.

Also disclosed herein is a tumor vaccine including a set of selected antigens selected by performing the method comprising the steps of: obtaining at least one of exome, transcriptome or whole genome tumor nucleotide sequencing and/or expression data from the tumor cell of the subject, wherein the tumor nucleotide sequencing and/or expression data is used to obtain data representing peptide sequences of each of a set of antigens, and wherein the peptide sequence of each antigen (e.g., in the case of neoantigens wherein the peptide sequence of each neoantigen comprises at least one alteration that makes it distinct from the corresponding wild-type peptide sequence or in other cases of shared antigens without a mutation where peptides are derived from any polypeptide known to or have been found to have altered expression in a tumor cell or cancerous tissue in comparison to a normal cell or tissue); inputting the peptide sequence of each antigen into one or more presentation models to generate a set of numerical likelihoods that each of the antigens is presented by one or more MHC alleles on the tumor cell surface of the tumor cell of the subject, the set of numerical likelihoods having been identified at least based on received mass spectrometry data; and selecting a subset of the set of antigens based on the set of numerical likelihoods to generate a set of selected antigens; and producing or having produced a tumor vaccine comprising the set of selected antigens.

The tumor vaccine may include one or more of a nucleotide sequence, a polypeptide sequence, RNA, DNA, a cell, a plasmid, or a vector.

The tumor vaccine may include one or more antigens presented on the tumor cell surface.

The tumor vaccine may include one or more antigens that is immunogenic in the subject.

The tumor vaccine may not include one or more antigens that induce an autoimmune response against normal tissue in the subject.

The tumor vaccine may include an adjuvant.

The tumor vaccine may include an excipient.

A method disclosed herein may also include selecting antigens that have an increased likelihood of being presented on the tumor cell surface relative to unselected antigens based on the presentation model.

A method disclosed herein may also include selecting antigens that have an increased likelihood of being capable of inducing a tumor-specific immune response in the subject relative to unselected antigens based on the presentation model.

A method disclosed herein may also include selecting antigens that have an increased likelihood of being capable of being presented to naïve T cells by professional antigen presenting cells (APCs) relative to unselected antigens based on the presentation model, optionally wherein the APC is a dendritic cell (DC).

A method disclosed herein may also include selecting antigens that have a decreased likelihood of being subject to inhibition via central or peripheral tolerance relative to unselected antigens based on the presentation model.

A method disclosed herein may also include selecting antigens that have a decreased likelihood of being capable of inducing an autoimmune response to normal tissue in the subject relative to unselected antigens based on the presentation model.

The exome or transcriptome nucleotide sequencing and/or expression data may be obtained by performing sequencing on the tumor tissue.

The sequencing may be next generation sequencing (NGS) or any massively parallel sequencing approach.

The set of numerical likelihoods may be further identified by at least MHC-allele interacting features comprising at least one of: the predicted affinity with which the MHC allele and the antigen encoded peptide bind; the predicted stability of the antigen encoded peptide-MHC complex; the sequence and length of the antigen encoded peptide; the probability of presentation of antigen encoded peptides with similar sequence in cells from other individuals expressing the particular MHC allele as assessed by mass-spectrometry proteomics or other means; the expression levels of the particular MHC allele in the subject in question (e.g. as measured by RNA-seq or mass spectrometry); the overall neoantigen encoded peptide-sequence-independent probability of presentation by the particular MHC allele in other distinct subjects who express the particular MHC allele; the overall neoantigen encoded peptide-sequence-independent probability of presentation by MHC alleles in the same family of molecules (e.g., HLA-A, HLA-B, HLA-C, HLA-DQ, HLA-DR, HLA-DP) in other distinct subjects.

The set of numerical likelihoods are further identified by at least MHC-allele noninteracting features comprising at least one of: the C- and N-terminal sequences flanking the neoantigen encoded peptide within its source protein sequence; the presence of protease cleavage motifs in the neoantigen encoded peptide, optionally weighted according to the expression of corresponding proteases in the tumor cells (as measured by RNA-seq or mass spectrometry); the turnover rate of the source protein as measured in the appropriate cell type; the length of the source protein, optionally considering the specific splice variants (“isoforms”) most highly expressed in the tumor cells as measured by RNA-seq or proteome mass spectrometry, or as predicted from the annotation of germline or somatic splicing mutations detected in DNA or RNA sequence data; the level of expression of the proteasome, immunoproteasome, thymoproteasome, or other proteases in the tumor cells (which may be measured by RNA-seq, proteome mass spectrometry, or immunohistochemistry); the expression of the source gene of the neoantigen encoded peptide (e.g., as measured by RNA-seq or mass spectrometry); the typical tissue-specific expression of the source gene of the neoantigen encoded peptide during various stages of the cell cycle; a comprehensive catalog of features of the source protein and/or its domains as can be found in e.g. uniProt or PDB http://www.rcsb.org/pdb/home/home.do; features describing the properties of the domain of the source protein containing the peptide, for example: secondary or tertiary structure (e.g., alpha helix vs beta sheet); alternative splicing; the probability of presentation of peptides from the source protein of the neoantigen encoded peptide in question in other distinct subjects; the probability that the peptide will not be detected or over-represented by mass spectrometry due to technical biases; the expression of various gene modules/pathways as measured by RNASeq (which need not contain the source protein of the peptide) that are informative about the state of the tumor cells, stroma, or tumor-infiltrating lymphocytes (TILs); the copy number of the source gene of the neoantigen encoded peptide in the tumor cells; the probability that the peptide binds to the TAP or the measured or predicted binding affinity of the peptide to the TAP; the expression level of TAP in the tumor cells (which may be measured by RNA-seq, proteome mass spectrometry, immunohistochemistry); presence or absence of tumor mutations, including, but not limited to: driver mutations in known cancer driver genes such as EGFR, KRAS, ALK, RET, ROS1, TP53, CDKN2A, CDKN2B, NTRK1, NTRK2, NTRK3, and in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome). Peptides whose presentation relies on a component of the antigen-presentation machinery that is subject to loss-of-function mutation in the tumor have reduced probability of presentation; presence or absence of functional germline polymorphisms, including, but not limited to: in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome); tumor type (e.g., NSCLC, melanoma); clinical tumor subtype (e.g., squamous lung cancer vs. non-squamous); smoking history; the typical expression of the source gene of the peptide in the relevant tumor type or clinical subtype, optionally stratified by driver mutation.

The at least one alteration may be a frameshift or nonframeshift indel, missense or nonsense substitution, splice site alteration, genomic rearrangement or gene fusion, or any genomic or expression alteration giving rise to a neoORF.

The tumor cell may be selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

A method disclosed herein may also include obtaining a tumor vaccine comprising the set of selected neoantigens or a subset thereof, optionally further comprising administering the tumor vaccine to the subject.

At least one of neoantigens in the set of selected neoantigens, when in polypeptide form, may include at least one of: a binding affinity with MHC with an IC50 value of less than 1000 nM, for MHC Class I polypeptides a length of 8-15, 8, 9, 10, 11, 12, 13, 14, or 15 amino acids, for MHC Class II polypeptides a length of 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the polypeptide in the parent protein sequence promoting proteasome cleavage, and presence of sequence motifs promoting TAP transport. For MHC Class II, presence of sequence motifs within or near the peptide promoting cleavage by extracellular or lysosomal proteases (e.g., cathepsins) or HLA-DM catalyzed HLA binding.

Disclosed herein is are methods for identifying one or more neoantigens that are likely to be presented on a tumor cell surface of a tumor cell, comprising executing the steps of: receiving mass spectrometry data comprising data associated with a plurality of isolated peptides eluted from major histocompatibility complex (MHC) derived from a plurality of fresh or frozen tumor samples; obtaining a training data set by at least identifying a set of training peptide sequences present in the tumor samples and presented on one or more MHC alleles associated with each training peptide sequence; obtaining a set of training protein sequences based on the training peptide sequences; and training a set of numerical parameters of a presentation model using the training protein sequences and the training peptide sequences, the presentation model providing a plurality of numerical likelihoods that peptide sequences from the tumor cell are presented by one or more MHC alleles on the tumor cell surface.

The presentation model may represent dependence between: presence of a pair of a particular one of the MHC alleles and a particular amino acid at a particular position of a peptide sequence; and likelihood of presentation on the tumor cell surface, by the particular one of the MHC alleles of the pair, of such a peptide sequence comprising the particular amino acid at the particular position.

A method disclosed herein can also include selecting a subset of neoantigens, wherein the subset of neoantigens is selected because each has an increased likelihood that it is presented on the cell surface of the tumor relative to one or more distinct tumor neoantigens.

A method disclosed herein can also include selecting a subset of neoantigens, wherein the subset of neoantigens is selected because each has an increased likelihood that it is capable of inducing a tumor-specific immune response in the subject relative to one or more distinct tumor neoantigens.

A method disclosed herein can also include selecting a subset of neoantigens, wherein the subset of neoantigens is selected because each has an increased likelihood that it is capable of being presented to naïve T cells by professional antigen presenting cells (APCs) relative to one or more distinct tumor neoantigens, optionally wherein the APC is a dendritic cell (DC).

A method disclosed herein can also include selecting a subset of neoantigens, wherein the subset of neoantigens is selected because each has a decreased likelihood that it is subject to inhibition via central or peripheral tolerance relative to one or more distinct tumor neoantigens.

A method disclosed herein can also include selecting a subset of neoantigens, wherein the subset of neoantigens is selected because each has a decreased likelihood that it is capable of inducing an autoimmune response to normal tissue in the subject relative to one or more distinct tumor neoantigens.

A method disclosed herein can also include selecting a subset of neoantigens, wherein the subset of neoantigens is selected because each has a decreased likelihood that it will be differentially post-translationally modified in tumor cells versus APCs, optionally wherein the APC is a dendritic cell (DC).

The practice of the methods herein will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### III. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in international patent application publications WO/2017/106638, WO/2018/195357, and WO/2018/208856, each herein incorporated by reference, in their entirety, for all purposes.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of nonframeshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle adds the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1 G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences. Shared neoantigens are shown in Table A (see SEQ ID NO:10,755-21,015) and in the AACR GENIE results (see SEQ ID NO: 21,016-29,357). Shared antigens are shown in Table 1.2 (see SEQ ID NO:57-10,754).

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be is immunogenic in a subject having a tumor, e.g., capable of eliciting a T cell response or a B cell response in the subject.

One or more antigens that induce an autoimmune response in a subject can be excluded from consideration in the context of vaccine generation for a subject having a tumor.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids—thus bypassing the need for computational or in vitro test-based selection of the strongest HLA-presented shorter peptide. In both cases, use of a longer peptide allows endogenous processing by patient cells and may lead to more effective antigen presentation and induction of T cell responses.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. The peptide contains the tumor specific mutation. In some aspects the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., a tumor-specific immune response. Vaccine compositions typically comprise one or a plurality of antigens, e.g., selected using a method described herein or as set forth in Table A, Table 1.2, or AACR GENIE Results. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain at least two repeats of an epitope-encoding nucleic acid sequence. A used herein, a “repeat” refers to two or more iterations of an identical nucleic acid epitope-encoding nucleic acid sequence (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two repeats of an epitope-encoding nucleic acid sequence. In further non-limiting examples, the antigen-encoding nucleic acid sequence portion of a cassette encodes more than one distinct epitope, and at least one of the distinct epitopes is encoded by at least two repeats of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having repeats of at least one of the distinct epitopes are illustrated by, but is not limited to, the formulas below:

Repeat of one distinct epitope (repeat of epitope A):

E_{A}-E_{B}-E_{C}-E_{A}; or

E_{A}-E_{A}-E_{B}-E_{C}

Repeat of multiple distinct epitopes (repeats of epitopes A, B, and C):

E_{A}-E_{B}-E_{C}-E_{A}-E_{B}-E_{C}; or

E_{A}-E_{A}-E_{B}-E_{B}-E_{C}-E_{C}

Multiple repeats of multiple distinct epitopes (repeats of epitopes A, B, and C):

E_{A}-E_{B}-E_{C}-E_{A}-E_{B}-E_{C}-E_{A}-E_{B}-E_{C}; or

E_{A}-E_{A}-E_{A}-E_{B}-E_{B}-E_{B}-E_{C}-E_{C}-E_{C}

The above examples are not limiting and the antigen-encoding nucleic acid sequences having repeats of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

(Eₓ-(E^{N}ₙ)_{y})_{z}

where E represents a nucleotide sequence comprising at least one of the at least one distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof.

Each E or E^{N} can independently comprise any epitope-encoding nucleic acid sequence described herein. For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein, e.g., see V.A. Antigen Cassette.

Repeats of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Repeats of an epitope-encoding nucleic acid sequences can be separated by one or more additional nucleotides sequences. In general, repeats of an epitope-encoding nucleic acid sequences can be separated by any size nucleotide sequence applicable for the compositions described herein. In one example, repeats of an epitope-encoding nucleic acid sequences can be separated by a separate distinct epitope-encoding nucleic acid sequence (e.g., E_{A}-E_{B}-E_{C}-E_{A} . . . , as illustrated above). In examples where repeats are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEM FVTAPDNLGYMYEVQWPGQTQPQIANCSVYDFFVWLHYYSVRDT encoding repeats of 25mer antigens Trp1 (VTNTEMFVTAPDNLGYMYEVQWPGQ) and Trp2 (TQPQIANCSVYDFFVWLHYYSVRDT), the repeats of Trp1 are separated by the 25mer Trp2 and thus the repeats of the Trp1 epitope-encoding nucleic acid sequences are separated the 75 nucleotide Trp2 epitope-encoding nucleic acid sequence. In examples where repeats are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 150, 225, 300, 375, 450, 525, 600, or 675 nucleotides, respectively.

In some instances, an antigen or epitope in a cassette encoding additional antigens and/or epitopes may be an immunodominant epitope relative to the others encoded. Immunodominance, in general, is the skewing of an immune response towards only one or a few specific immunogenic peptides. Immunodominance can be assessed as part of an immune monitoring protocol. For example, immunodominance can be assessed through evaluating T cell and/or B cell responses to the encoded antigens.

In some instances, it may be desired to avoid vaccine compositions containing immunodominant epitope. For example, it may be desired to avoid designing a vaccine cassette encoding an immunodominant epitope. Without wishing to be bound by theory, administering and/or encoding an immunodominant epitope together with additional epitope may reduce the immune response to the additional epitopes, including potentially ultimately reducing vaccine efficacy against the additional epitopes. As an illustrative non-limiting example, vaccine compositions including TP53-associated neoepitopes may have the immune response, e.g., a T cell response, skewed towards the TP53-associated neoepitope negatively impacting the immune response to other antigens or epitopes in the vaccine composition (e.g., one or more KRAS-associated neoepitopes in a vaccine composition.)

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can be capable of raising a specific cytotoxic T-cells response and/or a specific helper T-cell response.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more neoantigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### V.A. Antigen Cassette

The methods employed for the selection of one or more antigens, the cloning and construction of a “cassette” and its insertion into a viral vector are within the skill in the art given the teachings provided herein. By “antigen cassette” is meant the combination of a selected antigen or plurality of antigens and the other regulatory elements necessary to transcribe the antigen(s) and express the transcribed product. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. Cassettes can include one or more neoantigens shown in Table A and/or AACR GENIE Results, and/or one or more antigens shown in Table 1.2.

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As above stated, the antigen cassette can be located in the site of any selected deletion in the viral vector, such as the site of the E1 gene region deletion or E3 gene region deletion, among others which may be selected.

The antigen cassette can be described using the following formula to describe the ordered sequence of each element, from 5′ to 3′:

(Pₐ-(L5_{b}-N_{c}-L3_{d})_{X})_{Z}-(P2ₕ-(G5ₑ-U_{f})_{Y})_{W}-G3_{g}

wherein P and P2 comprise promoter nucleotide sequences, N comprises an MHC class I epitope encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding N_{c} is a epitope encoding nucleic acid sequence, where for each Y the corresponding U_{f} is an antigen-encoding nucleic acid sequence. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (i.e. only the promoter nucleotide sequence provided by the RNA alphavirus backbone is present), 20 MHC class I epitope are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to the RNA alphavirus backbone. Examples of linking the 3′ end of the antigen cassette to the RNA alphavirus backbone include linking directly to the 3′ UTR elements provided by the RNA alphavirus backbone, such as a 3′ 19-nt CSE. Examples of linking the 5′ end of the antigen cassette to the RNA alphavirus backbone include linking directly to a 26S promoter sequence, an alphavirus 5′ UTR, a 51-nt CSE, or a 24-nt CSE.

Other examples include: where a=1 describing where a promoter other than the promoter nucleotide sequence provided by the RNA alphavirus backbone is present; where a=1 and Z is greater than 1 where multiple promoters other than the promoter nucleotide sequence provided by the RNA alphavirus backbone are present each driving expression of 1 or more distinct MHC class I epitope encoding nucleic acid sequences; where h=1 describing where a separate promoter is present to drive expression of the MHC class II antigen-encoding nucleic acid sequences; and where g=0 describing the MHC class II antigen-encoding nucleic acid sequence, if present, is directly linked to the RNA alphavirus backbone.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

The promoter nucleotide sequences P and/or P2 can be the same as a promoter nucleotide sequence provided by the RNA alphavirus backbone. For example, the promoter sequence provided by the RNA alphavirus backbone, Pn and P2, can each comprise a 26S subgenomic promoter. The promoter nucleotide sequences P and/or P2 can be different from the promoter nucleotide sequence provided by the RNA alphavirus backbone, as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encodes a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

### V.B. Immune Checkpoints

Vectors described herein, such as C68 vectors described herein or alphavirus vectors described herein, can comprise a nucleic acid which encodes at least one antigen and the same or a separate vector can comprise a nucleic acid which encodes at least one immune modulator (e.g., an antibody such as an scFv) which binds to and blocks the activity of an immune checkpoint molecule. Vectors can comprise an antigen cassette and one or more nucleic acid molecules encoding a checkpoint inhibitor.

Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal Antibody (Anti-B7-H1; MED14736), ipilimumab, MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Antibody-encoding sequences can be engineered into vectors such as C68 using ordinary skill in the art. An exemplary method is described in Fang et al., Stable antibody expression at therapeutic levels using the 2A peptide. Nat Biotechnol. 2005 May; 23(5):584-90. Epub 2005 Apr. 17; herein incorporated by reference for all purposes.

### V.C. Additional Considerations for Vaccine Design and Manufacture

### V.C.1. Determination of a Set of Peptides that Cover all Tumor Subclones

Truncal peptides, meaning those presented by all or most tumor subclones, can be prioritized for inclusion into the vaccine.⁵³ Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by the vaccine.⁵⁴

### V.C.2. Antigen Prioritization

After all of the above antigen filters are applied, more candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that a tumor will escape immune attack via downregulation or mutation of HLA molecules)
7. Coverage of HLA classes (covering both HLA-I and HLA-II may increase the probability of therapeutic response and decrease the probability of tumor escape)

Additionally, optionally, antigens can be deprioritized (e.g., excluded) from the vaccination if they are predicted to be presented by HLA alleles lost or inactivated in either all or part of the patient's tumor. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### V.D. Alphavirus

### V.D.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbrial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbrial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### V.D.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying RNA (samRNA) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly illicit an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, elicits a robust immune response to antigen, does not elicit an immune response to the vector itself, and can be used in a safe manner. Furthermore, the antigen expression cassette can be designed to elicit different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### V.D.3. Alphavirus Production In Vitro

Alphavirus delivery vectors are generally positive-sense RNA polynucleotides. A convenient technique well-known in the art for RNA production is in vitro transcription IVT. In this technique, a DNA template of the desired vector is first produced by techniques well-known to those in the art, including standard molecular biology techniques such as cloning, restriction digestion, ligation, gene synthesis, and polymerase chain reaction (PCR). The DNA template contains a RNA polymerase promoter at the 5′ end of the sequence desired to be transcribed into RNA. Promoters include, but are not limited to, bacteriophage polymerase promoters such as T3, T7, or SP6. The DNA template is then incubated with the appropriate RNA polymerase enzyme, buffer agents, and nucleotides (NTPs). The resulting RNA polynucleotide can optionally be further modified including, but limited to, addition of a 5′ cap structure such as 7-methylguanosine or a related structure, and optionally modifying the 3′ end to include a polyadenylate (polyA) tail. The RNA can then be purified using techniques well-known in the field, such as phenol-chloroform extraction.

### V.D.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid eliciting immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluable vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target affects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

### V.E. Chimpanzee Adenovirus (ChAd

### V.E.1. Viral Delivery with Chimpanzee Adenovirus

Vaccine compositions for delivery of one or more antigens (e.g., via an antigen cassette and including one or more neoantigens shown in Table A and/or AACR GENIE Results, and/or one or more antigens shown in Table 1.2) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for eliciting an immune response in a mammalian host to treat cancer. The method can comprise the step of administering to the host an effective amount of a recombinant chimpanzee adenovirus, such as C68, comprising an antigen cassette that encodes one or more antigens from the tumor against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus ETA, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of ETA, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO: 1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

The partially deleted E4 gene can be the E4 gene sequence shown in SEQ ID NO:1 that lacks at least nucleotides 34,916 to 35,642 of the sequence shown in SEQ ID NO:1. The partially deleted E4 gene can be the E4 gene sequence shown in SEQ ID NO:1 that lacks the E4 gene sequence shown in SEQ ID NO:1 and that lacks at least nucleotides 34,916 to 34,942, nucleotides 34,952 to 35,305 of the sequence shown in SEQ ID NO: 1, and nucleotides 35,302 to 35,642 of the sequence shown in SEQ ID NO:1. The partially deleted E4 gene can be the E4 gene sequence shown in SEQ ID NO:1 and that lacks at least nucleotides 34,980 to 36,516 of the sequence shown in SEQ ID NO:1. The partially deleted E4 gene can be the E4 gene sequence shown in SEQ ID NO:1 and that lacks at least nucleotides 34,979 to 35,642 of the sequence shown in SEQ ID NO:1. The adenovirus vector having the partially deleted E4 gene can have a cassette, wherein the cassette comprises at least one payload nucleic acid sequence, and wherein the cassette comprises at least one promoter sequence operably linked to the at least one payload nucleic acid sequence. The adenovirus vector having the partially deleted E4 gene can have one or more genes or regulatory sequences of the ChAdV68 sequence shown in SEQ ID NO: 1, optionally wherein the one or more genes or regulatory sequences comprise at least one of the chimpanzee adenovirus inverted terminal repeat (ITR), E1A, E1B, E2A, E2B, E3, E4, L1, L2, L3, L4, and L5 genes of the sequence shown in SEQ ID NO: 1. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, and optionally the nucleotides 2 to 34,916 additionally lack nucleotides 577 to 3403 of the sequence shown in SEQ ID NO:1 corresponding to an E1 deletion and/or lack nucleotides 27,125 to 31,825 of the sequence shown in SEQ ID NO:1 corresponding to an E3 deletion. The adenovirus vector having the partially deleted E4 gene can have nucleotides 35,643 to 36,518 of the sequence shown in SEQ ID NO:1, and wherein the partially deleted E4 gene is 5′ of the nucleotides 35,643 to 36,518. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

The partially deleted E4 gene can be the E4 gene sequence shown in SEQ ID NO:1 that lacks at least nucleotides 34,916 to 35,642 of the sequence shown in SEQ ID NO:1, nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein, such as a ChAd vector or self-amplifying RNA vector engineered to express an antigen cassette.

Also disclosed herein is a method for producing an antigen comprising introducing a vector disclosed herein into a mammalian cell, culturing the cell under suitable conditions and producing the antigen.

### V.E.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

### V.E.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### V.E.4. Construction of the Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### V.E.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### V.E.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene Ela and delayed early gene Elb, so as to eliminate their normal biological functions. Replication-defective E1-deleted viruses are capable of replicating and producing infectious virus when grown on a chimpanzee adenovirus-transformed, complementation cell line containing functional adenovirus Ela and Elb genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### V.E.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### V.E.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### V.E.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a pharmaceutically effective amount, that is, an amount of recombinant adenovirus that is effective in a route of administration to transfect the desired cells and provide sufficient levels of expression of the selected gene to provide a vaccinal benefit, i.e., some measurable level of immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of inducing a tumor specific immune response in a subject, vaccinating against a tumor, treating and or alleviating a symptom of cancer in a subject by administering to the subject one or more antigens such as a plurality of antigens identified using methods disclosed herein.

In some aspects, a subject has been diagnosed with cancer or is at risk of developing cancer. A subject can have been previously treated for cancer, such as previously undergone surgery to remove a tumor and/or cancerous tissue, chemotherapy, immunotherapy (e.g., immune checkpoint inhibitor therapy), radiation therapy, or combinations thereof. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

An antigen can be administered in an amount sufficient to induce a CTL response.

An antigen can be administered alone or in combination with other therapeutic agents. The therapeutic agent is for example, a chemotherapeutic agent, radiation, or immunotherapy. Any suitable therapeutic treatment for a particular cancer can be administered. A therapeutically effective amount of the therapeutic agent can be administered. An amount of the therapeutic agent can be administered that alone is not generally considered a therapeutically effective amount but demonstrates a beneficial property when co-administered with any of the vaccine compositions described herein.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA-4 or PD-L1 by antibodies can enhance the immune response to cancerous cells in the patient. In particular, CTLA-4 blockade has been shown effective when following a vaccination protocol.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, cancer, and/or patient-specific. For instance, the exact selection of peptides can be guided by expression patterns of the parent proteins in a given tissue or guided by mutation status of a patient. The selection can be dependent on the specific type of cancer, the status of the disease, earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for cancer, antigens with similar normal self-peptides that are expressed in high amounts in normal tissues can be avoided or be present in low amounts in a composition described herein. On the other hand, if it is known that the tumor of a patient expresses high amounts of a certain antigen, the respective pharmaceutical composition for treatment of this cancer can be present in high amounts and/or more than one antigen specific for this particularly antigen or pathway of this antigen can be included.

Compositions comprising an antigen can be administered to an individual already suffering from cancer. In therapeutic applications, compositions are administered to a patient in an amount sufficient to stimulate an immune response, such as eliciting an effective CTL response to the tumor antigen and to cure or at least partially arrest symptoms and/or complications. An immune response can include a reduction in tumor size or volume. Reduction in tumor size or volume can include at least a 5%, at least a 10%, at least a 15%, at least a 20%, at least a 25%, at least a 30%, at least a 35%, at least a 40%, at least a 45%, at least a 50%, at least a 55%, at least a 60%, at least a 65%, at least a 70%, at least a 75%, at least a 80%, at least a 85%, at least a 90%, or at least a 95% reduction. Reduction in tumor size or volume can include at least a 15% reduction. Reduction in tumor size or volume can include at least a 20% reduction. An immune response can include stabilization of tumor size or volume. An immune response can result in amelioration of a subject's disease, such a complete response (CR), partial response (PR), or stable disease (SD) (e.g., as assessed by criteria set forth in a clinical study). An amount adequate to accomplish this is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of an antigen, it is possible and can be felt desirable by the treating physician to administer substantial excesses of these compositions.

For therapeutic use, administration can begin at the detection or surgical removal of tumors. This is followed by boosting doses until at least symptoms are substantially abated and for a period thereafter.

Compositions comprising an antigen (e.g., any of the compositions for delivery of a self-replicating alphavirus-based expression system or a chimpanzee adenovirus (ChAdV)-based expression system described herein) can be administered as an adjuvant therapy to a subject having already received a primary therapy. Compositions comprising an antigen can be administered 1, 2, 3, 4, 5, 6, 7, 8, 9, 10, 11, 12, 13, 14 days following a primary therapy, or 1, 2, 3, 4, 5, 6, 7, 8, 9, or 10 or more weeks following a primary therapy. For example, compositions comprising an antigen can be administered as an adjuvant therapy following surgery to remove tumors and/or cancerous tissues, including 1, 2, 3, 4, 5, 6, 7, 8, 9, 10, 11, 12, 13, 14, days following surgery, or 1, 2, 3, 4, 5, 6, 7, 8, 9, or 10 or more weeks following surgery. Compositions comprising an antigen can be administered as an adjuvant therapy as a combination therapy with an additional therapy, such as administered in combination with chemotherapy, immune checkpoint inhibitor therapy, radiation therapy, or combinations thereof.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at the site of surgical excision to induce a local immune response to the tumor. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a tumor vaccine, comprising performing the steps of a method disclosed herein; and producing a tumor vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VI. Antigen Use and Administration

A vaccination protocol can be used to dose a subject with one or more antigens. A priming vaccine and a boosting vaccine can be used to dose the subject. The priming vaccine can be based on C68 (e.g., the sequences shown in SEQ ID NO:1 or 2) or srRNA (e.g., the sequences shown in SEQ ID NO:3 or 4) and the boosting vaccine can be based on C68 (e.g., the sequences shown in SEQ ID NO:1 or 2) or srRNA (e.g., the sequences shown in SEQ ID NO:3 or 4). Each vector typically includes a cassette that includes antigens. Cassettes can include about 20 antigens, separated by spacers such as the natural sequence that normally surrounds each antigen or other non-natural spacer sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) a checkpoint inhibitor (CPI). CPI's can include those that inhibit CTLA4, PD1, and/or PDL1 such as antibodies or antigen-binding portions thereof. Such antibodies can include tremelimumab or durvalumab.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of self-amplifying RNA (SAM) at low vaccine dose selected from the range 0.001 to 1 ug RNA, in particular 0.1 or 1 ug can be used; or one or more injections of SAM at high vaccine dose selected from the range 1 to 1000 ug RNA, in particular 30 μg, 100 μg, or 300 μg RNA can be used. For ChAdV68 priming, 1×10¹² or less of viral particles can be administered. For ChAdV68 priming, 3×10¹¹ or less of the viral particles can be administered. For ChAdV68 priming, at least 1×10¹¹ of the viral particles can be administered. For ChAdV68 priming, between 1×10¹¹ and 1×10¹², between 3×10¹¹ and 1×10¹², or between 1×10¹¹ and 3×10¹¹ of the viral particles can be administered. For ChAdV68 priming, 1×10¹¹, 3×10¹¹, or 1×10¹² of the viral particles can be administered. For ChAdV68 priming, the viral particles can be at a concentration of at 5×10¹¹ vp/mL.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. Bilateral injections per dose can be used. For example, one or more injections of ChAdV68 (C68) can be used (e.g., total dose 1×10¹² viral particles); one or more injections of self-amplifying RNA (SAM) at low vaccine dose selected from the range 0.001 to 1 ug RNA, in particular 0.1 or 1 ug can be used; or one or more injections of SAM at high vaccine dose selected from the range 1 to 100 μg RNA, in particular 10 or 100 ug can be used. A SAM boost of between 10-30 μg, 10-100 μg, 10-300 μg, 30-100 μg, 30-300 μg, or 100-300 μg RNA can be administered. A SAM boost of between 10-500 μg, 10-1000 μg, 30-500 μg, 30-1000 μg, or 500-1000 μg RNA can be administered. A SAM boost of at least 400 μg, at least 500 μg, at least 600 μg, at least 700 μg, at least 800 μg, at least 900 μg, at least 1000 μg RNA can be administered. A SAM boost of 10 μg, 30 μg, 100 μg, or 300 μg RNA can be administered. A SAM boost of 300 μg RNA can be administered. A SAM boost of 100 μg RNA can be administered. A SAM boost of 30 μg RNA can be administered. A SAM boost of 10 μg RNA can be administered. A SAM boost of at least 300 μg RNA can be administered. A SAM boost of at least 100 μg RNA can be administered. A SAM boost of at least 30 μg RNA can be administered. A SAM boost of at least 10 μg RNA can be administered. A SAM boost of less than or equal to 300 μg RNA can be administered.

Anti-CTLA-4 (e.g., tremelimumab) can also be administered to the subject. For example, anti-CTLA4 can be administered subcutaneously near the site of the intramuscular vaccine injection (ChAdV68 prime or srRNA low doses) to ensure drainage into the same lymph node. Tremelimumab is a selective human IgG2 mAb inhibitor of CTLA-4. Target Anti-CTLA-4 (tremelimumab) subcutaneous dose is typically 70-75 mg (in particular 75 mg) with a dose range of, e.g., 1-100 mg or 5-420 mg.

In certain instances an anti-PD-L1 antibody can be used such as durvalumab (MEDI 4736). Durvalumab is a selective, high affinity human IgG1 mAb that blocks PD-L1 binding to PD-1 and CD80. Durvalumab is generally administered at 20 mg/kg i.v. every 4 weeks.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

T cell responses can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA).

Disease status of a subject can be monitored following administration of any of the vaccine compositions described herein. For example, disease status may be monitored using isolated cell-free DNA (cfDNA) from a subject. In addition, the efficacy of a vaccine therapy may be monitored using isolated cfDNA from a subject. cfDNA monitoring can include the steps of: a. isolating or having isolated cfDNA from a subject; b. sequencing or having sequenced the isolated cfDNA; c. determining or having determined a frequency of one or more mutations in the cfDNA relative to a wild-type germline nucleic acid sequence of the subject, and d. assessing or having assessed from step (c) the status of a disease in the subject. The method can also include, following step (c) above, d. performing more than one iteration of steps (a)-(c) for the given subject and comparing the frequency of the one or more mutations determined in the more than one iterations; and f. assessing or having assessed from step (d) the status of a disease in the subject. The more than one iterations can be performed at different time points, such as a first iteration of steps (a)-(c) performed prior to administration of the vaccine composition and a second iteration of steps (a)-(c) is performed subsequent to administration of the vaccine composition. Step (c) can include comparing: the frequency of the one or more mutations determined in the more than one iterations, or the frequency of the one or more mutations determined in the first iteration to the frequency of the one or more mutations determined in the second iteration. An increase in the frequency of the one or more mutations determined in subsequent iterations or the second iteration can be assessed as disease progression. A decrease in the frequency of the one or more mutations determined in subsequent iterations or the second iteration can be assessed as a response. In some aspects, the response is a Complete Response (CR) or a Partial Response (PR). A therapy can be administered to a subject following an assessment step, such as where assessment of the frequency of the one or more mutations in the cfDNA indicates the subject has the disease. The cfDNA isolation step can use centrifugation to separate cfDNA from cells or cellular debris. cfDNA can be isolated from whole blood, such as by separating the plasma layer, buffy coat, and red bloods. cfDNA sequencing can use next generation sequencing (NGS), Sanger sequencing, duplex sequencing, whole-exome sequencing, whole-genome sequencing, de novo sequencing, phased sequencing, targeted amplicon sequencing, shotgun sequencing, or combinations thereof, and may include enriching the cfDNA for one or more polynucleotide regions of interest prior to sequencing (e.g., polynucleotides known or suspected to encode the one or more mutations, coding regions, and/or tumor exome polynucleotides). Enriching the cfDNA may include hybridizing one or more polynucleotide probes, which may be modified (e.g., biotinylated), to the one or more polynucleotide regions of interest. In general, any number of mutations may be monitored simultaneously or in parallel.

### VIII. Antigen Identification

### VIII.A. Antigen Candidate Identification

Research methods for NGS analysis of tumor and normal exome and transcriptomes have been described and applied in the antigen identification space.^{6,14,15} Certain optimizations for greater sensitivity and specificity for antigen identification in the clinical setting can be considered. These optimizations can be grouped into two areas, those related to laboratory processes and those related to the NGS data analysis. Examples of optimizations are known to those skilled in the art, for example the methods described in more detail in international patent application publications WO/2017/106638, WO/2018/195357, and WO/2018/208856, each herein incorporated by reference, in their entirety, for all purposes.

### VIII.B. Isolation and Detection of HLA Peptides

Isolation of HLA-peptide molecules was performed using classic immunoprecipitation (IP) methods after lysis and solubilization of the tissue sample (55-58). A clarified lysate was used for HLA specific IP.

Immunoprecipitation was performed using antibodies coupled to beads where the antibody is specific for HLA molecules. For a pan-Class I HLA immunoprecipitation, a pan-Class I CR antibody is used, for Class II HLA-DR, an HLA-DR antibody is used. Antibody is covalently attached to NHS-sepharose beads during overnight incubation. After covalent attachment, the beads were washed and aliquoted for IP. (59, 60) Immunoprecipitations can also be performed with antibodies that are not covalently attached to beads. Typically this is done using sepharose or magnetic beads coated with Protein A and/or Protein G to hold the antibody to the column. Some antibodies that can be used to selectively enrich MHC/peptide complex are listed below.

| Antibody Name | Specificity |
|---|---|
| W6/32 | Class I HLA-A, B, C |
| L243 | Class II-HLA-DR |
| Tu36 | Class II-HLA-DR |
| LN3 | Class II-HLA-DR |
| Tu39 | Class II-HLA-DR, DP, DQ |
| SPVL3 | Class II-HLA-DQ |
| B7/21 | Class II-HLA-DP |

The clarified tissue lysate is added to the antibody beads for the immunoprecipitation. After immunoprecipitation, the beads are removed from the lysate and the lysate stored for additional experiments, including additional IPs. The IP beads are washed to remove non-specific binding and the HLA/peptide complex is eluted from the beads using standard techniques. The protein components are removed from the peptides using a molecular weight spin column or C18 fractionation. The resultant peptides are taken to dryness by SpeedVac evaporation and in some instances are stored at −20C prior to MS analysis. HLA IPs can also be performed in 96 well plate format using plates that contain filter bottoms. Use of the plates allows for multiple IPs to be performed in tandem.

Dried peptides are reconstituted in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 low resolution scans collected in the ion trap detector after HCD fragmentation of the selected ion. Additionally, MS2 spectra can be obtained using either CID or ETD fragmentation methods or any combination of the three techniques to attain greater amino acid coverage of the peptide. MS2 spectra can also be measured with high resolution mass accuracy in the Orbitrap detector. MS2 spectra from each analysis are searched against a protein database using Comet (61, 62) and the peptide identification are scored using Percolator (63-65). Additional sequencing is performed using PEAKS studio (Bioinformatics Solutions Inc.) and other search engines or sequencing methods can be used including spectral matching and de novo sequencing (97).

### VIII.B.1. MS Limit of Detection Studies in Support of Comprehensive HLA Peptide Sequencing

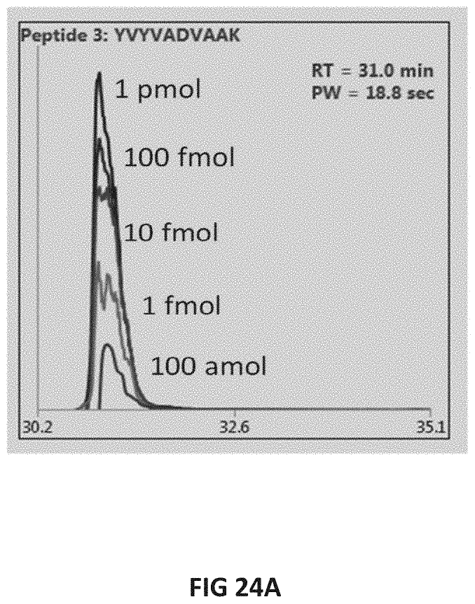

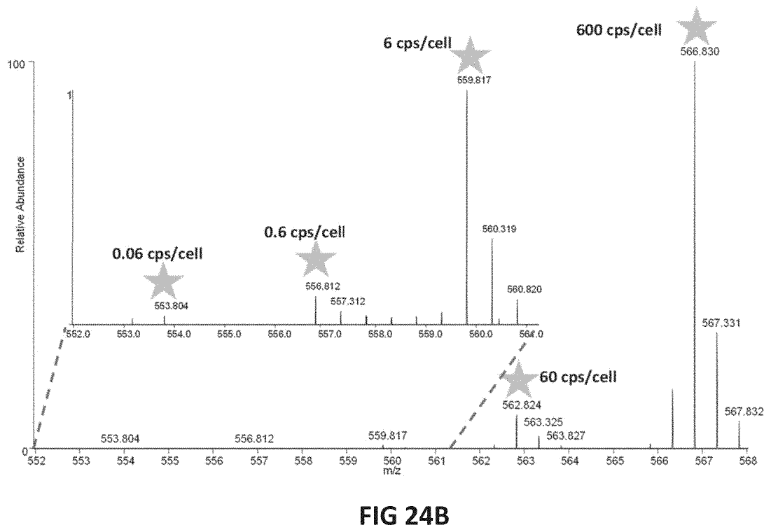

Using the peptide YVYVADVAAK (SEQ ID NO: 29364) it was determined what the limits of detection are using different amounts of peptide loaded onto the LC column. The amounts of peptide tested were 1 pmol, 100 fmol, 10 fmol, 1 fmol, and 100 amol. (Table 1) The results are shown in FIGS. 24A and 24B. These results indicate that the lowest limit of detection (LoD) is in the attomol range (10⁻¹⁸), that the dynamic range spans five orders of magnitude, and that the signal to noise appears sufficient for sequencing at low femtomol ranges (10⁻¹⁵).

TABLE 1

| Peptide m/z | Loaded on Column | Copies/Cell in 1e9cells |
|---|---|---|
| 566.830 | 1 pmol | 600 |
| 562.823 | 100 fmol | 60 |
| 559.816 | 10 fmol | 6 |
| 556.810 | 1 fmol | 0.6 |
| 553.802 | 100 amol | 0.06 |

### IX. Presentation Model

Presentation models can be used to identify likelihoods of peptide presentation in patients. Various presentation models are known to those skilled in the art, for example the presentation models described in more detail in international patent application publications WO/2017/106638, WO/2018/195357, WO/2018/208856, WO2016187508, and US patent application US20110293637, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in international patent application publications WO/2017/106638, WO/2018/195357, and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from tumor tissue cells of patients. A prediction module may identify candidate neoantigens that are mutated peptide sequences by comparing sequence data extracted from normal tissue cells of a patient with the sequence data extracted from tumor tissue cells of the patient to identify portions containing one or more mutations. A prediction module may identify candidate antigens that have altered expression in a tumor cell or cancerous tissue in comparison to a normal cell or tissue by comparing sequence data extracted from normal tissue cells of a patient with the sequence data extracted from tumor tissue cells of the patient to identify improperly expressed candidate antigens.

A presentation module can apply one or more presentation model to processed peptide sequences to estimate presentation likelihoods of the peptide sequences. Specifically, the prediction module may select one or more candidate antigen peptide sequences that are likely to be presented on tumor HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where Nis generally the maximum number of epitopes that can be delivered in a vaccine). A vaccine including the selected candidate antigens for a given patient can be injected into the patient to induce immune responses.

### XI.B. Cassette Design Module

### XI.B.1 Overview

A cassette design module can be used to generate a vaccine cassette sequence based on selected candidate peptides for injection into a patient. Various cassette design modules are known to those skilled in the art, for example the cassette design modules described in more detail in international patent application publications WO/2017/106638, WO/2018/195357, and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in international patent application publications WO/2017/106638, WO/2018/195357, and WO/2018/208856, each herein incorporated by reference, in their entirety, for all purposes.

### XI.B.2 Shared Antigen Vaccine Sequence Selection

Shared antigen sequences for inclusion in a shared antigen vaccine and appropriate patients for treatment with such vaccine can be chosen by one of skill in the art using the detailed disclosure provided herein. For example, Tables: A, 1.2, Additional MS Validated Neoantigens, or AACR GENIE Results can be used for sequence selection. In certain instances a particular mutation and HLA allele combination can be preferred (e.g., based on sequencing data available from a given subject indicating that each are present in the subject) and subsequently used in combination together to identify a shared neoantigen sequence using Table A, Additional MS Validated Neoantigens, or AACR GENIE Results for inclusion in a vaccine. Exemplary mutations and their matched HLA alleles are shown in Tables 32A, 32B, and 34.

For example, for KRAS_G13D, a shared neoantigen or shared neoantigen-encoding sequence for inclusion in a vaccine can be selected by reference to Table A or AACR GENIE Results, where each relevant sequence considered for inclusion is selected by identifying all rows that list KRAS_G13D and C0802 and A1101.

For example, for KRAS_Q61K or NRAS_Q61K, a shared neoantigen or shared neoantigen-encoding sequence for inclusion in a vaccine can be selected by reference to Table A or AACR GENIE Results, where each relevant sequence considered for inclusion is selected by identifying all rows that list (1) KRAS_Q61K and A0101; or (2) NRAS Q61K, and A0101.

For example, for TP53_R249M, a shared neoantigen or shared neoantigen-encoding sequence for inclusion in a vaccine can be selected by reference to Table A or AACR GENIE Results, where each relevant sequence considered for inclusion is selected by identifying all rows that list TP53_R249M and at least one of B3512, B3503, and B3501.

For example, for CTNNB1_S45P, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list CTNNB1_S45P and at least one of A0301, A6801, A0302, and A1101. For example, see relevant sequences shown in Table 32A and Table 32B.

For example, for CTNNB1_S45F, a shared neoantigen or shared neoantigen-encoding sequence for inclusion in a vaccine can be selected by reference to Table A or AACR GENIE Results, where each relevant sequence considered for inclusion is selected by identifying all rows that list CTNNB1_S45F and at least one of A0301, A1101, and A6801.

For example, for ERBB2_Y772_A775dup, a shared neoantigen or shared neoantigen-encoding sequence for inclusion in a vaccine can be selected by reference to Table A or AACR GENIE Results, where each relevant sequence considered for inclusion is selected by identifying all rows that list ERBB2_Y772_A775dup and B1801.

For example, for KRAS_G12D or NRAS_G12D, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list (1) KRAS_G12D and at least one of A1101, A0301, and C0802; or (2) NRAS_G12D and at least one of A1101, A0301, and C0802. For example, see relevant sequences shown in Table 32A or Table 32B.

For example, for KRAS_Q61R or NRAS_Q61R, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list (1) KRAS_Q61R and A0101; or (2) NRAS_Q61R and A0101. For example, see relevant sequence shown in Table 32B.

For example, for CTNNB1_T41A, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list CTNNB1_T41A and at least one of A0301, A0302, A1101, B1510, C0303, and C0304. For example, see relevant sequence shown in Table 32B.

For example, for TP53_K132N, a shared neoantigen or shared neoantigen-encoding sequence for inclusion in a vaccine can be selected by reference to Table A or AACR GENIE Results, where each relevant sequence considered for inclusion is selected by identifying all rows that list TP53_K132N and at least one of A2402 and A2301. For example, see relevant sequence shown in Table 32A.

For example, for KRAS_G12A, a shared neoantigen or shared neoantigen-encoding sequence for inclusion in a vaccine can be selected by reference to Table A or AACR GENIE Results, where each relevant sequence considered for inclusion is selected by identifying all rows that list KRAS_G12A and at least one of A0301 and A1101.

For example, for KRAS_Q61L or NRAS_Q61L, a shared neoantigen or shared neoantigen-encoding sequence for inclusion in a vaccine can be selected by reference to Table A or AACR GENIE Results, where each relevant sequence considered for inclusion is selected by identifying all rows that list (1) KRAS_Q61L and A0101; or (2) NRAS_Q61L and A0101.

For example, for TP53_R213L, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list TP53_R213L and at least one of A0207, C0802, and A0201. For example, see relevant sequence shown in Table 32B.

For example, for BRAF_G466V, a shared neoantigen or shared neoantigen-encoding sequence for inclusion in a vaccine can be selected by reference to Table A or AACR GENIE Results, where each relevant sequence considered for inclusion is selected by identifying all rows that list BRAF_G466V and at least one of B1501 and B1503.

For example, for KRAS_G12V, a shared neoantigen or shared neoantigen-encoding sequence for inclusion in a vaccine can be selected by reference to Table A, Additional MS Validated Neoantigens, or AACR GENIE Results, where each relevant sequence considered for inclusion is selected by identifying all rows that list KRAS_G12V and at least one of A0301, A1101, C0102, and A0302. For example, see relevant sequences shown in Table 32A and Table 32B.

For example, for KRAS_Q61H or NRAS_Q61H, a shared neoantigen or shared neoantigen-encoding sequence for inclusion in a vaccine can be selected by reference to Table A or AACR GENIE Results, where each relevant sequence considered for inclusion is selected by identifying all rows that list (1) KRAS_Q61H and A0101; or (2) NRAS_Q61H and A0101.

For example, for CTNNB1_S37F, a shared neoantigen or shared neoantigen-encoding sequence for inclusion in a vaccine can be selected by reference to Table A or AACR GENIE Results, where each relevant sequence considered for inclusion is selected by identifying all rows that list CTNNB1_S37F and at least one of A0101, A2301, A2402, B1510, B3906, C0501, C1402, and C1403.

For example, for TP53_S127Y, a shared neoantigen or shared neoantigen-encoding sequence for inclusion in a vaccine can be selected by reference to Table A or AACR GENIE Results, where each relevant sequence considered for inclusion is selected by identifying all rows that list TP53_S127Y and at least one of A1101 and A0301.

For example, for TP53_K132E, a shared neoantigen or shared neoantigen-encoding sequence for inclusion in a vaccine can be selected by reference to Table A or AACR GENIE Results, where each relevant sequence considered for inclusion is selected by identifying all rows that list TP53_K132E and at least one of A2402, C1403, and A2301.

For example, for KRAS_G12C or NRAS_G12C, a shared neoantigen or shared neoantigen-encoding sequence for inclusion in a vaccine can be selected by reference to Table A or AACR GENIE Results, where each relevant sequence considered for inclusion is selected by identifying all rows that list (1) KRAS_G12C and at least one of A0201, A0301, and A1101; or (2) NRAS_G12C and at least one of A0201, A0301, and A1101. For example, see relevant sequences shown in Table 32A.

### XII. Example Computer

A computer can be used for any of the computational methods described herein. One skilled in the art will recognize a computer can have different architectures. Examples of computers are known to those skilled in the art, for example the computers described in more detail in international patent application publications WO/2017/106638, WO/2018/195357, and WO/2018/208856, each herein incorporated by reference, in their entirety, for all purposes.

### XIV. Antigen Delivery Vector Example

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### XIV.A. Neoantigen Cassette Design

Through vaccination, multiple class I MHC restricted tumor-specific neoantigens (TSNAs) that stimulate the corresponding cellular immune response(s) can be delivered. In one example, a vaccine cassette was engineered to encode multiple epitopes as a single gene product where the epitopes were either embedded within their natural, surrounding peptide sequence or spaced by non-natural linker sequences. Several design parameters were identified that could potentially impact antigen processing and presentation and therefore the magnitude and breadth of the TSNA specific CD8 T cell responses. In the present example, several model cassettes were designed and constructed to evaluate: (1) whether robust T cell responses could be generated to multiple epitopes incorporated in a single expression cassette; (2) what makes an optimal linker placed between the TSNAs within the expression cassette—that leads to optimal processing and presentation of all epitopes; (3) if the relative position of the epitopes within the cassette impact T cell responses; (4) whether the number of epitopes within a cassette influences the magnitude or quality of the T cell responses to individual epitopes; (5) if the addition of cellular targeting sequences improves T cell responses.

Two readouts were developed to evaluate antigen presentation and T cell responses specific for marker epitopes within the model cassettes: (1) an in vitro cell-based screen which allowed assessment of antigen presentation as gauged by the activation of specially engineered reporter T cells (Aarnoudse et al., 2002; Nagai et al., 2012); and (2) an in vivo assay that used HLA-A2 transgenic mice (Vitiello et al., 1991) to assess post-vaccination immunogenicity of cassette-derived epitopes of human origin by their corresponding epitope-specific T cell responses (Comet et al., 2006; Depla et al., 2008; Ishioka et al., 1999).

### XIV.B. Antigen Cassette Design Evaluation

### XIV.B.1. Methods and Materials

### TCR and Cassette Design and Cloning

The selected TCRs recognize peptides NLVPMVATV (SEQ ID NO: 29365) (PDB #5D2N), CLGGLLTMV (SEQ ID NO: 29366) (PDB #3REV), GILGFVFTL (SEQ ID NO: 29367) (PDB #1OGA) LLFGYPVYV (SEQ ID NO: 29368) (PDB #1AO7) when presented by A*0201. Transfer vectors were constructed that contain 2A peptide-linked TCR subunits (beta followed by alpha), the EMCV IRES, and 2A-linked CD8 subunits (beta followed by alpha and by the puromycin resistance gene). Open reading frame sequences were codon-optimized and synthesized by GeneArt.

### Cell Line Generation for In Vitro Epitope Processing and Presentation Studies

Peptides were purchased from ProImmune or Genscript diluted to 10 mg/mL with 10 mM tris(2-carboxyethyl)phosphine (TCEP) in water/DMSO (2:8, v/v). Cell culture medium and supplements, unless otherwise noted, were from Gibco. Heat inactivated fetal bovine serum (FBShi) was from Seradigm. QUANTI-Luc Substrate, Zeocin, and Puromycin were from InvivoGen. Jurkat-Lucia NFAT Cells (InvivoGen) were maintained in RPMI 1640 supplemented with 10% FBShi, Sodium Pyruvate, and 100 μg/mL Zeocin. Once transduced, these cells additionally received 0.3 μg/mL Puromycin. T2 cells (ATCC CRL-1992) were cultured in Iscove's Medium (IMDM) plus 20% FBShi. U-87 MG (ATCC HTB-14) cells were maintained in MEM Eagles Medium supplemented with 10% FBShi.

Jurkat-Lucia NFAT cells contain an NFAT-inducible Lucia reporter construct. The Lucia gene, when activated by the engagement of the T cell receptor (TCR), causes secretion of a coelenterazine-utilizing luciferase into the culture medium. This luciferase can be measured using the QUANTI-Luc luciferase detection reagent. Jurkat-Lucia cells were transduced with lentivirus to express antigen-specific TCRs. The HIV-derived lentivirus transfer vector was obtained from GeneCopoeia, and lentivirus support plasmids expressing VSV-G (pCMV-VsvG), Rev (pRSV-Rev) and Gag-pol (pCgpV) were obtained from Cell Design Labs.

Lentivirus was prepared by transfection of 50-80% confluent T75 flasks of HEK293 cells with Lipofectamine 2000 (Thermo Fisher), using 40 μl of lipofectamine and 20 μg of the DNA mixture (4:2:1:1 by weight of the transfer plasmid:pCgpV:pRSV-Rev:pCMV-VsvG). 8-10 mL of the virus-containing media were concentrated using the Lenti-X system (Clontech), and the virus resuspended in 100-200 μl of fresh medium. This volume was used to overlay an equal volume of Jurkat-Lucia cells (5×10E4-1×10E6 cells were used in different experiments). Following culture in 0.3 μg/ml puromycin-containing medium, cells were sorted to obtain clonality. These Jurkat-Lucia TCR clones were tested for activity and selectivity using peptide loaded T2 cells.

### In Vitro Epitope Processing and Presentation Assay

T2 cells are routinely used to examine antigen recognition by TCRs. T2 cells lack a peptide transporter for antigen processing (TAP deficient) and cannot load endogenous peptides in the endoplasmic reticulum for presentation on the MHC. However, the T2 cells can easily be loaded with exogenous peptides. The five marker peptides (NLVPMVATV (SEQ ID NO: 29365), CLGGLLTMV (SEQ ID NO: 29366), GLCTLVAML (SEQ ID NO: 29369), LLFGYPVYV (SEQ ID NO: 29368), GILGFVFTL (SEQ ID NO: 29367)) and two irrelevant peptides (WLSLLVPFV (SEQ ID NO: 29370), FLLTRICT (SEQ ID NO: 29371)) were loaded onto T2 cells. Briefly, T2 cells were counted and diluted to 1×106 cells/mL with IMDM plus 1% FBShi. Peptides were added to result in 10 μg peptide/1×106 cells. Cells were then incubated at 37° C. for 90 minutes. Cells were washed twice with IMDM plus 20% FBShi, diluted to 5×10E5 cells/mL and 100 μL plated into a 96-well Costar tissue culture plate. Jurkat-Lucia TCR clones were counted and diluted to 5×10E5 cells/mL in RPMI 1640 plus 10% FBShi and 100 μL added to the T2 cells. Plates were incubated overnight at 37° C., 5% CO2. Plates were then centrifuged at 400 μg for 3 minutes and 20 μL supernatant removed to a white flat bottom Greiner plate. QUANTI-Luc substrate was prepared according to instructions and 50 μL/well added. Luciferase expression was read on a Molecular Devices SpectraMax iE3×.

To test marker epitope presentation by the adenoviral cassettes, U-87 MG cells were used as surrogate antigen presenting cells (APCs) and were transduced with the adenoviral vectors. U-87 MG cells were harvested and plated in culture media as 5×10E5 cells/100 μl in a 96-well Costar tissue culture plate. Plates were incubated for approximately 2 hours at 37° C. Adenoviral cassettes were diluted with MEM plus 10% FBShi to an MOI of 100, 50, 10, 5, 1 and 0 and added to the U-87 MG cells as 5 μl/well. Plates were again incubated for approximately 2 hours at 37° C. Jurkat-Lucia TCR clones were counted and diluted to 5×10E5 cells/mL in RPMI plus 10% FBShi and added to the U-87 MG cells as 100 μL/well. Plates were then incubated for approximately 24 hours at 37° C., 5% CO2. Plates were centrifuged at 400 μg for 3 minutes and 20 μL supernatant removed to a white flat bottom Greiner plate. QUANTI-Luc substrate was prepared according to instructions and 50 μL/well added. Luciferase expression was read on a Molecular Devices SpectraMax iE3×.

### Mouse Strains for Immunogenicity Studies

Transgenic HLA-A2.1 (HLA-A2 Tg) mice were obtained from Taconic Labs, Inc. These mice carry a transgene consisting of a chimeric class I molecule comprised of the human HLA-A2.1 leader, α1, and α2 domains and the murine H2-Kb α3, transmembrane, and cytoplasmic domains (Vitiello et al., 1991). Mice used for these studies were the first generation offspring (F1) of wild type BALB/cAnNTac females and homozygous HLA-A2.1 Tg males on the C57B1/6 background.

### Adenovirus Vector (Ad5v) Immunizations

HLA-A2 Tg mice were immunized with 1×10¹⁰ to 1×10⁶ viral particles of adenoviral vectors via bilateral intramuscular injection into the tibialis anterior. Immune responses were measured at 12 days post-immunization.

### Lymphocyte Isolation

Lymphocytes were isolated from freshly harvested spleens and lymph nodes of immunized mice. Tissues were dissociated in RPMI containing 10% fetal bovine serum with penicillin and streptomycin (complete RPMI) using the GentleMACS tissue dissociator according to the manufacturer's instructions.

### Ex Vivo Enzyme-Linked Immunospot (ELISpot) Analysis

ELISpot analysis was performed according to ELISpot harmonization guidelines (Janetzki et al., 2015) with the mouse IFNg ELISpotPLUS kit (MABTECH). 1×10⁵ splenocytes were incubated with 10 uM of the indicated peptides for 16 hours in 96-well IFNg antibody coated plates. Spots were developed using alkaline phosphatase. The reaction was timed for 10 minutes and was quenched by running the plate under tap water. Spots were counted using an AID vSpot Reader Spectrum. For ELISpot analysis, wells with saturation >50% were recorded as “too numerous to count”. Samples with deviation of replicate wells >10% were excluded from analysis. Spot counts were then corrected for well confluency using the formula: spot count+2 x (spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### Ex Vivo Intracellular Cytokine Staining (ICS) and Flow Cytometry Analysis

Freshly isolated lymphocytes at a density of 2-5×10⁶ cells/mL were incubated with 10 uM of the indicated peptides for 2 hours. After two hours, brefeldin A was added to a concentration of 5 ug/ml and cells were incubated with stimulant for an additional 4 hours. Following stimulation, viable cells were labeled with fixable viability dye eFluor780 according to manufacturer's protocol and stained with anti-CD8 APC (clone 53-6.7, BioLegend) at 1:400 dilution. Anti-IFNg PE (clone XMG1.2, BioLegend) was used at 1:100 for intracellular staining. Samples were collected on an Attune NxT Flow Cytometer (Thermo Scientific). Flow cytometry data was plotted and analyzed using FlowJo. To assess degree of antigen-specific response, both the percent IFNg+ of CD8+ cells and the total IFNg+ cell number/1×10⁶ live cells were calculated in response to each peptide stimulant.

### XIV.B.2. In Vitro Evaluation of Antigen Cassette Designs

As an example of antigen cassette design evaluation, an in vitro cell-based assay was developed to assess whether selected human epitopes within model vaccine cassettes were being expressed, processed, and presented by antigen-presenting cells (FIG. 1). Upon recognition, Jurkat-Lucia reporter T cells that were engineered to express one of five TCRs specific for well-characterized peptide-HLA combinations become activated and translocate the nuclear factor of activated T cells (NFAT) into the nucleus which leads to transcriptional activation of a luciferase reporter gene. Antigenic stimulation of the individual reporter CD8 T cell lines was quantified by bioluminescence.

Individual Jurkat-Lucia reporter lines were modified by lentiviral transduction with an expression construct that includes an antigen-specific TCR beta and TCR alpha chain separated by a P2A ribosomal skip sequence to ensure equimolar amounts of translated product (Banu et al., 2014). The addition of a second CD8 beta-P2A-CD8 alpha element to the lentiviral construct provided expression of the CD8 co-receptor, which the parent reporter cell line lacks, as CD8 on the cell surface is crucial for the binding affinity to target pMHC molecules and enhances signaling through engagement of its cytoplasmic tail (Lyons et al., 2006; Yachi et al., 2006).

After lentiviral transduction, the Jurkat-Lucia reporters were expanded under puromycin selection, subjected to single cell fluorescence assisted cell sorting (FACS), and the monoclonal populations tested for luciferase expression. This yielded stably transduced reporter cell lines for specific peptide antigens 1, 2, 4, and 5 with functional cell responses. (Table 2).

TABLE 2
Development of an in vitro T cell activation assay. Peptide-specific T
cell recognition as measured by induction of luciferase indicates
effective processing and presentation of the vaccine cassette antigens.

| | Short Cassette Design |
|---|---|
| Epitope | AAY |
| 1 | 24.5 ± 0.5 |
| 2 | 11.3 ± 0.4 |
| 3* | n/a |
| 4 | 26.1 ± 3.1 |
| 5 | 46.3 ± 1.9 |

*Reporter T cell for epitope 3 not yet generated

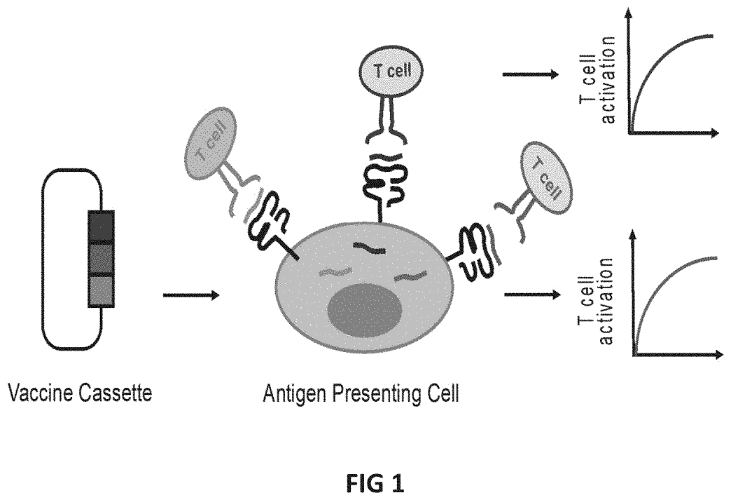

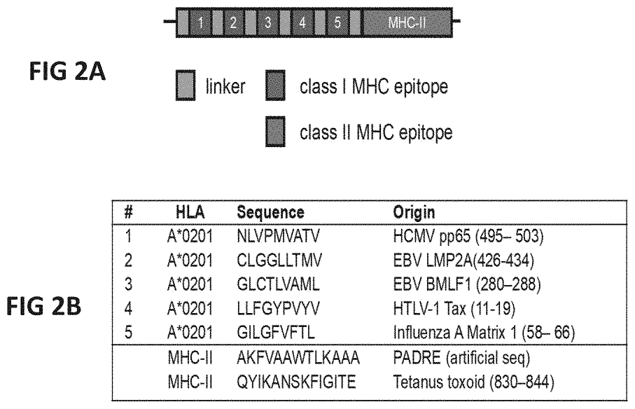

In another example, a series of short cassettes, all marker epitopes were incorporated in the same position (FIG. 2A) and only the linkers separating the HLA-A*0201 restricted epitopes (FIG. 2B) were varied. Reporter T cells were individually mixed with U-87 antigen-presenting cells (APCs) that were infected with adenoviral constructs expressing these short cassettes, and luciferase expression was measured relative to uninfected controls. All four antigens in the model cassettes were recognized by matching reporter T cells, demonstrating efficient processing and presentation of multiple antigens. The magnitude of T cell responses follow largely similar trends for the natural and AAY-linkers. The antigens released from the RR-linker based cassette show lower luciferase inductions (Table 3). The DPP-linker, designed to disrupt antigen processing, produced a vaccine cassette that led to low epitope presentation (Table 3).

TABLE 3
Evaluation of linker sequences in short cassettes. Luciferase induction in the in vitro
T cell activation assay indicated that, apart from the DPP-based cassette, all linkers facilitated
efficient release of the cassette antigens. T cell epitope only (no linker) = 9AA, natural linker
one side = 17AA, natural linker both sides = 25AA, non-natural linkers = AAY, RR, DPP

| | Short Cassette Designs | | | | | |
|---|---|---|---|---|---|---|
| Epitope | 9AA | 17AA | 25AA | AAY | RR | DPP |
| 1 | 33.6 ± 0.9 | 42.8 ± 2.1 | 42.3 ± 2.3 | 24.5 ± 0.5 | 21.7 ± 0.9 | 0.9 ± 0.1 |
| 2 | 12.0 ± 0.9 | 10.3 ± 0.6 | 14.6 ± 04 | 11.3 ± 0.4 | 8.5 ± 0.3 | 1.1 ± 0.2 |
| 3* | n/a | n/a | n/a | n/a | n/a | n/a |
| 4 | 26.6 ± 2.5 | 16.1 ± 0.6 | 16.6 ± 0.8 | 26.1 ± 3.1 | 12.5 ± 0.8 | 1.3 ± 0.2 |
| 5 | 29.7 ± 0.6 | 21.2 ± 0.7 | 24.3 ± 1.4 | 46.3 ± 1.9 | 19.7 ± 0.4 | 1.3 ± 0.1 |

*Reporter T cell for epitope 3 not yet generated

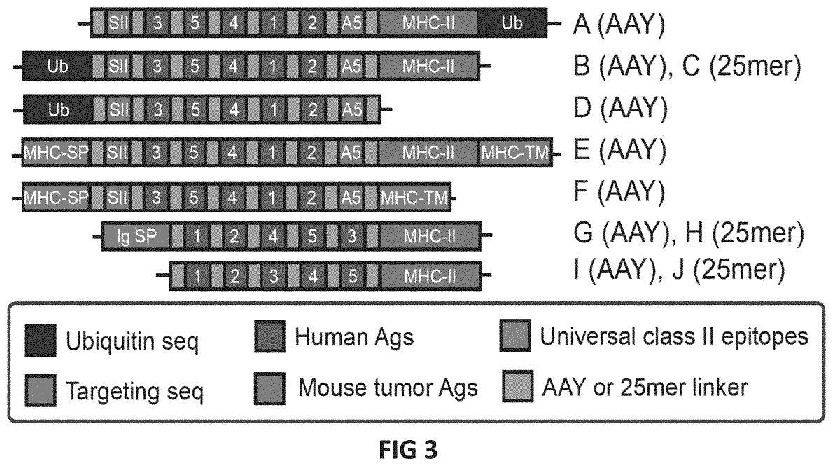

In another example, an additional series of short cassettes were constructed that, besides human and mouse epitopes, contained targeting sequences such as ubiquitin (Ub), MHC and Ig-kappa signal peptides (SP), and/or MHC transmembrane (TM) motifs positioned on either the N- or C-terminus of the cassette. (FIG. 3). When delivered to U-87 APCs by adenoviral vector, the reporter T cells again demonstrated efficient processing and presentation of multiple cassette-derived antigens. However, the magnitude of T cell responses were not substantially impacted by the various targeting features (Table 4).

TABLE 4
Evaluation of cellular targeting sequences added to model vaccine cassettes.
Employing the in vitro T cell activation assay demonstrated that the four HLA-A*0201
restricted marker epitopes are liberated efficiently from the model cassettes and targeting
sequences did not substantially improve T cell recognition and activation.

| | Short Cassette Designs | | | | | | | | | |
|---|---|---|---|---|---|---|---|---|---|---|
| Epitope | A | B | C | D | E | F | G | H | 1 | J |
| 1 | 32.5 ± 1.5 | 31.8 ± 0.8 | 29.1 ± 1.2 | 29.1 ± 1.1 | 28.4 ± 0.7 | 20.4 ± 0.5 | 35.0 ± 1.3 | 30.3 ± 2.0 | 22.5 ± 0.9 | 38.1 ± 1.6 |
| 2 | 6.1 ± 0.2 | 6.3 ± 0.2 | 7.6 ± 0.4 | 7.0 ± 0.5 | 5.9 ± 0.2 | 3.7 ± 0.2 | 7.6 ± 0.4 | 5.4 ± 0.3 | 6.2 ± 0.4 | 6.4 ± 0.3 |
| 3* | n/a | n/a | n/a | n/a | n/a | n/a | n/a | n/a | n/a | n/a |
| 4 | 12.3 ± 1.1 | 14.1 ± 0.7 | 12.2 ± 0.8 | 13.7 ± 1.0 | 11.7 ± 0.8 | 10.6 ± 0.4 | 11.0 ± 0.6 | 7.6 ± 0.6 | 16.1 ± 0.5 | 8.7 ± 0.5 |
| 5 | 44.4 ± 2.8 | 53.6 ± 1.6 | 49.9 ± 3.3 | 50.5 ± 2.8 | 41.7 ± 2.8 | 36.1 ± 1.1 | 46.5 ± 2.1 | 31.4 ± 0.6 | 75.4 ± 1.6 | 35.7 ± 2.2 |

* Reporter^{r} [ cell for epitope 3 not yet generated

### XIV.B.3. In Vivo Evaluation of Antigen Cassette Designs

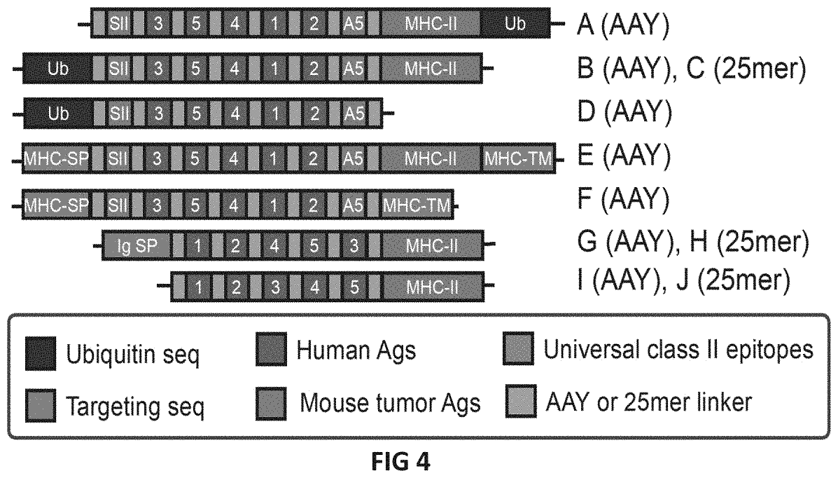

As another example of antigen cassette design evaluation, vaccine cassettes were designed to contain 5 well-characterized human class I MHC epitopes known to stimulate CD8 T cells in an HLA-A*02:01 restricted fashion (FIG. 2A, 3, 5A). For the evaluation of their in vivo immunogenicity, vaccine cassettes containing these marker epitopes were incorporated in adenoviral vectors and used to infect HLA-A2 transgenic mice (FIG. 4). This mouse model carries a transgene consisting partly of human HLA-A*0201 and mouse H2-Kb thus encoding a chimeric class I MHC molecule consisting of the human HLA-A2.1 leader, α1 and α2 domains ligated to the murine α3, transmembrane and cytoplasmic H2-Kb domain (Vitiello et al., 1991). The chimeric molecule allows HLA-A*02:01-restricted antigen presentation whilst maintaining the species-matched interaction of the CD8 co-receptor with the α3 domain on the MHC.

For the short cassettes, all marker epitopes generated a T cell response, as determined by IFN-gamma ELISpot, that was approximately 10-50× stronger of what has been commonly reported (Comet et al., 2006; Depla et al., 2008; Ishioka et al., 1999). Of all the linkers evaluated, the concatamer of 25mer sequences, each containing a minimal epitope flanked by their natural amino acids sequences, generated the largest and broadest T cell response (Table 5). Intracellular cytokine staining (ICS) and flow cytometry analysis revealed that the antigen-specific T cell responses are derived from CD8 T cells.

TABLE 5
In vivo evaluation of linker sequences in short cassettes. ELISpot data indicated that
HLA-A2 transgenic mice, 17 days post-infection with lell adenovirus viral particles,
generated a T cell response to all class I MHC restricted epitopes in the cassette.

| | Short Cassette Designs | | | | | |
|---|---|---|---|---|---|---|
| Epitope | 9AA | 17AA | 25AA | AAY | RR | DPP |
| 1 | 2020 +/− 583 | 2505 +/− 1281 | 6844 +/− 956 | 1489 +/− 762 | 1675 +/− 690 | 1781 +/− 774 |
| 2 | 4472 +/− 755 | 3792 +/− 1319 | 7629 +/− 996 | 3851 +/− 1748 | 4726 +/− 1715 | 5868 +/− 1427 |
| 3 | 5830 +/− 315 | 3629 +/− 862 | 7253 +/− 491 | 4813 +/− 1761 | 6779 +/− 1033 | 7328 +/− 1700 |
| 4 | 5536 +/− 375 | 2446 +/− 955 | 2961 +/− 1487 | 4230 +/− 1759 | 6518 +/− 909 | 7222 +/− 1824 |
| 5 | 8800 +/− 0 | 7943 +/− 821 | 8423+7-442 | 8312 +/− 696 | 8800 +/− 0 | 1836 +/− 328 |

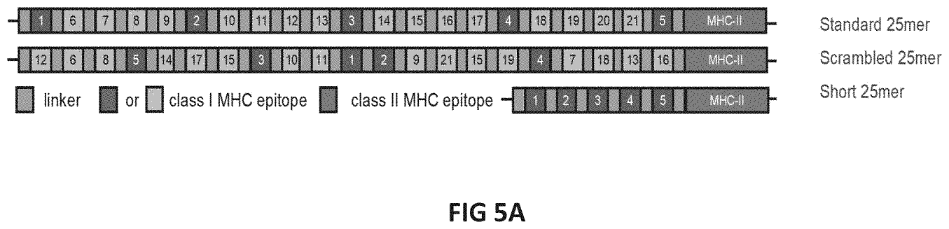

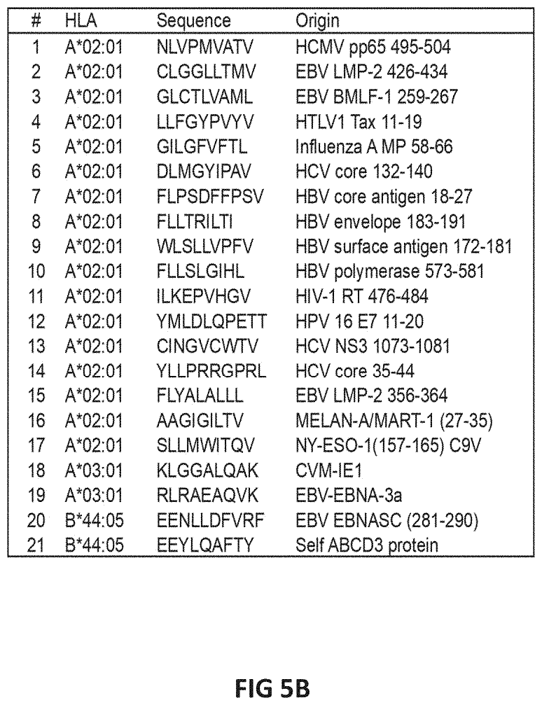

In another example, a series of long vaccine cassettes was constructed and incorporated in adenoviral vectors that, next to the original 5 marker epitopes, contained an additional 16 HLA-A*02:01, A*03:01 and B*44:05 epitopes with known CD8 T cell reactivity (FIG. 5A, B). The size of these long cassettes closely mimicked the final clinical cassette design, and only the position of the epitopes relative to each other was varied. The CD8 T cell responses were comparable in magnitude and breadth for both long and short vaccine cassettes, demonstrating that (a) the addition of more epitopes did not substantially impact the magnitude of immune response to the original set of epitopes, and (b) the position of an epitope in a cassette did not substantially influence the ensuing T cell response to it (Table 6).

TABLE 6
In vivo evaluation of the impact of epitope position in long cassettes.
ELISpot data indicated that HLA-A2 transgenic mice, 17 days
post-infection with 5e10 adenovirus viral particles, generated a T cell
response comparable in magnitude for both long and short vaccine cassettes.

| | Long Cassette Designs | | |
|---|---|---|---|
| Epitope | Standard | Scrambled | Short |
| 1 | 863 +/− 1080 | 804 +/− 1113 | 1871 +/− 2859 |
| 2 | 6425 +/− 1594 | 28 +/− 62 | 5390 +/− 1357 |
| 3* | 23 +/− 30 | 36 +/− 18 | 0 +/− 48 |
| 4 | 2224 +/− 1074 | 2727 +/− 644 | 2637 +/− 1673 |
| 5 | 7952 +/− 297 | 8100 +/− 0 | 8100 +/− 0 |

*Suspected technical error caused an absence of a T cell response.

### XIV.B.4. Antigen Cassette Design for Immunogenicity and Toxicology Studies

In summary, the findings of the model cassette evaluations (FIG. 2-5, Tables 2-6) demonstrated that, for model vaccine cassettes, robust immunogenicity was achieved when a “string of beads” approach was employed that encodes around 20 epitopes in the context of an adenovirus-based vector. The epitopes were assembled by concatenating 25mer sequences, each embedding a minimal CD8 T cell epitope (e.g. 9 amino acid residues) that were flanked on both sides by its natural, surrounding peptide sequence (e.g. 8 amino acid residues on each side). As used herein, a “natural” or “native” flanking sequence refers to the N- and/or C-terminal flanking sequence of a given epitope in the naturally occurring context of that epitope within its source protein. For example, the HCMV pp65 MHC I epitope NLVPMVATV (SEQ ID NO: 29365) is flanked on its 5′ end by the native 5′ sequence WQAGILAR (SEQ ID NO: 29372) and on its 3′ end by the native 3′ sequence QGQNLKYQ (SEQ ID NO: 29373), thus generating the WQAGILARNLVPMVATVQGQNLKYQ (SEQ ID NO: 29374) 25mer peptide found within the HCMV pp65 source protein. The natural or native sequence can also refer to a nucleotide sequence that encodes an epitope flanked by native flanking sequence(s). Each 25mer sequence is directly connected to the following 25mer sequence. In instances where the minimal CD8 T cell epitope is greater than or less than 9 amino acids, the flanking peptide length can be adjusted such that the total length is still a 25mer peptide sequence. For example, a 10 amino acid CD8 T cell epitope can be flanked by an 8 amino acid sequence and a 7 amino acid. The concatamer was followed by two universal class II MHC epitopes that were included to stimulate CD4 T helper cells and improve overall in vivo immunogenicity of the vaccine cassette antigens. (Alexander et al., 1994; Panina-Bordignon et al., 1989) The class II epitopes were linked to the final class I epitope by a GPGPG amino acid linker (SEQ ID NO:56). The two class II epitopes were also linked to each other by a GPGPG amino acid linker (SEQ ID NO: 56), as a well as flanked on the C-terminus by a GPGPG amino acid linker (SEQ ID NO: 56). Neither the position nor the number of epitopes appeared to substantially impact T cell recognition or response. Targeting sequences also did not appear to substantially impact the immunogenicity of cassette-derived antigens.

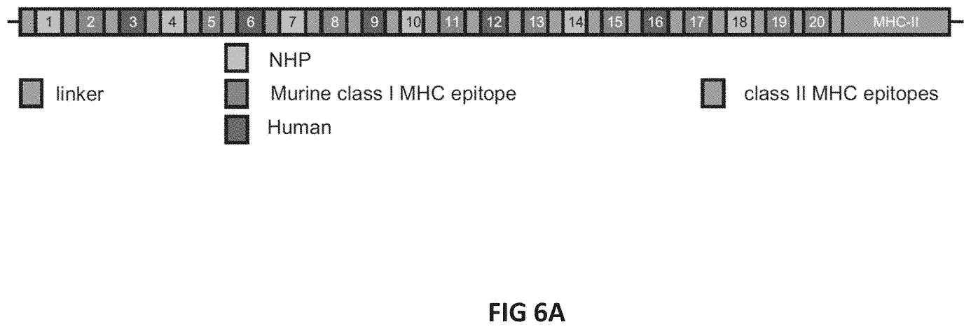

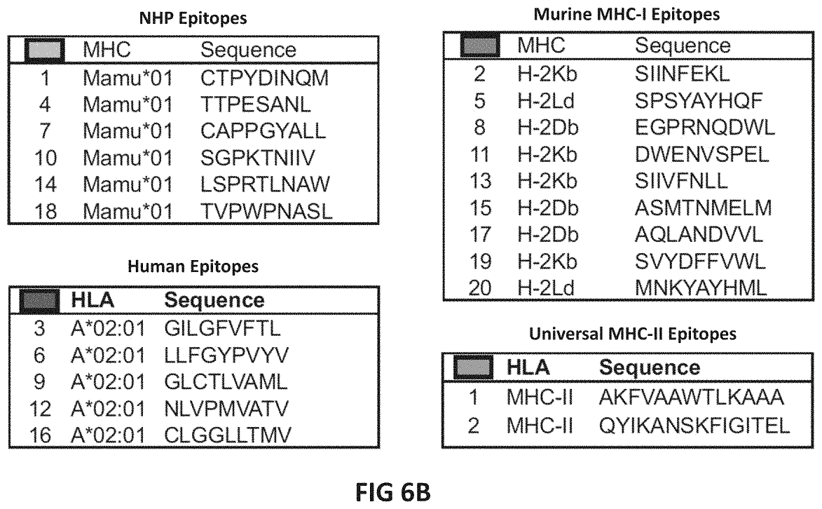

As a further example, based on the in vitro and in vivo data obtained with model cassettes (FIG. 2-5, Tables 2-6), a cassette design was generated that alternates well-characterized T cell epitopes known to be immunogenic in nonhuman primates (NHPs), mice and humans. The 20 epitopes, all embedded in their natural 25mer sequences, are followed by the two universal class II MHC epitopes that were present in all model cassettes evaluated (FIG. 6). This cassette design was used to study immunogenicity as well as pharmacology and toxicology studies in multiple species.

### XIV.B.5. Antigen Cassette Design and Evaluation for 30, 40, and 50 Antigens

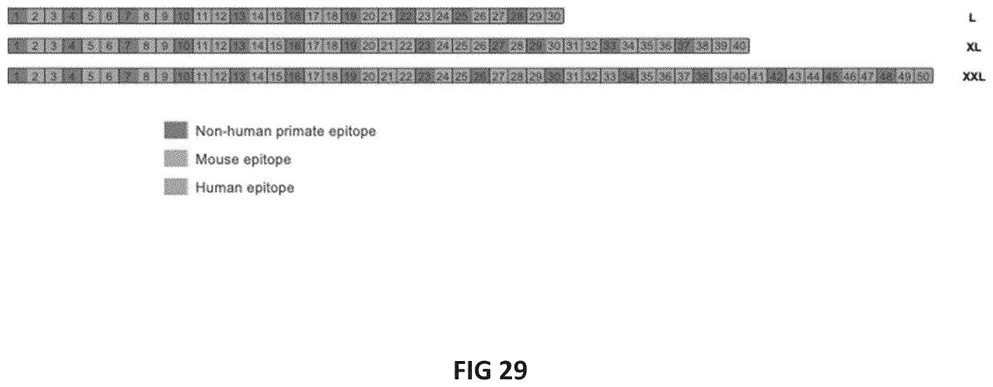

Large antigen cassettes were designed that had either 30 (L), 40 (XL) or 50 (XXL) epitopes, each 25 amino acids in length. The epitopes were a mix of human, NHP and mouse epitopes to model disease antigens including tumor antigens. FIG. 29 illustrates the general organization of the epitopes from the various species. The model antigens used are described in Tables 37, 38 and 39 for human, primate, and mouse model epitopes, respectively. Each of Tables 37, 38 and 39 described the epitope position, name, minimal epitope description, and MHC class.

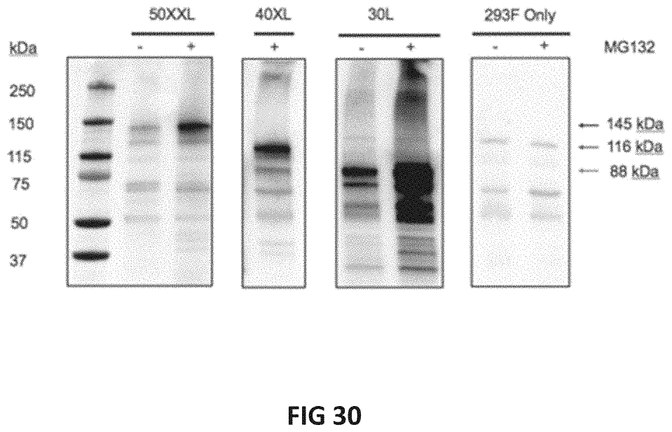

These cassettes were cloned into the chAd68 and alphavirus vaccine vectors as described to evaluate the efficacy of longer multiple-epitope cassettes. FIG. 30 shows that each of the large antigen cassettes were expressed from a ChAdV vector as indicated by at least one major band of the expected size by Western blot.

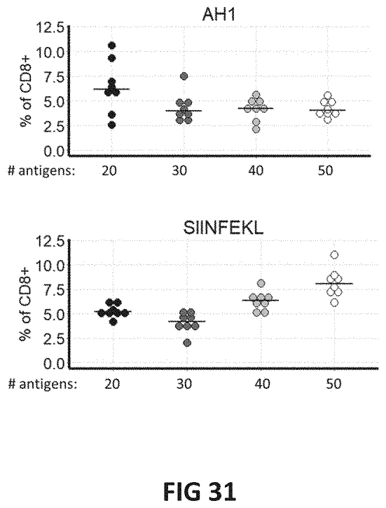

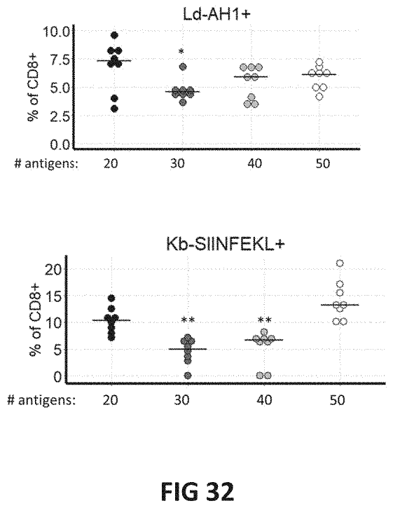

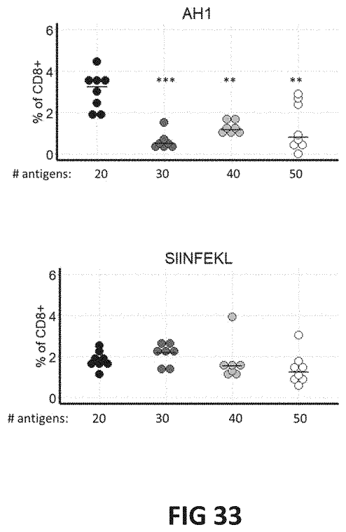

Mice were immunized as described to evaluate the efficacy of the large cassettes. T cell responses were analyzed by ICS and tetramer staining following immunization with a chAd68 vector (FIG. 31/Table 40 and FIG. 32/Table 41, respectively) and by ICS following immunization with a srRNA vector (FIG. 33/Table 42) for epitopes AH1 (top panels) and SIINFEKL (SEQ ID NO: 29362) (bottom panels). Immunizations using chAd68 and srRNA vaccine vectors expressing either 30 (L), 40 (XL) or 50 (XXL) epitopes induced CD8+ immune responses to model disease epitopes.

TABLE 37
Human epitopes in large cassettes (SEQ ID NO 29367-29369, 29365-29366,
29402-29414, 29374, and 29415-29425, respectively, in order of columns)

| Epitope position | | | | | | | | | |
|---|---|---|---|---|---|---|---|---|---|
| in each cassette | | | | Minimal | | | | | |
| L | XL | XXL | Name | epitope | 25 mer | MHC | Restriction | Strain | Species |
| 3 | 3 | 3 | 5.influenza | GILGFVFTL | PILSPLTKGILGF | Class 1 | A*02:01 | Human | Human |
| | | | M | | VFTLTVPSERGL | | | | |
| 6 | 6 | 6 | 4.HTLV-1 Tax | LLFGYPVYV | HFPGFGQSLLFGY | Class 1 | A*02:01 | Human | Human |
| | | | | | PVYVFGDCVQGD | | | | |
| 9 | 9 | 9 | 3.EBV BMLF1 | GLCTLVAML | RMQAIQNAGLCTL | Class 1 | A*02:01 | Human | Human |
| | | | | | VAMLEETIFWLQ | | | | |
| 12 | 12 | 12 | 1.HCMV pp65 | NLVPMVATV | WQAGILARNLVPM | Class 1 | A*02:01 | Human | Human |
| | | | | | VATVQGQNLKYQ | | | | |
| 15 | 15 | 15 | 2.EBV LMP2A | CLGGLLTMV | RTYGPVFMCLGGL | Class 1 | A*02:01 | Human | Human |
| | | | | | LTMVAGAVWLTV | | | | |
| 18 | 18 | 18 | CT83 | NTDNNLAVY | SSSGLINSNTDNN | Class 1 | A*01:01 | Human | Human |
| | | | | | LAVYDLSRDILN | | | | |
| | 21 | 21 | MAGEA6 | EVDPIGHVY | LVFGIELMEVDPI | Class 1 | B*35:01 | Human | Human |
| | | | | | GHVYIFATCLGL | | | | |
| 21 | 25 | 25 | CT83 | LLASSILCA | MNFYLLLASSILC | Class 1 | A*02:01 | Human | Human |
| | | | | | ALIVFWKYRRFQ | | | | |
| 24 | 31 | 28 | FOXE1 | AIFPGAVPAA | AAAAAAAAIFPGA | Class 1 | A*02:01 | Human | Human |
| | | | | | VPAARPPYPGAV | | | | |
| 27 | 35 | 32 | CT83 | VYDLSRDIL | SNTDNNLAVYDLS | Class 1 | A*24:02 | Human | Human |
| | | | | | RDILNNFPHSIA | | | | |
| 30 | 38 | 36 | MAGE3/6 | ASSLPTTMNY | DPPQSPQGASSLP | Class 1 | A*01:01 | Human | Human |
| | | | | | TTMNYPLWSQSY | | | | |
| | 40 | 40 | Influenza | PKYVKQNTLK | ITYGACPKYVKQN | Class 11 | DRB1*0101 | Human | Human |
| | | | HA | LAT | TLKLATGMRNVP | | | | |
| | | 44 | CMV pp65 | LPLKMLNIPS | SIYVYALPLKMLN | Class 11 | DRBl*0101 | Human | Human |
| | | | | INVH | IPSINVHHYPSA | | | | |
| | | 47 | EBV EBNA3A | PEQWMFQGAP | EGPWVPEQWMFQG | Class 11 | DRBl*0102 | Human | Human |
| | | | | PSQGT | APPSQGTDVVQH | | | | |
| | | 50 | CMV pp65 | EHPTFTSQYR | RGPQYSEHPTFTS | Class II | DRBl*1101 | Human | Human |
| | | | | IQGKL | QYRIQGKLEYRH | | | | |

TABLE 38
NHP epitopes in large cassettes (SEQ ID NO 29426-29455,
respectively, in order of columns)

| Epitope position | | | | | | | | | |
|---|---|---|---|---|---|---|---|---|---|
| in each cassette | | | | Minimal | | | | | |
| L | XL | XXL | Name | epitope | 25 mer | MHC | Restriction | Strain | Species |
| 1 | 1 | 1 | Gag CM9 | CTPYDINQM | MFQALSEGCTPYD | Class I | Mamu-A*01 | Rhesus | NHP |
| | | | | | INQMLNVLGDHQ | | | | |
| 4 | 4 | 4 | Tat TL8 | TTPESANL | SCISEADATTPES | Class I | Mamu-A*01 | Rhesus | NHP |
| | | | | | ANLGEEILSQLY | | | | |
| 7 | 7 | 7 | EnvCL9 | CAPPGYALL | WDAIRFRYCAPPG | Class I | Mamu-A*01 | Rhesus | NHP |
| | | | | | YALLRCNDTNYS | | | | |
| 10 | 10 | 10 | Pol SV9 | SGPKTNIIV | AFLMALTDSGPKT | Class I | Mamu-A*01 | Rhesus | NHP |
| | | | | | NIIVDSQYVMGI | | | | |
| 13 | 13 | 13 | Gag LW9 | LSPRTLNAW | GNVWVHTPLSPRT | Class I | Mamu-A*01 | Rhesus | NHP |
| | | | | | LNAWVKAVEEKK | | | | |
| | | 16 | Env_TL9 | TVPWPNASL | AFRQVCHTTVPWP | Class I | Mamu-A*01 | Rhesus | NHP |
| | | | | | NASLTPKWNNET | | | | |
| 16 | 16 | 19 | Ag85B | PNGTHSWEYW | VFNFPPNGTHSWE | Class II | Mamu-DR*W | Rhesus | NHP |
| | | | | GAQLN | YWGAQLNAMKGD | | | | |
| 19 | 19 | 23 | HIV-1 Env | YKYKVVKIEP | NWRSELYKYKVVK | Class II | Mamu-DR*W | Rhesus | NHP |
| | | | | LGV | IEPLGVAPTKAK | | | | |
| | | 26 | Gag TE15 | TEEAKQIVQR | EKVKHTEEAKQIV | Class II | Mamu-DRB* | Rhesus | NHP |
| | | | | HLVVE | QRHLVVETGTTE | | | | |
| | 23 | 30 | CFP-10 36-48 | AGSLQGQWRG | DQVESTAGSLQGQ | Class II | Mafa-DRB1* | Cyno | NHP |
| | | | | AAG | WRGAAGTAAQAA | | | | |
| | 27 | 34 | CFP-10 71-86 | EISTNIRQAG | QELDEISTNIRQA | Class II | Mafa-DRBI* | Cyno | NHP |
| | | | | VQYSRA | GVQYSRADEEQQ | | | | |
| 22 | 29 | 38 | Env 338-346 | RPKQAWCWF | FHSQPINERPKQA | Class I | Mafa-A1*06 | Cyno | NHP |
| | | | | | WCWFGGSWKEAI | | | | |
| 25 | 33 | 42 | Nef 103-111 | RPKVPLRTM | DDIDEEDDDLVGV | Class I | Mafa-A1*06 | Cyno | NHP |
| | | | | | SVRPKVPLRTMS | | | | |
| 28 | 37 | 45 | Gag 386-394 | GPRKPIKCW | PFAAAQQRGPRKP | Class I | Mafa-A1*06 | Cyno | NHP |
| | | | | | IKCWNCGKEGHS | | | | |
| | | 48 | Nef LT9 | LNMADKKET | RRLTARGLLNMAD | Class I | Mafa-B*104 | Cyno | NHP |
| | | | | | KKETRTPKKAKA | | | | |

TABLE 39
Mouse epitopes in large cassettes (SEQ ID NOS 29362, 29456-29458,
29363, 29459-29493, respectively, in order of columns)

| Epitope position | | | | | | | | | |
|---|---|---|---|---|---|---|---|---|---|
| in each cassette | | | | Minimal | | | | | |
| L | XL | XXL | Name | epitope | 25 mer | MHC | Restriction | Strain | Species |
| 2 | 2 | 2 | OVA257 | SIINFEKL | VSGLEQLESIINF | Class I | H2-Kb | B6 | Mouse |
| | | | | | EKLTEWTSSNVM | | | | |
| | | 5 | B16-EGP | EGPRNQDWL | ALLAVGALEGPRN | Class I | H2-Db | B6 | Mouse |
| | | | | | QDWLGVPRQLVT | | | | |
| | | 8 | B16-TRP1 | TAPDNLGYM | VTNTEMFVTAPDN | Class I | H2-Db | B6 | Mouse |
| | | | 455-463 | | LGYMYEVQWPGQ | | | | |
| | | 11 | Trp2180-188 | SVYDFFVWL | TQPQIANCSVYDF | Class I | H2-Kb | B6 | Mouse |
| | | | | | FVWLHYYSVRDT | | | | |
| 5 | 5 | 14 | CT26 AH1-A5 | SPSYAYHQF | LWPRVTYHSPSYA | Class I | H2-Ld | Balb/C | Mouse |
| | | | | | YHQFERRAKYKR | | | | |
| | 8 | 17 | CT26 AH1-39 | MNKYAYHML | LWPRVTYHMNKYA | Class I | H2-Ld | Balb/C | Mouse |
| | | | | | YHMLERRAKYKR | | | | |
| | 11 | 20 | MC38 Dpagt1 | SIIVFNLL | GQSLVISASIIVF | Class I | H2-Kb | B6 | Mouse |
| | | | | | NLLELEGDYRDD | | | | |
| | 14 | 22 | MC38 Adpgk | ASMTNMELM | GIPVHLELASMTN | Class I | H2-Db | B6 | Mouse |
| | | | | | MELMSSIVHQQV | | | | |
| | 17 | 24 | MC38 Reps1 | AQLANDVVL | RVLELFRAAQLAN | Class I | H2-Db | B6 | Mouse |
| | | | | | DVVLQIMELCGA | | | | |
| 8 | 20 | 27 | P815 P1A | LPYLGWLVF | HRYSLEEILPYLG | Class I | H2-Ld | DBA/2 | Mouse |
| | | | 35-44 | | WLVFAVVTTSFL | | | | |
| 11 | 22 | 29 | P815 P1E | GYCGLRGTGV | YLSKNPDGYCGLR | Class I | H2-Kd | DBA/2 | Mouse |
| | | | | | GTGVSCPMAIKK | | | | |
| 14 | 24 | 31 | Panc02 | LSIFKHKL | NEIPFTYEQLSIF | Class I | H2-Kb | B6 | Mouse |
| | | | Mesothelir | | KHKLDKTYPQGY | | | | |
| 17 | 26 | 33 | Panc02 | LIWIPALL | SRASLLGPGFVLI | Class I | H2-Kb | B6 | Mouse |
| | | | Mesothelir | | WIPALLPALRLS | | | | |
| 20 | 28 | 35 | ID8 FRa | SSGHNECPV | NWHKGWNWSSGHN | Class I | H2-Kb | B6 | Mouse |
| | | | 161-169 | | ECPVGASCHPFT | | | | |
| 23 | 30 | 37 | ID8 | GQKMNAQAI | KTLLKVSKGQKMN | Class I | H2-Db | B6 | Mouse |
| | | | Mesothelin | | AQAIALVACYLR | | | | |
| | | | 40 | | | | | | |
| 26 | 32 | 39 | OVA-II | ISQAVHAAHA | ESLKISQAVHAAH | Class II | I-Ab, I-Ad | B6 | Mouse |
| | | | | EINEAGR | AEINEAGREVVG | | | | |
| 29 | 34 | 41 | ESAT-6 | MTEQQWNFAG | MTEQQWNFAGIEA | Class II | I-Ab | B6 | Mouse |
| | | | | IEAAASAIQ | AASAIQGNVTSI | | | | |
| | 36 | 43 | TT p30 | FNNFTVSFWL | DMFNNFTVSFWLR | Class II | I-Ab | Balb/C | Mouse |
| | | | | RVPKVSASHL | VPKVSASHLEQY | | | | |
| | 39 | 46 | HEL | DGSTDYGILQ | TNRNTDGSTDYGI | Class II | I-Ak | CBA | Mouse |
| | | | | INSRW | LQINSRWWCNDG | | | | |
| | | 49 | MOG | MEVGWYRSPF | TGMEVGWYRSPFS | Class II | I-Ab | B6 | Mouse |
| | | | | SRVVHLYRN | RVVHLYRNGKDQ | | | | |

TABLE 40
Average IFNg+ cells in response to AH1 and SIINFEKL (SEQ ID NO: 29362)
peptides in ChAd large cassette treated mice. Data is presented as % of total
CD8 cells. Shown is average and standard deviation per group and p-value by
ANOVA with Tukey’s test. All p-values compared to MAG 20-antigen cassette.

| # antigens | Antigen | Average | Standard deviation | p-value | N |
|---|---|---|---|---|---|
| 20 | SIINFEKL | 5.308 | 0.660 | n/a | 8 |
| 30 | SIINFEKL | 4.119 | 1.019 | 0.978 | 8 |
| 40 | SIINFEKL | 6.324 | 0.954 | 0.986 | 8 |
| 50 | SIINFEKL | 8.169 | 1.469 | 0.751 | 8 |
| 20 | AH1 | 6.405 | 2.664 | n/a | 8 |
| 30 | AH1 | 4.373 | 1.442 | 0.093 | 8 |
| 40 | AH1 | 4.126 | 1.135 | 0.050 | 8 |
| 50 | AH1 | 4.216 | 0.808 | 0.063 | 8 |

TABLE 41
Average tetramer+ cells for AH1 and SIINFEKL (SEQ ID NO: 29362) antigens in
ChAd large cassette treated mice. Data is presented as % of total CD8 cells.
Shown is average and standard deviation per group and p-value by ANOVA
with Tukey’s test. All p-values compared to MAG 20-antigen cassette.

| # antigens | Antigen | Average | Standard deviation | p-value | N |
|---|---|---|---|---|---|
| 20 | SIINFEKL | 10.314 | 2.384 | n/a | 8 |
| 30 | SIINFEKL | 4.551 | 2.370 | 0.003 | 8 |
| 40 | SIINFEKL | 5.186 | 3.254 | 0.009 | 8 |
| 50 | SIINFEKL | 14.113 | 3.660 | 0.072 | 8 |
| 20 | AH1 | 6.864 | 2.207 | n/a | 8 |
| 30 | AH1 | 4.713 | 0.922 | 0.036 | 8 |
| 40 | AH1 | 5.393 | 1.452 | 0.223 | 8 |
| 50 | AH1 | 5.860 | 1.041 | 0.543 | 8 |

TABLE 42
Average IFNg+ cells in response to AH1 and SIINFEKL (SEQ ID NO: 29362)
peptides in SAM large cassette treated mice. Data is presented as % of total
CD8 cells. Shown is average and standard deviation per group and p-value by ANOVA
with Tukey’s test. All p-values compared to MAG 20-antigen cassette.

| # antigens | Antigen | Average | Standard deviation | p-value | N |
|---|---|---|---|---|---|
| 20 | SIINFEKL | 1.843 | 0.422 | n/a | 8 |
| 30 | SIINFEKL | 2.112 | 0.522 | 0.879 | 7 |
| 40 | SIINFEKL | 1.754 | 0.978 | 0.995 | 7 |
| 50 | SIINFEKL | 1.409 | 0.766 | 0.606 | 8 |
| 20 | AH1 | 3.050 | 0.909 | n/a | 8 |
| 30 | AH1 | 0.618 | 0.427 | 1.91E−05 | 7 |
| 40 | AH1 | 1.286 | 0.284 | 0.001 | 7 |
| 50 | AH1 | 1.309 | 1.149 | 0.001 | 8 |

### XV. ChAd Antigen Cassette Delivery Vector

### XV.A. ChAd Antigen Cassette Delivery Vector Construction

In one example, Chimpanzee adenovirus (ChAd) was engineered to be a delivery vector for antigen cassettes. In a further example, a full-length ChAdV68 vector was synthesized based on AC_000011.1 (sequence 2 from U.S. Pat. No. 6,083,716) with E1 (nt 457 to 3014) and E3 (nt 27,816-31,332) sequences deleted. Reporter genes under the control of the CMV promoter/enhancer were inserted in place of the deleted E1 sequences. Transfection of this clone into HEK293 cells did not yield infectious virus. To confirm the sequence of the wild-type C68 virus, isolate VR-594 was obtained from the ATCC, passaged, and then independently sequenced (SEQ ID NO:10). When comparing the AC_000011.1 sequence to the ATCC VR-594 sequence (SEQ ID NO:10) of wild-type ChAdV68 virus, 6 nucleotide differences were identified. In one example, a modified ChAdV68 vector was generated based on AC_000011.1, with the corresponding ATCC VR-594 nucleotides substituted at five positions (ChAdV68.5WTnt SEQ ID NO:1).

In another example, a modified ChAdV68 vector was generated based on AC_000011.1 with E1 (nt 577 to 3403) and E3 (nt 27,816-31,332) sequences deleted and the corresponding ATCC VR-594 nucleotides substituted at four positions. A GFP reporter (ChAdV68.4WTnt.GFP; SEQ ID NO:11) or model neoantigen cassette (ChAdV68.4WTnt.MAG25mer; SEQ ID NO:12) under the control of the CMV promoter/enhancer was inserted in place of deleted E1 sequences.

In another example, a modified ChAdV68 vector was generated based on AC_000011.1 with E1 (nt 577 to 3403) and E3 (nt 27,125-31,825) sequences deleted and the corresponding ATCC VR-594 nucleotides substituted at five positions. A GFP reporter (ChAdV68.5WTnt.GFP; SEQ ID NO:13) or model neoantigen cassette (ChAdV68.5WTnt.MAG25mer; SEQ ID NO:2) under the control of the CMV promoter/enhancer was inserted in place of deleted E1 sequences.

Relevant vectors are described below:
Full-Length ChAdVC68 sequence “ChAdV68.5WTnt” (SEQ ID NO:1); AC_000011.1 sequence with corresponding ATCC VR-594 nucleotides substituted at five positions.
ATCC VR-594 C68 (SEQ ID NO:10); Independently sequenced; Full-Length C68
ChAdV68.4WTnt.GFP (SEQ ID NO:11); AC_000011.1 with E1 (nt 577 to 3403) and E3 (nt 27,816-31,332) sequences deleted; corresponding ATCC VR-594 nucleotides substituted at four positions; GFP reporter under the control of the CMV promoter/enhancer inserted in place of deleted E1
ChAdV68.4WTnt.MAG25mer (SEQ ID NO:12); AC_000011.1 with E1 (nt 577 to 3403) and E3 (nt 27,816-31,332) sequences deleted; corresponding ATCC VR-594 nucleotides substituted at four positions; model neoantigen cassette under the control of the CMV promoter/enhancer inserted in place of deleted E1
ChAdV68.5WTnt.GFP (SEQ ID NO:13); AC_000011.1 with E1 (nt 577 to 3403) and E3 (nt 27,125-31,825) sequences deleted; corresponding ATCC VR-594 nucleotides substituted at five positions; GFP reporter under the control of the CMV promoter/enhancer inserted in place of deleted E1

### XV.B. ChAd Antigen Cassette Delivery Vector Testing

### XV.B.1. ChAd Vector Evaluation Methods and Materials

### Transfection of HEK293A Cells Using Lipofectamine

DNA for the ChAdV68 constructs (ChAdV68.4WTnt.GFP, ChAdV68.5WTnt.GFP, ChAdV68.4WTnt.MAG25mer and ChAdV68.5WTnt.MAG25mer) was prepared and transfected into HEK293A cells using the following protocol.

10 ug of plasmid DNA was digested with PacI to liberate the viral genome. DNA was then purified using GeneJet DNA cleanup Micro columns (Thermo Fisher) according to manufacturer's instructions for long DNA fragments, and eluted in 20 ul of pre-heated water; columns were left at 37 degrees for 0.5-1 hours before the elution step.

HEK293A cells were introduced into 6-well plates at a cell density of 10⁶ cells/well 14-18 hours prior to transfection. Cells were overlaid with 1 ml of fresh medium (DMEM-10% hiFBS with pen/strep and glutamate) per well. 1-2 ug of purified DNA was used per well in a transfection with twice the ul volume (2-4 ul) of Lipofectamine2000, according to the manufacturer's protocol. 0.5 ml of OPTI-MEM medium containing the transfection mix was added to the 1 ml of normal growth medium in each well, and left on cells overnight.

Transfected cell cultures were incubated at 37° C. for at least 5-7 days. If viral plaques were not visible by day 7 post-transfection, cells were split 1:4 or 1:6, and incubated at 37° C. to monitor for plaque development. Alternatively, transfected cells were harvested and subjected to 3 cycles of freezing and thawing and the cell lysates were used to infect HEK293A cells and the cells were incubated until virus plaques were observed.

Transfection of ChAdV68 Vectors into HEK293A Cells Using Calcium Phosphate and Generation of the Tertiary Viral Stock

DNA for the ChAdV68 constructs (ChAdV68.4WTnt.GFP, ChAdV68.5WTnt.GFP, ChAdV68.4WTnt.MAG25mer, ChAdV68.5WTnt.MAG25mer) was prepared and transfected into HEK293A cells using the following protocol.

HEK293A cells were seeded one day prior to the transfection at 10⁶ cells/well of a 6 well plate in 5% BS/DMEM/1×P/S, 1× Glutamax. Two wells are needed per transfection. Two to four hours prior to transfection the media was changed to fresh media. The ChAdV68.4WTnt.GFP plasmid was linearized with PacI. The linearized DNA was then phenol chloroform extracted and precipitated using one tenth volume of 3M Sodium acetate pH 5.3 and two volumes of 100% ethanol. The precipitated DNA was pelleted by centrifugation at 12,000×g for 5 min before washing 1× with 70% ethanol. The pellet was air dried and re-suspended in 50 μL of sterile water. The DNA concentration was determined using a NanoDrop™ (ThermoFisher) and the volume adjusted to 5 μg of DNA/50 μL.

169 μL of sterile water was added to a microfuge tube. 5 μL of 2M CaCl₂) was then added to the water and mixed gently by pipetting. 50 μL of DNA was added dropwise to the CaCl₂) water solution. Twenty six L of 2M CaCl₂) was then added and mixed gently by pipetting twice with a micro-pipetor. This final solution should consist of 5 μg of DNA in 250 μL of 0.25M CaCl₂). A second tube was then prepared containing 250 μL of 2×HBS (Hepes buffered solution). Using a 2 mL sterile pipette attached to a Pipet-Aid air was slowly bubbled through the 2×HBS solution. At the same time the DNA solution in the 0.25M CaCl₂) solution was added in a dropwise fashion. Bubbling was continued for approximately 5 seconds after addition of the final DNA droplet. The solution was then incubated at room temperature for up to 20 minutes before adding to 293A cells. 250 μL of the DNA/Calcium phosphate solution was added dropwise to a monolayer of 293A cells that had been seeded one day prior at 10⁶ cells per well of a 6 well plate. The cells were returned to the incubator and incubated overnight. The media was changed 24 h later. After 72 h the cells were split 1:6 into a 6 well plate. The monolayers were monitored daily by light microscopy for evidence of cytopathic effect (CPE). 7-10 days post transfection viral plaques were observed and the monolayer harvested by pipetting the media in the wells to lift the cells. The harvested cells and media were transferred to a 50 mL centrifuge tube followed by three rounds of freeze thawing (at −80° C. and 37° C.). The subsequent lysate, called the primary virus stock was clarified by centrifugation at full speed on a bench top centrifuge (4300×g) and a proportion of the lysate 10-50%) used to infect 293A cells in a T25 flask. The infected cells were incubated for 48 h before harvesting cells and media at complete CPE. The cells were once again harvested, freeze thawed and clarified before using this secondary viral stock to infect a T150 flask seeded at 1.5×10⁷ cells per flask. Once complete CPE was achieved at 72 h the media and cells were harvested and treated as with earlier viral stocks to generate a tertiary stock.

### Production in 293F Cells

ChAdV68 virus production was performed in 293F cells grown in 293 FreeStyle™ (ThermoFisher) media in an incubator at 8% C02. On the day of infection cells were diluted to 10⁶ cells per mL, with 98% viability and 400 mL were used per production run in 1 L Shake flasks (Corning). 4 mL of the tertiary viral stock with a target MOI of >3.3 was used per infection. The cells were incubated for 48-72 h until the viability was <70% as measured by Trypan blue. The infected cells were then harvested by centrifugation, full speed bench top centrifuge and washed in 1×PBS, re-centrifuged and then re-suspended in 20 mL of 10 mM Tris pH7.4. The cell pellet was lysed by freeze thawing 3× and clarified by centrifugation at 4,300×g for 5 minutes.

### Purification by CsCl Centrifugation

Viral DNA was purified by CsCl centrifugation. Two discontinuous gradient runs were performed. The first to purify virus from cellular components and the second to further refine separation from cellular components and separate defective from infectious particles.

10 mL of 1.2 (26.8 μg CsCl dissolved in 92 mL of 10 mM Tris pH 8.0) CsCl was added to polyallomer tubes. Then 8 mL of 1.4 CsCl (53 μg CsCl dissolved in 87 mL of 10 mM Tris pH 8.0) was carefully added using a pipette delivering to the bottom of the tube. The clarified virus was carefully layered on top of the 1.2 layer. If needed more 10 mM Tris was added to balance the tubes. The tubes were then placed in a SW-32Ti rotor and centrifuged for 2 h 30 min at 10° C. The tube was then removed to a laminar flow cabinet and the virus band pulled using an 18 gauge needle and a 10 mL syringe. Care was taken not to remove contaminating host cell DNA and protein. The band was then diluted at least 2× with 10 mM Tris pH 8.0 and layered as before on a discontinuous gradient as described above. The run was performed as described before except that this time the run was performed overnight. The next day the band was pulled with care to avoid pulling any of the defective particle band. The virus was then dialyzed using a Slide-a-Lyzer™ Cassette (Pierce) against ARM buffer (20 mM Tris pH 8.0, 25 mM NaCl, 2.5% Glycerol). This was performed 3×, 1 h per buffer exchange. The virus was then aliquoted for storage at −80° C.

### Viral Assays

VP concentration was performed by using an OD 260 assay based on the extinction coefficient of 1.1×10¹² viral particles (VP) is equivalent to an Absorbance value of 1 at OD260 nm. Two dilutions (1:5 and 1:10) of adenovirus were made in a viral lysis buffer (0.1% SDS, 10 mM Tris pH 7.4, 1 mM EDTA). OD was measured in duplicate at both dilutions and the VP concentration/mL was measured by multiplying the OD260 value X dilution factor X 1.1×10¹²VP.

An infectious unit (IU) titer was calculated by a limiting dilution assay of the viral stock. The virus was initially diluted 100× in DMEM/5% NS/1×PS and then subsequently diluted using 10-fold dilutions down to 1×10⁻⁷. 100 μL of these dilutions were then added to 293A cells that were seeded at least an hour before at 3e5 cells/well of a 24 well plate. This was performed in duplicate. Plates were incubated for 48 h in a CO2 (5%) incubator at 37° C. The cells were then washed with 1×PBS and were then fixed with 100% cold methanol (−20° C.). The plates were then incubated at −20° C. for a minimum of 20 minutes. The wells were washed with 1×PBS then blocked in 1×PBS/0.1% BSA for 1 h at room temperature. A rabbit anti-Ad antibody (Abcam, Cambridge, Mass.) was added at 1:8,000 dilution in blocking buffer (0.25 ml per well) and incubated for 1 h at room temperature. The wells were washed 4× with 0.5 mL PBS per well. A HRP conjugated Goat anti-Rabbit antibody (Bethyl Labs, Montgomery Texas) diluted 1000× was added per well and incubated for 1 h prior to a final round of washing. 5 PBS washes were performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells were developed for 5 min prior to counting. Cells were counted under a 10× objective using a dilution that gave between 4-40 stained cells per field of view. The field of view that was used was a 0.32 mm² grid of which there are equivalent to 625 per field of view on a 24 well plate. The number of infectious viruses/mL can be determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### Immunizations

C57BL/6J female mice and Balb/c female mice were injected with 1×10⁸ viral particles (VP) of ChAdV68.5WTnt.MAG25mer in 100 uL volume, bilateral intramuscular injection (50 uL per leg).

### Splenocyte Dissociation

Spleen and lymph nodes for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Attune N×T flow cytometer (Thermo Fisher) using propidium iodide staining to exclude dead and apoptotic cells. Cell were then adjusted to the appropriate concentration of live cells for subsequent analysis.

### Ex Vivo Enzyme-Linked Immunospot (ELISpot) Analysis

ELISpot analysis was performed according to ELISpot harmonization guidelines {DOI: 10.1038/nprot.2015.068} with the mouse IFNg ELISpotPLUS kit (MABTECH). 5×10⁴ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISpot analysis, wells with saturation >50% were recorded as “too numerous to count”. Samples with deviation of replicate wells >10% were excluded from analysis. Spot counts were then corrected for well confluency using the formula: spot count+2 x (spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### XV.B.2. Production of ChAdV68 Viral Delivery Particles after DNA Transfection

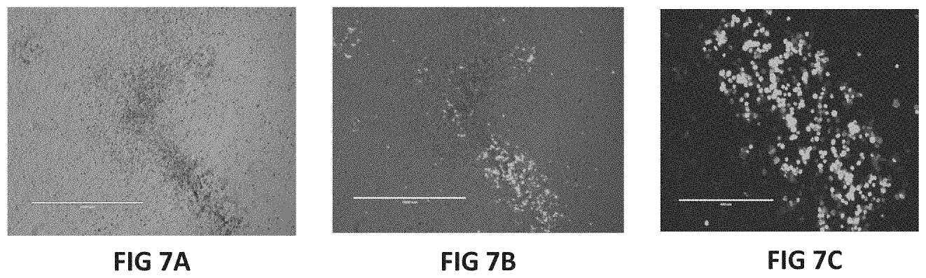

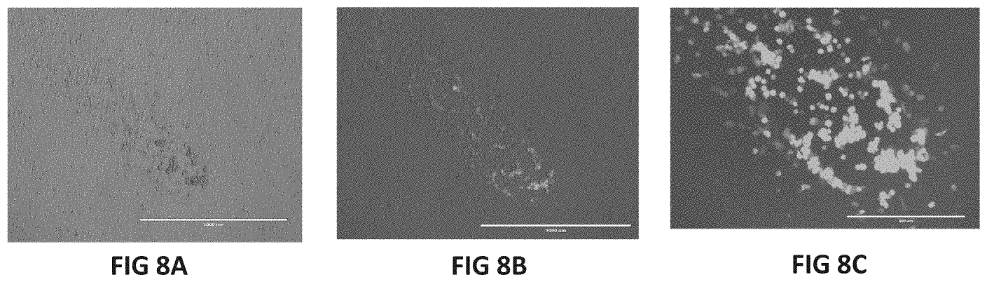

In one example, ChAdV68.4WTnt.GFP (FIG. 7) and ChAdV68.5WTnt.GFP (FIG. 8) DNA was transfected into HEK293A cells and virus replication (viral plaques) was observed 7-10 days after transfection. ChAdV68 viral plaques were visualized using light (FIGS. 7A and 8A) and fluorescent microscopy (FIG. 7B-C and FIG. 8B-C). GFP denotes productive ChAdV68 viral delivery particle production.

### XV.B.3. ChAdV68 Viral Delivery Particles Expansion

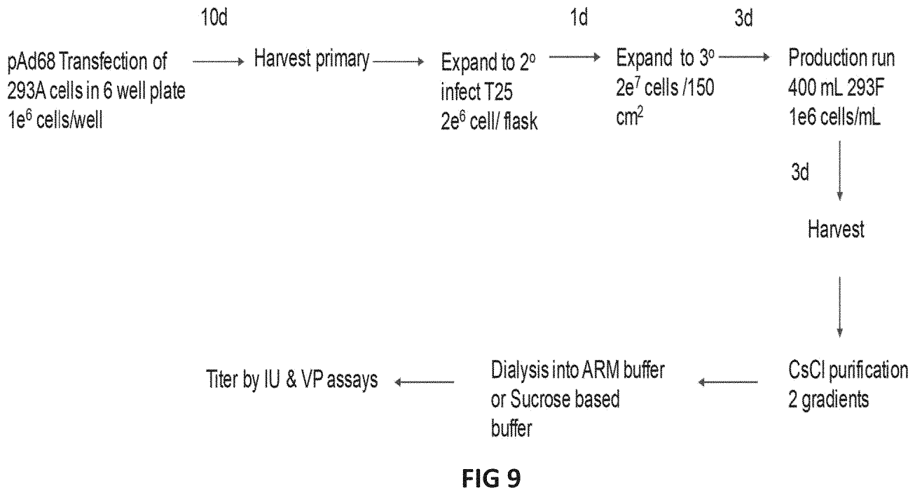

In one example, ChAdV68.4WTnt.GFP, ChAdV68.5WTnt.GFP, and ChAdV68.5WTnt.MAG25mer viruses were expanded in HEK293F cells and a purified virus stock produced 18 days after transfection (FIG. 9). Viral particles were quantified in the purified ChAdV68 virus stocks and compared to adenovirus type 5 (Ad5) and ChAdVY25 (a closely related ChAdV; Dicks, 2012, PloS ONE 7, e40385) viral stocks produced using the same protocol. ChAdV68 viral titers were comparable to Ad5 and ChAdVY25 (Table 7).

TABLE 7
Adenoviral vector production in 293F suspension cells

| Construct | Average VP/cell +/− SD |
|---|---|
| Ad5-Vectors (Multiple vectors) | 2.96e4 +/− 2.26e4 |
| Ad5-GFP | 3.89e4 |
| chAdY25-GFP | 1.75e3 +/− 6.03e1 |
| ChAdV68.4WTnt.GFP | 1.2e4 +/− 6.5e3 |
| ChAdV68.5WTnt.GFP | 1.8e3 |
| ChAdV68.5WTnt.MAG25mer | 1.39e3 +/− 1.1e3 |

*SD is only reported where multiple Production runs have been performed

### XV.B.4. Evaluation of Immunogenicity in Tumor Models

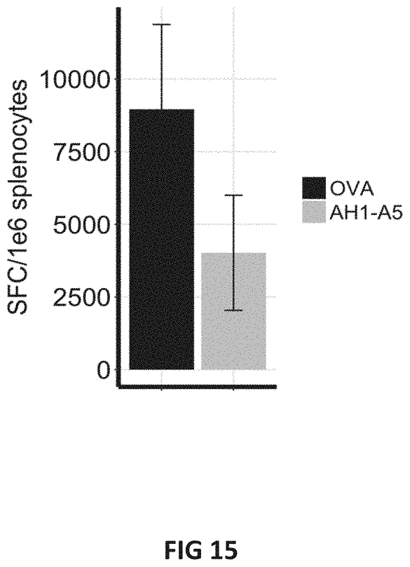

C68 vector expressing mouse tumor antigens were evaluated in mouse immunogenicity studies to demonstrate the C68 vector elicits T-cell responses. T-cell responses to the MHC class I epitope SIINFEKL (SEQ ID NO: 29362) were measured in C57BL/6J female mice and the MHC class I epitope AH1-A5 (Slansky et al., 2000, Immunity 13:529-538) measured in Balb/c mice. As shown in FIG. 15, strong T-cell responses relative to control were measured after immunization of mice with ChAdV68.5WTnt.MAG25mer. Mean cellular immune responses of 8957 or 4019 spot forming cells (SFCs) per 10⁶ splenocytes were observed in ELISpot assays when C57BL/6J or Balb/c mice were immunized with ChAdV68.5WTnt.MAG25mer, respectively, 10 days after immunization.

Tumor infiltrating lymphocytes were also evaluated in CT26 tumor model evaluating ChAdV and co-administration of an anti-CTLA4 antibody. Mice were implanted with CT26 tumors cells and 7 days after implantation, were immunized with ChAdV vaccine and treated with anti-CTLA4 antibody (clone 9D9) or IgG as a control. Tumor infiltrating lymphocytes were analyzed 12 days after immunization. Tumors from each mouse were dissociated using the gentleMACS Dissociator (Miltenyi Biotec) and mouse tumor dissociation kit (Miltenyi Biotec). Dissociated cells were filtered through a 30 micron filter and resuspended in complete RPMI. Cells were counted on the Attune N×T flow cytometer (Thermo Fisher) using propidium iodide staining to exclude dead and apoptotic cells. Cell were then adjusted to the appropriate concentration of live cells for subsequent analysis. Antigen specific cells were identified by MHC-tetramer complexes and co-stained with anti-CD8 and a viability marker. Tumors were harvested 12 days after prime immunization.

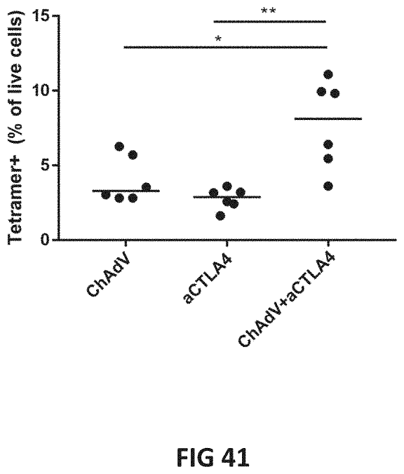

Antigen-specific CD8+ T cells within the tumor comprised a median of 3.3%, 2.2%, or 8.10% of the total live cell population in ChAdV, anti-CTLA4, and ChAdV+anti-CTLA4 treated groups, respectively (FIG. 41 and Table 45). Treatment with anti-CTLA in combination with active ChAdV immunization resulted in a statistically significant increase in the antigen-specific CD8+ T cell frequency over both ChAdV alone and anti-CTLA4 alone demonstrating anti-CTLA4, when co-administered with the chAd68 vaccine, increased the number of infiltrating T cells within a tumor.

TABLE 45
Tetramer+ infiltrating CD8 T cell
frequencies in CT26 tumors

| Treatment | Median % tetramer+ |
|---|---|
| ChAdV68.5 WTnt.MAG25mer | 3.3 |
| (ChAdV) | |
| Anti-CTLA4 | 2.2 |
| ChAdV68.5 WTnt.MAG25mer | 8.1 |
| (ChAdV) + anti-CTLA4 | |

### XVI. Alphavirus Antigen Cassette Delivery Vector

### XVI.A. Alphavirus Delivery Vector Evaluation Materials and Methods

### In Vitro Transcription to Generate RNA

For in vitro testing: plasmid DNA was linearized by restriction digest with PmeI, column purified following manufacturer's protocol (GeneJet DNA cleanup kit, Thermo) and used as template. In vitro transcription was performed using the RiboMAX Large Scale RNA production System (Promega) with the m⁷G cap analog (Promega) according to manufacturer's protocol. mRNA was purified using the RNeasy kit (Qiagen) according to manufacturer's protocol.

For in vivo studies: RNA was generated and purified by TriLInk Biotechnologies and capped with Enzymatic Cap1.

### Transfection of RNA

HEK293A cells were seeded at 6e4 cells/well for 96 wells and 2e5 cells/well for 24 wells, ˜16 hours prior to transfection. Cells were transfected with mRNA using MessengerMAX lipofectamine (Invitrogen) and following manufacturer's protocol. For 96-wells, 0.15 uL of lipofectamine and 10 ng of mRNA was used per well, and for 24-wells, 0.75 uL of lipofectamine and 150 ng of mRNA was used per well. A GFP expressing mRNA (TriLink Biotechnologies) was used as a transfection control.

### Luciferase Assay

Luciferase reporter assay was performed in white-walled 96-well plates with each condition in triplicate using the ONE-Glo luciferase assay (Promega) following manufacturer's protocol. Luminescence was measured using the SpectraMax.

qRT-PCR

Transfected cells were rinsed and replaced with fresh media 2 hours post transfection to remove any untransfected mRNA. Cells were then harvested at various timepoints in RLT plus lysis buffer (Qiagen), homogenized using a QiaShredder (Qiagen) and RNA was extracted using the RNeasy kit (Qiagen), all according to manufacturer's protocol. Total RNA was quantified using a Nanodrop (Thermo Scientific). qRT-PCR was performed using the Quantitect Probe One-Step RT-PCR kit (Qiagen) on the qTower³ (Analytik Jena) according to manufacturer's protocol, using 20 ng of total RNA per reaction. Each sample was run in triplicate for each probe. Actin or GusB were used as reference genes. Custom primer/probes were generated by IDT (Table 8).

TABLE 8
qPCR primers/probes

| Target | | |
|---|---|---|
| Luci | Primer1 | GTGGTGTGCAGCGAGAATAG |
| | | (SEQ ID NO: 29376) |
| | Primer2 | CGCTCGTTGTAGATGTCGTTAG |
| | | (SEQ ID NO: 29377) |
| | Probe | /56-FAM/TTGCAGTTC/ZEN/ |
| | | TTCATGCCCGTGTTG/3IABkFQ/ |
| | | (SEQ ID NO: 29378) |
| GusB | Primer1 | GTTTTTGATCCAGACCCAGATG |
| | | (SEQ ID NO: 29379) |
| | Primer2 | GCCCATTATTCAGAGCGAGTA |
| | | (SEQ ID NO: 29380) |
| | Probe | /56-FAM/TGCAGGGTT/ZEN/ |
| | | TCACCAGGATCCAC/3IABkFQ/ |
| | | (SEQ ID NO: 29381) |
| ActB | Primer1 | CCTTGCACATGCCGGAG |
| | | (SEQ ID NO: 29382) |
| | Primer2 | ACAGAGCCTCGCCTTTG |
| | | (SEQ ID NO: 29383) |
| | Probe | /56-FAM/TCATCCATG/ |
| | | ZEN/GTGAGCTGGCGG/3IABkFQ/ |
| | | (SEQ ID NO: 29384) |
| MAG-25mer | Primer1 | CTGAAAGCTCGGTTTGCTAATG |
| | | (SEQ ID NO: 29385) |
| Set1 | Primer2 | CCATGCTGGAAGAGACAATCT |
| | | (SEQ ID NO: 29386) |
| | Probe | /56-FAM/CGTTTCTGA/ZEN/ |
| | | TGGCGCTGACCGATA/3IABkFQ/ |
| | | (SEQ ID NO: 29387) |
| MAG-25mer | Primer1 | TATGCCTATCCTGTCTCCTCTG |
| | | (SEQ ID NO: 29388) |
| Set2 | Primer2 | GCTAATGCAGCTAAGTCCTCTC |
| | | (SEQ ID NO: 29389) |
| | Probe | /56-FAM/TGTTTACCC/ZEN/ |
| | | TGACCGTGCCTTCTG/3IABkFQ/ |
| | | (SEQ ID NO: 29390) |

### B16-OVA Tumor Model

C57BL/6J mice were injected in the lower left abdominal flank with 10⁵ B16-OVA cells/animal. Tumors were allowed to grow for 3 days prior to immunization.

### CT26 Tumor Model

Balb/c mice were injected in the lower left abdominal flank with 10⁶ CT26 cells/animal. Tumors were allowed to grow for 7 days prior to immunization.

### Immunizations

For srRNA vaccine, mice were injected with 10 ug of RNA in 100 uL volume, bilateral intramuscular injection (50 uL per leg). For Ad5 vaccine, mice were injected with 5×10¹⁰ viral particles (VP) in 100 uL volume, bilateral intramuscular injection (50 uL per leg). Animals were injected with anti-CTLA-4 (clone 9D9, BioXcell), anti-PD-1 (clone RMP1-14, BioXcell) or anti-IgG (clone MPC-11, BioXcell), 250 ug dose, 2 times per week, via intraperitoneal injection.

### In Vivo Bioluminescent Imaging

At each timepoint mice were injected with 150 mg/kg luciferin substrate via intraperitoneal injection and bioluminescence was measured using the IVIS In vivo imaging system (PerkinElmer) 10-15 minutes after injection.

### Splenocyte Dissociation

Spleen and lymph nodes for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Attune N×T flow cytometer (Thermo Fisher) using propidium iodide staining to exclude dead and apoptotic cells. Cell were then adjusted to the appropriate concentration of live cells for subsequent analysis.

### Ex Vivo Enzyme-Linked Immunospot (ELISpot) Analysis

ELISpot analysis was performed according to ELISpot harmonization guidelines {DOI: 10.1038/nprot.2015.068} with the mouse IFNg ELISpotPLUS kit (MABTECH). 5×10⁴ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISpot analysis, wells with saturation >50% were recorded as “too numerous to count”. Samples with deviation of replicate wells >10% were excluded from analysis. Spot counts were then corrected for well confluency using the formula: spot count+2 x (spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### XVI.B. Alphavirus Vector

### XVI.B.1. Alphavirus Vector In Vitro Evaluation

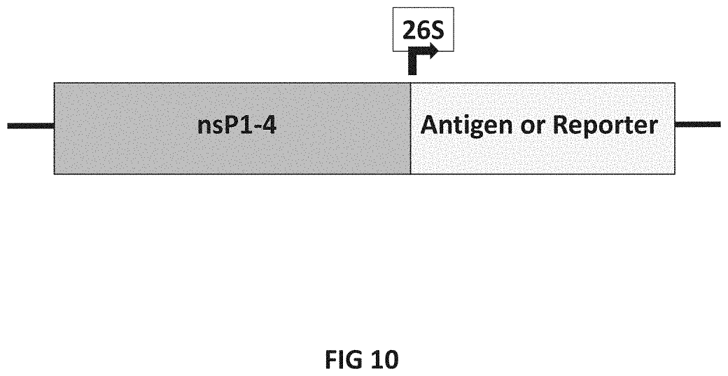

In one implementation of the present invention, a RNA alphavirus backbone for the antigen expression system was generated from a Venezuelan Equine Encephalitis (VEE) (Kinney, 1986, Virology 152: 400-413) based self-replicating RNA (srRNA) vector. In one example, the sequences encoding the structural proteins of VEE located 3′ of the 26S subgenomic promoter were deleted (VEE sequences 7544 to 11,175 deleted; numbering based on Kinney et al 1986; SEQ ID NO:6) and replaced by antigen sequences (SEQ ID NO:14 and SEQ ID NO:4) or a luciferase reporter (e.g., VEE-Luciferase, SEQ ID NO:15) (FIG. 10). RNA was transcribed from the srRNA DNA vector in vitro, transfected into HEK293A cells and luciferase reporter expression was measured. In addition, an (non-replicating) mRNA encoding luciferase was transfected for comparison. An ˜30,000-fold increase in srRNA reporter signal was observed for VEE-Luciferase srRNA when comparing the 23 hour measurement vs the 2 hour measurement (Table 9). In contrast, the mRNA reporter exhibited a less than 10-fold increase in signal over the same time period (Table 9).

TABLE 9
Expression of luciferase from VEE self-replicating vector increases
over time. HEK293A cells transfected with 10 ng of VEE-Luciferase
srRNA or 10 ng of non-replicating luciferase mRNA (TriLink L-6307)
per well in 96 wells. Luminescence was measured at various times post
transfection. Luciferase expression is reported as relative luminescence
units (RLU). Each data point is the mean +/− SD of 3 transfected wells.

| | | | Standard Dev |
|---|---|---|---|
| Construct | Timepoint (hr) | Mean RLU | (triplicate wells) |
| mRNA | 2 | 878.6666667 | 120.7904522 |
| mRNA | 5 | 1847.333333 | 978.515372 |
| mRNA | 9 | 4847 | 868.3271273 |
| mRNA | 23 | 8639.333333 | 751.6816702 |
| SRRNA | 2 | 27 | 15 |
| SRRNA | 5 | 4884.333333 | 2955.158935 |
| SRRNA | 9 | 182065.5 | 16030.81784 |
| SRRNA | 23 | 783658.3333 | 68985.05538 |

In another example, replication of the srRNA was confirmed directly by measuring RNA levels after transfection of either the luciferase encoding srRNA (VEE-Luciferase) or an srRNA encoding a multi-epitope cassette (VEE-MAG25mer) using quantitative reverse transcription polymerase chain reaction (qRT-PCR). An ˜150-fold increase in RNA was observed for the VEE-luciferase srRNA (Table 10), while a 30-50-fold increase in RNA was observed for the VEE-MAG25mer srRNA (Table 11). These data confirm that the VEE srRNA vectors replicate when transfected into cells.

TABLE 10
Direct measurement of RNA replication in VEE-Luciferase srRNA transfected cells
HEK293A cells transfected with VEE-Luciferase srRNA (150 ng per well, 24-well) and RNA
levels quantified by qRT-PCR at various times after transfection. Each measurement was
normalized based on the Actin reference gene and fold-change relative to the 2 hour timepoint
is presented.

| Timepoint | | | | | | Relative Fold |
|---|---|---|---|---|---|---|
| (hr) | Luciferase Ct | Actin Ct | dCt | RefdCt | ddCt | change |
| 2 | 20.51 | 18.14 | 2.38 | 2.38 | 0.00 | 1.00 |
| 4 | 20.09 | 18.39 | 1.70 | 2.38 | −0.67 | 1.59 |
| 6 | 15.50 | 18.19 | −2.69 | 2.38 | 5.07 | 33.51 |
| 8 | 13.51 | 18.36 | −4.85 | 2.38 | −7.22 | 149.43 |

TABLE 11
Direct measurement of RNA replication in VEE-MAG25mer srRNA transfected
cells. HEK293 cells transfected with VEE-MAG25mer srRNA (150 ng per well, 24-well) and
RNA levels quantified by qRT−PCR at various times after transfection. Each measurement was
normalized based on the GusB reference gene and fold-change relative to the 2 hour timepoint
is presented. Different lines on the graph represent 2 different qPCR primer/probe sets, both of
which detect the epitope cassette region of the srRNA.

| Primer/ | Timepoint | | GusB | | | | Relative |
|---|---|---|---|---|---|---|---|
| probe | (hr) | Ct | Ct | dCt | RefdCt | ddCt | Fold-Change |
| Setl | 2 | 18.96 | 22.41 | −3.45 | −3.45 | 0.00 | 1.00 |
| Setl | 4 | 17.46 | 22.27 | −4.81 | −3.45 | −1.37 | 2.58 |
| Setl | 6 | 14.87 | 22.04 | −7.17 | −3.45 | −3.72 | 13.21 |
| Setl | 8 | 14.16 | 22.19 | −8.02 | −3.45 | −4.58 | 23.86 |
| Setl | 24 | 13.16 | 22.01 | −8.86 | −3.45 | −5.41 | 42.52 |
| Setl | 36 | 13.53 | 22.63 | −9.10 | −3.45 | −5.66 | 50.45 |
| Set2 | 2 | 17.75 | 22.41 | −4.66 | −4.66 | 0.00 | 1.00 |
| Set2 | 4 | 16.66 | 22.27 | −5.61 | −4.66 | −0.94 | 1.92 |
| Set2 | 6 | 14.22 | 22.04 | −7.82 | −4.66 | −3.15 | 8.90 |
| Set2 | 8 | 13.18 | 22.19 | −9.01 | −4.66 | −4.35 | 20.35 |
| Set2 | 24 | 12.22 | 22.01 | −9.80 | −4.66 | −5.13 | 35.10 |
| Set2 | 36 | 13.08 | 22.63 | −9.55 | −4.66 | −4.89 | 29.58 |

### XVI.B.2. Alphavirus Vector In Vivo Evaluation

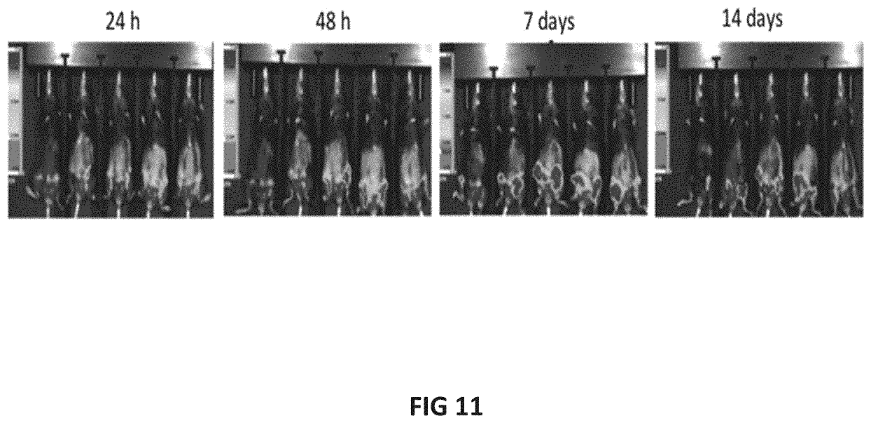

In another example, VEE-Luciferase reporter expression was evaluated in vivo. Mice were injected with 10 ug of VEE-Luciferase srRNA encapsulated in lipid nanoparticle (MC3) and imaged at 24 and 48 hours, and 7 and 14 days post injection to determine bioluminescent signal. Luciferase signal was detected at 24 hours post injection and increased over time and appeared to peak at 7 days after srRNA injection (FIG. 11).

### XVI.B.3. Alphavirus Vector Tumor Model Evaluation

In one implementation, to determine if the VEE srRNA vector directs antigen-specific immune responses in vivo, a VEE srRNA vector was generated (VEE-UbAAY, SEQ ID NO:14) that expresses 2 different MHC class I mouse tumor epitopes, SIINFEKL (SEQ ID NO: 29362) and AH1-A5 (Slansky et al., 2000, Immunity 13:529-538). The SFL (SIINFEKL; SEQ ID NO: 29362) epitope is expressed by the B16-OVA melanoma cell line, and the AH1-A5 (SPSYAYHQF (SEQ ID NO: 29363); Slansky et al., 2000, Immunity) epitope induces T cells targeting a related epitope (AH1/SPSYVYHQF (SEQ ID NO: 29391); Huang et al., 1996, Proc Natl Acad Sci USA 93:9730-9735) that is expressed by the CT26 colon carcinoma cell line. In one example, for in vivo studies, VEE-UbAAY srRNA was generated by in vitro transcription using T7 polymerase (TriLink Biotechnologies) and encapsulated in a lipid nanoparticle (MC3).

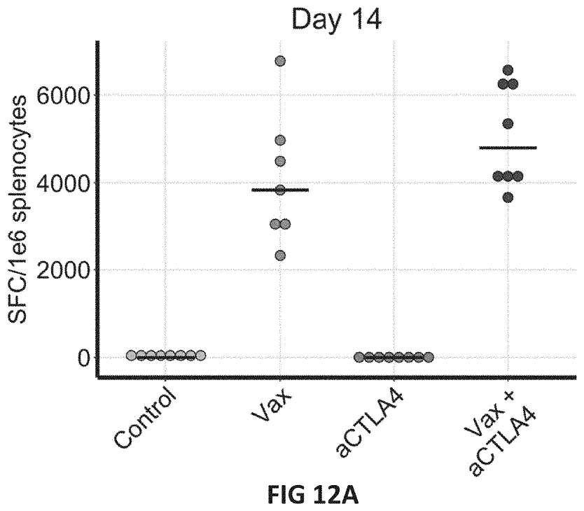

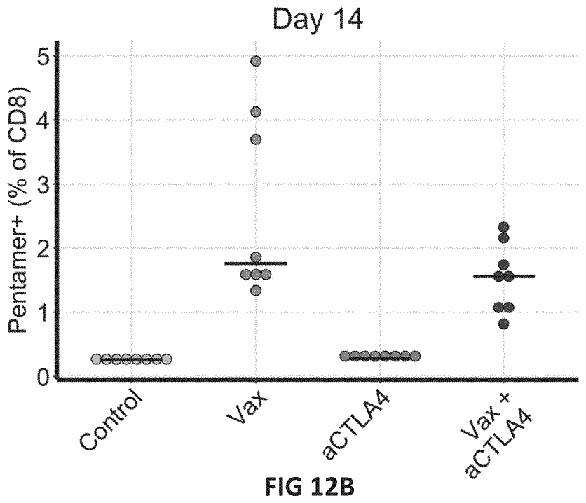

A strong antigen-specific T-cell response targeting SFL, relative to control, was observed two weeks after immunization of B16-OVA tumor bearing mice with MC3 formulated VEE-UbAAY srRNA. In one example, a median of 3835 spot forming cells (SFC) per 10⁶ splenocytes was measured after stimulation with the SFL peptide in ELISpot assays (FIG. 12A, Table 12) and 1.8% (median) of CD8 T-cells were SFL antigen-specific as measured by pentamer staining (FIG. 12B, Table 12). In another example, co-administration of an anti-CTLA-4 monoclonal antibody (mAb) with the VEE srRNA vaccine resulted in a moderate increase in overall T-cell responses with a median of 4794.5 SFCs per 10⁶ splenocytes measured in the ELISpot assay (FIG. 12A, Table 12).

TABLE 12
Results of ELISpot and MHCI-pentamer staining assays 14 days post VEE srRNA
immunization in B16-0VA tumor bearing C57BL/6J mice.

| | | | Pentamer | | | | Pentamer |
|---|---|---|---|---|---|---|---|
| | | SFC/1e6 | positive (% | | | SFC/1e6 | positive (% |
| Group | Mouse | splenocytes | of CD8) | Group | Mouse | splenocytes | of CD8) |
| Control | 1 | 47 | 0.22 | Vax | 1 | 6774 | 4.92 |
| | 2 | 80 | 0.32 | | 2 | 2323 | 1.34 |
| | 3 | 0 | 0.27 | | 3 | 2997 | 1.52 |
| | 4 | 0 | 0.29 | | 4 | 4492 | 1.86 |
| | 5 | 0 | 0.27 | | 5 | 4970 | 3.7 |
| | 6 | 0 | 0.25 | | 6 | | 4.13 |
| | 7 | 0 | 0.23 | | 7 | 3835 | 1.66 |
| | 8 | 87 | 0.25 | | 8 | 3119 | 1.64 |
| aCTLA4 | 1 | 0 | 0.24 | Vax + | 1 | 6232 | 2.16 |
| | 2 | 0 | 0.26 | aCTLA4 | 2 | 4242 | 0.82 |
| | 3 | 0 | 0.39 | | 3 | 5347 | 1.57 |
| | 4 | 0 | 0.28 | | 4 | 6568 | 2.33 |
| | 5 | 0 | 0.28 | | 5 | 6269 | 1.55 |
| | 6 | 0 | 0.28 | | 6 | 4056 | 1.74 |
| | 7 | 0 | 0.31 | | 7 | 4163 | 1.14 |
| | 8 | 6 | 0.26 | | 8 | 3667 | 1.01 |

* Note that results from mouse #6 in the Vax group were excluded from analysis due to high variability between triplicate wells.

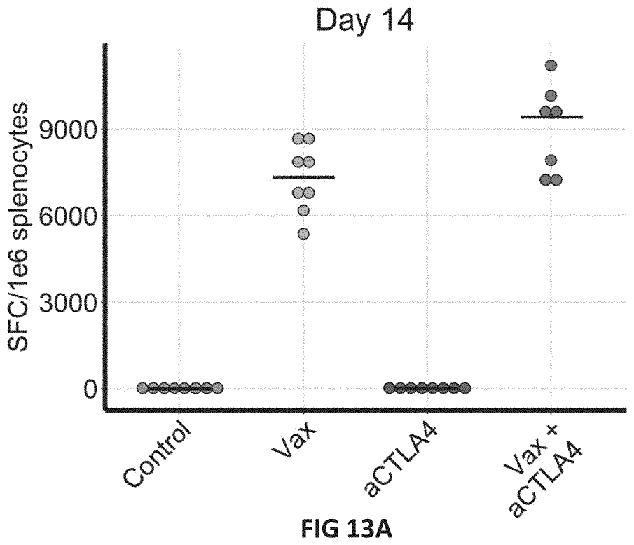

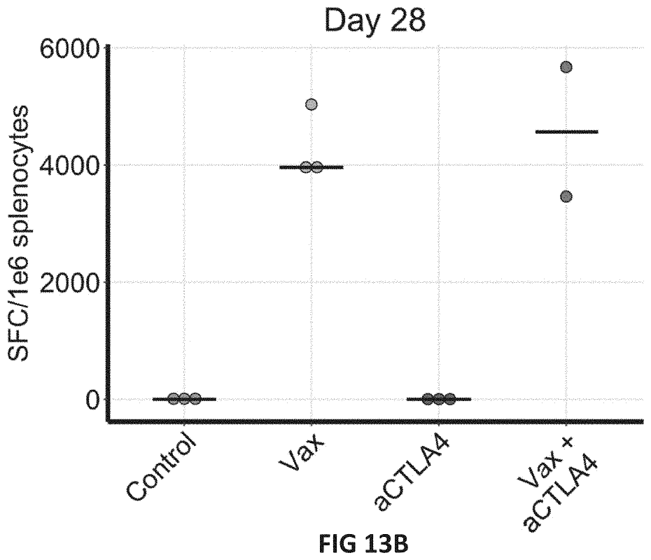

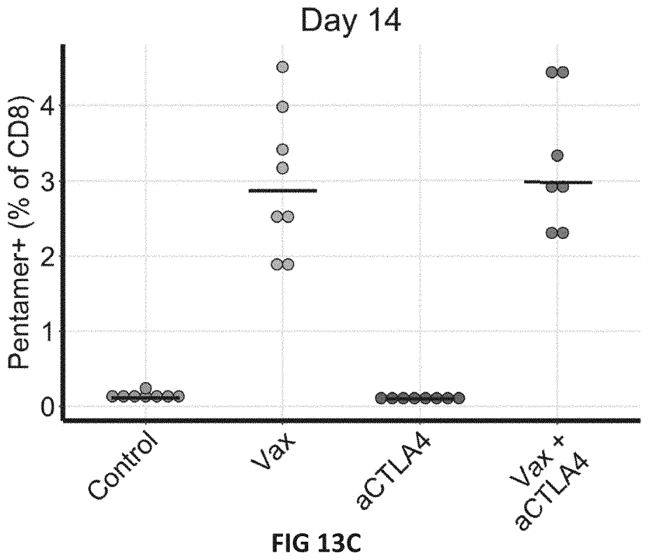

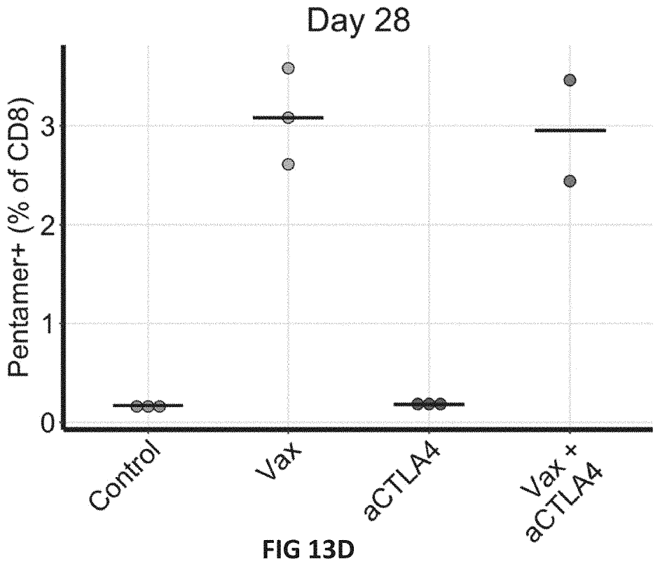

In another implementation, to mirror a clinical approach, a heterologous prime/boost in the B16-OVA and CT26 mouse tumor models was performed, where tumor bearing mice were immunized first with adenoviral vector expressing the same antigen cassette (Ad5-UbAAY), followed by a boost immunization with the VEE-UbAAY srRNA vaccine 14 days after the Ad5-UbAAY prime. In one example, an antigen-specific immune response was induced by the Ad5-UbAAY vaccine resulting in 7330 (median) SFCs per 10⁶ splenocytes measured in the ELISpot assay (FIG. 13A, Table 13) and 2.9% (median) of CD8 T-cells targeting the SFL antigen as measured by pentamer staining (FIG. 13C, Table 13). In another example, the T-cell response was maintained 2 weeks after the VEE-UbAAY srRNA boost in the B16-OVA model with 3960 (median) SFL-specific SFCs per 10⁶ splenocytes measured in the ELISpot assay (FIG. 131B, Table 13) and 3.1% (median) of CD8 T-cells targeting the SFL antigen as measured by pentamer staining (FIG. 13D, Table 13).

TABLE 13
Immune monitoring of B16-OVA mice following heterologous prime/boost with
Ad5 vaccine prime and srRNA boost.

| | | | | | | | |
|---|---|---|---|---|---|---|---|
| | | | Pentamer | | | | Pentamer |
| | | SFC/1e6 | positive | | | SFC/1e6 | positive |
| Group | Mouse | splenocytes | (% of CD8) | Group | Mouse | splenocytes | (% of CD8) |

Day 14

| | | | | | | | |
|---|---|---|---|---|---|---|---|
| Control | 1 | 0 | 0.10 | Vax | 1 | 8514 | 1.87 |
| | 2 | 0 | 0.09 | | 2 | 7779 | 1.91 |
| | 3 | 0 | 0.11 | | 3 | 6177 | 3.17 |
| | 4 | 46 | 0.18 | | 4 | 7945 | 3.41 |
| | 5 | 0 | 0.11 | | 5 | 8821 | 4.51 |
| | 6 | 16 | 0.11 | | 6 | 6881 | 2.48 |
| | 7 | 0 | 0.24 | | 7 | 5365 | 2.57 |
| | 8 | 37 | 0.10 | | 8 | 6705 | 3.98 |
| aCTLA4 | 1 | 0 | 0.08 | Vax + | 1 | 9416 | 2.35 |
| | 2 | 29 | 0.10 | aCTLA4 | 2 | 7918 | 3.33 |
| | 3 | 0 | 0.09 | | 3 | 10153 | 4.50 |
| | 4 | 29 | 0.09 | | 4 | 7212 | 2.98 |
| | 5 | 0 | 0.10 | | 5 | 11203 | 4.38 |
| | 6 | 49 | 0.10 | | 6 | 9784 | 2.27 |
| | 7 | 0 | 0.10 | | 8 | 7267 | 2.87 |

| | | |
|---|---|---|
| 8 | 31 | 0.14 |

Day 28

| | | | | | | | |
|---|---|---|---|---|---|---|---|
| Control | 2 | 0 | 0.17 | Vax | 1 | 5033 | 2.61 |
| | 4 | 0 | 0.15 | | 2 | 3958 | 3.08 |
| | 6 | 20 | 0.17 | | 4 | 3960 | 3.58 |
| aCTLA4 | 1 | 7 | 0.23 | Vax + | 4 | 3460 | 2.44 |
| | 2 | 0 | 0.18 | aCTLA4 | 5 | 5670 | 3.46 |

| | | |
|---|---|---|
| 3 | 0 | 0.14 |

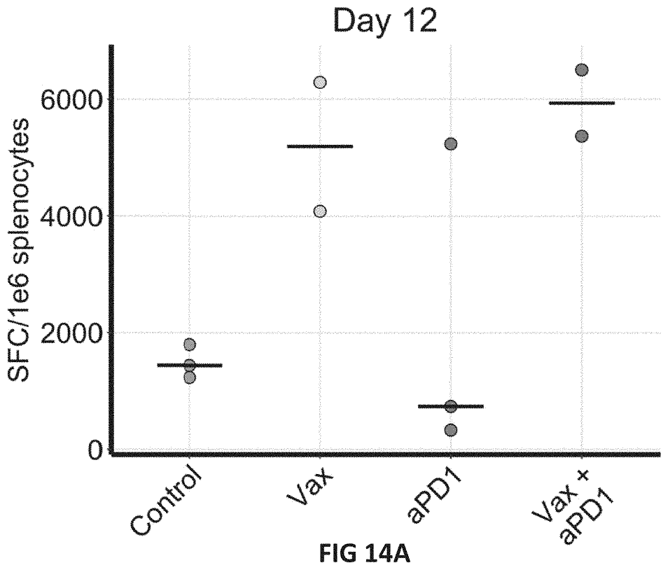

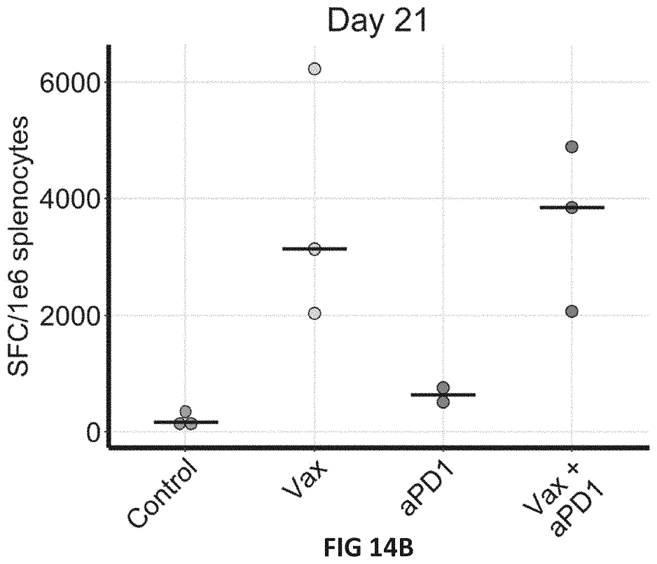

In another implementation, similar results were observed after an Ad5-UbAAY prime and VEE-UbAAY srRNA boost in the CT26 mouse model. In one example, an AH1 antigen-specific response was observed after the Ad5-UbAAY prime (day 14) with a mean of 5187 SFCs per 10⁶ splenocytes measured in the ELISpot assay (FIG. 14A, Table 14) and 3799 SFCs per 10⁶ splenocytes measured in the ELISpot assay after the VEE-UbAAY srRNA boost (day 28) (FIG. 141B, Table 14).

TABLE 14
Immune monitoring after heterologous prime/boost in
CT26 tumor mouse model

| Day 12 | | | Day 21 | | |
|---|---|---|---|---|---|
| | | SFC/1e6 | | | SFC/1e6 |
| Group | Mouse | splenocytes | Group | Mouse | splenocytes |
| Control | 1 | 1799 | Control | 9 | 167 |
| | 2 | 1442 | | 10 | 115 |
| | 3 | 1235 | | 11 | 347 |
| aPD1 | 1 | 737 | aPD1 | 8 | 511 |
| | 2 | 5230 | | 11 | 758 |
| | 3 | 332 | Vax | 9 | 3133 |
| Vax | 1 | 6287 | | 10 | 2036 |
| | 2 | 4086 | | 11 | 6227 |
| Vax + | 1 | 5363 | Vax + | 8 | 3844 |
| aPD1 | 2 | 6500 | aPD1 | 9 | 2071 |

| | |
|---|---|
| 11 | 4888 |

### XVII. ChAdV/srRNA Combination Tumor Model Evaluation

Various dosing protocols using ChAdV68 and self-replicating RNA (srRNA) were evaluated in murine CT26 tumor models.

### XVII.A ChAdV/srRNA Combination Tumor Model Evaluation

### Methods and Materials

### Tumor Injection

Balb/c mice were injected with the CT26 tumor cell line. 7 days after tumor cell injection, mice were randomized to the different study arms (28-40 mice per group) and treatment initiated. Balb/c mice were injected in the lower left abdominal flank with 10⁶ CT26 cells/animal. Tumors were allowed to grow for 7 days prior to immunization. The study arms are described in detail in Table 15.

TABLE 15
ChAdV/srRNA Combination Tumor Model Evaluation Study Arms

| Group | N | Treatment | Dose | | Volume | | Schedule | Route |
|---|---|---|---|---|---|---|---|---|
| 1 | 40 | chAd68 control | 1e11 | vp | 2× 50 | uL | day 0 | IM |
| | | srRNA control | 10 | ug | 50 | uL | day 14, 28, 42 | IM |
| | | Anti-PD1 | 250 | ug | 100 | uL | 2×/week (start day 0) | IP |
| 2 | 40 | chAd68 control | 1e11 | vp | 2× 50 | uL | day 0 | IM |
| | | srRNA control | 10 | ug | 50 | uL | day 14, 28, 42 | IM |
| | | Anti-IgG | 250 | ug | 100 | uL | 2×/week (start day 0) | IP |
| 3 | 28 | chAd68 vaccine | 1e11 | vp | 2× 50 | uL | day 0 | IM |
| | | srRNA vaccine | 10 | ug | 50 | uL | day 14, 28, 42 | IM |
| | | Anti-PD1 | 250 | ug | 100 | uL | 2×/week (start day 0) | IP |
| 4 | 28 | chAd68 vaccine | 1e11 | vp | 2× 50 | uL | day 0 | IM |
| | | srRNA vaccine | 10 | ug | 50 | uL | day 14, 28, 42 | IM |
| | | Anti-IgG | 250 | ug | 100 | uL | 2×/week (start day 0) | IP |
| 5 | 28 | srRNA vaccine | 10 | ug | 50 | uL | day 0, 28, 42 | IM |
| | | chAd68 vaccine | 1e11 | vp | 2× 50 | uL | day 14 | IM |
| | | Anti-PD1 | 250 | ug | 100 | uL | 2×/week (start day 0) | IP |
| 6 | 28 | srRNA vaccine | 10 | ug | 50 | uL | day 0, 28, 42 | IM |
| | | chAd68 vaccine | 1e11 | vp | 2× 50 | uL | day 14 | IM |
| | | Anti-IgG | 250 | ug | 100 | uL | 2×/week (start day 0) | IP |
| 7 | 40 | srRNA vaccine | 10 | ug | 50 | uL | day 0, 14, 28, 42 | IM |
| | | Anti-PD1 | 250 | ug | 100 | uL | 2×/week (start day 0) | IP |
| 8 | 40 | srRNA vaccine | 10 | ug | 50 | uL | day 0, 14, 28, 42 | IM |
| | | Anti-IgG | 250 | ug | 100 | uL | 2×/week (start day 0) | IP |

### Immunizations

For srRNA vaccine, mice were injected with 10 ug of VEE-MAG25mer srRNA in 100 uL volume, bilateral intramuscular injection (50 uL per leg). For C68 vaccine, mice were injected with 1×10¹¹ viral particles (VP) of ChAdV68.5WTnt.MAG25mer in 100 uL volume, bilateral intramuscular injection (50 uL per leg). Animals were injected with anti-PD-1 (clone RMP1-14, BioXcell) or anti-IgG (clone MPC-11, BioXcell), 250 ug dose, 2 times per week, via intraperitoneal injection.

### Splenocyte Dissociation

Spleen and lymph nodes for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Attune N×T flow cytometer (Thermo Fisher) using propidium iodide staining to exclude dead and apoptotic cells. Cell were then adjusted to the appropriate concentration of live cells for subsequent analysis.

### Ex Vivo Enzyme-Linked Immunospot (ELISpot) Analysis

ELISpot analysis was performed according to ELISpot harmonization guidelines {DOI: 10.1038/nprot.2015.068} with the mouse IFNg ELISpotPLUS kit (MABTECH). 5×10⁴ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISpot analysis, wells with saturation >50% were recorded as “too numerous to count”. Samples with deviation of replicate wells >10% were excluded from analysis. Spot counts were then corrected for well confluency using the formula: spot count+2 x (spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### XVI.B ChAdV/srRNA Combination Evaluation in a CT26 Tumor Model

The immunogenicity and efficacy of the ChAdV68.5WTnt.MAG25mer/VEE-MAG25mer srRNA heterologous prime/boost or VEE-MAG25mer srRNA homologous prime/boost vaccines were evaluated in the CT26 mouse tumor model. Balb/c mice were injected with the CT26 tumor cell line. 7 days after tumor cell injection, mice were randomized to the different study arms and treatment initiated. The study arms are described in detail in Table 15 and more generally in Table 16.

TABLE 16
Prime/Boost Study Arms

| Group | Prime | Boost |
|---|---|---|
| 1 | Control | Control |
| 2 | Control + anti-PD-1 | Control + anti-PD-1 |
| 3 | ChAdV68.5WTnt.MAG25mer | VEE-MAG25mer srRNA |
| 4 | ChAdV68.5WTnt.MAG25mer + anti-PD-1 | VEE-MAG25mer srRNA + anti-PD-1 |
| 5 | VEE-MAG25mer srRNA | ChAdV68.5WTnt.MAG25mer |
| 6 | VEE-MAG25mer srRNA + anti-PD-1 | ChAdV68.5WTnt.MAG25mer + anti-PD-1 |
| 7 | VEE-MAG25mer srRNA | VEE-MAG25mer srRNA |
| 8 | VEE-MAG25mer srRNA + anti-PD-1 | VEE-MAG25mer srRNA + anti-PD-1 |

Spleens were harvested 14 days after the prime vaccination for immune monitoring. Tumor and body weight measurements were taken twice a week and survival was monitored. Strong immune responses relative to control were observed in all active vaccine groups.

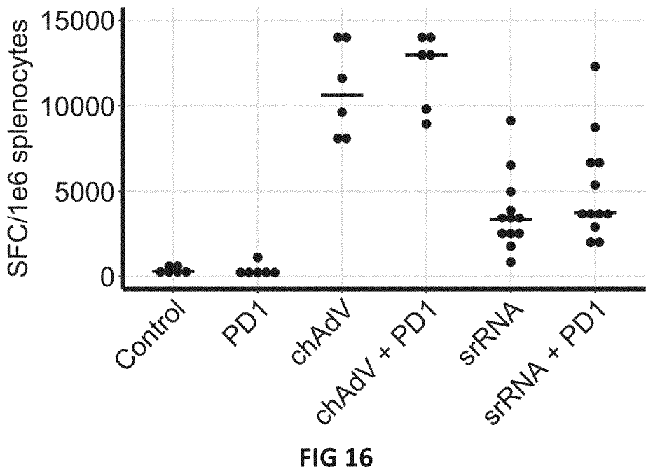

Median cellular immune responses of 10,630, 12,976, 3319, or 3745 spot forming cells (SFCs) per 10⁶ splenocytes were observed in ELISpot assays in mice immunized with ChAdV68.5WTnt.MAG25mer (ChAdV/group 3), ChAdV68.5WTnt.MAG25mer+anti-PD-1 (ChAdV+PD-1/group 4), VEE-MAG25mer srRNA (srRNA/median for groups 5 & 7 combined), or VEE-MAG25mer srRNA+anti-PD-1 (srRNA+PD-1/median for groups 6 & 8 combined), respectively, 14 days after the first immunization (FIG. 16 and Table 17). In contrast, the vaccine control (group 1) or vaccine control with anti-PD-1 (group 2) exhibited median cellular immune responses of 296 or 285 SFC per 10⁶ splenocytes, respectively.

TABLE 17
Cellular immune responses in a CT26 tumor model

| | Median SFC/ |
|---|---|
| Treatment | 10⁶ Splenocytes |
| Control | 296 |
| PD1 | 285 |
| ChAdV68.5WTnt.MAG25mer | 10630 |
| (ChAdV) | |
| ChAdV68.5WTnt.MAG25mer + | 12976 |
| PD1 (ChAdV + PD-1) | |
| VEE-MAG25mer srRNA | 3319 |
| (srRNA) | |
| VEE-MAG25mer srRNA + | 3745 |
| PD-1 (srRNA + PD1) | |

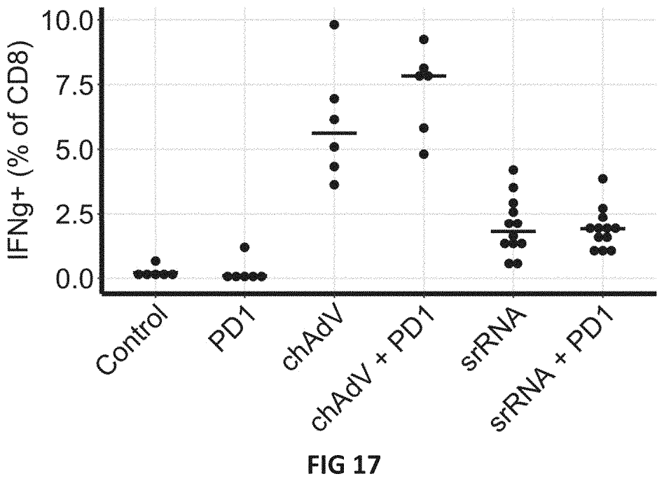

Consistent with the ELISpot data, 5.6, 7.8, 1.8 or 1.9% of CD8 T cells (median) exhibited antigen-specific responses in intracellular cytokine staining (ICS) analyses for mice immunized with ChAdV68.5WTnt.MAG25mer (ChAdV/group 3), ChAdV68.5WTnt.MAG25mer+anti-PD-1 (ChAdV+PD-1/group 4), VEE-MAG25mer srRNA (srRNA/median for groups 5 & 7 combined), or VEE-MAG25mer srRNA+anti-PD-1 (srRNA+PD-1/median for groups 6 & 8 combined), respectively, 14 days after the first immunization (FIG. 17 and Table 18). Mice immunized with the vaccine control or vaccine control combined with anti-PD-1 showed antigen-specific CD8 responses of 0.2 and 0.1%, respectively.

TABLE 18
CD8 T-Cell responses in a CT26 tumor model

| | Median % CD8 IFN- |
|---|---|
| Treatment | gamma Positive |
| Control | 0.21 |
| PD1 | 0.1 |
| ChAdV68.5WTnt.MAG25mer | 5.6 |
| (ChAdV) | |
| ChAdV68.5WTnt.MAG25mer + | 7.8 |
| PD1 (ChAdV + PD-1) | |
| VEE-MAG25mer srRNA | 1.8 |
| (srRNA) | |
| VEE-MAG25mer srRNA + | 1.9 |
| PD-1 (srRNA + PD1) | |

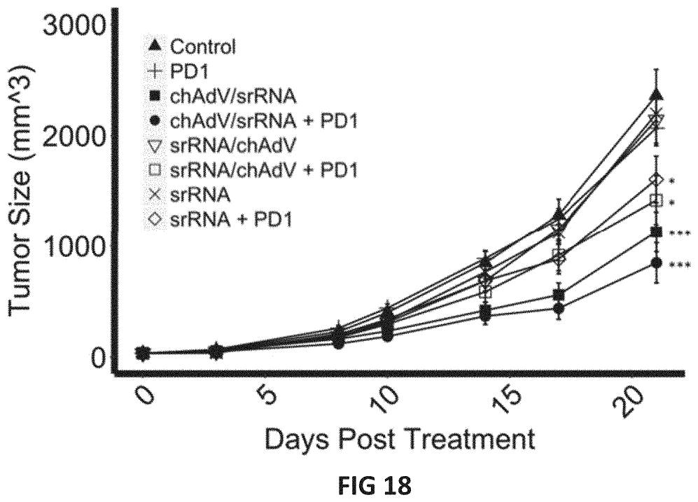

Tumor growth was measured in the CT26 colon tumor model for all groups, and tumor growth up to 21 days after treatment initiation (28 days after injection of CT-26 tumor cells) is presented. Mice were sacrificed 21 days after treatment initiation based on large tumor sizes (>2500 mm³); therefore, only the first 21 days are presented to avoid analytical bias. Mean tumor volumes at 21 days were 1129, 848, 2142, 1418, 2198 and 1606 mm³ for ChAdV68.5WTnt.MAG25mer prime/VEE-MAG25mer srRNA boost (group 3), ChAdV68.5WTnt.MAG25mer prime/VEE-MAG25mer srRNA boost+anti-PD-1 (group 4), VEE-MAG25mer srRNA prime/ChAdV68.5WTnt.MAG25mer boost (group 5), VEE-MAG25mer srRNA prime/ChAdV68.5WTnt.MAG25mer boost+anti-PD-1 (group 6), VEE-MAG25mer srRNA prime/VEE-MAG25mer srRNA boost (group 7) and VEE-MAG25mer srRNA prime/VEE-MAG25mer srRNA boost+anti-PD-1 (group 8), respectively (FIG. 18 and Table 19). The mean tumor volumes in the vaccine control or vaccine control combined with anti-PD-1 were 2361 or 2067 mm³, respectively. Based on these data, vaccine treatment with ChAdV68.5WTnt.MAG25mer/VEE-MAG25mer srRNA (group 3), ChAdV68.5WTnt.MAG25mer/VEE-MAG25mer srRNA+anti-PD-1 (group 4), VEE-MAG25mer srRNA/ChAdV68.5WTnt.MAG25mer+anti-PD-1 (group 6) and VEE-MAG25mer srRNA/VEE-MAG25mer srRNA+anti-PD-1 (group 8) resulted in a reduction of tumor growth at 21 days that was significantly different from the control (group 1).

TABLE 19
Tumor size at day 21 measured in the CT26 model

| Treatment | Tumor Size (mm³) | SEM |
|---|---|---|
| Control | 2361 | 235 |
| PD1 | 2067 | 137 |
| chAdV/srRNA | 1129 | 181 |
| chAdV/srRNA + | 848 | 182 |
| PD1 | | |
| srRNA/chAdV | 2142 | 233 |
| srRNA/chAdV + | 1418 | 220 |
| PD1 | | |
| srRNA | 2198 | 134 |
| srRNA + PDl | 1606 | 210 |

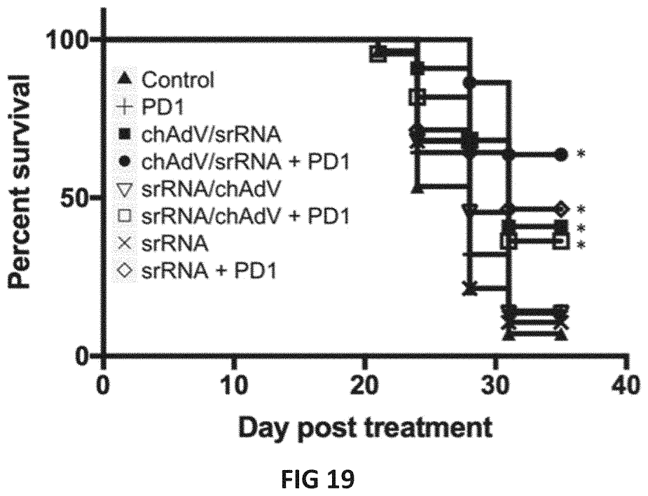

Survival was monitored for 35 days after treatment initiation in the CT-26 tumor model (42 days after injection of CT-26 tumor cells). Improved survival was observed after vaccination of mice with 4 of the combinations tested. After vaccination, 64%, 46%, 41% and 36% of mice survived with ChAdV68.5WTnt.MAG25mer prime/VEE-MAG25mer srRNA boost in combination with anti-PD-1 (group 4; P<0.0001 relative to control group 1), VEE-MAG25mer srRNA prime/VEE-MAG25mer srRNA boost in combination with anti-PD-1 (group 8; P=0.0006 relative to control group 1), ChAdV68.5WTnt.MAG25mer prime/VEE-MAG25mer srRNA boost (group 3; P=0.0003 relative to control group 1) and VEE-MAG25mer srRNA prime/ChAdV68.5WTnt.MAG25mer boost in combination with anti-PD-1 (group 6; P=0.0016 relative to control group 1), respectively (FIG. 19 and Table 20). Survival was not significantly different from the control group 1 (<14%) for the remaining treatment groups [VEE-MAG25mer srRNAprime/ChAdV68.5WTnt.MAG25mer boost (group 5), VEE-MAG25mer srRNA prime/VEE-MAG25mer srRNA boost (group 7) and anti-PD-1 alone (group 2)].

TABLE 20
Survival in the CT26 model

| | | | | chAdV/ | | srRNA/ | | |
|---|---|---|---|---|---|---|---|---|
| | | | chAdV/ | srRNA + | srRNA/ | chAdV + | | srRNA + |
| Timepoint | Control | PD1 | srRNA | PD1 | chAdV | PD1 | srRNA | PD1 |
| 0 | 100 | 100 | 100 | 100.00 | 100.00 | 100 | 100 | 100 |
| 21 | 96 | 100 | 100 | 100 | 100 | 95 | 100 | 100 |
| 24 | 54 | 64 | 91 | 100 | 68 | 82 | 68 | 71 |
| 28 | 21 | 32 | 68 | 86 | 45 | 68 | 21 | 64 |
| 31 | 7 | 14 | 41 | 64 | 14 | 36 | 11 | 46 |
| 35 | 7 | 14 | 41 | 64 | 14 | 36 | 11 | 46 |

In conclusion, ChAdV68.5WTnt.MAG25mer and VEE-MAG25mer srRNA elicited strong T-cell responses to mouse tumor antigens encoded by the vaccines, relative to control. Administration of a ChAdV68.5WTnt.MAG25mer prime and VEE-MAG25mer srRNA boost with or without co-administration of anti-PD-1, VEE-MAG25mer srRNA prime and ChAdV68.5WTnt.MAG25mer boost in combination with anti-PD-1 or administration of VEE-MAG25mer srRNA as a homologous prime boost immunization in combination with anti-PD-1 to tumor bearing mice resulted in improved survival.

### XVIII. Non-Human Primate Studies

Various dosing protocols using ChAdV68 and self-replicating RNA (srRNA) were evaluated in non-human primates (NHP).

### Materials and Methods

A priming vaccine was injected intramuscularly (IM) in each NHP to initiate the study (vaccine prime). One or more boosting vaccines (vaccine boost) were also injected intramuscularly in each NHP. Bilateral injections per dose were administered according to groups outlined in tables and summarized below.

### Immunizations

Mamu-A*01 Indian rhesus macaques were immunized bilaterally with 1×10¹² viral particles (5×10¹¹ viral particles per injection) of ChAdV68.5WTnt.MAG25mer, 30 ug of VEE-MAG25MER srRNA, 100 ug of VEE-MAG25mer srRNA or 300 ug of VEE-MAG25mer srRNA formulated in LNP-1 or LNP-2. Vaccine boosts of 30 ug, 100 ug or 300 ug VEE-MAG25mer srRNA were administered intramuscularly at the indicated time after prime vaccination.

### Immune Monitoring

PBMCs were isolated at indicated times after prime vaccination using Lymphocyte Separation Medium (LSM, MP Biomedicals) and LeucoSep separation tubes (Greiner Bio-One) and resuspended in RPMI containing 10% FBS and penicillin/streptomycin. Cells were counted on the Attune N×T flow cytometer (Thermo Fisher) using propidium iodide staining to exclude dead and apoptotic cells. Cell were then adjusted to the appropriate concentration of live cells for subsequent analysis. For each monkey in the studies, T cell responses were measured using ELISpot or flow cytometry methods. T cell responses to 6 different rhesus macaque Mamu-A*01 class I epitopes encoded in the vaccines were monitored from PBMCs by measuring induction of cytokines, such as IFN-gamma, using ex vivo enzyme-linked immunospot (ELISpot) analysis. ELISpot analysis was performed according to ELISpot harmonization guidelines {DOI: 10.1038/nprot.2015.068} with the monkey IFNg ELISpotPLUS kit (MABTECH). 200,000 PBMCs were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISpot analysis, wells with saturation >50% were recorded as “too numerous to count”. Samples with deviation of replicate wells >10% were excluded from analysis. Spot counts were then corrected for well confluency using the formula: spot count+2 x (spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

Specific CD4 and CD8 T cell responses to 6 different rhesus macaque Mamu-A*01 class I epitopes encoded in the vaccines were monitored from PBMCs by measuring induction of intracellular cytokines, such as IFN-gamma, using flow cytometry. The results from both methods indicate that cytokines were induced in an antigen-specific manner to epitopes.

Immunogenicity in Rhesus Macaques

This study was designed to (a) evaluate the immunogenicity and preliminary safety of VEE-MAG25mer srRNA 30 μg and 100 μg doses as a homologous prime/boost or heterologous prime/boost in combination with ChAdV68.5WTnt.MAG25mer; (b) compare the immune responses of VEE-MAG25mer srRNA in lipid nanoparticles using LNP1 versus LNP2; (c) evaluate the kinetics of T-cell responses to VEE-MAG25mer srRNA and ChAdV68.5WTnt.MAG25mer immunizations.

The study arm was conducted in Mamu-A*01 Indian rhesus macaques to demonstrate immunogenicity. Select antigens used in this study are only recognized in Rhesus macaques, specifically those with a Mamu-A*01 MHC class I haplotype. Mamu-A*01 Indian rhesus macaques were randomized to the different study arms (6 macaques per group) and administered an IM injection bilaterally with either ChAdV68.5WTnt.MAG25mer or VEE-MAG25mer srRNA vector encoding model antigens that includes multiple Mamu-A*01 restricted epitopes. The study arms were as described below.

TABLE 21
Non-GLP immunogenicity study in Indian Rhesus Macaques

| Group | Prime | Boost 1 | Boost 2 |
|---|---|---|---|
| 1 | VEE-MAG25mer | VEE-MAG25mer | VEE-MAG25mer |
| | srRNA-LNP1 | srRNA-LNP1 | srRNA-LNP1 |
| | (30 μg) | (30 μg) | (30 μg) |
| 2 | VEE-MAG25mer | VEE-MAG25mer | VEE-MAG25mer |
| | srRNA-LNP1 | srRNA-LNP1 | srRNA-LNP1 |
| | (100 μg) | (100 μg) | (100 μg) |
| 3 | VEE-MAG25mer | VEE-MAG25mer | VEE-MAG25mer |
| | srRNA-LNP2 | srRNA-LNP2 | srRNA-LNP2 |
| | (100 μg) | (100 μg) | (100 μg) |
| 4 | ChAdV68.5WTnt. | VEE-MAG25mer | VEE-MAG25mer |
| | MAG25mer | srRNA-LNP1 | srRNA-LNP1 |
| | | (100 μg) | (100 μg) |

PBMCs were collected prior to immunization and on weeks 1, 2, 3, 4, 5, 6, 8, 9, and 10 after the initial immunization for immune monitoring.

Results

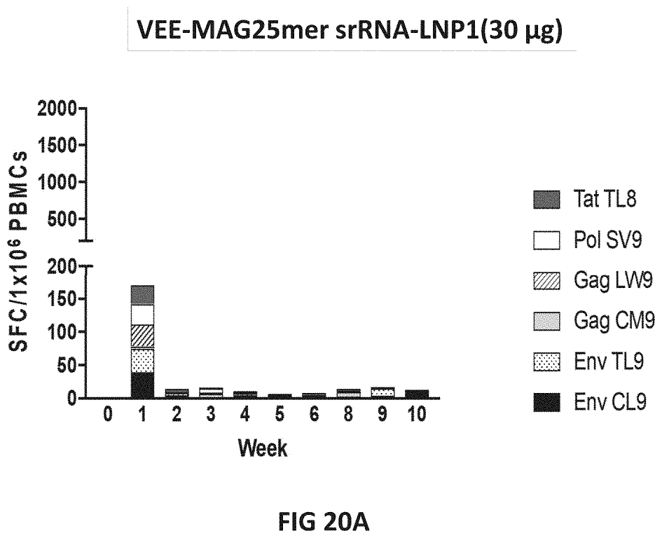

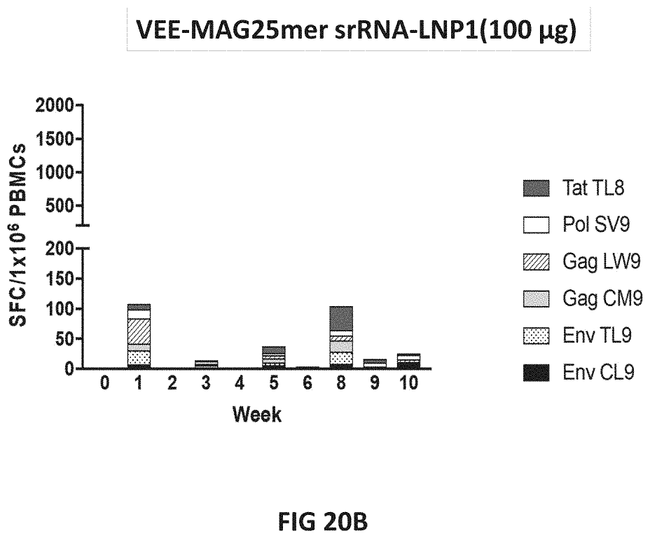

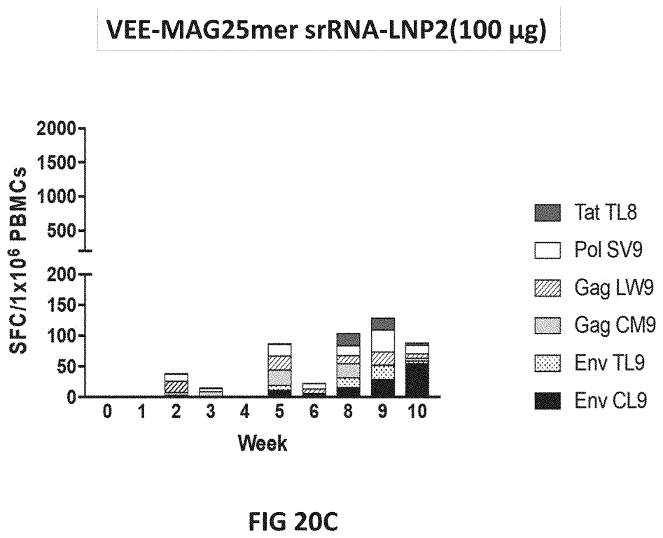

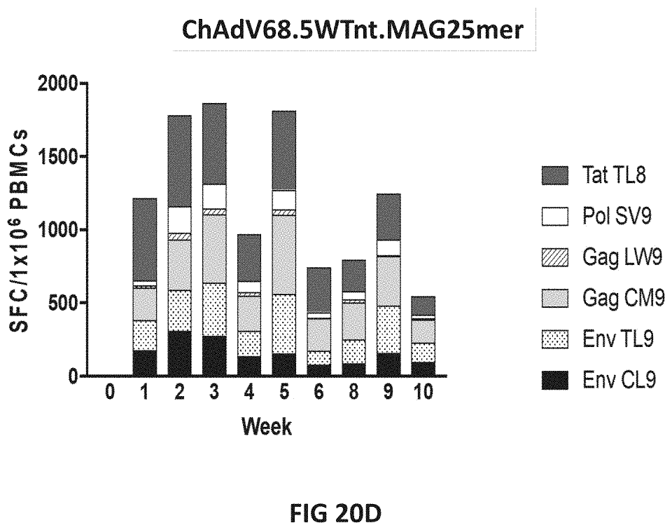

Antigen-specific cellular immune responses in peripheral blood mononuclear cells (PBMCs) were measured to six different Mamu-A*01 restricted epitopes prior to immunization and 1, 2, 3, 4, 5, 6, 8, 9, and 10 weeks after the initial immunization. Animals received a boost immunization with VEE-MAG25mer srRNA on weeks 4 and 8 with either 30 μg or 100 μg doses, and either formulated with LNP1 or LNP2, as described in Table 21. Combined immune responses to all six epitopes were plotted for each immune monitoring timepoint (FIG. 20A-D and Tables 22-25).

Combined antigen-specific immune responses were observed at all measurements with 170, 14, 15, 11, 7, 8, 14, 17, 12 SFCs per 10⁶ PBMCs (six epitopes combined) 1, 2, 3, 4, 5, 6, 8, 9, or 10 weeks after an initial VEE-MAG25mer srRNA-LNP1 (30 μg) prime immunization, respectively (FIG. 20A). Combined antigen-specific immune responses were observed at all measurements with 108, −3, 14, 1, 37, 4, 105, 17, 25 SFCs per 10⁶ PBMCs (six epitopes combined) 1, 2, 3, 4, 5, 6, 8, 9, or 10 weeks after an initial VEE-MAG25mer srRNA-LNP1 (100 μg) prime immunization, respectively (FIG. 20B). Combined antigen-specific immune responses were observed at all measurements with −17, 38, 14, −2, 87, 21, 104, 129, 89 SFCs per 10⁶ PBMCs (six epitopes combined) 1, 2, 3, 4, 5, 6, 8, 9, or 10 weeks after an initial VEE-MAG25mer srRNA-LNP2 (100 μg) prime immunization, respectively (FIG. 20C). Negative values are a result of normalization to pre-bleed values for each epitope/animal.

Combined antigen-specific immune responses were observed at all measurements with 1218, 1784, 1866, 973, 1813, 747, 797, 1249, and 547 SFCs per 10⁶ PBMCs (six epitopes combined) 1, 2, 3, 4, 5, 6, 8, 9, or 10 weeks after an initial ChAdV68.5WTnt.MAG25mer prime immunization, respectively (FIG. 20D). The immune response showed the expected profile with peak immune responses measured ˜2-3 weeks after the prime immunization followed by a contraction in the immune response after 4 weeks. Combined antigen-specific cellular immune responses of 1813 SFCs per 10⁶ PBMCs (six epitopes combined) were measured 5 weeks after the initial immunization with ChAdV68.5WTnt.MAG25mer (i.e., 1 week after the first boost with VEE-MAG25mer srRNA). The immune response measured 1 week after the first boost with VEE-MAG25mer srRNA (week 5) was comparable to the peak immune response measured for the ChAdV68.5WTnt.MAG25mer prime immunization (week 3) (FIG. 20D). Combined antigen-specific cellular immune responses of 1249 SFCs per 10⁶ PBMCs (six epitopes combined) was measured 9 weeks after the initial immunization with ChAdV68.5WTnt.MAG25mer, respectively (i.e., 1 week after the second boost with VEE-MAG25mer srRNA). The immune responses measured 1 week after the second boost with VEE-MAG25mer srRNA (week 9) was ˜2-fold higher than that measured just before the boost immunization (FIG. 20D).

TABLE 22
Mean spot forming cells (SFC) per 10⁶ PBMCs for each epitope ± SEM for
VEE-MAG25mer srRNA-LNP1 (30 μg) (Group 1)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | 39.7 ± 22.7 | 35.4 ± 25.1 | 3.2 ± 3.6 | 33 ± 28.1 | 30.9 ± 20.3 | 28.3 ± 17.5 |
| 3 | 2 ± 2.4 | 0.2 ± 1.8 | 1.8 ± 2.4 | 3.7 ± 1.9 | 1.7 ± 2.8 | 4.9 ± 2.3 |
| 4 | 1 ± 1.8 | 0.3 ± 1.2 | 5.5 ± 3.6 | 2.3 ± 2.2 | 5.7 ± 2.7 | 0.8 ± 0.8 |
| 5 | 0.5 ± 0.9 | 1.4 ± 3.8 | 3.1 ± 1.6 | 2.3 ± 2.7 | 1.9 ± 2 | 1.4 ± 1.2 |
| 6 | 1.9 ± 1.8 | −0.3 ± 3 | 1.7 ± 1.2 | 1.4 ± 1.4 | 0.8 ± 1.1 | 1.1 ± 1 |
| 8 | −0.4 ± 0.8 | −0.9 ± 2.9 | 0.5 ± 1.3 | 3 ± 1.1 | 2.2 ± 2.1 | 3.7 ± 2 |
| 9 | 1 ± 1.7 | 1.2 ± 4.2 | 7.2 ± 3.9 | 0.5 ± 0.7 | 1.6 ± 3 | 3 ± 1 |
| 10 | 3.8 ± 1.8 | 11 ± 5 | −1.1 ± 1.1 | 1.9 ± 0.9 | 1.3 ± 1.6 | 0.2 ± 0.5 |

TABLE 23
Mean spot forming cells (SFC) per 10⁶ PBMCs for each epitope ± SEM for
VEE-MAG25mer srRNA-LNP1 (100 μg) (Group 2)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | 7.9 ± 17.2 | 23.2 ± 17.4 | 11.4 ± 4.9 | 41.7 ± 16.5 | 15 ± 13.5 | 8.9 ± 6.2 |
| 3 | −3.1 ± 4.6 | −7.2 ± 6.5 | 2.3 ± 2.3 | −0.3 ± 2.7 | 2.7 ± 5.1 | 2.2 ± 1.4 |
| 4 | 1.9 ± 3.8 | −6.2 ± 7.6 | 10.5 ± 4.1 | 1.2 ± 2.9 | 5.6 ± 4.9 | 1.1 ± 0.8 |
| 5 | −2.6 ± 7 | −8 ± 5.9 | 1.5 ± 1.7 | 6.4 ± 2.3 | 0.7 ± 4.3 | 3.3 ± 1.3 |
| 6 | 6.3 ± 6.3 | 4.4 ± 8.3 | 6.6 ± 4.4 | 5.2 ± 5.2 | 3.9 ± 5 | 10.8 ± 6.9 |
| 8 | −3.6 ± 7.2 | −6.8 ± 7.3 | −0.8 ± 1.2 | 3.4 ± 4.2 | 6.4 ± 7.5 | 5.7 ± 2.7 |
| 9 | 8.1 ± 2.4 | 20.6 ± 23.4 | 18.9 ± 5.7 | 8.1 ± 8.9 | 9 ± 11.2 | 40 ± 17.6 |
| 10 | 3.1 ± 8 | −3.9 ± 8.5 | 3.3 ± 1.8 | 0.6 ± 2.9 | 7.4 ± 6.4 | 6.1 ± 2.5 |

TABLE 24
Mean spot forming cells (SFC) per 10⁶ PBMCs for each epitope ± SEM
for VEE-MAG25mer srRNA-LNP2 (100 μg) (Group 3)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | −5.9 ± 3.8 | −0.3 ± 0.5 | −0.5 ± 1.5 | −5.7 ± 6.1 | −1 ± 1.3 | −3.2 ± 5.5 |
| 3 | 0.7 ± 5.2 | 3.4 ± 2.4 | 4.2 ± 4.6 | 18.3 ± 15.5 | 11.9 ± 5.1 | −0.4 ± 8.2 |
| 4 | −3.8 ± 5.5 | 2.3 ± 1.8 | 11.3 ± 6.1 | −3.1 ± 5.6 | 8.5 ± 4 | −1.5 ± 6.1 |
| 5 | −3.7 ± 5.7 | −0.1 ± 0.7 | −0.2 ± 1.6 | 3.4 ± 8.5 | 3 ± 3.1 | −4.6 ± 5 |
| 6 | 12.3 ± 15 | 7.8 ± 4.9 | 24.7 ± 19.8 | 23.2 ± 22.5 | 18.7 ± 15.8 | 0.5 ± 6.2 |
| 8 | 5.9 ± 12.3 | −0.1 ± 0.7 | −0.5 ± 1.3 | 8.8 ± 14.4 | 8.7 ± 8 | −1.3 ± 4 |
| 9 | 16.1 ± 13.4 | 16.5 ± 4 | 22.9 ± 4.2 | 13 ± 13.2 | 16.4 ± 7.8 | 19.6 ± 9.2 |
| 10 | 29.9 ± 21.8 | 22 ± 19.5 | 0.5 ± 2.6 | 22.2 ± 22.6 | 35.3 ± 15.8 | 19.4 ± 17.3 |

TABLE 25
Mean spot forming cells (SFC) per 10⁶ PBMCs for each epitope ± SEM for
ChAdV68.5WTnt.MAG25mer prime

| | Antigen | | | | | |
|---|---|---|---|---|---|---|
| Wk | Env CL9 | Env TL9 | Gag CM9 | Gag LW9 | Pol SV9 | Tat TL8 |
| 1 | 178 ± 68.7 | 206.5 ± 94.8 | 221.2 ± 120 | 15.4 ± 16.7 | 33.3 ± 25.9 | 563.5 ± 174.4 |
| 2 | 311.2 ± 165.5 | 278.8 ± 100.9 | 344.6 ± 110.8 | 46.3 ± 13.5 | 181.6 ± 76.8 | 621.4 ± 220.9 |
| 3 | 277.3 ± 101.1 | 359.6 ± 90.5 | 468.2 ± 106.6 | 41.7 ± 11.1 | 169.8 ± 57.8 | 549.4 ± 115.7 |
| 4 | 140 ± 46.5 | 169.6 ± 46.8 | 239.4 ± 37 | 26.5 ± 11.4 | 75 ± 31.6 | 322.2 ± 50.7 |
| 5 | 155.6 ± 62.1 | 406.7 ± 96.4 | 542.7 ± 143.3 | 35.1 ± 16.6 | 134.2 ± 53.7 | 538.5 ± 91.9 |
| 6 | 78.9 ± 42.5 | 95.5 ± 29.4 | 220.9 ± 75.3 | −1.4 ± 5.3 | 43.4 ± 19.6 | 308.1 ± 42.6 |
| 8 | 88.4 ± 30.4 | 162.1 ± 30.3 | 253.4 ± 78.6 | 21.4 ± 11.2 | 53.7 ± 22.3 | 217.8 ± 45.2 |
| 9 | 158.5 ± 69 | 322.3 ± 87.2 | 338.2 ± 137.1 | 5.6 ± 12.4 | 109.2 ± 17.9 | 314.8 ± 43.4 |
| 10 | 97.3 ± 32.5 | 133.2 ± 27 | 154.9 ± 59.2 | 10 ± 6 | 26 ± 16.7 | 125.5 ± 27.7 |

Non-GLP RNA Dose Ranging Study (Higher Doses) in Indian Rhesus Macaques

This study was designed to (a) evaluate the immunogenicity of VEE-MAG25mer srRNA at a dose of 300 μg as a homologous prime/boost or heterologous prime/boost in combination with ChAdV68.5WTnt.MAG25mer; (b) compare the immune responses of VEE-MAG25mer srRNA in lipid nanoparticles using LNP1 versus LNP2 at the 300 μg dose; and (c) evaluate the kinetics of T-cell responses to VEE-MAG25mer srRNA and ChAdV68.5WTnt.MAG25mer immunizations.

The study arm was conducted in Mamu-A*01 Indian rhesus macaques to demonstrate immunogenicity. Vaccine immunogenicity in nonhuman primate species, such as Rhesus, is the best predictor of vaccine potency in humans. Furthermore, select antigens used in this study are only recognized in Rhesus macaques, specifically those with a Mamu-A*01 MHC class I haplotype. Mamu-A*01 Indian rhesus macaques were randomized to the different study arms (6 macaques per group) and administered an IM injection bilaterally with either ChAdV68.5-WTnt.MAG25mer or VEE-MAG25mer srRNA encoding model antigens that includes multiple Mamu-A*01 restricted antigens. The study arms were as described below.

PBMCs were collected prior to immunization and 4, 5, 6, 7, 8, 10, 11, 12, 13, 14, 15, 16, 17, 18, 19, 20, 21, 22, 23 or 24 weeks after the initial immunization for immune monitoring for group 1 (heterologous prime/boost). PBMCs were collected prior to immunization and 4, 5, 7, 8, 10, 11, 12, 13, 14, or 15 weeks after the initial immunization for immune monitoring for groups 2 and 3 (homologous prime/boost).

TABLE 26
Non-GLP immunogenicity study in Indian Rhesus Macaques

| Group | Prime | Boost 1 | Boost 2 | Boost 3 |
|---|---|---|---|---|
| 1 | ChAdV68.5WTnt. | VEE-MAG25mer | VEE-MAG25mer | VEE-MAG25mer |
| | MAG25mer | srRNA-LNP2 | srRNA-LNP2 | srRNA-LNP2 |
| | | (300 μg) | (300 μg) | (300 μg) |
| 2 | VEE-MAG25mer | VEE-MAG25mer | VEE-MAG25mer | |
| | srRNA-LNP2 | srRNA-LNP2 | srRNA-LNP2 | |
| | (300 μg) | (300 μg) | (300 μg) | |
| 3 | VEE-MAG25mer | VEE-MAG25mer | VEE-MAG25mer | |
| | srRNA-LNP1 | srRNA-LNP1 | srRNA-LNP1 | |
| | (300 μg) | (300 μg) | (300 μg) | |

Results

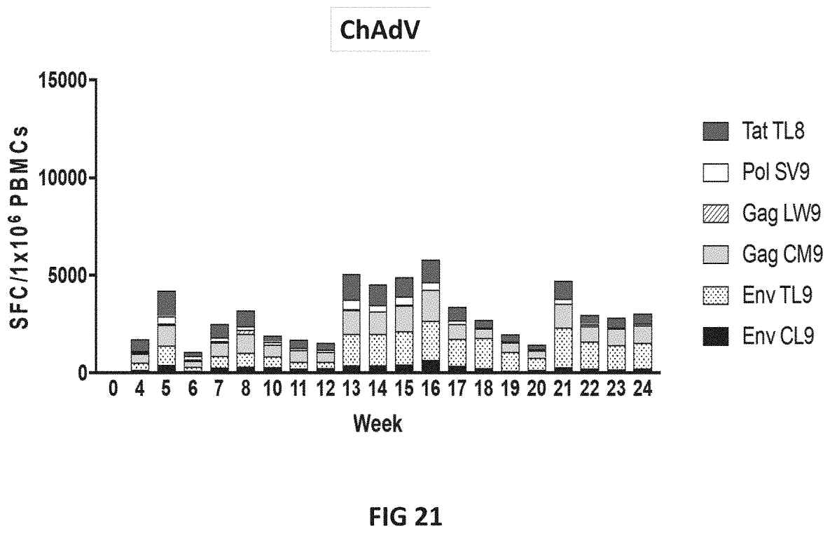

Mamu-A*01 Indian rhesus macaques were immunized with ChAdV68.5-WTnt.MAG25mer. Antigen-specific cellular immune responses in peripheral blood mononuclear cells (PBMCs) were measured to six different Mamu-A*01 restricted epitopes prior to immunization and 4, 5, 6, 7, 8, 10, 11, 12, 13, 14, 15, 16, 17, 18, 19, 20, 21, 22, 23 or 24 weeks after the initial immunization (FIG. 21 and Table 27). Animals received boost immunizations with VEE-MAG25mer srRNA using the LNP2 formulation on weeks 4, 12, and 20. Combined antigen-specific immune responses of 1750, 4225, 1100, 2529, 3218, 1915, 1708, 1561, 5077, 4543, 4920, 5820, 3395, 2728, 1996, 1465, 4730, 2984, 2828, or 3043 SFCs per 10⁶ PBMCs (six epitopes combined) were measured 4, 5, 6, 7, 8, 10, 11, 12, 13, 14, 15, 16, 17, 18, 19, 20, 21, 22, 23 or 24 weeks after the initial immunization with ChAdV68.5WTnt.MAG25mer (FIG. 21). Immune responses measured 1 week after the second boost immunization (week 13) with VEE-MAG25mer srRNA were ˜3-fold higher than that measured just before the boost immunization (week 12). Immune responses measured 1 week after the third boost immunization (week 21) with VEE-MAG25mer srRNA, were ˜3-fold higher than that measured just before the boost immunization (week 20), similar to the response observed for the second boost.

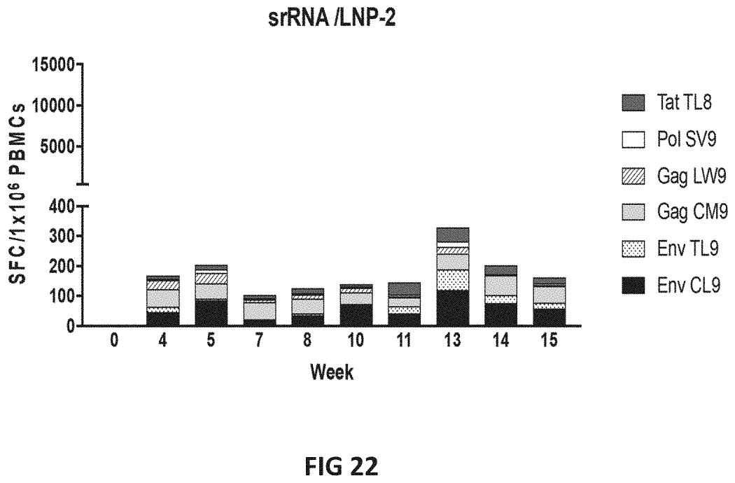

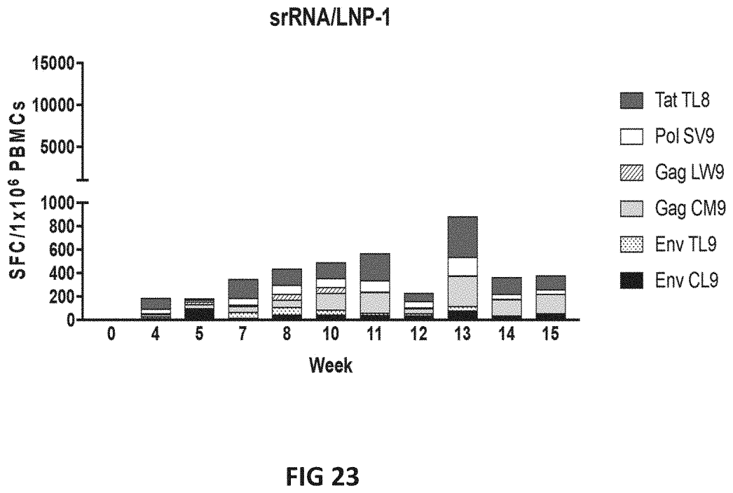

Mamu-A*01 Indian rhesus macaques were also immunized with VEE-MAG25mer srRNA using two different LNP formulations (LNP1 and LNP2). Antigen-specific cellular immune responses in peripheral blood mononuclear cells (PBMCs) were measured to six different Mamu-A*01 restricted epitopes prior to immunization and 4, 5, 6, 7, 8, 10, 11, 12, 13, 14, or 15 weeks after the initial immunization (FIGS. 22 and 23, Tables 28 and 29). Animals received boost immunizations with VEE-MAG25mer srRNA using the respective LNP1 or LNP2 formulation on weeks 4 and 12. Combined antigen-specific immune responses of 168, 204, 103, 126, 140, 145, 330, 203, and 162 SFCs per 106 PBMCs (six epitopes combined) were measured 4, 5, 7, 8, 10, 11, 13, 14, 15 weeks after the immunization with VEE-MAG25mer srRNA-LNP2 (FIG. 22). Combined antigen-specific immune responses of 189, 185, 349, 437, 492, 570, 233, 886, 369, and 381 SFCs per 10⁶ PBMCs (six epitopes combined) were measured 4, 5, 7, 8, 10, 11, 12, 13, 14, 15 weeks after the immunization with VEE-MAG25mer srRNA-LNP1 (FIG. 23).

TABLE 27
Mean spot forming cells (SFC) per 10⁶ PBMCs for each epitope ± SEM for priming
vaccination with ChAdV68.5WTnt.MAG25mer (Group 1)

| | Antigen | | | | | |
|---|---|---|---|---|---|---|
| Wk | Env CL9 | Env TL9 | Gag CM9 | Gag LW9 | Pol SV9 | Tat TL8 |
| 4 | 173 ± 41.6 | 373.5 ± 87.3 | 461.4 ± 74.2 | 38.4 ± 26.1 | 94.5 ± 26 | 609.2 ± 121.9 |
| 5 | 412.7 ± 138.4 | 987.8 ± 283.3 | 1064.4 ± 266.9 | 85.6 ± 31.2 | 367.2 ± 135.2 | 1306.8 ± 332.8 |
| 6 | 116.2 ± 41.2 | 231.1 ± 46.3 | 268.3 ± 90.7 | 86.1 ± 42 | 174.3 ± 61 | 223.9 ± 38.1 |
| 7 | 287.4 ± 148.7 | 588.9 ± 173.9 | 693.2 ± 224.8 | 92.1 ± 33.5 | 172.9 ± 55.6 | 694.6 ± 194.8 |
| 8 | 325.4 ± 126.6 | 735.8 ± 212 | 948.9 ± 274.5 | 211.3 ± 62.7 | 179.1 ± 50 | 817.3 ± 185.2 |
| 10 | 312 ± 129.7 | 543.2 ± 188.4 | 618.6 ± 221.7 | −5.7 ± 4.1 | 136.5 ± 51.3 | 309.9 ± 85.6 |
| 11 | 248.5 ± 81.1 | 348.7 ± 129.8 | 581.1 ± 205.5 | −3.1 ± 4.4 | 119 ± 51.2 | 413.7 ± 144.8 |
| 12 | 261.9 ± 68.2 | 329.9 ± 83 | 486.5 ± 118.6 | −1.2 ± 5.1 | 132.8 ± 31.8 | 350.9 ± 69.3 |
| 13 | 389.3 ± 167.7 | 1615.8 ± 418.3 | 1244.3 ± 403.6 | 1.3 ± 8.1 | 522.5 ± 155 | 1303.3 ± 385.6 |
| 14 | 406.3 ± 121.6 | 1616 ± 491.7 | 1142.3 ± 247.2 | 6.6 ± 11.1 | 322.7 ± 94.1 | 1048.6 ± 215.6 |
| 15 | 446.8 ± 138.7 | 1700.8 ± 469.1 | 1306.3 ± 294.4 | 43 ± 24.5 | 421.2 ± 87.9 | 1001.5 ± 236.4 |
| 16 | 686.8 ± 268.8 | 1979.5 ± 541.7 | 1616.8 ± 411.8 | 2.4 ± 7.8 | 381.9 ± 116.4 | 1152.8 ± 352.7 |
| 17 | 375.8 ± 109.3 | 1378.6 ± 561.2 | 773.1 ± 210.3 | −1.4 ± 4.3 | 177.6 ± 93.7 | 691.7 ± 245 |
| 18 | 255.9 ± 499.7 | 1538.4 ± 498.1 | 498.7 ± 152.3 | −5.3 ± 3.3 | 26.2 ± 13.4 | 413.9 ± 164.8 |
| 19 | 133 ± 62.6 | 955.9 ± 456.8 | 491.1 ± 121.8 | −5.7 ± 4.1 | 50.3 ± 25.4 | 371.2 ± 123.7 |
| 20 | 163.7 ± 55.8 | 641.7 ± 313.5 | 357.9 ± 91.1 | 2.6 ± 7.5 | 41.4 ± 24.2 | 257.8 ± 68.9 |
| 21 | 319.9 ± 160.5 | 2017.1 ± 419.9 | 1204.8 ± 335.2 | −3.7 ± 5.1 | 268.1 ± 109.6 | 924.1 ± 301 |
| 22 | 244.7 ± 105.6 | 1370.9 ± 563.5 | 780.3 ± 390 | −3.6 ± 5.1 | 118.2 ± 68.1 | 473.3 ± 249.3 |
| 23 | 176.7 ± 81.8 | 1263.7 ± 527.3 | 838.6 ± 367.9 | −5.7 ± 4.1 | 73.6 ± 49 | 480.9 ± 163.9 |
| 24 | 236.5 ± 92 | 1324.7 ± 589.3 | 879.7 ± 321 | −0.4 ± 5.7 | 104 ± 53.1 | 498 ± 135.8 |

TABLE 28
Mean spot forming cells (SFC) per 10⁶ PBMCs for each epitope ± SEM for
priming vaccination with VEE-MAG25mer srRNA-LNP2 (300 μg) (Group 2)

| | Antigen | | | | | |
|---|---|---|---|---|---|---|
| Wk | Env CL9 | Env TL9 | Gag CM9 | Gag LW9 | Pol SV9 | Tat TL8 |
| 4 | 46 ± 27.1 | 18.4 ± 6.8 | 58.3 ± 45.8 | 29.9 ± 20.8 | 4.9 ± 2.3 | 10.7 ± 4 |
| 5 | 85.4 ± 54 | 5.2 ± 5.8 | 52.4 ± 51.2 | 34.5 ± 35 | 11.8 ± 12.2 | 14.4 ± 7.9 |
| 7 | 18.6 ± 32.5 | 1.9 ± 1.7 | 59.4 ± 55.7 | 9.3 ± 10.7 | 3.3 ± 3 | 10.7 ± 6.1 |
| 8 | 36.6 ± 39.4 | 6.3 ± 3.9 | 48.7 ± 39.9 | 13.5 ± 8.8 | 3.8 ± 3.6 | 17.2 ± 9.7 |
| 10 | 69.1 ± 59.1 | 4.4 ± 1.9 | 39.3 ± 38 | 14.7 ± 10.8 | 4.4 ± 5.3 | 8.5 ± 5.3 |
| 11 | 43 ± 38.8 | 22.6 ± 21.1 | 30.2 ± 26.2 | 3.3 ± 2.2 | 5.8 ± 3.5 | 40.3 ± 25.5 |
| 13 | 120.4 ± 78.3 | 68.2 ± 43.9 | 54.2 ± 36.8 | 21.8 ± 7.4 | 17.7 ± 6.1 | 47.4 ± 27.3 |
| 14 | 76 ± 44.8 | 28 ± 19.5 | 65.9 ± 64.3 | −0.3 ± 1.3 | 2.5 ± 2 | 31.1 ± 26.5 |
| 15 | 58.9 ± 41.4 | 19.5 ± 15.1 | 55.4 ± 51 | 2.5 ± 2 | 5.5 ± 3.6 | 20.1 ± 15.7 |

TABLE 29
Mean spot forming cells (SFC) per 10⁶ PBMCs for each epitope ± SEM for priming
vaccination with VEE-MAG25mer srRNA-LNP1 (300 μg) (Group 3)

| | Antigen | | | | | |
|---|---|---|---|---|---|---|
| Wk | Env CL9 | Env TL9 | Gag CM9 | Gag LW9 | Pol SV9 | Tat TL8 |
| 4 | 19.5 ± 8.7 | 13.3 ± 13.1 | 16.5 ± 15.3 | 10.5 ± 7.3 | 35.9 ± 24.8 | 92.9 ± 91.6 |
| 5 | 87.9 ± 43.9 | 12.7 ± 11.7 | 37.2 ± 31.9 | 21.1 ± 23.8 | 13.2 ± 13.7 | 12.6 ± 13.7 |
| 7 | 21.1 ± 13.3 | 48.8 ± 48.4 | 51.7 ± 39.5 | 9.1 ± 10.5 | 58.6 ± 55.8 | 159.4 ± 159 |
| 8 | 47.7 ± 21.7 | 66.4 ± 52.2 | 59.8 ± 57.4 | 49.4 ± 28 | 79.4 ± 63 | 133.8 ± 132.1 |
| 10 | 49 ± 30.2 | 42.2 ± 41.1 | 139.3 ± 139.3 | 51.6 ± 51.2 | 78.2 ± 75.8 | 131.7 ± 131.6 |
| 11 | 42 ± 26.8 | 20.9 ± 21.4 | 177.1 ± 162 | −6.3 ± 4.3 | 104.3 ± 104.1 | 231.5 ± 230.1 |
| 12 | 40.2 ± 19 | 20.3 ± 11.9 | 42.2 ± 46.7 | 3.7 ± 6.7 | 57 ± 44.7 | 70 ± 69.2 |
| 13 | 81.2 ± 48.9 | 38.2 ± 37.6 | 259.4 ± 222.2 | −4 ± 4.1 | 164.1 ± 159.3 | 347.3 ± 343.5 |
| 14 | 34.5 ± 31.8 | 5.3 ± 11.6 | 138.6 ± 137.3 | −4.7 ± 5.2 | 52.3 ± 52.9 | 142.6 ± 142.6 |
| 15 | 49 ± 24 | 6.7 ± 9.8 | 167.1 ± 163.8 | −6.4 ± 4.2 | 47.8 ± 42.3 | 116.6 ± 114.5 |

srRNA Dose Ranging Study

In one implementation of the present invention, an srRNA dose ranging study can be conducted in mamu A01 Indian rhesus macaques to identify which srRNA dose to progress to NHP immunogenicity studies. In one example, Mamu A01 Indian rhesus macaques can be administered with an srRNA vector encoding model antigens that includes multiple mamu A01 restricted epitopes by IM injection. In another example, an anti-CTLA-4 monoclonal antibody can be administered SC proximal to the site of IM vaccine injection to target the vaccine draining lymph node in one group of animals. PBMCs can be collected every 2 weeks after the initial vaccination for immune monitoring. The study arms are described in below (Table 30).

TABLE 30
Non-GLP RNA dose ranging study in Indian Rhesus Macaques

| Group | Prime | Boost 1 | Boost 2 |
|---|---|---|---|
| 1 | srRNA-LNP (Low Dose) | srRNA-LNP (Low Dose) | srRNA-LNP (Low Dose) |
| 2 | srRNA-LNP (Mid Dose) | srRNA-LNP (Mid Dose) | srRNA-LNP (Mid Dose) |
| 3 | SrRNA-LNP (High Dose) | srRNA-LNP (High Dose) | srRNA-LNP (High Dose) |
| 4 | srRNA-LNP (High Dose) + | srRNA-LNP (High Dose) + | srRNA-LNP (High Dose) + |
| | anti-CTLA-4 | anti-CTLA-4 | anti-CTLA-4 |

* Dose range of srRNA to be determined with the high dose ≤300 μg.

Immunogenicity Study in Indian Rhesus Macaques

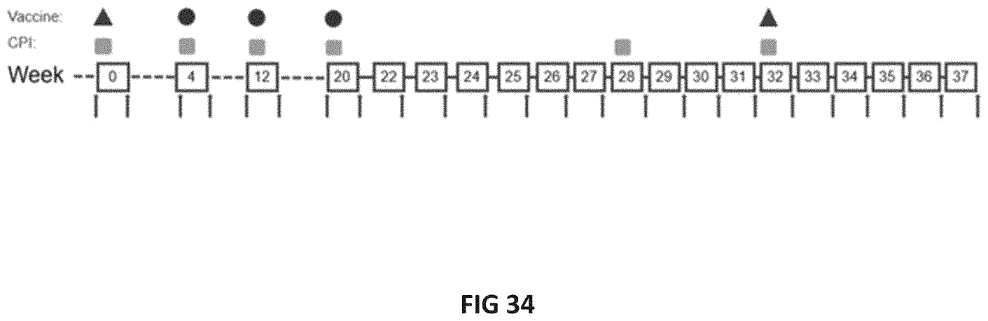

Vaccine studies were conducted in mamu A01 Indian rhesus macaques (NHPs) to demonstrate immunogenicity using the antigen vectors. FIG. 34 illustrates the vaccination strategy. Three groups of NHPs were immunized with ChAdV68.5-WTnt.MAG25mer and either with the checkpoint inhibitor anti-CTLA-4 antibody Ipilimumab (Groups 5 & 6) or without the checkpoint inhibitor (Group 4). The antibody was administered either intravenously (group 5) or subcutaneously (group 6). Triangles indicate chAd68 vaccination (1ₑ12 vp/animal) at weeks 0 & 32. Circles represent alphavirus vaccination at weeks 0, 4, 12, 20, 28 and 32.

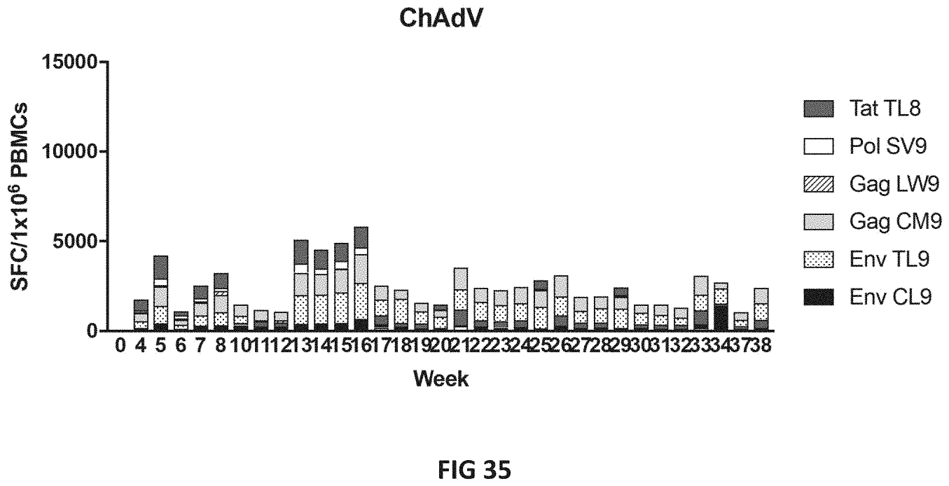

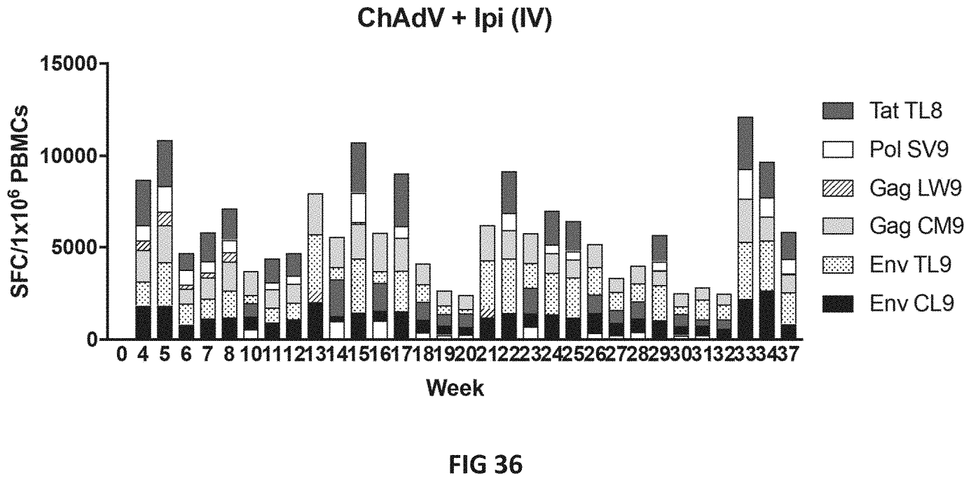

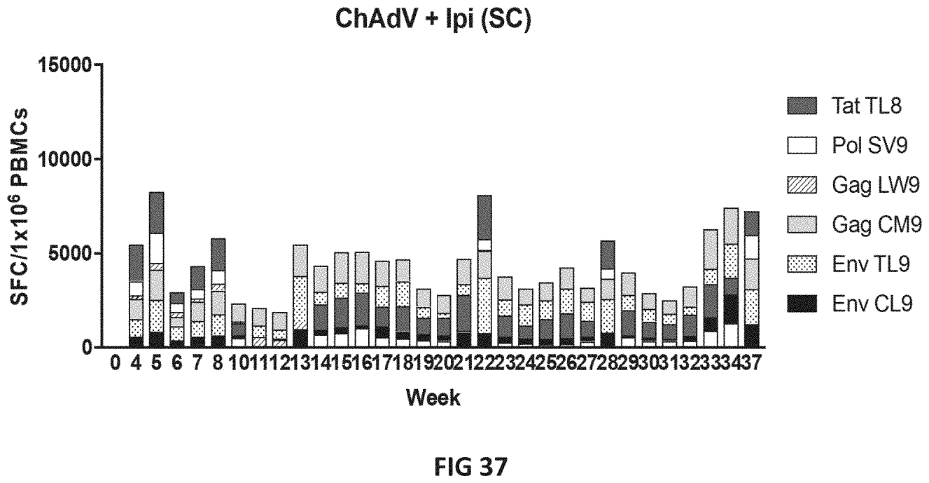

The time course of CD8+ anti-epitope responses in the immunized NHPs are presented for chAd-MAG immunization alone (FIG. 35 and Table 31A), chAd-MAG immunization with the checkpoint inhibitor delivered IV (FIG. 36 and Table 31B), and chAd-MAG immunization with the checkpoint inhibitor delivered SC (FIG. 37 and Table 31C). The results demonstrate chAd68 vectors efficiently primed CD8+ responses in primates, alphavirus vectors efficiently boosted the chAD68 vaccine priming response, checkpoint inhibitor whether delivered IV or SC amplified both priming and boosting responses, and chAd vectors readministered post vaccination to effectively boosted the immune responses.

TABLE 31A
CD8+ anti-epitope responses in Rhesus Macaques dosed with chAd-MAG (Group
4). Mean SFC/1e6 splenocytes +/− the standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 173 ± 41.6 | 373.5 ± 87.3 | 461.4 ± 74.2 | 38.4 ± 26.1 | 94.5 ± 26 | 609.2 ± 121.9 |
| 5 | 412.7 ± 138.4 | 987.8 ± 283.3 | 1064.4 ± 266.9 | 85.6 ± 31.2 | 367.2 ± 135.2 | 1306.8 ± 332.8 |
| 6 | 116.2 ± 41.2 | 231.1 ± 46.3 | 268.3 ± 90.7 | 86.1 ± 42 | 174.3 ± 61 | 223.9 ± 38.1 |
| 7 | 287.4 ± 148.7 | 588.9 ± 173.9 | 693.2 ± 224.8 | 92.1 ± 33.5 | 172.9 ± 55.6 | 694.6 ± 194.8 |
| 8 | 325.4 ± 126.6 | 735.8 ± 212 | 948.9 ± 274.5 | 211.3 ± 62.7 | 179.1 ± 50 | 817.3 ± 185.2 |
| 10 | 312 ± 129.7 | 543.2 ± 188.4 | 618.6 ± 221.7 | −5.7 ± 4.1 | 136.5 ± 51.3 | 309.9 ± 85.6 |
| 11 | 248.5 ± 81.1 | 348.7 ± 129.8 | 581.1 ± 205.5 | −3.1 ± 4.4 | 119 ± 51.2 | 413.7 ± 144.8 |
| 12 | 261.9 ± 68.2 | 329.9 ± 83 | 486.5 ± 118.6 | −1.2 ± 5.1 | 132.8 ± 31.8 | 350.9 ± 69.3 |
| 13 | 389.3 ± 167.7 | 1615.8 ± 418.3 | 1244.3 ± 403.6 | 1.3 ± 8.1 | 522.5 ± 155 | 1303.3 ± 385.6 |
| 14 | 406.3 ± 121.6 | 1616 ± 491.7 | 1142.3 ± 247.2 | 6.6 ± 11.1 | 322.7 ± 94.1 | 1048.6 ± 215.6 |
| 15 | 446.8 ± 138.7 | 1700.8 ± 469.1 | 1306.3 ± 294.4 | 43 ± 24.5 | 421.2 ± 87.9 | 1001.5 ± 236.4 |
| 16 | 686.8 ± 268.8 | 1979.5 ± 541.7 | 1616.8 ± 411.8 | 2.4 ± 7.8 | 381.9 ± 116.4 | 1152.8 ± 352.7 |
| 17 | 375.8 ± 109.3 | 1378.6 ± 561.2 | 773.1 ± 210.3 | −1.4 ± 4.3 | 177.6 ± 93.7 | 691.7 ± 245 |
| 18 | 255.9 ± 99.7 | 1538.4 ± 498.1 | 498.7 ± 152.3 | −5.3 ± 3.3 | 26.2 ± 13.4 | 413.9 ± 164.8 |
| 19 | 133 ± 62.6 | 955.9 ± 456.8 | 491.1 ± 121.8 | −5.7 ± 4.1 | 50.3 ± 25.4 | 371.2 ± 123.7 |
| 20 | 163.7 ± 55.8 | 641.7 ± 313.5 | 357.9 ± 91.1 | 2.6 ± 7.5 | 41.4 ± 24.2 | 257.8 ± 68.9 |
| 21 | 319.9 ± 160.5 | 2017.1 ± 419.9 | 1204.8 ± 335.2 | −3.7 ± 5.1 | 268.1 ± 109.6 | 924.1 ± 301 |
| 22 | 244.7 ± 105.6 | 1370.9 ± 563.5 | 780.3 ± 390 | −3.6 ± 5.1 | 118.2 ± 68.1 | 473.3 ± 249.3 |
| 23 | 176.7 ± 81.8 | 1263.7 ± 527.3 | 838.6 ± 367.9 | −5.7 ± 4.1 | 73.6 ± 49 | 480.9 ± 163.9 |
| 24 | 236.5 ± 92 | 1324.7 ± 589.3 | 879.7 ± 321 | −0.4 ± 5.7 | 104 ± 53.1 | 498 ± 135.8 |
| 25 | 136.4 ± 52.6 | 1207.1 ± 501.6 | 924 ± 358.5 | 6.2 ± 10.5 | 74.1 ± 34.4 | 484.6 ± 116.7 |
| 26 | 278.2 ± 114.4 | 1645 ± 661.7 | 1170.2 ± 469.9 | −2.9 ± 5.7 | 80.6 ± 55.8 | 784.4 ± 214.1 |
| 27 | 159 ± 56.8 | 961.7 ± 547.1 | 783.6 ± 366.4 | −5 ± 4.3 | 63.6 ± 27.5 | 402.9 ± 123.4 |
| 28 | 189.6 ± 75.7 | 1073.1 ± 508.8 | 668.3 ± 312.5 | −5.7 ± 4.1 | 80.3 ± 38.3 | 386.4 ± 122 |
| 29 | 155.3 ± 69.1 | 1102.9 ± 606.1 | 632.9 ± 235 | 34.5 ± 24.2 | 80 ± 35.5 | 422.5 ± 122.9 |
| 30 | 160.2 ± 59.9 | 859 ± 440.9 | 455 ± 209.1 | −3 ± 5.3 | 60.5 ± 28.4 | 302.7 ± 123.2 |
| 31 | 122.2 ± 49.7 | 771.1 ± 392.7 | 582.2 ± 233.5 | −5.7 ± 4.1 | 55.1 ± 27.3 | 295.2 ± 68.3 |
| 32 | 119.3 ± 28.3 | 619.4 ± 189.7 | 566 ± 222.1 | −3.7 ± 5.1 | 21.9 ± 4.5 | 320.5 ± 76.4 |
| 33 | 380.5 ± 122 | 1636.1 ± 391.4 | 1056.2 ± 205.7 | −5.7 ± 4.1 | 154.5 ± 38.5 | 988.4 ± 287.7 |
| 34 | 1410.8 ± 505.4 | 972.4 ± 301.5 | 319.6 ± 89.6 | −4.8 ± 4.2 | 141.1 ± 49.8 | 1375.5 ± 296.7 |
| 37 | 130.8 ± 29.2 | 500 ± 156.9 | 424.9 ± 148.9 | −3.5 ± 4.7 | 77.7 ± 24.6 | 207.1 ± 42.4 |
| 38 | 167.7 ± 54.8 | 1390.8 ± 504.7 | 830.4 ± 329.1 | −5.5 ± 4.1 | 111.8 ± 43.2 | 516 ± 121.7 |

TABLE 31B
CD8+ anti-epitope responses in Rhesus Macaques dosed with chAd-MAG plus
anti-CTLA4 antibody (Ipilimumab) delivered IV. (Group 5). Mean SFC/1e6 splenocytes +/− the
standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 1848.1 ± 432.2 | 1295.7 ± 479.7 | 1709.8 ± 416.9 | 513.7 ± 219.8 | 838.5 ± 221.1 | 2514.6 ± 246.5 |
| 5 | 1844.1 ± 410.2 | 2367.5 ± 334.4 | 1983.1 ± 370.7 | 732.1 ± 249.4 | 1429.7 ± 275.3 | 2517.7 ± 286.5 |
| 6 | 822.4 ± 216.7 | 1131.2 ± 194.7 | 796.8 ± 185.8 | 226.8 ± 70 | 802.2 ± 101.4 | 913.5 ± 222.7 |
| 7 | 1147.2 ± 332.9 | 1066 ± 311.2 | 1149.8 ± 467.3 | 267.4 ± 162.6 | 621.5 ± 283.2 | 1552.2 ± 395.1 |
| 8 | 1192.7 ± 188.8 | 1461.5 ± 237.7 | 1566.9 ± 310.5 | 522.5 ± 118.6 | 662.3 ± 142.4 | 1706 ± 216.7 |
| 10 | 1249 ± 220.3 | 1170.6 ± 227.7 | 1297.3 ± 264.7 | −0.3 ± 4.4 | 551.8 ± 90.5 | 1425.3 ± 142.6 |
| 11 | 934.2 ± 221.7 | 808 ± 191.3 | 1003.1 ± 293.4 | 1.9 ± 4.3 | 364.2 ± 76.6 | 1270.8 ± 191.6 |
| 12 | 1106.2 ± 216.6 | 896.7 ± 190.7 | 1020.1 ± 243.3 | 1.3 ± 3.9 | 436.6 ± 90 | 1222 ± 155.4 |
| 13 | 2023.8 ± 556.3 | 3696.7 ± 1.7 | 2248.5 ± 436.4 | −4.5 ± 3.5 | 2614 ± 406.1 | 3700 ± 0 |
| 14 | 1278.7 ± 240 | 2639.5 ± 387 | 1654.6 ± 381.1 | −6 ± 2.1 | 988.8 ± 197.9 | 2288.3 ± 298.7 |
| 15 | 1458.9 ± 281.8 | 2932.5 ± 488.7 | 1893.4 ± 499 | 74.6 ± 15.6 | 1657.8 ± 508.9 | 2709.1 ± 428.7 |
| 16 | 1556.8 ± 243 | 2143.8 ± 295.2 | 2082.8 ± 234.2 | −5.8 ± 2.5 | 1014.6 ± 161.4 | 2063.7 ± 86.7 |
| 17 | 1527 ± 495.1 | 2213 ± 677.1 | 1767.7 ± 391.8 | 15.1 ± 5.9 | 633.8 ± 133.9 | 2890.8 ± 433.9 |
| 18 | 1068.2 ± 279.9 | 1940.9 ± 204.1 | 1114.1 ± 216.1 | −5.8 ± 2.5 | 396.6 ± 77.6 | 1659.4 ± 171.7 |
| 19 | 760.7 ± 362.2 | 1099.5 ± 438.4 | 802.7 ± 192.5 | −2.4 ± 3.3 | 262.2 ± 62.2 | 1118.6 ± 224.2 |
| 20 | 696.3 ± 138.2 | 954.9 ± 198 | 765.1 ± 248.4 | −1.4 ± 4.4 | 279.6 ± 89.3 | 1139 ± 204.5 |
| 21 | 1201.4 ± 327.9 | 3096 ± 1.9 | 1901 ± 412.1 | −5.8 ± 2.5 | 1676.3 ± 311.5 | 2809.3 ± 195.8 |
| 22 | 1442.5 ± 508.3 | 2944.7 ± 438.6 | 1528.4 ± 349.6 | 2.8 ± 5.1 | 940.7 ± 160.5 | 2306.3 ± 218.6 |
| 23 | 1400.4 ± 502.2 | 2757.1 ± 452.9 | 1604.2 ± 450.1 | −5.1 ± 2.3 | 708.1 ± 162.6 | 2100.4 ± 362.9 |
| 24 | 1351 ± 585.1 | 2264.5 ± 496 | 1080.6 ± 253.8 | 0.3 ± 6.5 | 444.2 ± 126.4 | 1823.7 ± 306.5 |
| 25 | 1211.5 ± 505.2 | 2160.4 ± 581.8 | 970.8 ± 235.9 | 2.5 ± 3.8 | 450.4 ± 126.9 | 1626.2 ± 261.3 |
| 26 | 1443 ± 492.5 | 2485 ± 588 | 1252.5 ± 326.4 | −0.2 ± 6 | 360.2 ± 92.3 | 2081.9 ± 331.1 |
| 27 | 896.2 ± 413.3 | 1686 ± 559.5 | 751 ± 192.1 | −3.7 ± 2.5 | 247.4 ± 82.8 | 1364.1 ± 232 |
| 28 | 1147.8 ± 456.9 | 1912.1 ± 417.1 | 930.3 ± 211.4 | −5.8 ± 2.5 | 423.9 ± 79.6 | 1649.3 ± 315 |
| 29 | 1038.5 ± 431.9 | 1915.2 ± 626.1 | 786.8 ± 205.9 | 23.5 ± 8.3 | 462.8 ± 64 | 1441.5 ± 249.7 |
| 30 | 730.5 ± 259.3 | 1078.6 ± 211.5 | 699.1 ± 156.2 | −4.4 ± 2.7 | 234.4 ± 43.9 | 1160.6 ± 112.6 |
| 31 | 750.4 ± 328.3 | 1431 ± 549.9 | 650.6 ± 141.1 | −5.2 ± 3 | 243.4 ± 56.4 | 868.9 ± 142.8 |
| 32 | 581.4 ± 227.7 | 1326.6 ± 505.2 | 573.3 ± 138 | −3.2 ± 4.2 | 160.8 ± 49.2 | 936.4 ± 110.4 |
| 33 | 2198.4 ± 403.8 | 3093.4 ± 123.3 | 2391.8 ± 378.4 | 7.1 ± 8.5 | 1598.1 ± 343.1 | 2827.5 ± 289.5 |
| 34 | 2654.3 ± 337 | 2709.9 ± 204.3 | 1297.5 ± 291.4 | 0.4 ± 4.2 | 1091.8 ± 242.9 | 1924 ± 245.7 |
| 37 | 846.8 ± 301.7 | 1706.9 ± 196 | 973.6 ± 149.3 | 50.5 ± 45.2 | 777.3 ± 140.2 | 1478.8 ± 94.3 |

TABLE 31C
CD8+ anti-epitope responses in Rhesus Macaques dosed with chAd-MAG plus
anti-CTLA4 antibody (Ipilimumab) delivered SC (Group 6). Mean SFC/1e6 splenocytes +/−
the standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 598.3 ± 157.4 | 923.7 ± 306.8 | 1075.6 ± 171.8 | 180.5 ± 74.1 | 752.3 ± 245.8 | 1955.3 ± 444.4 |
| 5 | 842.2 ± 188.5 | 1703.7 ± 514.2 | 1595.8 ± 348.4 | 352.7 ± 92.3 | 1598.9 ± 416.8 | 2163.7 ± 522.1 |
| 6 | 396.4 ± 45.3 | 728.3 ± 232.7 | 503.8 ± 151.9 | 282 ± 69 | 463.1 ± 135.7 | 555.2 ± 191.5 |
| 7 | 584.2 ± 177 | 838.3 ± 254.9 | 1013.9 ± 349.4 | 173.6 ± 64.3 | 507.4 ± 165.7 | 1222.8 ± 368 |
| 8 | 642.9 ± 134 | 1128.6 ± 240.6 | 1259.1 ± 163.8 | 366.1 ± 72.8 | 726.7 ± 220.9 | 1695.6 ± 359.4 |
| 10 | 660.4 ± 211.4 | 746.9 ± 222.7 | 944.8 ± 210.2 | −1.2 ± 1.9 | 523.4 ± 230.7 | 787.3 ± 308.3 |
| 11 | 571.2 ± 162 | 609.4 ± 194.3 | 937.9 ± 186.5 | −8.9 ± 2.3 | 511.6 ± 229.6 | 1033.3 ± 315.7 |
| 12 | 485.3 ± 123.7 | 489.4 ± 142.7 | 919.3 ± 214.1 | −8.9 ± 2.3 | 341.6 ± 139.4 | 1394.7 ± 432.1 |
| 13 | 986.9 ± 154.5 | 2811.9 ± 411.3 | 1687.7 ± 344.3 | −4.1 ± 5.1 | 1368.5 ± 294.2 | 2751 ± 501.9 |
| 14 | 945.9 ± 251.4 | 2027.7 ± 492.8 | 1386.7 ± 326.7 | −5.7 ± 2.8 | 708.9 ± 277.1 | 1588.2 ± 440.1 |
| 15 | 1075.2 ± 322.4 | 2386 ± 580.7 | 1606.3 ± 368.1 | −5.4 ± 3.2 | 763.3 ± 248.8 | 1896.5 ± 507.8 |
| 16 | 1171.8 ± 341.6 | 2255.1 ± 439.6 | 1672.2 ± 342.3 | −7.8 ± 2.4 | 1031.6 ± 228.8 | 1896.4 ± 419.9 |
| 17 | 1118.2 ± 415.4 | 2156.3 ± 476 | 1345.3 ± 377.7 | −1.1 ± 6.7 | 573.7 ± 118.8 | 1614.4 ± 382.3 |
| 18 | 861.3 ± 313.8 | 2668.2 ± 366.8 | 1157.2 ± 259.6 | −8.9 ± 2.3 | 481.2 ± 164 | 1725.8 ± 511.4 |
| 19 | 719.2 ± 294.2 | 1447.2 ± 285 | 968 ± 294.5 | −2.2 ± 4.6 | 395.6 ± 106.1 | 1199.6 ± 289.2 |
| 20 | 651.6 ± 184 | 1189.8 ± 242.8 | 947.4 ± 249.8 | −8.9 ± 2.3 | 355 ± 106.3 | 1234.7 ± 361.7 |
| 21 | 810.3 ± 301.9 | 2576.2 ± 283.7 | 1334 ± 363.1 | −8.9 ± 2.3 | 892.2 ± 305 | 1904.4 ± 448.1 |
| 22 | 775 ± 196.4 | 2949 ± 409.7 | 1421.8 ± 309.7 | 38 ± 27.8 | 577 ± 144.2 | 2330.6 ± 572.3 |
| 23 | 584.9 ± 240.2 | 1977.9 ± 361.4 | 1209.8 ± 405.1 | −7.3 ± 3.2 | 273.7 ± 93.3 | 1430.6 ± 363.9 |
| 24 | 485.4 ± 194.4 | 1819.8 ± 325.5 | 837.2 ± 261.4 | −3.4 ± 4.1 | 234.4 ± 71.1 | 943.9 ± 243.3 |
| 25 | 452.3 ± 175 | 2072 ± 405.7 | 957.1 ± 293.1 | −8.9 ± 2.3 | 163 ± 43.2 | 1341.2 ± 394.7 |
| 26 | 517.9 ± 179.1 | 2616 ± 567.5 | 1126.6 ± 289 | −8.3 ± 2.3 | 199.9 ± 89.2 | 1615.7 ± 385.6 |
| 27 | 592.8 ± 171.7 | 1838.3 ± 372.4 | 749.3 ± 170.4 | −7.3 ± 2.5 | 325.5 ± 98.7 | 1110.7 ± 308.8 |
| 28 | 793 ± 228.5 | 1795.4 ± 332.3 | 1068.7 ± 210.3 | 2.5 ± 4.1 | 553.1 ± 144.3 | 1480.8 ± 357.1 |
| 29 | 661.8 ± 199.9 | 2140.6 ± 599.3 | 1202.7 ± 292.2 | −8.7 ± 2.8 | 558.9 ± 279.2 | 1424.2 ± 408.6 |
| 30 | 529.1 ± 163.3 | 1528.2 ± 249.8 | 840.5 ± 218.3 | −8.9 ± 2.3 | 357.7 ± 149.4 | 1029.3 ± 335 |
| 31 | 464.8 ± 152.9 | 1332.2 ± 322.7 | 726.3 ± 194.3 | −8.9 ± 2.3 | 354.3 ± 158.6 | 884.4 ± 282 |
| 32 | 612.9 ± 175.3 | 1584.2 ± 390.2 | 1058.3 ± 219.8 | −8.7 ± 2.8 | 364.6 ± 149.8 | 1388.8 ± 467.3 |
| 33 | 1600.2 ± 416.7 | 2597.4 ± 367.9 | 2086.4 ± 414.8 | −6.3 ± 3.3 | 893.8 ± 266 | 2490.6 ± 416.4 |
| 34 | 2814.6 ± 376.2 | 2713.6 ± 380.8 | 1888.8 ± 499.4 | −7.5 ± 3.1 | 1288.9 ± 438.9 | 2428.1 ± 458.9 |
| 37 | 1245.9 ± 471.7 | 1877.7 ± 291.2 | 1606.6 ± 441.9 | 14.2 ± 13 | 1227.5 ± 348.1 | 1260.7 ± 342 |

Memory Phenotyping in Indian Rhesus Macaques

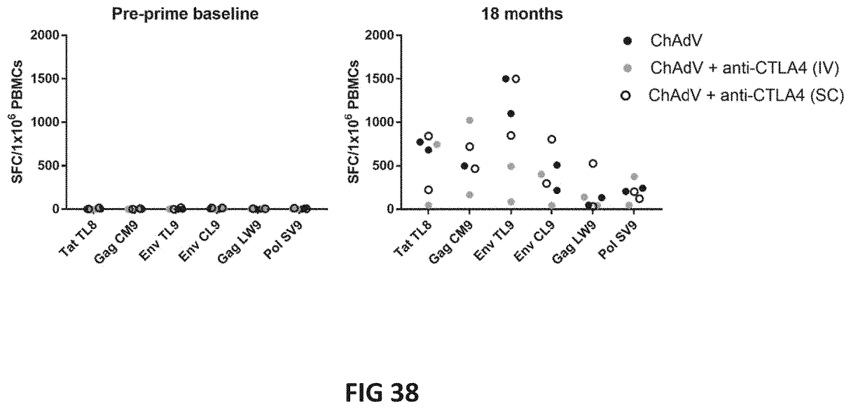

Rhesus macaque were immunized with ChAdV68.5WTnt.MAG25mer NEE-MAG25mer srRNA heterologous prime/boost regimen with or without anti-CTLA4, and boosted again with ChAdV68.5WTnt.MAG25mer. Groups were assessed 11 months after the final ChAdV68 administration (study month 18). by ELISpot was performed as described. FIG. 38 and Table 43 shows cellular responses to six different Mamu-A*01 restricted epitopes as measured by ELISpot both pre-immunization (left panel) and after 18 months (right panel). The detection of responses to the restricted epitopes demonstrates antigen-specific memory responses were generated by ChAdV68/samRNA vaccine protocol.

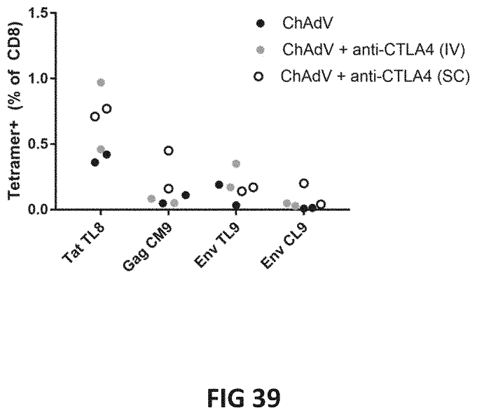

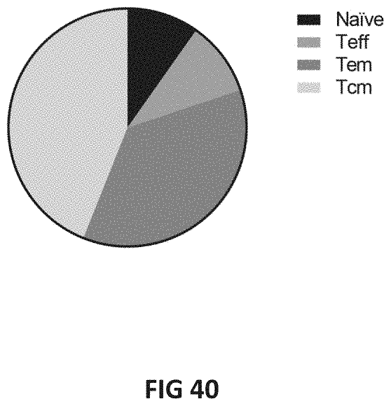

To assess memory, CD8+ T-cells recognizing 4 different rhesus macaque Mamu-A*01 class I epitopes encoded in the vaccines were monitored using dual-color Mamu-A*01 tetramer labeling, with each antigen being represented by a unique double positive combination, and allowed the identification of all 4 antigen-specific populations within a single sample. Memory cell phenotyping was performed by co-staining with the cell surface markers CD45RA and CCR7. FIG. 39 and Table 44 shows the results of the combinatorial tetramer staining and CD45RA/CCR7 co-staining for memory T-cells recognizing four different Mamu-A*01 restricted epitopes. The T cell phenotypes were also assessed by flow cytometry. FIG. 40 shows the distribution of memory cell types within the sum of the four Mamu-A*01 tetramer+CD8+ T-cell populations at study month 18. Memory cells were characterized as follows: CD45RA+CCR7+=naïve, CD45RA+CCR7-=effector (Teff), CD45RA-CCR7+=central memory (Tcm), CD45RA-CCR7-=effector memory (Tem). Collectively, the results demonstrate that memory responses were detected at least one year following the last boost indicating long lasting immunity, including effector, central memory, and effector memory populations.

TABLE 43
Mean spot forming cells (SFC) per 10⁶ PBMCs for each animal at both pre-prime and
memory assessment time points (18 months).

| | Pre-prime baseline | | | | | | 18 months | | | | | |
|---|---|---|---|---|---|---|---|---|---|---|---|---|
| | Tat | Gag | Env | Env | Gag | Pol | Tat | Gag | Env | Env | Gag | Pol |
| Animal | TL8 | CM9 | TL9 | CL9 | LW9 | SV9 | TL8 | CM9 | TL9 | CL9 | LW9 | SV9 |
| 1 | 1.7 | 0.0 | 0.0 | 5.0 | 0.0 | 13.7 | 683.0 | 499.2 | 1100.3 | 217.5 | 47.7 | 205.3 |
| 2 | 0.0 | 0.0 | 0.0 | 0.2 | 0.1 | 0.0 | 773.4 | ND | 1500.0 | 509.3 | 134.5 | 242.5 |
| 3 | 0.0 | 0.0 | 6.7 | 6.8 | 10.2 | 3.3 | 746.3 | 167.5 | 494.1 | 402.8 | 140.6 | 376.0 |
| 4 | 0.0 | 0.0 | 0.0 | 0.0 | 0.0 | 0.0 | 47.6 | 1023.9 | 85.1 | 44.2 | 44.2 | 47.6 |
| 5 | 15.3 | 6.7 | 18.6 | 15.6 | 5.2 | 12.1 | 842.4 | 467.7 | 1500.0 | 805.9 | 527.8 | 201.8 |
| 6 | 3.1 | 0.0 | 0.0 | 15.5 | 6.9 | 5.3 | 224.3 | 720.3 | 849.0 | 296.9 | 32.4 | 121.9 |

ND = not determined due to technical exclusion

TABLE 44
Percent Mamu-A*01 tetramer positive out of live CD8+ cells

| Animal | Tat TL8 | Gag CM9 | Env TL9 | Env CL9 |
|---|---|---|---|---|
| 1 | 0.42 | 0.11 | 0.19 | 0.013 |
| 2 | 0.36 | 0.048 | 0.033 | 0.00834 |
| 3 | 0.97 | 0.051 | 0.35 | 0.048 |
| 4 | 0.46 | 0.083 | 0.17 | 0.028 |
| 5 | 0.77 | 0.45 | 0.14 | 0.2 |
| 6 | 0.71 | 0.16 | 0.17 | 0.04 |

### XIX. Identification of MHC/Peptide Target-Reactive T Cells and TCRs

Target reactive T cells and TCRs are identified for one or more of the antigen/HLA peptides pairs described in Table A, AACR GENIE Results, Table 1.2, and/or Additional MS Validated Neoantigens (see SEQ ID NO: 57-29,357 and SEQ ID NO: 29,512-29,519 and below)

T cells can be isolated from blood, lymph nodes, or tumors of patients. T cells can be enriched for antigen-specific T cells, e.g., by sorting antigen-MHC tetramer binding cells or by sorting activated cells stimulated in an in vitro co-culture of T cells and antigen-pulsed antigen presenting cells. Various reagents are known in the art for antigen-specific T cell identification including antigen-loaded tetramers and other MHC-based reagents.

Antigen-relevant alpha-beta (or gamma-delta) TCR dimers can be identified by single cell sequencing of TCRs of antigen-specific T cells. Alternatively, bulk TCR sequencing of antigen-specific T cells can be performed and alpha-beta pairs with a high probability of matching can be determined using a TCR pairing method known in the art.

Alternatively or in addition, antigen-specific T cells can be obtained through in vitro priming of naïve T cells from healthy donors. T cells obtained from PBMCs, lymph nodes, or cord blood can be repeatedly stimulated by antigen-pulsed antigen presenting cells to prime differentiation of antigen-experienced T cells. TCRs can then be identified similarly as described above for antigen-specific T cells from patients.

### XX. Identification of Shared Neoantigens

We identified shared neoantigens using a series of steps. We obtained a list of common driver mutations classified as “confirmed somatic” from the COSMIC database. For each mutation, we generated candidate neoepitopes (8 to 11-mer peptides), used a TPM of 100, and ran our EDGE prediction model (a deep learning model trained on HLA presented peptides sequenced by MS/MS, as described in international patent application publications WO/2017/106638, WO/2018/195357, and WO/2018/208856, each herein incorporated by reference, in their entirety, for all purposes) across all modeled HLA alleles. Note that each peptide contained at least one mutant amino acid and was not a self-peptide. We then recorded any peptide with an HLA allele that has an EDGE score >0.001. The results are shown in Table A. A total of 10261 shared neoantigen sequences were thus identified and are described in SEQ ID NO: 10,755-21,015. The corresponding HLA allele(s) for each sequence are shown.

The initial list provided in Table A was further analyzed for level of neoantigen/HLA prevalence in the patient population. “Antigen/HLA prevalence” is calculated as the frequency of antigen (A) in a given population multiplied by the frequency of an HLA allele (B) in the given population. Antigen/HLA prevalence can also refer to mutation/HLA prevalence or neoantigen/HLA prevalence. As part of this analysis, for each mutation, its (A) frequency was obtained across common tumor types in TCGA and recorded at its highest frequency amongst tumor types. (B) For each HLA allele in EDGE, the HLA allele frequency TCGA (a predominantly Caucasian population) was recorded. HLA allele frequencies are described in more detail in Shukla, S. A. et al. (Nat. Biotechnol. 33, 1152-1158 2015), herein incorporated by reference for all purposes. The neoantigen/HLA prevalence was calculated as (A) multiplied by (B). Any neoepitope/HLA pair in Table A that is >0.10% prevalence using this methodology is identified with a “Most Common 1” (2387/10261).

Additionally, we characterized the prevalence of cancer driver mutations across a large cohort of patient samples representative of the advanced cancer patient population relevant for potential clinical studies. EDGE prediction was performed using the publicly-released AACR Genie v4.1 dataset, which has over 40,000 patients sequenced on NGS cancer gene panels ranging from 50 to 500 genes, from major academic cancer centers including Dana-Farber, Johns Hopkins, M D Anderson, MSKCC, and Vanderbilt. We selected base substitution and indel mutations in lung, microsatellite stable colon, and pancreatic cancers, and required coverage across multiple gene panels. We analyzed each neoantigen peptide paired with each of greater than 90 Class I HLA alleles covered in our EDGE antigen presentation prediction model and recorded the epitopes and corresponding HLA alleles with an EDGE probability of HLA presentation score of >0.001. We then determined the neoantigen/HLA prevalence of those peptide with an EDGE score >0.001, calculated as A*B, where A is the highest frequency of the mutation amongst the three tumor types and B is the HLA allele frequency. We used HLA allele frequencies representative of the population in the USA by examining HLA alleles from the TCGA population and tabulating the frequency for each HLA allele (Shukla, S. A. et al.). Peptides and corresponding HLA alleles that demonstrated a neoantigen/HLA prevalence >0.01% from the analysis are described in SEQ ID NO:21,016-29,357 and referred to as AACR GENIE Results.

In addition, we analyzed the EDGE prediction run on the available AACR GENIE database of tumor mutations for specific cancer subsets across the following modeled HLA alleles: A0101; A0201; A0205; A0206; A0301; A1101; A2301; A2402; A2501; A2601; A2902; A3001; A3002; A3101; A3201; A3301; A3303; A6801; A6802; B0702; B0801; B1302; B1401; B1402; B1501; B1503; B1510; B1801; B2705; B3501; B3502; B3503; B3508; B3701; B3801; B3901; B3906; B4001; B4002; B4402; B4403; B4901; B5001; B5101; B5501; B5701; B5801; C0102; C0202; C0302; C0303; C0304; C0401; C0501; C0602; C0701; C0702; C0704; C0801; C0802; C1203; C1402; C1502; C1601; C1701

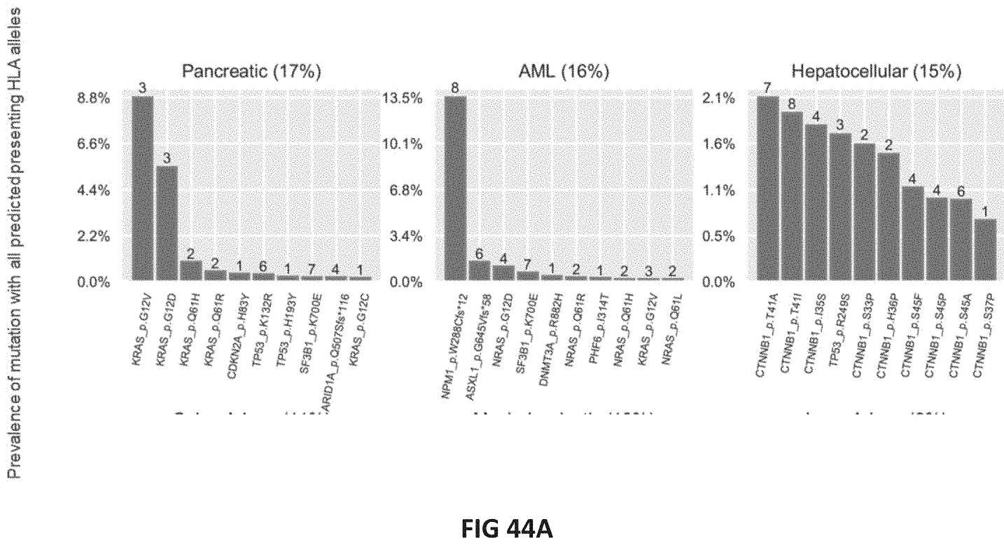

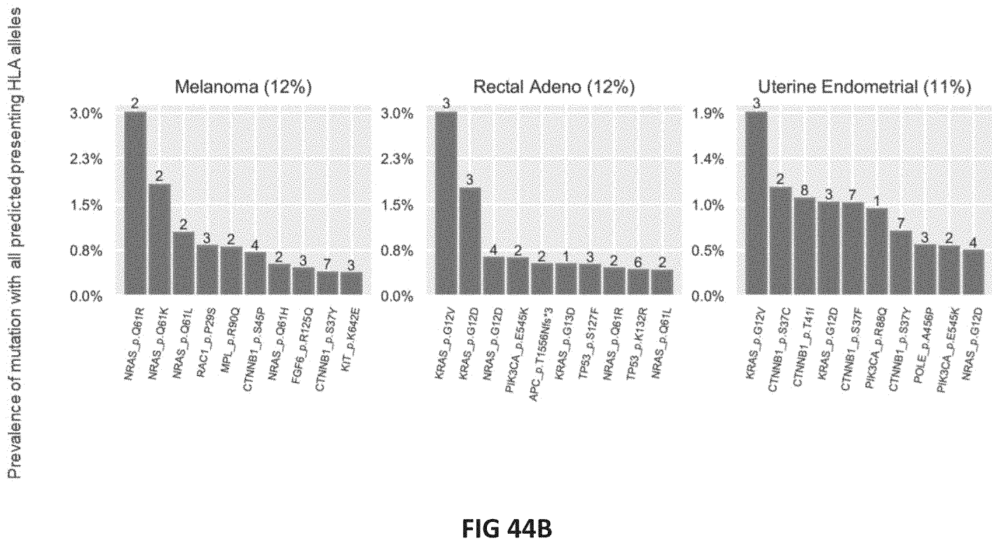

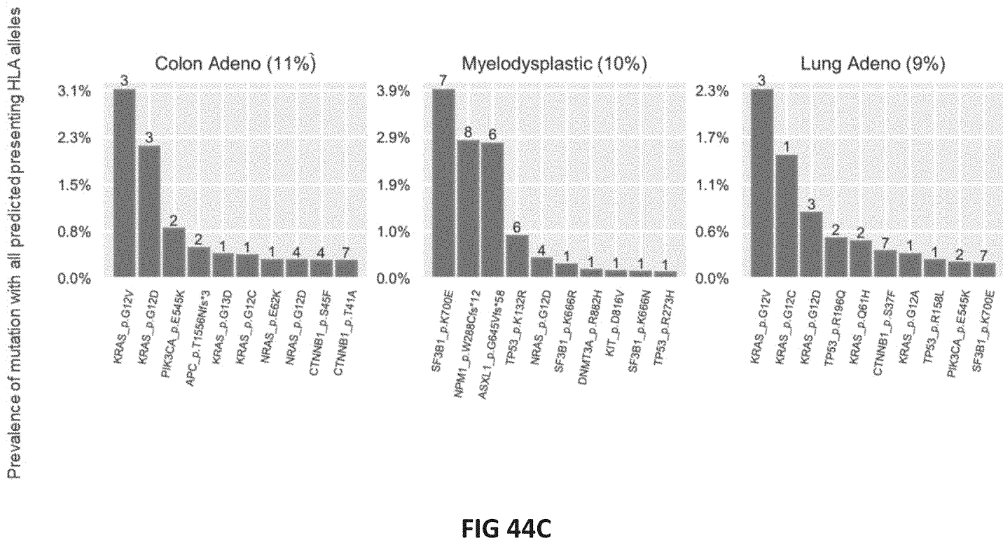

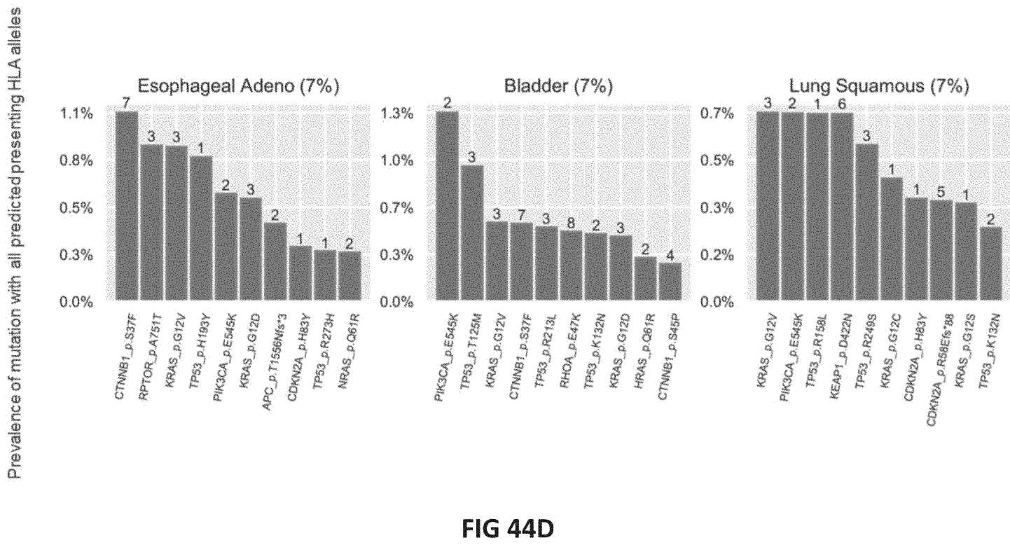

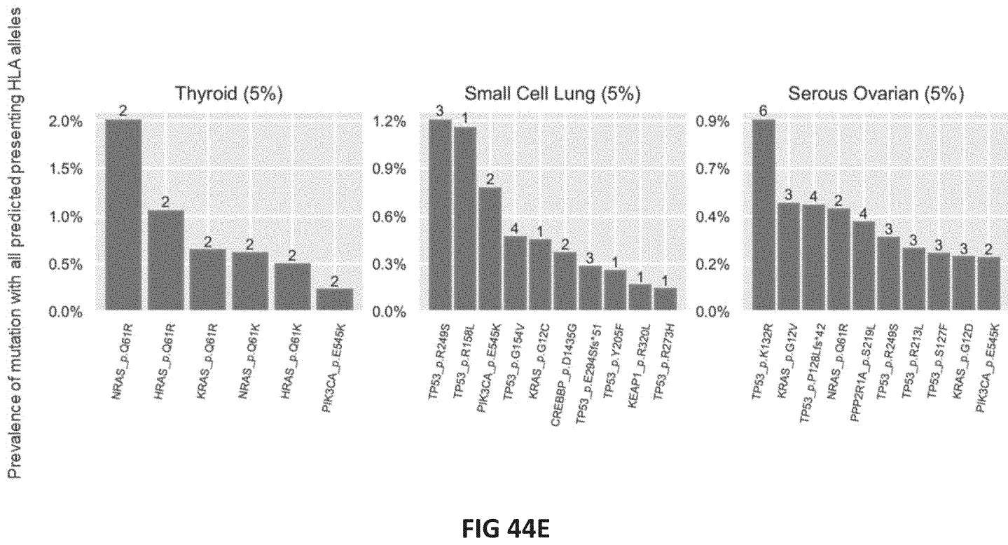

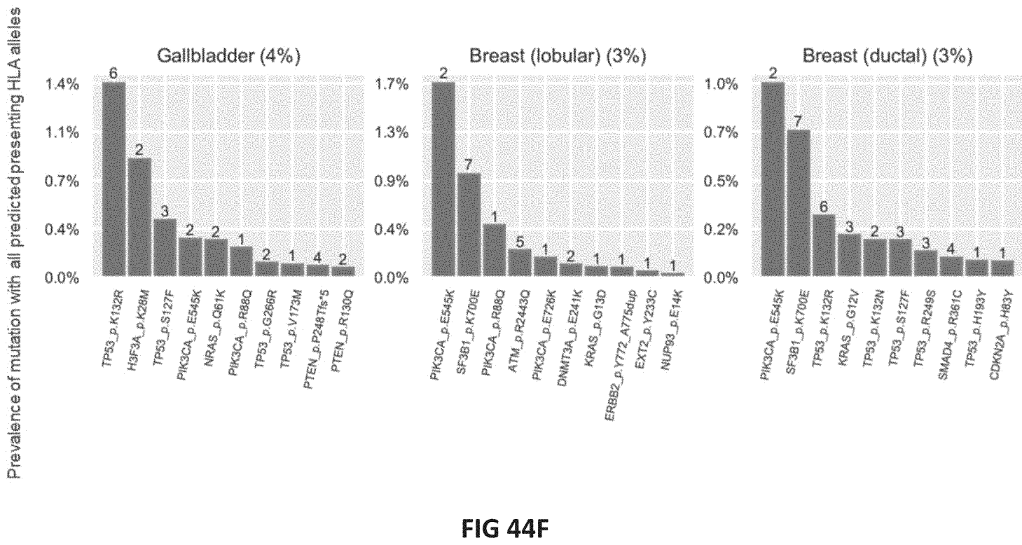

We then recorded any peptide with an HLA allele that has an EDGE score >0.1. The results are shown in FIG. 44, with antigen/HLA prevalence (the frequency of antigen (A) in a given population multiplied by the frequency of an HLA allele (B) in the given population) plotted for each of the mutations shown in the following cancer subsets analyzed:
FIG. 44A: Pancreatic, AML, Hepatocellular (left, middle, right panels, respectively)
FIG. 44B: Melanoma, Rectal Adeno, Uterine Endometrial (left, middle, right panels, respectively)
FIG. 44C: Colon Adeno, Myelodysplastic, Lung Adeno (left, middle, right panels, respectively)
FIG. 44D: Esophageal Adeno, Bladder, Lung Squamous (left, middle, right panels, respectively)
FIG. 44E: Thyroid, Small Cell Lung, Serous Ovarian (left, middle, right panels, respectively)
FIG. 44F: Gallbladder, Breast [lobular], Breast [ductal] (left, middle, right panels, respectively)
   The total Antigen/HLA prevalence of all shared neoantigens combined is indicated (see percentage next to given cancer). Also indicated are the number of distinct HLAs predicted to present the given neoantigen (see number above bar for each specific mutation).As determined from analysis of FIG. 44: (1) a large minority of patients with cancer may harbor Class I shared neoantigens (up to ˜10-15% in multiple indications); (2) shared neoantigens are most prevalent in indications driven by KRAS, and many shared neoantigens are shared across indications; and (3) certain indications also have distinct shared neoantigens profiles such CTNNB1 in HCC and NPM1 in AML. Accordingly, the analysis demonstrates shared neoantigens provide and important source of targets for cancer immunotherapies.

### XXI. A. Validation of Shared Neoantigen Presentation in Human Tumors

Mass spectrometry (MS) validation of candidate shared neoantigens was performed using targeted mass spectrometry methods. Nearly 500 frozen resected lung, colorectal and pancreatic tumor samples were homogenized and used for both RNASeq transcriptome sequencing and immunoprecipitation of the HLA/peptide complexes. A peptide target list was generated for each sample by analysis of the transcriptome, whereby recurrent cancer driver mutations, as defined in the AACR Genie v4.1 dataset, were identified and RNA expression levels assessed. The EDGE model of antigen presentation was then applied to the mutation sequence and expression data to prioritize peptides for the targeting list. The peptides from the HLA molecules were eluted and collected using size exclusion to isolate the presented peptides prior to mass spectrometry. Synthetic heavy labeled peptide with the same amino acid sequence was co-loaded with each sample for targeted mass spectrometry. Both coelution of the heavy labeled peptide with the experimental peptide and analysis of the fragmentation pattern we were used to validate a candidate epitope. Mass spectrometry analysis methods are described in more detail in Gillete et al. (Nat Methods. 2013 January;10(1):28-34), herein incorporated by reference in its entirety for all purposes. Shared neoantigen epitopes from driver mutations validated in this manner with sufficient prevalence for further consideration are summarized in Table 32A below, along with sample tumor type and associated HLA alleles.

TABLE 32A
MS-validated neoantigen neoepitopes in human tumors

| | | | | Peptide | |
|---|---|---|---|---|---|
| Patient | Tumor | Presenting | Gene | Elution | Targeted |
| ID | Type | HLA* | Mutation | Time (min.) | Peptide |
| A0002082 | CRC | HLA-A* | CTNNB1_S45P | 17.2 | TTAPPLSGK |
| | | 03:01 | | | (SEQ ID NO: |
| | | | | | 29393) |
| A0001877 | Lung | HLA-A* | KRAS_G12C | 46.7 | KLVVVGACGV |
| | | 02:01 | | | (SEQ ID NO: |
| | | | | | 29395) |
| A0002129 | Lung | HLA-A* | KRAS_G12C | 46.5 | KLVVVGACGV |
| | | 02:01 | | | (SEQ ID NO: |
| | | | | | 29395) |
| A0001947 | CRC | HLA-A* | KRAS_G12D | 12 | VVGADGVGK |
| | | 11:01 | | (pep. #1) | (SEQ ID NO: |
| | | | | | 29396) |
| A0001947 | CRC | HLA-A* | KRAS_G12D | 23.4 | WVGADGVGK |
| | | 11:01 | | (pep. #2) | (SEQ ID NO: |
| | | | | | 29397) |
| A0001474 | Gastric | HLA-A* | KRAS_G12V | 30.4 | WVGAVGVGK |
| | | 11:01 | | | (SEQ ID NO: |
| | | | | | 29398) |
| A0001730 | Pancreatic | HLA-A* | KRAS_G12V | 18.4 | VVGAVGVGK |
| | | 11:01 | | (pep. #1) | (SEQ ID NO: |
| | | | | | 29399) |
| A0001730 | Pancreatic | HLA-A* | KRAS_G12V | 31.6 | WVGAVGVGK |
| | | 11:01 | | (pep. #2) | (SEQ ID NO: |
| | | | | | 29398) |
| A0001896 | Lung | HLA-C* | KRAS_G12V | 23.6 | AVGVGKSAL |
| | | 01:02 | | | (SEQ ID NO: |
| | | | | | 29400) |
| A0001966 | CRC | HLA-A* | KRAS_G12V | 27.8 | WVGAVGVGK |
| | | 03:01 | | | (SEQ ID NO: |
| | | | | | 29398) |
| A0001973 | Ovarian | HLA-A* | TP53_K132N | 97.7 | TYSPALNNMF |
| | | 24:02 | | | (SEQ ID NO: |
| | | | | | 29401) |
| A0001983 | Ovarian | HLA-A* | CTNNB1_S37Y | 50.9 | YLDSGIHYGA |
| | | 02:01 | | | |
| A0000799 | CRC | HLA-B* | CHD4_K73fs | 43.3 | TVRAATIL |
| | | 08:01 | | | |

*When the same peptide was predicted to be presented by multiple HLA alleles for a patient and detected by MS/MS, it was inferred to be presented by the highest scoring HLA allele by EDGE or both alleles if the scores were sufficiently close

We further evaluated the MS data with respect to mutations for which peptides were not detected in order to assess the value of narrowly targeting patients with specific HLAs for treatment, e.g., requiring patients to have at least one validated or predicted HLA allele that presents a neoantigen contained in a vaccine cassette.

For example, in the case of KRAS, we counted the number of patient samples in which KRAS epitope peptides for particular HLA alleles were detected or not detected. (When the same peptide was predicted to be presented by multiple HLA alleles for a patient and detected by MS/MS, it was inferred to be presented by the highest scoring HLA allele by EDGE or both alleles if the scores were sufficiently close). Results are presented in Table 33. Based on these results, several common HLA alleles are not expected to present a given KRAS neoantigen and these KRAS neoantigen/HLA pair can be excluded for purposes of selection criteria for a vaccine cassette design and patient selection in this instance. For example, Table 34 directed to a specific vaccine cassette (see Section XXII below) does not include predicted neoantigen/HLA pair G12D/A*02:01 on the basis that the peptide was not detected in 17 samples tested, and likewise did not include G12V/A*02:01 on the basis that the peptide was not detected in 9 samples tested. In contrast, neoantigen/HLA pair G12D/A*11:01 was considered validated on the basis that the peptide was detected in 1/5 samples tested, and likewise G12V/A*11:01 was considered validated on the basis that the peptide was detected in 2/6 samples tested.

These results highlight the importance of identifying the relevant neoantigen/HLA pairs for proper HLA type selection in patient selection for treatment with a shared neoantigen vaccine, such as that described in Table 34. Specifically, several common KRAS neoantigen/HLA pairs were excluded for purposes of selection criteria in this case as the MS data suggested a shared neoantigen vaccine would unlikely provide a benefit to a patient with that predicted KRAS neoantigen/HLA pair (e.g., G12D/A*02:01 or G12V/A*02:01).

TABLE 33
Presentation of KRAS Neoantigens

| | | HLA Peptide | Number of tested |
|---|---|---|---|
| | | confirmation | patient samples |
| Genemutation | HLA | by MS/MS | with MS/MS result |
| KRAS_G12A | HLA-A*02:01 | N | 2 |
| KRAS_G12A | HLA-A*02:06 | N | 1 |
| KRAS_G12A | HLA-A*03:01 | N | 3 |
| KRAS_G12A | HLA-B*27:05 | N | 1 |
| KRAS_G12A | HLA-B*35:01 | N | 1 |
| KRAS_G12A | HLA-B*41:02 | N | 1 |
| KRAS_G12A | HLA-B*48:01 | N | 1 |
| KRAS_G12A | HLA-C*08:03 | N | 1 |
| KRAS_G12C | HLA-A*02:01 | Y | 2 |
| KRAS_G12C | HLA-A*02:01 | N | 2 |
| KRAS_G12C | HLA-A*03:01 | N | 8 |
| KRAS_G12C | HLA-A*03:02 | N | 1 |
| KRAS_G12C | HLA-A*68:01 | N | 1 |
| KRAS_G12C | HLA-B*27:05 | N | 1 |
| KRAS_G12D | HLA-A*02:01 | N | 17 |
| KRAS_G12D | HLA-A*02:05 | N | 2 |
| KRAS_G12D | HLA-A*03:01 | N | 4 |
| KRAS_G12D | HLA-A*11:01 | Y | 1 |
| KRAS_G12D | HLA-A*11:01 | N | 4 |
| KRAS_G12D | HLA-A*26:01 | N | 2 |
| KRAS_G12D | HLA-A*31:01 | N | 4 |
| KRAS_G12D | HLA-A*68:01 | N | 3 |
| KRAS_G12D | HLA-B*07:02 | N | 4 |
| KRAS_G12D | HLA-B*08:01 | N | 1 |
| KRAS_G12D | HLA-B*13:02 | N | 1 |
| KRAS_G12D | HLA-B*15:01 | N | 5 |
| KRAS_G12D | HLA-B*27:05 | N | 2 |
| KRAS_G12D | HLA-B*35:01 | N | 2 |
| KRAS_G12D | HLA-B*37:01 | N | 1 |
| KRAS_G12D | HLA-B*38:01 | N | 2 |
| KRAS_G12D | HLA-B*40:01 | N | 3 |
| KRAS_G12D | HLA-B*40:02 | N | 3 |
| KRAS_G12D | HLA-B*44:02 | N | 1 |
| KRAS_G12D | HLA-B*44:03 | N | 4 |
| KRAS_G12D | HLA-B*48:01 | N | 1 |
| KRAS_G12D | HLA-B*50:01 | N | 1 |
| KRAS_G12D | HLA-B*57:01 | N | 1 |
| KRAS_G12D | HLA-C*01:02 | N | 1 |
| KRAS_G12D | HLA-C*02:02 | N | 1 |
| KRAS_G12D | HLA-C*03:03 | N | 3 |
| KRAS_G12D | HLA-C*03:04 | N | 2 |
| KRAS_G12D | HLA-C*04:01 | N | 8 |
| KRAS_G12D | HLA-C*05:01 | N | 2 |
| KRAS_G12D | HLA-C*07:04 | N | 1 |
| KRAS_G12D | HLA-C*08:02 | N | 2 |
| KRAS_G12D | HLA-C*08:03 | N | 1 |
| KRAS_G12D | HLA-C*16:01 | N | 1 |
| KRAS_G12D | HLA-C*17:01 | N | 1 |
| KRAS_G12R | HLA-B*41:02 | N | 1 |
| KRAS_G12R | HLA-C*07:04 | N | 1 |
| KRAS_G12V | HLA-A*02:01 | N | 9 |
| KRAS_G12V | HLA-A*02:05 | N | 1 |
| KRAS_G12V | HLA-A*02:06 | N | 1 |
| KRAS_G12V | HLA-A*03:01 | Y | 1 |
| KRAS_G12V | HLA-A*03:01 | N | 4 |
| KRAS_G12V | HLA-A*11:01 | Y | 2 |
| KRAS_G12V | HLA-A*11:01 | N | 4 |
| KRAS_G12V | HLA-A*25:01 | N | 3 |
| KRAS_G12V | HLA-A*26:01 | N | 2 |
| KRAS_G12V | HLA-A*30:01 | N | 2 |
| KRAS_G12V | HLA-A*31:01 | N | 2 |
| KRAS_G12V | HLA-A*32:01 | N | 1 |
| KRAS_G12V | HLA-A*68:02 | N | 1 |
| KRAS_G12V | HLA-B*07:02 | N | 6 |
| KRAS_G12V | HLA-B*08:01 | N | 1 |
| KRAS_G12V | HLA-B*13:02 | N | 2 |
| KRAS_G12V | HLA-B*14:02 | N | 1 |
| KRAS_G12V | HLA-B*15:01 | N | 2 |
| KRAS_G12V | HLA-B*27:05 | N | 2 |
| KRAS_G12V | HLA-B*39:01 | N | 1 |
| KRAS_G12V | HLA-B*40:01 | N | 1 |
| KRAS_G12V | HLA-B*40:02 | N | 1 |
| KRAS_G12V | HLA-B*41:02 | N | 1 |
| KRAS_G12V | HLA-B*44:05 | N | 1 |
| KRAS_G12V | HLA-B*50:01 | N | 1 |
| KRAS_G12V | HLA-B*51:01 | N | 1 |
| KRAS_G12V | HLA-C*01:02 | Y | 1 |
| KRAS_G12V | HLA-C*01:02 | N | 1 |
| KRAS_G12V | HLA-C*03:03 | N | 1 |
| KRAS_G12V | HLA-C*03:04 | N | 2 |
| KRAS_G12V | HLA-C*08:02 | N | 2 |
| KRAS_G12V | HLA-C*14:02 | N | 1 |
| KRAS_G12V | HLA-C*17:01 | N | 2 |
| KRAS_G13D | HLA-A*02:01 | N | 2 |
| KRAS_G13D | HLA-B*07:02 | N | 1 |
| KRAS_G13D | HLA-B*08:01 | N | 1 |
| KRAS_G13D | HLA-B*35:01 | N | 1 |
| KRAS_G13D | HLA-B*35:03 | N | 1 |
| KRAS_G13D | HLA-B*35:08 | N | 1 |
| KRAS_G13D | HLA-B*38:01 | N | 1 |
| KRAS_G13D | HLA-C*04:01 | N | 3 |
| KRAS_Q61H | HLA-A*01:01 | N | 1 |
| KRAS_Q61H | HLA-A*02:01 | N | 2 |
| KRAS_Q61H | HLA-A*23:01 | N | 2 |
| KRAS_Q61H | HLA-A*29:01 | N | 1 |
| KRAS_Q61H | HLA-A*30:02 | N | 1 |
| KRAS_Q61H | HLA-A*33:01 | N | 1 |
| KRAS_Q61H | HLA-A*68:01 | N | 1 |
| KRAS_Q61H | HLA-B*07:02 | N | 1 |
| KRAS_Q61H | HLA-B*08:01 | N | 1 |
| KRAS_Q61H | HLA-B*18:01 | N | 1 |
| KRAS_Q61H | HLA-B*35:01 | N | 1 |
| KRAS_Q61H | HLA-B*38:01 | N | 1 |
| KRAS_Q61H | HLA-B*40:01 | N | 1 |
| KRAS_Q61H | HLA-B*44:02 | N | 1 |
| KRAS_Q61H | HLA-C*03:04 | N | 1 |
| KRAS_Q61H | HLA-C*05:01 | N | 2 |
| KRAS_Q61H | HLA-C*08:02 | N | 1 |

### XXL B. Validation of Shared Neoantigen Presentation Using and In Vitro System

Mass spectrometry (MS) validation for HLA-presentation for candidate shared neoantigens was also performed using targeted mass spectrometry methods and an in vitro single-HLA expression system. Briefly, cell lines were engineered to express a single specific HLA alleles and inducibly express a candidate shared neoantigen.

Single HLA Allele expressing K562 cell lines were created by traditional transfection methods using reagent kits and the instructions provided. Details are provided below for the methods used but other similar methods could be used.

To create virus particles for transduction of the HLA genes into K562 cells the plasmids were transfection into Phoenix-ampho cells. Phoenix-ampho cells were introduced into 6 well plates at a density of 5×10⁵ cells per well and incubated at 37C overnight prior to transfection. 10 ug of purified DNA was mixed with 10 uL Plus Reagent and brought to 100 uL with pre-warmed Opti-MEM media. Lipofectamine reagent was prepared by mixing 8 uL of Lipofectamine with 92 uL of the pre-warmed Opti-MEM. Both mixtures were incubated at room temperature for 15 prior to mixing the 100 uL of Lipofectamine reagent with the 100 uL of DNA solution and allowing the combined solution to incubate at room temperature for another 15 min. The Phoenix-ampho cells were washed gently by aspirating the media and adding 6 mL of pre-warmed Opti-MEM media to wash the cells. The media was removed from the plated cells. 800 uL of the pre-warmed Opti-MEM was added to the DNA/Lipofectamine mixture to make 1 mL and that solution was added to the plated cells. After the plate was incubated for 3 hrs at 37C, 3 mL of complete media was added and the cells were incubated overnight at 37 C. The complete media was exchanged after the incubation and the cells incubated for another 2 days. Virus particles were collected after the supernatant was passed through a 45 um filter into a new 6 well plate. 20 uL of Plus reagent and 8 uL of Lipofectamine was added to each well with a 15 min room temperature incubation after each addition.

K562 cells were suspended complete media at a concentration of 5×10⁶ per mL. 100 uL of K562 cells were added to each well of the 6 well plate containing the virus particles. The plate was centrifuged at 700×g for 20 min and the cells were incubated for 6 hrs at 37 C. Cells and virus were collected and transferred to T25 flasks with the addition of 7 mL of complete media. The cells were incubated for 3 days prior to a media change which included selection with Puromyocin antibiotic. Live cells were collected and passaged to create stocks of K562 cells expressing a single HLA allele. Overall 25 of these cell lines were created, each with a different HLA expressed, to provide a reagent pool for future experiments.

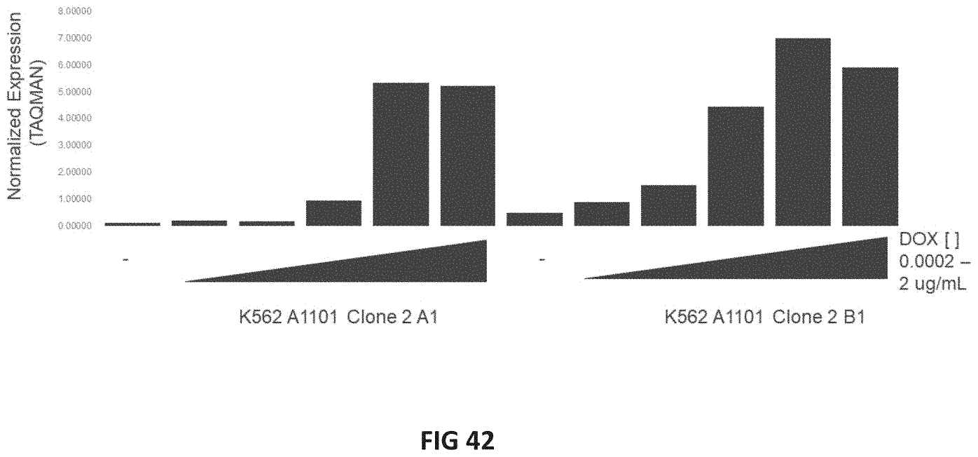

A shared neoantigen cassette was created to express 20 shared neoantigens with the mutation centered in a 25mer amino acid chain and was created with no linkers between the entries. This cassette was subcloned into a lentiviral Tet-One Inducible Expression vector system (Clonetech) and lenitvirus was produced in 293T cells by contransfecting the shared neoantigen expression vector with ViraPower (Thermo) packaging plasmids according to manufacturer's specifications. Single HLA Allele expressing K562 cell lines were then transduced with this virus as described above and single cell clones were characterized for shared neoantigen expression. Briefly, expression of the shared neoantigen cassette was placed under control of a doxycycline (DOX)-controlled TRE3G promoter, where administration of DOX leads to expression of the neoantigens via stabilization of the Tet-On 3G transactivator protein that is constitutively expressed on the same plasmid. The TREG3 promoter—Tet-On 3G transactivator system allows titration of DOX to control the level of expression. As shown in FIG. 42, expression of a representative neoantigen increased as the concentration of DOX administered increased, demonstrating regulatable expression.

Cells containing both the single HLA allele and the shared neoantigen cassette were grown to ˜2.5×10⁸ cells and pelleted into 15 mL vials. Additionally, cells were plated in limited dilution to prepare single clones of the HLA/Cassette pairing. These single clones were tested to achieve a variety of expression levels of the cassette. Use of cell lines with differing expression levels of the cassette allows for analysis of the system at close to endogenous expression levels. Single clones were also grown to ˜2.5×10⁸ cells and pelleted into 15 mL vials. All pellets were washed 2× with cold PBS and frozen to allow for processing for mass spectrometry detection of HLA peptides. Expression levels of the HLA and the cassette was performed using SmartSeq or Tagman assays with appropriate probes.

For isolation of HLA peptides, each cell pellet was lysed with lysis buffer and centrifuged at 20,000×g for 1 hr to clarify the lysate and the HLA peptide complexes were enriched as previously described (see Section VIIIB. Isolation and Detection of HLA Peptides). Heavy peptides—peptides synthesized with amino acids containing isotopically heavy amino acids—were added to the peptides prior to analysis by MS to aid in confirmation of the identity of the peptides detected.

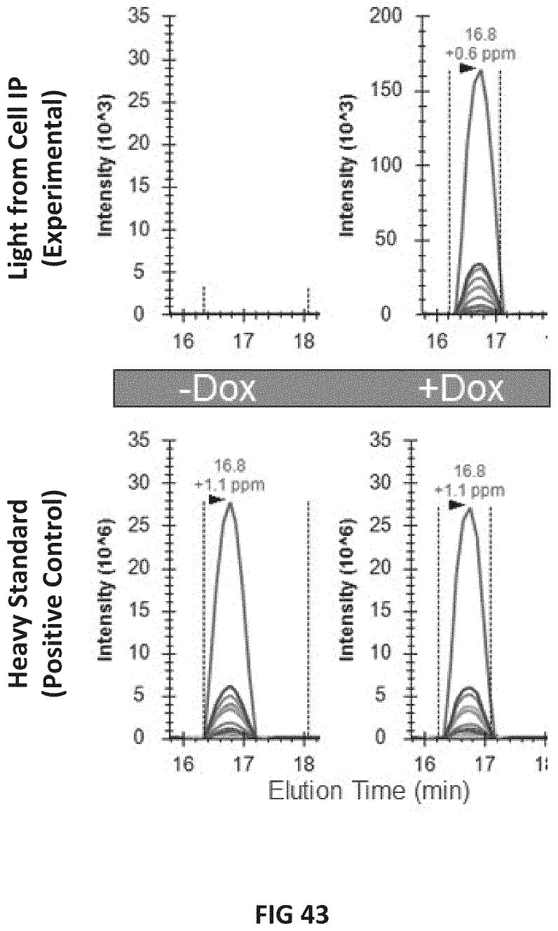

As shown in FIG. 43, a representative KRAS G12V peptide VVGAVGVGK was observed by mass-spectrometry in a HLA-A*11:01 expressing K562 cell line, in a DOX-dependent manner (FIG. 43, top panels). Detection of the heavy peptide control standard was equivalent (FIG. 43, bottom panels). Thus, validation of HLA-specific presentation of predicted neoantigens was demonstrated using the single-HLA K562 in vitro system.

The in vitro system described above was used to validate HLA-specific presentation of predicted neoantigens. Results demonstrating validated HLA-specific presentation of predicted neoantigens are shown in Table 32B.

TABLE 32B
MS-validated neoantigen neoepitopes
in a single-HLA K562 in vitro system

| Gene | HLAType | Size | Sequence |
|---|---|---|---|
| CTNNB1S45P | A*ll:01 | 9-mer | TTAPPLSGK |
| CTNNB1T41A | A*ll:01 | 9-mer | ATAPSLSGK |
| KRASG12D | A*ll:01 | 10-mer | VVVGADGVGK |
| KRASG12V | A*03:01 | 9-mer | VVGAVGVGK |
| KRASG12V | A*03:01 | 10-mer | VVVGAVGVGK |
| KRASG12V | A*ll:01 | 9-mer | VVGAVGVGK |
| KRASG12V | A*ll:01 | 10-mer | VVVGAVGVGK |
| KRASQ61R | A*01:01 | 10-mer | ILDTAGREEY |
| TP53R213L | A*02:01 | 9-mer | YLDDRNTFL |

### XXII. A. Selection of Shared Neoantigens for Vaccine Cassette

A vaccine cassette (“GO-005”) containing 20 shared neoantigens was constructed. Table 34 describes features of the neoantigens selected for the cassette. Shared neoantigens directly detected on the surface of tumor cells by mass spectrometry, as described above in Table 32A, were included in the cassette and the HLA of the epitope was added to the eligible HLA list for the mutations. Neoantigens not independently verified as being presented in our assays were considered validated and added to the cassette if there was compelling literature evidence of tumor presentation (e.g., tumor-infiltrating lymphocytes (TIL) recognizing the neoantigen). KRAS G12D presented by HLA-C*08:02 was considered validated and added based on literature evidence of adoptive cell therapy targeting this neoantigen causing tumor regression in a patient with CRC (Tran et al. N Engl J Med. 2016 Dec. 8; 375(23): 2255-2262). Neoantigens with validated HLA alleles occupied 6 out of 20 slots.

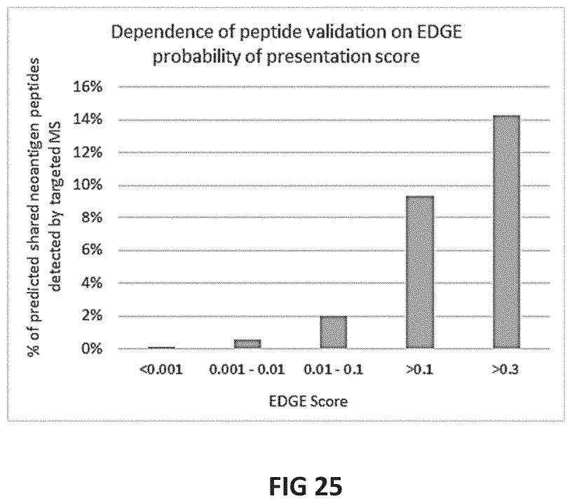

Additional, rarer neoantigens predicted to be presented by tumor cells, but not yet validated by MS, were used to complement the initial set. Mutations with high EDGE scores were prioritized for inclusion as predicted neoantigens given the strong dependence we observed between EDGE score and probability of detection of candidate shared neoantigen peptides by targeted mass spectrometry (MS) validation experiments (see Section XXI above). Results showing the correlation between EDGE score and the probability of detection of candidate shared neoantigen peptides by targeted MS are shown in FIG. 25. Specifically, the remaining slots were filled with predicted neoantigens with an EDGE HLA presentation score of at least 0.3 and the highest cumulative neoantigen/HLA prevalence across NSCLC, CRC and Pancreatic cancer. For each slot in the cassette, combined HLA frequency was required to be at least 5-10% (e.g., there are 11% of the American population harboring HLA alleles B1501 or B1503). Of note, as KRAS and NRAS harbors the same cassette sequence around codons 12, 13, and 61, incorporation of prevalent NRAS mutations did not require additional slots. Validated HLAs, predicted HLAs with an EDGE score of at least 0.3, the mean EDGE score of the predicted HLAs, and neoantigen/HLA prevalence in the three cancer populations are also presented in Table 34.

TABLE 34
Selected Shared Neoantigens in Vaccine Cassette GO-005

| | | | | | Neoantigen/ | Neoantigen/ | Neoantigen/ |
|---|---|---|---|---|---|---|---|
| | | | | | HLA | HLA | HLA |
| | | Validated | Predicted | Mean Predicted | Prevalence in | Prevalence | Prevalence |
| Slot | Mutation | HLA | HLA | EDGE Score | Lung | in CRC | in Pancreas |
| 1 | KRAS_G13D | A1101 | C0802 | 0.306 | 0.07% | 0.39% | 0.06% |
| 2 | KRAS_Q61K | A0101 | — | N/A - validated only | 0.05% | 0.37% | 0.00% |
| | NRAS_Q61K | | | | | | |
| 3 | TP53_R249M | — | B3512, | 0.524 | 0.04% | 0.00% | 0.00% |
| | | | B3503, | | | | |
| | | | B3501 | | | | |
| 4 | CTNNB1_S45P | A0301, | A6801, | 0.894 | 0.13% | 0.00% | 0.00% |
| | | A1101 | A0302, | | | | |
| 5 | CTNNB1_S45F | A1101 | A0301, | 0.478 | 0.08% | 0.27% | 0.00% |
| | | | A6801 | | | | |
| 6 | ERBB2_Y772_A775dup^{a} | — | B1801 | 0.758 | 0.11% | 0.00% | 0.00% |
| 7 | KRAS_G12D | A1101, | — | N/A - validated only | 0.79% | 2.28% | 5.45% |
| | NRAS_G12D | A0301, | | | | | |
| | | C0802 | | | | | |
| 8 | KRAS_Q61R | A0101 | — | N/A - validated only | 0.06% | 0.33% | 0.47% |
| | NRAS_Q61R | | | | | | |
| 9 | CTNNB1_T41A | A1101 | A0301, | 0.545 | 0.00% | 0.27% | 0.00% |
| | | | A0302, | | | | |
| | | | B1510, | | | | |
| | | | C0303, | | | | |
| | | | C0304 | | | | |
| 10 | TP53_K132N | A2402 | A2301 | 0.659 | 0.04% | 0.00% | 0.00% |
| 11 | KRAS_G12A | A0301, | — | N/A - validated only | 0.58% | 0.49% | 0.00% |
| | | A1101 | | | | | |
| 12 | KRAS_Q61L | — | A0101 | 0.792 | 0.22% | 0.08% | 0.00% |
| | NRAS_Q61L | | | | | | |
| 13 | TP53_R213L | A0201 | A0207, | 0.558 | 0.09% | 0.18% | 0.00% |
| | | | C0802 | | | | |
| 14 | BRAF_G466V | — | B1501, | 0.604 | 0.03% | 0.05% | 0.00% |
| | | | B1503 | | | | |
| 15 | KRAS_G12V | A0301, | A0302 | 0.759 | 2.56% | 3.43% | 9.89% |
| | | A1101, | | | | | |
| | | C0102 | | | | | |
| 16 | KRAS_Q61H | A0101 | — | N/A - validated only | 0.42% | 0.28% | 0.91% |
| | NRAS_Q61H | | | | | | |
| 17 | CTNNB1_S37F | A0101 | A2301, | 0.554 | 0.29% | 0.00% | 0.00% |
| | | | A2402 | | | | |
| | | | B1510, | | | | |
| | | | B3906 | | | | |
| | | | C0501, | | | | |
| | | | C1402 | | | | |
| | | | C1403 | | | | |
| 18 | TP53_S127Y | — | A1101, | 0.522 | 0.04% | 0.00% | 0.00% |
| | | | A0301 | | | | |
| 19 | TP53K132E | — | A2402, | 0.445 | 0.00% | 0.05% | 0.00% |
| | | | C1403, | | | | |
| | | | A2301 | | | | |
| 20 | KRAS_G12C | A0201, | — | N/A - validated only | 5.00% | 1.46% | 0.63% |
| | NRAS_G12C | A0301, | | | | | |
| | | A1101 | | | | | |

^{a}ERBB2_Y772_A775dup is also commonly annotated as A775_G776insYVMA, M774_A775insAYVM, and E770delinsEAYVM. Also, ERBB2 may be referred to as HER2

Additionally, we determined the total population of patients with at least one HLA allele identified (i.e., either validated or predicted) to present at least one shared neoantigen contained in the shared neoantigen vaccine GO-005 and compared it to the population of patients with the mutations agnostic of whether the patient had the identified allele. Given the GO-005 vaccine cassette from Table 34, to estimate the GO-005 targeted patient population, we collected patient mutation data from AACR Genie. As such patients do not have matching HLA alleles, we sampled HLA alleles from the TCGA population and paired it to the AACR Genie dataset. Then given a tumor type, any patient from AACR Genie with matching both mutation and HLA is labeled positive, and any patient that doesn't meet the criteria is labeled negative. The percent positives give the overall addressable patient population, per tumor type, in Table 35.

It can be readily appreciated from Table 35 that only a subset of patients who carry a particular mutation also carry the HLA allele likely to present that mutation as a neoantigen. Patients with the mutation, but without the appropriate HLA allele are less likely to benefit from therapy. As an example, whereas an estimated ˜60% of pancreatic cancer patients carry appropriate mutations/neoantigens, more than 2 out of 3 of these patients do not carry the corresponding HLA allele(s). Therefore, a vaccination strategy that considers the relevant mutation and HLA allele pairs as proposed will target just those patients who may benefit. Thus, consideration of epitope presentation by validated or high-scoring predicted HLA is an important step in determining the potential efficacy of a shared neoantigen vaccine.

TABLE 35
Neoantigen/HLA Prevalence in Target Populations

| | MSS CRC | Pancreas | Lung |
|---|---|---|---|
| GO-005 Targeted Patient | 9.0% | 17.4% | 10.6% |
| Population (cumulative | | | |
| neoantigen/HLA prevalence) | | | |
| HLA agnostic patient population | 35.1% | 60.8% | 32.0% |
| (mutation frequency only) | | | |

### XXI. B. Shared Neoantigens Vaccine Cassette Sequence Selection

Shared neoantigen sequences for inclusion in a shared neoantigen vaccine were chosen.

For KRAS_G13D, the shared neoantigen-encoding sequence for inclusion in the vaccine was selected by reference to Table A or AACR GENIE Results, where each relevant sequence considered for inclusion was selected by identifying all rows that list KRAS_G13D and C0802.

For KRAS_Q61K or NRAS_Q61K, the shared neoantigen-encoding sequence for inclusion in the vaccine was selected by reference to Table A or AACR GENIE Results, where each relevant sequence considered for inclusion was selected by identifying all rows that list (1) KRAS_Q61K and A0101; or (2) NRAS Q61K, and A0101.

For TP53_R249M, the shared neoantigen-encoding sequence for inclusion in the vaccine was selected by reference to Table A or AACR GENIE Results, where each relevant sequence considered for inclusion was selected by identifying all rows that list TP53_R249M and at least one of B3512, B3503, and B3501.

For CTNNB1_S45P, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list CTNNB1_S45P and at least one of A0301, A6801, A0302, and A1101. For example, see relevant sequences shown in Table 32A and Table 32B.

For CTNNB1_S45F, the shared neoantigen-encoding sequence for inclusion in the vaccine was selected by reference to Table A or AACR GENIE Results, where each relevant sequence considered for inclusion was selected by identifying all rows that list CTNNB1_S45F and at least one of A0301, A1101, and A6801.

For ERBB2_Y772_A775dup, the shared neoantigen-encoding sequence for inclusion in the vaccine was selected by reference to Table A or AACR GENIE Results, where each relevant sequence considered for inclusion was selected by identifying all rows that list ERBB2_Y772_A775dup and B1801.

For KRAS_G12D or NRAS_G12D, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list (1) KRAS_G12D and at least one of A1101 and C0802; or (2) NRAS_G12D and at least one of A1101 and C0802. For example, see relevant sequences shown in Table 32A or Table 32B.

For KRAS_Q61R or NRAS_Q61R, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list (1) KRAS_Q61R and A0101; or (2) NRAS_Q61R and A0101. For example, see relevant sequence shown in Table 32B.

For CTNNB1_T41A, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list CTNNB1_T41A and at least one of A0301, A0302, A1101, B1510, C0303, and C0304. For example, see relevant sequence shown in Table 32B.

For TP53_K132N, the shared neoantigen-encoding sequence for inclusion in the vaccine was selected by reference to Table A or AACR GENIE Results, where each relevant sequence considered for inclusion was selected by identifying all rows that list TP53_K132N and at least one of A2402 and A2301. For example, see relevant sequence shown in Table 32A.

For KRAS_G12A, the shared neoantigen-encoding sequence for inclusion in the vaccine was selected by reference to Table A or AACR GENIE Results, where each relevant sequence considered for inclusion was selected by identifying all rows that list KRAS_G12A.

For KRAS_Q61L or NRAS_Q61L, the shared neoantigen-encoding sequence for inclusion in the vaccine was selected by reference to Table A or AACR GENIE Results, where each relevant sequence considered for inclusion was selected by identifying all rows that list (1) KRAS_Q61L and A0101; or (2) NRAS_Q61L and A0101.

For TP53_R213L, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list TP53_R213L and at least one of A0207, C0802, and A0201. For example, see relevant sequence shown in Table 32B.

For BRAF_G466V, the shared neoantigen-encoding sequence for inclusion in the vaccine was selected by reference to Table A or AACR GENIE Results, where each relevant sequence considered for inclusion was selected by identifying all rows that list BRAF_G466V and at least one of B1501 and B1503.

For KRAS_G12V, the shared neoantigen-encoding sequence for inclusion in the vaccine was selected by reference to Table A, Additional MS Validated Neoantigens, or AACR GENIE Results, where each relevant sequence considered for inclusion was selected by identifying all rows that list KRAS_G12V and at least one of A0301, A1101, C0102, and A0302. For example, see relevant sequences shown in Table 32A and Table 32B.

For KRAS_Q61H or NRAS_Q61H, the shared neoantigen-encoding sequence for inclusion in the vaccine was selected by reference to Table A or AACR GENIE Results, where each relevant sequence considered was selected by identifying all rows that list (1) KRAS_Q61H and A0101; or (2) NRAS_Q61H and A0101.

For CTNNB1_S37F, the shared neoantigen-encoding sequence for inclusion in the vaccine was selected by reference to Table A or AACR GENIE Results, where each relevant sequence considered was selected by identifying all rows that list CTNNB1_S37F and at least one of A2301, A2402, B1510, B3906, C0501, C1402, and C1403.

For TP53_S127Y, the shared neoantigen-encoding sequence for inclusion in the vaccine was selected by reference to Table A or AACR GENIE Results, where each relevant sequence considered was selected by identifying all rows that list TP53_S127Y and at least one of A1101 and A0301.

For TP53_K132E, the shared neoantigen-encoding sequence for inclusion in the vaccine was selected by reference to Table A or AACR GENIE Results, where each relevant sequence considered was selected by identifying all rows that list TP53_K132E and at least one of A2402, C1403, and A2301.

For KRAS_G12C or NRAS_G12C, the shared neoantigen-encoding sequence for inclusion in the vaccine was selected by reference to Table A 32, or AACR GENIE Results, where each relevant sequence considered was selected by identifying all rows that list (1) KRAS_G12C and A0201; or (2) NRAS_G12C and A0201. For example, see relevant sequences shown in Table 32A.

### XXIII. Evaluation of T Cell Recognition of Shared Neoantigens

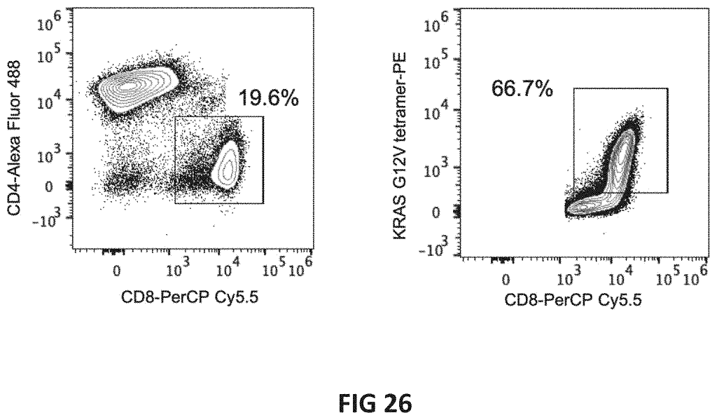

We evaluated whether neoantigens induce an immune response in patients. We obtained dissociated tumor cells from a patient with lung adenocarcinoma. Tumor cells were sequenced to determine the patient's HLA and identify mutations. The patient expressed HLA-A*1101 and we identified the KRAS G12V mutation in the tumor. Simultaneously, we sorted and expanded CD45+ cells from the tumor which represent tumor infiltrating lymphocytes (TIL). Expanded TILs were stained with mutated peptide HLA-A*11:01 tetramers to assess immunogenicity of this mutation in the patient. FIG. 26 shows the flow cytometry gating strategy on CD8+ cells (left panel) and the staining of CD8+ cells by KRAS-G12V/HLA-A*11:01 tetramer (right panel). A large portion (greater than 66%) of CD8+ T cells demonstrate binding to the KRAS G12V:HLA*1101 tetramer, indicating the ability of CD8+ T cells to recognize the neoantigen and indicating a pre-existing immune response to the neoantigen.

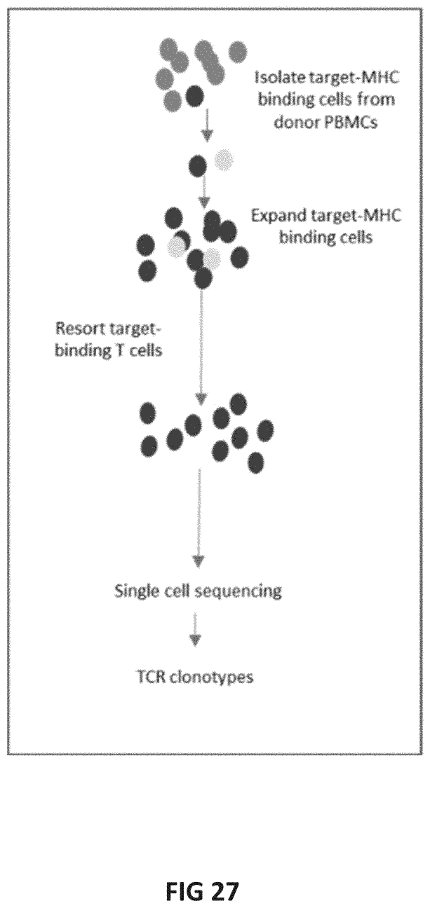

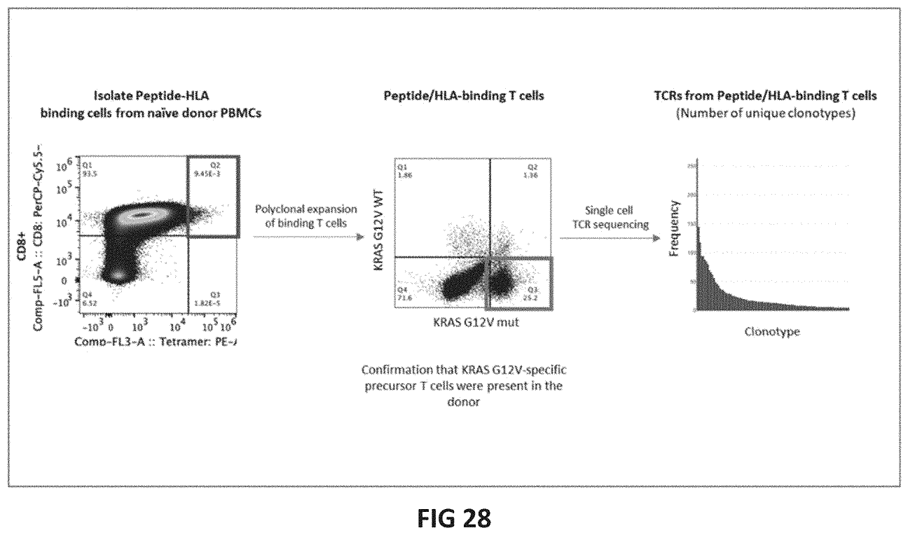

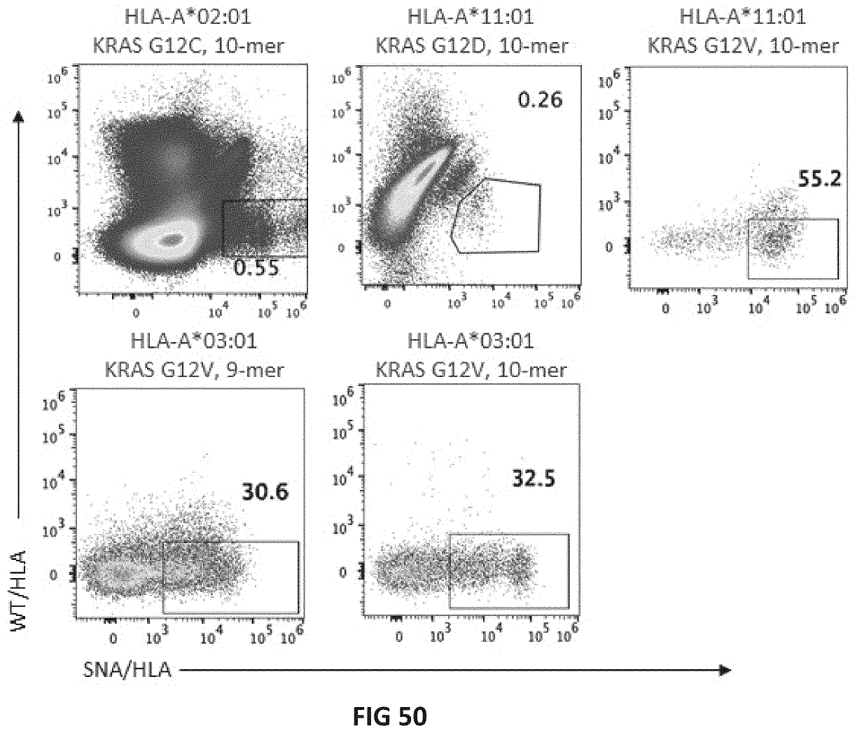

Additionally, we evaluated the presence of T cell precursors in the naïve T cell repertoire of healthy donors capable of recognizing a shared neoantigen. Peripheral blood mononuclear cells (PBMCs) were enriched for naïve CD8+ T cells and stained with MHC multimers presenting several of the shared neoantigen candidates present in the vaccine cassette GO-005. As shown in FIG. 50, HLA-peptide binding cells were sorted, expanded and their specificity for the neoantigen was confirmed by tetramer stainging using flow-cytometry. TCR sequencing of neoantigen-specific T cells was also performed. FIG. 27 illustrates the general TCR sequencing strategy and workflow. FIG. 28 shows a representive example of TCR sequencing strategy for KRAS-G12V/HLA-A*11:01 tetramer. FIG. 50 demonstrates results from 3 healthy donors and shows naïve CD8 T-cell precursors that were enriched by tetramer binding and expanded for two weeks. Shared neoantigen-reactive precursor T cells were found for 3 KRAS mutations and 3 HLA alleles. Among G12C and G12V KRAS mutation-responding T cells sequenced, the median number of T-cell clonotypes to each shared neoantigen per donor was 110, and the range was 48-898 clonotypes. Thus, the the naïve T cell repertoire analysis suggests these neoantigens are expected to induce an immune response in select patients when administered by vaccination.

TABLE 36
Assessment of Neoantigen Reactive Naïve T cells Precursors

| | | | Number of healthy donors | Number of |
|---|---|---|---|---|
| Mutation/ | | | with precursors | neo antigen-specific |
| neoantigen | Peptide | HLA-restriction | (% among donors tested) | clonotypes per donor |
| KRAS G12V | 1 | HLA-A*0301 | 1 (100%) | 46 |
| KRAS G12V | 2 | HLA-A*0301 | 2 (100%) | Donor 1: 25, Donor 2: |
| | | | | No sequence data |
| KRAS G12V | 1 | HLA-A*1101 | 2 (100%) | No sequence data |
| KRAS G12V | 2 | HLA-A*1101 | 2 (100%) | Donor 1: 100, Donor |
| | | | | 2: No sequence data |
| KRAS G12C | 3 | HLA-A*0201 | 1 (100%) | No sequence data |
| CTNNB1 S45P | 4 | HLA-A*0301 | 1 (100%) | 987 |

### XXIV. Selection of Shared Neoantigens and Patient Populations

One or more of the antigens provided in Table 34, Table A, Table 1.2, Additional MS Validated Neoantigens, or the AACR GENIE Results described herein (SEQ ID NOs: 57-29,364) are used to formulate a vaccine composition as described herein. The vaccine is administered to a patient, e.g., to treat cancer. In certain instances the patient is selected, e.g., using a companion diagnostic or a commonly use cancer gene panel NGS assay such as FoundationOne, FoundationOne CDx, Guardant 360, Guardant OMNI, or MSK IMPACT. Exemplary patient selection criteria are described below. An exemplary shared neoantigen vaccine composition GO-005 targets the mutations described in Table 34.

### Patient Selection

Patient selection for shared neoantigen vaccination is performed by consideration of tumor gene expression, somatic mutation status, and patient HLA type. Specifically, a patient is considered eligible for the vaccine therapy if:

(a) the patient carries an HLA allele predicted or known to present an epitope included in a vaccine and the patient tumor expresses a gene with the epitope sequence, or

(b) the patient carries an HLA allele predicted or known to present an epitope included in a vaccine, and the patient tumor carries the mutation giving rise to the epitope sequence, or

(c) Same as (b), but also requiring that the patient tumor expresses the gene with the mutation above a certain threshold (e.g., 1 TPM or 10 TPM), or

(d) Same as (b), but also requiring that the patient tumor expresses the mutation above a certain threshold (e.g., at least 1 mutated read observed at the level of RNA)

(e) Same as (b), but also requiring both additional criteria in (c) and (d)

(f) Any of the above, but also optionally requiring that loss of the presenting HLA allele is not detected in the tumor

Gene expression is measured at the RNA or protein level by any of the established methods including RNASeq, microarray, PCR, Nanostring, ISH, Mass spectrometry, or IHC. Thresholds for positivity of gene expression is established by several methods, including: (1) predicted probability of presentation of the epitope by the HLA allele at various gene expression levels, (2) correlation of gene expression and HLA epitope presentation as measured by mass spectrometry, and/or (3) clinical benefits of vaccination attained for patients expressing the genes at various levels. Patient selection is further extended to require positivity for greater than 1 epitope, for examples, at least 2, 3, 4 or 5 epitopes included in the vaccine.

Somatic mutational status is assessed by any of the established methods, including exome sequencing (NGS DNASeq), targeted exome sequencing (panel of genes), transcriptome sequencing (RNASeq), Sanger sequencing, PCR-based genotyping assays (e.g., Tagman or droplet digital PCR), Mass-spectrometry based methods (e.g., by Sequenom), or any other method known to those skilled in the art.

Additional new shared neoantigens are identified using any of the methods described, e.g., by mass spectrometry. These newly identified shared neoantigens are incorporated into the vaccine cassettes described herein.

Previously validated neoantigens are additionally validated as being presented by additional HLA alleles and informs neoantigen selection for the vaccine cassette and/or expands the potential treatable population.

Inclusions of a new neoantigen enables the broadening of addressable tumor type (eg, EGFR mutated NSCLC) or inclusion of patients with a new tumor type.

### XXV. Identification of Shared Antigens

We identified shared antigen gene-based targets using three computational steps: First, we identified genes with low or no expression in most normal tissues using data available through the Genotype-Tissue Expression (GTEx) Project [1]. We obtained aggregated gene expression data from the Genotype-Tissue Expression (GTEx) Project (version V7p2). This dataset comprised greater than 11,000 post-mortem samples from greater than 700 individuals and greater than 50 different tissue types. Expression was measured using RNA-Seq and computationally processed according to the GTEx standard pipeline (https://www.gtexportal.org/home/documentationPage). Gene expression was calculated using the sum of isoform expressions that were calculated using RSEM v1.2.22 [2].

Next, we identified which of those genes are aberrantly expressed in cancer samples using data from The Cancer Genome Atlas (TCGA) Research Network: http://cancergenome.nih.gov/. We examined greater than 11,000 samples available from TCGA (Data Release 6.0).

Finally, in these genes, we identified which peptides are likely to be presented as cell surface antigens by MHC Class I proteins using a deep learning model trained on HLA presented peptides sequenced by MS/MS, as described in international patent application no. PCT/US2016/067159, herein incorporated by reference, in its entirety, for all purposes.

To identify the common tumor antigens (CTAs; shared antigens), we sought to define criteria to exclude genes that were expressed in normal tissue that were strict enough to ensure tumor specificity, but would account for potential artifacts such as read misalignment. Genes were eligible for inclusion as CTAs if they met the following criteria: The median GTEx expression in each organ that was a part of the brain, heart, or lung was less than 0.1 transcripts per million (TPM) with no one sample exceeding 5 TPM. The median GTEx expression in other essential organs was less than 2 TPM with no one sample exceeding 10 TPM. Expression was ignored for organs classified as non-essential (testis, thyroid, and minor salivary gland). Genes were considered expressed in tumor samples if they had expression in TCGA of greater than 10 TPM in at least 30 samples. Based on literature review, we also added the genes MAGEB4 and MAGEB6.

We also added the gene CTAG1A/CTAG1B (NY-ESO-1). As it's expression is not accurately quantified using the computational methodology in TCGA Data Release 6.0, we relied on the RSEM calculations available in the TCGA legacy archive (https.//portal.gdc.cancer.gov/legacy-archive) which accounts for multiply mapped reads.

We then examined the distribution of the expression of the remaining genes across TCGA samples. When we examined the known CTAs, e.g. the MAGE family of genes, we observed that the expression of these genes in log space was generally characterized by a bimodal distribution. This distribution included a left mode around a lower expression value and a right mode (or thick tail) at a higher expression level. This expression pattern is consistent with a biological model in which some minimal expression can be detected at baseline in many samples and higher expression of the gene is observed in a subset of tumors experiencing epigenetic dysregulation. We reviewed the distribution of expression of each gene across TCGA samples and discarded those where we observed only a unimodal distribution with no significant right-hand tail. A small number of genes were eliminated by hand curation, for example, if they were likely to be expressed in a tissue not available in GTEx. This resulted in a set of 59 genes. See Table 46. In Table 46 an X is used to indicate cancers in which the gene is expressed at greater than 10 TPM in at least 1% of cases.

TABLE 46
Analysis of Expression Levels in Cancer Subtypes

| | | | | | | Cervival | | | |
|---|---|---|---|---|---|---|---|---|---|
| | | | | | | Squamous | | | |
| | | | | | | Cell | | | |
| | | | | Brain | | Carcinoma | | | |
| | | | | | | and | | | |
| | Acute | | Bladder | Lower | Breast | Endocervical | | Colon | |
| | Myeloid | Adrenocortical | Urothelial | Grade | Invasive | Adeno- | Cholanglo- | Adeno- | Esophegal |
| Gene | Leukemia | Carcinoma | Carcinoma | Glioma | Carcinoma | carcinoma | carcinoma | carcinoma | Carcinoma |
| ACTL8 | | X | X | | X | X | | X | X |
| ADAM2 | | | | | X | | | | |
| ADAM7 | | | | | | | | | |
| AMELX | | | | | | | | X | |
| BPIFA2 | | X | | | | | | | |
| CRYGC | | | | | | | | | |
| CT83 | | X | X | | X | X | X | X | X |
| CTAG1A/ | | X | X | | X | X | X | X | X |
| CTAG 1B | | | | | | | | | |
| CTCFL | | | | | | X | | | X |
| DCAF12L1 | | | | | | X | | | |
| DCAF4L2 | | X | X | | | | X | | X |
| DEFB126 | | X | X | | | | | | X |
| DMRT1 | | | | | | X | X | | |
| DPPA2 | | | X | | | | | | X |
| FMR1NB | | | | | | | | | X |
| FTHL17 | | | X | | X | X | | | X |
| GAG El | | | X | | | | | | |
| GAGE12J | | | X | | | | | | X |
| GAGE2A | | | X | | | X | | | X |
| GLYATL3 | | | | | X | X | | | |
| GPRC6A | | X | | | | | | | |
| GSG1L2 | | | | | | | | | |
| IFNK | X | | X | | | | | X | |
| IL22RA2 | X | | | | X | X | | | X |
| INSL4 | | | | | | X | | X | |
| INSL6 | X | | X | | X | X | | | |
| KCNU1 | | | | | | | | | |
| LIN28A | | | | | X | | X | | |
| LUZP4 | | | | | | | | | |
| MAGEA1 | | X | X | | X | X | X | X | X |
| MAGEA10 | | | X | | X | X | | | X |
| MAGEAll | | | X | | | X | | X | X |
| MAGEA3 | | X | X | | X | X | X | X | X |
| MAGEA4 | | | X | | X | X | | X | X |
| MAGEA6 | | X | X | | X | X | X | X | X |
| MAGEA9 | | | X | | | | | | X |
| MAGEB1 | | | | | | | | | X |
| MAGEB2 | | X | X | | X | X | | X | X |
| MAGEB4 | | | | | X | | | | |
| MAGEB6 | | X | | | | | | | |
| MAGECI | | X | X | | X | | | | X |
| MAGEC2 | | X | X | | X | X | | | X |
| MORC1 | | X | | | | | | | |
| PAG El | | | X | | | X | | | X |
| PAGE5 | | X | X | | X | | X | | X |
| PASD1 | | | X | | | | | | X |
| PRDM7 | | | | | | | | | |
| PROKRI | | | | | | | | | |
| R3HDML | | | | | | | | X | |
| SLC7A13 | | | | | | | | | |
| SMCIB | X | | X | | X | X | | | X |
| SMR3A | | | | | | | | | |
| SSXI | | | X | | X | | X | | X |
| STRA8 | | | X | | X | X | | | X |
| TFDP3 | | | | | X | | | | |
| XAGE3 | | | X | | | | | | |
| XAGE5 | | | | | | | | | |
| ZFP42 | | | X | | X | X | X | | X |
| ZNF560 | X | X | X | X | | | | | |
| | | Head | | | | | | | Lymphoid |
| | | and | | Kidney | Kidney | | | | Neoplasm |
| | | Neck | | Renal | Renal | | | Lung | Diffuse |
| | | Squamous | | Clear | Papillary | Liver | Lung | Squamous | Large |
| | Gliobatoma | Cell | Kidney | Cell | Cell | Hepatocellular | Adeno- | Cell | B-cell |
| Gene | Multiforme | Carcinoma | Chromophobe | Carcinoma | Carcinoma | Carcinoma | carcinoma | Carcinoma | Lymphoma |
| ACTL8 | | X | | | | X | X | X | X |
| ADAM2 | | | X | | | | | | |
| ADAM7 | | | | | X | | | | |
| AMELX | | | | | | | | | |
| BPIFA2 | | X | X | | | X | X | X | |
| CRYGC | | | | | | | | | |
| CT83 | | X | | | X | X | X | X | |
| CTAG1A/ | | X | | | X | X | X | X | X |
| CTAG 1B | | | | | | | | | |
| CTCFL | | X | | | | | | X | X |
| DCAF12L1 | | | | X | X | | | | |
| DCAF4L2 | | X | | | | X | X | X | |
| DEFB126 | | | | | | | | X | |
| DMRT1 | | | | | | | | X | X |
| DPPA2 | | X | | | X | | X | X | |
| FMR1NB | | | | | | | X | | |
| FTHL17 | | X | | | | X | X | X | |
| GAG El | | | | | | X | | X | |
| GAGE12J | | | | | | | | X | |
| GAGE2A | | X | X | | X | X | X | X | |
| GLYATL3 | | | | | | | | X | |
| GPRC6A | | | X | X | | | | | |
| GSG1L2 | | | | X | | | | | |
| IFNK | | X | | | | | X | | |
| IL22RA2 | | X | | | | | X | X | X |
| INSL4 | | | | | | | X | X | |
| INSL6 | | | | | | X | X | | |
| KCNU1 | | | X | | | X | | | |
| LIN28A | | | | | | | | | |
| LUZP4 | | | | | | | | | |
| MAGEA1 | | X | X | | | X | X | X | X |
| MAGEA10 | | X | | | | | X | X | |
| MAGEAll | | X | | | | X | X | X | |
| MAGEA3 | | X | X | | X | X | X | X | |
| MAGEA4 | | X | | | | X | X | X | X |
| MAGEA6 | | X | X | | X | X | X | X | |
| MAGEA9 | | X | | | | | | X | X |
| MAGEB1 | | X | | | | X | X | X | X |
| MAGEB2 | | X | X | X | | X | X | X | X |
| MAGEB4 | | | | | | | | | |
| MAGEB6 | | | | | | | | | |
| MAGECI | | X | X | | | X | X | X | X |
| MAGEC2 | X | X | X | X | X | X | X | X | |
| MORC1 | | | | | | | | | |
| PAG El | | | X | | X | X | X | X | |
| PAGE5 | | X | X | | X | X | X | X | X |
| PASD1 | | | | | | | X | X | |
| PRDM7 | | | | | | | | | |
| PROKRI | | | | | | | | | |
| R3HDML | | | | | | | | | |
| SLC7A13 | | | X | X | X | | | | |
| SMCIB | | X | X | | | X | X | X | X |
| SMR3A | | X | | | | X | | | |
| SSXI | | | X | | | X | X | X | |
| STRA8 | | X | | X | X | | X | X | |
| TFDP3 | | | | | | X | | | |
| XAGE3 | | | | | | | | X | |
| XAGE5 | | | | | | X | | X | |
| ZFP42 | | X | | | | | X | X | |
| ZNF560 | X | | | | | | X | X | |
| | | Ovarian | | Pheochromo- | | | | | |
| | | Serous | Pancreatic | cytoma | Prostate | Rectum | | Skin | Stomach |
| | | Cystadeno- | Adeno- | and | Adeno- | Adeno- | | Cutaneous | Adeno- |
| Gene | Mesothelioma | carcinoma | carcinoma | Paraganglioma | carcinoma | carcinoma | Sarcoma | Melanoma | carcinoma |
| ACTL8 | X | X | X | X | | X | X | X | X |
| ADAM2 | | | | | X | | | | |
| ADAM7 | | | | | X | | | | |
| AMELX | | | | | | X | X | | |
| BPIFA2 | X | | X | | | | | | X |
| CRYGC | | X | | | | | | | |
| CT83 | | X | X | | | X | | X | X |
| CTAG1A/ | X | X | | X | X | X | X | X | X |
| CTAG 1B | | | | | | | | | |
| CTCFL | | X | | | | | | X | X |
| DCAF12L1 | | X | | | | | X | X | |
| DCAF4L2 | | | | | | | | | X |
| DEFB126 | | X | | | | | | | |
| DMRT1 | | X | | | | | | | |
| DPPA2 | | X | | | | | | | |
| FMR1NB | | | | | | | | | |
| FTHL17 | | | | | | | | | |
| GAG El | | X | | | | | X | X | X |
| GAGE12J | | X | | | | | | X | X |
| GAGE2A | X | X | | | | | X | X | X |
| GLYATL3 | | | X | | X | | | | |
| GPRC6A | | | | | | | | | |
| GSG1L2 | | | | | | | | | |
| IFNK | | | | | | | | | |
| IL22RA2 | | | | | | | | | |
| INSL4 | | | X | | | | | | |
| INSL6 | | X | | | | | | | X |
| KCNU1 | | | | | | | | X | |
| LIN28A | | | | | | | | X | |
| LUZP4 | | | | | | | | X | |
| MAGEA1 | X | X | X | X | | X | X | X | X |
| MAGEA10 | | X | | | | X | X | X | X |
| MAGEAll | | X | | | | X | X | X | X |
| MAGEA3 | X | X | X | X | X | X | X | X | X |
| MAGEA4 | | X | | | | X | X | X | X |
| MAGEA6 | X | X | X | X | | X | X | X | X |
| MAGEA9 | | | | | | | | | |
| MAGEB1 | | | | | | | X | X | X |
| MAGEB2 | X | X | | | | X | X | X | X |
| MAGEB4 | | | | | | | | | |
| MAGEB6 | | | | | | | | | |
| MAGECI | | X | | | | X | X | X | X |
| MAGEC2 | | X | | X | X | X | X | X | X |
| MORC1 | | | | | | | | X | |
| PAG El | X | X | | | | X | X | X | X |
| PAGE5 | | X | | X | | | X | X | X |
| PASD1 | X | X | | | | | | X | X |
| PRDM7 | | | | | | | | X | |
| PROKRI | | | | X | | | | | |
| R3HDML | | | | | | X | | | |
| SLC7A13 | | | | | | | | | |
| SMCIB | | X | | | | | X | X | |
| SMR3A | | | | | | | | | |
| SSXI | | X | | | | | X | X | X |
| STRA8 | | X | | | | | | | X |
| TFDP3 | | | | | | | | | |
| XAGE3 | | X | | | | | X | X | |
| XAGE5 | | | | | | | X | X | |
| ZFP42 | X | X | | | | | | X | X |
| ZNF560 | X | X | | X | X | | X | X | |
| | | | | | | | Uterine | | |
| | | | | | | Uterine | Corpus | | |
| | | | | | Thyroid | Carcino- | Endometrial | Uveal | Total |
| | | | Gene | Thymoma | Carcinoma | sarcoma | Carcinoma | Melanoma | Indicarions |
| | | | ACTL8 | | | X | X | X | 22 |
| | | | ADAM2 | | | | | | 3 |
| | | | ADAM7 | | | | | | 2 |
| | | | AMELX | | | X | | | 4 |
| | | | BPIFA2 | | | X | | | 10 |
| | | | CRYGC | | | | X | | 2 |
| | | | CT83 | | | X | X | | 19 |
| | | | CTAG1A/ | | | X | X | X | 24 |
| | | | CTAG 1B | | | | | | |
| | | | CTCFL | | | X | X | | 10 |
| | | | DCAF12L1 | | | X | X | | 8 |
| | | | DCAF4L2 | | | X | | | 10 |
| | | | DEFB126 | | | | X | | 6 |
| | | | DMRT1 | | | X | X | | 7 |
| | | | DPPA2 | | | | X | | 8 |
| | | | FMR1NB | | | X | | | 3 |
| | | | FTHL17 | | | | | | 8 |
| | | | GAG El | | | X | | | 8 |
| | | | GAGE12J | | | | | | 6 |
| | | | GAGE2A | | | X | X | | 16 |
| | | | GLYATL3 | | | X | X | | 7 |
| | | | GPRC6A | | | | | | 3 |
| | | | GSG1L2 | | | | | | 1 |
| | | | IFNK | | | | | | 5 |
| | | | IL22RA2 | | | | | | 8 |
| | | | INSL4 | | | X | X | | 7 |
| | | | INSL6 | | | X | X | | 10 |
| | | | KCNU1 | | | | | | 3 |
| | | | LIN28A | | | X | X | | 5 |
| | | | LUZP4 | | | X | | | 2 |
| | | | MAGEA1 | X | | X | X | | 24 |
| | | | MAGEA10 | | | X | X | | 14 |
| | | | MAGEAll | | | X | X | | 15 |
| | | | MAGEA3 | | | X | X | | 24 |
| | | | MAGEA4 | X | | X | X | | 18 |
| | | | MAGEA6 | | | X | X | | 23 |
| | | | MAGEA9 | | | X | | | 6 |
| | | | MAGEB1 | | | X | | | 10 |
| | | | MAGEB2 | | | X | X | | 21 |
| | | | MAGEB4 | | | | | | 1 |
| | | | MAGEB6 | | | | | | 1 |
| | | | MAGECI | | | X | X | | 17 |
| | | | MAGEC2 | | | X | X | | 22 |
| | | | MORC1 | | | X | | | 2 |
| | | | PAG El | | | X | X | | 16 |
| | | | PAGE5 | | | X | X | | 19 |
| | | | PASD1 | | | X | X | | 10 |
| | | | PRDM7 | | | | | | 1 |
| | | | PROKRI | | | X | | | 2 |
| | | | R3HDML | | | | | | 2 |
| | | | SLC7A13 | | | | | | 3 |
| | | | SMCIB | | | | X | | 15 |
| | | | SMR3A | | X | | X | | 4 |
| | | | SSXI | | X | X | | X | 15 |
| | | | STRA8 | | | | X | | 12 |
| | | | TFDP3 | X | | | | | 3 |
| | | | XAGE3 | | | X | X | | 7 |
| | | | XAGE5 | | | X | | | 5 |
| | | | ZFP42 | | | X | X | | 14 |
| | | | ZNF560 | X | X | X | | X | 17 |

To identify peptides that are likely to be presented as cell surface antigens by MHC Class I proteins, we used a sliding window to parse each of these proteins into its constituent 8-11 amino acid sequences. We processed these peptides and their flanking sequences with the HLA peptide presentation deep learning model to calculate the probability of presentation of each peptide at the 99.9^{th} percentile expression level observed for this gene in TCGA. We considered a peptide likely to be presented (i.e., a candidate target) if its quantile normalized probability of presentation calculated by our model was greater than 0.001.

To prioritize genes that are likely to be relevant to a given indication, we selected genes where the gene is expressed at a level of at least 10 TPM in at least 0.98% of cancer cases.

The results are shown in Table 1.2. A total of 10698 shared antigen sequences were identified. The corresponding HLA allele(s) for each sequence are shown.

### XXVI. Heterologous Prime/Boost Stimulates CD8+ T-Cell Responses in Human Subjects

An open-label, multi-center, multi-dose Phase 1/2 study was performed to assess the dose, safety and tolerability, immunogenicity, and early clinical activity of a heterologous prime/boost vaccination strategy. Two vaccine programs, GRANITE and SLATE, were assessed.

A personalized neoantigen cancer vaccine (“GRANITE”) was administered in combination with immune checkpoint blockade in patients with advanced cancer. The GRANITE heterologous prime/boost vaccine regimen included (1) a ChAdV that is used as a prime vaccination [GRT-C901] and (2) a SAM formulated in a LNP that is used for boost vaccinations [GRT-R902] following GRT-C901. The ChAdV vector is based on a modified ChAdV68 sequence having the sequence of SEQ ID NO:1 with an E1 (nt 577 to 3403) deletion and an E3 (nt 27,125-31,825) deletion. The SAM vector is based on an RNA alphavirus backbone having the nucleic acid sequence set forth in SEQ ID NO:6. Both GRT-C901 and GRT-R902 expressed the same 20 personalized neoantigens as well as two universal CD4 T-cell epitopes (PADRE and Tetanus Toxoid). Tumors were used for whole-exome and transcriptome sequencing to detect somatic mutations, and blood was used for HLA typing and detection/subtraction of germline exome variants to generate the personalized neoantigen cassette using the EDGE algorithm for 10 subjects (Patients 1-10, referred to herein as patients G1-G10). The neoantigens included in each subject's cassette is shown in Table 47A and the full-length cassette is shown in Table 47B. The determined HLAs of GRANITE subjects are shown in Table 48.

TABLE 47A
GRANITE Subject-Specific Neoantigens

| Ag | Patient G1 | Patient G2 | Patient G3 | Patient G4 |
|---|---|---|---|---|
| 1 | QLQAEQKGLTEVIQ | YPLAAHTYTPITGS | ERYHFLEERLARLG | KRYRVRSYEAVAV |
| | SLKMENEEFKK | VSTIRQYPVSA | LTEVYQWYLDL | AAAASAYNYAEQ |
| 2 | GLFEGDEYATLMM | RYYDRVTGVSSFH | KQGKDLTVLDHTL | EDPYLEDSEASLVP |
| | QCKEGAQKEGLM | VTWVSQASADQR | PTALTSPELSSW | LAYQPIPFSQS |
| 3 | APFPAWSAFGKEE | VKELRRLEDQLASL | MASMGTLAFHEYG | ERAQGELFPDRASS |
| | HDEALKNTWNLH | QQELAALALKQ | RPFLIIKDQDRK | SWGNAAAAAGD |
| 4 | LAEQVQKADEFIQ | SSLDAVLSQDLILA | NWVQQQAYGMLA | DVKKLITDEFVKH |
| | ANATNKLTVIAE | DGPAVEVGDSL | GIPVTICTYPFVF | KYLDYARVPNSN |
| 5 | EEQKLPEADVAVL | RRMNRVGNESLNP | CSPPSPSALIQEMEE | HLLQSKSGTQPHTA |
| | RTFLRQQASILS | AVAKAAETIIDT | RLWEKEQEVA | TRLSLQQPRAE |
| 6 | VSQKQIADRAAHN | LYSLSVLYKGEAR | VKGGYQESSLLRV | KIPASQHTQNWSTT |
| | ESPGNNVVDISV | VVLLKAAYDVSS | LVTLNDLEPKAA | WTKDSKRRDKR |
| 7 | LNTAQDELVTFSDE | IMEIQLNGGTVAKK | PNYADTLISQESFE | PKPFRPILGLHLNL |
| | LAQLYHHVCLC | VAWAQARLEKQ | KKGFLSAPQSL | GILYYVYMGLL |
| 8 | HNMKLSISYLREYA | GFAAEFKLVDQSRP | HSSFVAVTEEGTKA | AILSFGELIFCGMPA |
| | KANDWLQFIIH | PHEPKFVYQAK | AAATGIGFTVT | EMLDFFNDCG |
| 9 | GPKVDIDVPDVNFE | DQAVGAYLPLTTA | AVKLLIERATNQG | ANMCRCLVTLKVL |
| | APEGKLKGPKF | RYSTLLAGNLRI | WMLRVKWLYHLA | MADKPGNTVNFR |
| 10 | NFDVAGYIVGANT | KRQELESENKKLN | FFGMHVQEYGSDW | SLQHQLPQPGAQH |
| | ETYLLEKSRAIR | NDLNELRKAVAD | PPPNTRRVYISY | FATRGYPMEDMK |
| 11 | DTCVCSTVAEFSHQ | DVTRQADCKMKL | DTRIAKRMAMLIY | TEPGDYNINILFTDT |
| | CSHAGGRPGNW | ADFVKYYYSGKRE | TDFLCMAPISFF | HIPGSPFKAH |
| 12 | QGDGCSGHKNQNQ | LRNDAKNLYAANS | VRASSDGEGTMSL | YKCLSCTKTFPNVP |
| | IDTLQEVRLRFL | IGAVLAHKGYFR | PASVDGSPVSPS | RAARHAATHGP |
| 13 | PMNTIAEAVIEMVN | DMFDCVFPTRTALF | SKELVEKSEAVRK | PLKIFAQDGEGQHI |
| | RGQIQITINGF | GSALVPTGNLQ | MSSSGKLWKPRQ | DIQMKNRMDGT |
| 14 | GKISRHEMLQVLH | MSNLRQVGLKKPM | MDARRVPPKDLRV | VYCDFSTGETCIQA |
| | LMVGVQVTEEQL | ERSSVLDRYPPA | KKNLKKFRYVKL | QPENIPAKNWY |
| 15 | TDAYLLYTPSQIVL | QLMPLKTFPAAIW | VKKNRQNGMHTL | NSLATVTMEDLIQP |
| | TAILSSASRAG | GVIQSELNYSVI | YLLDIKVKEQSLE | WFPEFSEARAI |
| 16 | GIGWLITFCSKGED | SEVSKAIINIFFFYG | LFIYQSLDAIDGRQ | KVHLAKSSGSALM |
| | CLTSQTRLRLS | PPQKIIMDQR | ARRTNSSSPLG | VWGTGNPRRQFI |
| 17 | GQLSQDRDFIMTLN | LQMTDIVQACHDSI | YLSLLRMYLSPPCI | SFVIPFIHLPEIINLY |
| | TSLHRSWWMEN | KAALDISIKSD | HCLGPIKLELL | NLSEQNDVF |
| 18 | SEEAEVDVREREIQ | LEAFLTQKAKVGK | SLSLAEKSILHEECI | SYQPAVTAPEPDSS |
| | RDREPKRARDL | LKDDDFERISEL | KSPVVETVPA | PRLAVDFALPK |
| 19 | QGRVKDLESLFHQ | KLPGLNTATILLMG | PPDPLEPPSLPAQRP | VFYIEACESGSMVN |
| | SEVELAAALSDK | TEDALLQQLAD | GPPTPAAAHS | HLPDNINVYAT |
| 20 | CLAFKNDATEILHS | SNLKVLNHSPMSG | GDGGLVCPYMEFL | RDSVKSCRVHVPG |
| | HVVKPVPAHPS | ASVNFDYKSPSP | KNENNELPKLQW | KTLTCGKGRRDF |
| Ag | Patient G5 | Patient G6 | Patient G7 | Patient G8 |
| 1 | RIEVMALPKPGGTH | EQTEAAAREEILTN | QIGALILATDISCQN | GSRKERQVYSKAL |
| | SLALVTVPSMG | GGSLSHHHGVG | EYLSLFRSHL | NRLFGVEASGRR |
| 2 | VDPEDQSQVTLAS | FQYNYEYLGNSGQ | HMSSMEHTEEGLQ | PYHRSQSSSSVLNN |
| | YGFAFRYCPSGK | LVITPLTDRCYM | ERLADAMAESPS | KSMDSINYPSD |
| 3 | STLYGRVLAPLPER | MDKDGYFWFVAR | REKVDNVWTEESS | NANFDLRTSQSVR |
| | AGGAASGGGGN | SDDVILSSGYRIG | NQFLQELETGQR | AMLAYNMRDQHL |
| 4 | LRKRHRKHTQQRV | AVRTPYTMGYGEP | LRDVDAKALVRSH | EVLLLSMRDMNIT |
| | AALSLSTLASPK | MYYNDAYGALDY | FLLVYGDVISNI | KLTSVDAPLFNA |
| 5 | SARIPEQLDVTAHG | MTEYKLVVVGAV | IIAKLLADELISLKD | AQPHVNGGPLYSK |
| | VYAPEDVYRFR | GVGKSALTIQLIQ | IDPVQRYVPL | NSISIAPPPPPM |
| 6 | GTGGSGPGLSRTRA | IEMWISILELNELEY | VAASEKSTGKIFLY | AKMKWTNLLYKA |
| | LSTGALPPLAP | AAVELHQAKD | DGRGDNQPLHI | GGVKIRDDERLLQ |
| 7 | VEEDESAMPKKDS | LLLAAHRFLATELS | MFEVCFESKGTGPI | EQSAERCVSTLLNL |
| | RTLLARFNEQIE | QFSPSLISEKI | PDQLVILDMKH | IQTKVNYVVQE |
| 8 | GASAPGKSMEASM | ELKHMVMSFRVSA | QDVCENFWMVWS | SYQHKFQDDDQTH |
| | DVSAPKVEADVS | LQVLLGFAGRNK | DAMAHGCFLNAKR | VKGSLKAGFFGT |
| 9 | MNCREVTFVPGLC | SGSKKKGPPDADSP | IIGVTLFSSTKEDFQ | EHKVLLTGTPLQKT |
| | KIFDEILVNAAD | LYLPYKTLVST | QYAAALQAGM | VEELFSLLHFL |
| 10 | PTMASVPALQLTLP | STLATIQALASGCS | EALRSQAALGRVL | PGHDKMLSPNFDV |
| | THHPRRCPIPK | LPITSLDATGN | LDCGASCAADPG | HHTAMLTRGEQH |
| 11 | KLGTRLVPAERRK | NKQWDQHFRSMK | DSQFLAPDVTSTPV | PPAVISQMLLLDSP |
| | KLKTSRDKLRKS | HQYEQKITELRQK | NTVVSGALDRL | HKEPIRLRYKL |
| 12 | SSDQTNDSEGASRP | LLSSRSPLAEHRPD | TSSTSTPSSTPGMT | LSDQENAPPILPRRP |
| | APFEMPSSVSE | AYQDVSMVLLP | WILTELTTAAT | SEGLGPSPHL |
| 13 | IEDLLPASYFSTILL | FIQRDYSSGTRCLF | ASLLTSGLELYCKK | PKVRERHFSESTYI |
| | GVQTDQRVLR | QTKFPAELENR | GLSMTVEADPA | DNALSRLTLGN |
| 14 | SKPDLTAALRDIW | KPKEDKLRLAQEV | CAMLRRLEKRILID | PPACSSSSSSLFLAV |
| | AQYETIAAKNIS | SDMVIYCKSVHF | LPSREARQAMI | VARRLGRGSV |
| 15 | EIAVLELAKSCPHV | HYNYMCNSSCMGS | MTEYKLVVVGAGR | GIALVMRVDGAVS |
| | INLHEVYENTS | MNRRPILTIITL | VGKSALTIQLIQ | SCFRQLFLAQQR |
| 16 | MPAQRLLLLLTLLL | EKKKPHIKKPLNVF | ELYQRMLKGPPPP | LHKSLKLYQVIFKG |
| | PGLGIFGSTST | MLYMKEMRAKV | AARARSQRRPRH | EIGKGNLGGIA |
| 17 | LSTMERSELAKKT | KQQPQDNFKNNVQ | SDNSGCDAPGNSY | APRLGSYSGTTILH |
| | GTTPDIILDDLL | KSQLPVQLDLGG | PSLSVPSSAESE | LLNSTSNNLYL |
| 18 | MSGREGGKRKPLK | KVTQTELMRESFT | EHRKDLGLTTVWK | APAPPARTSRGQVS |
| | QPKKQAKEMDEE | QKQEATESLKCQ | DQLSYLLSPALA | ERANEAGGQVG |
| 19 | LRQITVNDLPVGHS | MAAADGALLEAA | NCPTKKYMPAVTL | MTEYKLVVVGAD |
| | VDEALRLVQAF | ALEQPAELPASVR | TPTVNQHETSTS | GVGKSALTIQLIQ |
| 20 | SLHEICSKIMAEWK | DEMVNTICDVLQK | ELYQRMLKGPPPPP | KDLAKRLLVGKSD |
| | NAKALACSSLQ | PEQFPLVQGVAI | PEPAASAAQGT | SVDAEKSMLSKL |

| Ag | Patient G9 | Patient G10 |
|---|---|---|
| 1 | KVAKPKKAAKSAT | TSGSQGQSTQSSGY |
| | KAVKPKAAKPKV | SSSYGYPPSSL |
| 2 | ECTDGSSFVDEVEK | ISHLIEPLANAAQA |
| | VMKCGCTRCVS | EASQLGHKVSQ |
| 3 | SVLSPPPEKAGATA | PQTLLDQKVKVVN |
| | ATLLPHEVAPL | VARNAKDVAVSY |
| 4 | RCRQGATRLPLPLL | YCKFKNTEDITFSS |
| | LRDYLLLRVEG | VYVGLKDKLSG |
| 5 | EAHVAKGKSVFLH | VDSYEDEWGRLHE |
| | QMKKFVEWLQNA | SLWDSAGVLREG |
| 6 | ALKEKYDSAAAMG | AGAQRVGLPGPPA |
| | LNSRGKLVQVNS | PPGPPGKPGQDG |
| 7 | AKLLGLLAELRSLN | ADTERFYTRESTQF |
| | EAYGYQIQHIQ | LQQNPVTEYMK |
| 8 | FEHLKWNDMMEE | KESRPPRKFPSDIIF |
| | VYQTLFLLQHLWS | EAISSMFPDK |
| 9 | LLLNLAENPAMTSE | LQFSQIYPVVCVLS |
| | LLRAQVPSSLG | SFFIGDSGIPL |
| 10 | RQLFLSENRRKEIL | EALLHPFFAGLTLE |
| | QRIIVELVEFI | ERSFHTSRNPS |
| 11 | ISLLKLTQGETLCK | VDQSPKPLIIGPVED |
| | LYEQHHVVQDM | YDPGYFNNES |
| 12 | EEHLSIEDFTQASG | STPCWIELHLNGLL |
| | MTPAAFSALPR | QWLDKVLTQMG |
| 13 | ELDSLITAITTNR | QSLESSSLEGSHMG |
| | AHPSKCVTIQRT | VYFSAHWCPPC |
| 14 | EESREYTEDGQVT | GEVLSRRCVNLLN |
| | KETRYSYNTEWR | TALRPDMWPKSE |
| 15 | TNITTETSKILLR | HLCIVDDWICEEVI |
| | KNMQIANHTLKY | TGTDALLRRML |
| 16 | PPARKKFVIPLDVD | GGFGLALGTHANC |
| | EVPPGVAKPLF | LDSPPMFAGAGL |
| 17 | TLVQNLVNNGYV | LRGNHESRQITQFY |
| | WDETVRAAPYDW | GFYDECQTKYG |
| | R | |
| 18 | NLHGDGIALWYTL | NEQACEDMDILKL |
| | DRLVPGPVFGSK | ESYGTVVRISPQ |
| 19 | YMCNSSCMGGMN | PRSAPSLRPKDYDV |
| | QRPILTIITLEDS | DATLKSLNNQI |
| 20 | LSTSRPPQHLGGLK | QTNHCYIAILNISQ |
| | KPTYDPVSEDQ | GEVDPTQVHKS |

TABLE 47B
GRANITE Subject-Specific Neoantigen Cassettes

| Patient | Cassette Sequence |
|---|---|
| G1 | LAEQVQKADEFIQANATNKL |
| | TVIAEQGDGCSGHKNQNQID |
| | TLQEVRLRFLGQLSQDRDFI |
| | MTLNTSLHRSWWMENDTCVC |
| | STVAEFSHQCSHAGGRPGNW |
| | PMNTIAEAVIEMVNRGQIQI |
| | TINGFVSQKQIADRAAHNES |
| | PGNNVVDISVSEEAEVDVRE |
| | REIQRDREPKRARDLQLQAE |
| | QKGLTEVIQSLKMENEEFKK |
| | QGRVKDLESLFHQSEVELAA |
| | ALSDKGKISRHEMLQVLHLM |
| | VGVQVTEEQLGLFEGDEYAT |
| | LMMQCKEGAQKEGLMCLAFK |
| | NDATEILHSHVVKPVPAHPS |
| | GIGWLITFCSKGEDCLTSQT |
| | RLRLSHNMKLSISYLREYAK |
| | ANDWLQFIIHLNTAQDELVT |
| | FSDELAQLYHHVCLCAPFPA |
| | WSAFGKEEHDEALKNTWNLH |
| | TDAYLLYTPSQIVLTAILSS |
| | ASRAGGPKVDIDVPDVNFEA |
| | PEGKLKGPKFNFDVAGYIVG |
| | ANTETYLLEKSRAIREEQKL |
| | PEADVAVLRTFLRQQASILS |
| G2 | LEAFLTQKAKVGKLKDDDFE |
| | RISELYPLAAHTYTPITGSV |
| | STIRQYPVSAIMEIQLNGGT |
| | VAKKVAWAQARLEKQKLPGL |
| | NTATILLMGTEDALLQQLAD |
| | GFAAEFKLVDQSRPPHEPKF |
| | VYQAKMSNLRQVGLKKPMER |
| | SSVLDRYPPAQLMPLKTFPA |
| | AIWGVIQSELNYSVILYSLS |
| | VLYKGEARVVLLKAAYDVSS |
| | DMFDCVFPTRTALFGSALVP |
| | TGNLQSNLKVLNHSPMSGAS |
| | VNFDYKSPSPDQAVGAYLPL |
| | TTARYSTLLAGNLRIRYYDR |
| | VTGVSSFHVTWVSQASADQR |
| | DVTRQADCKMKLADFVKYYY |
| | SGKRESEVSKAIINIFFFYG |
| | PPQKIIMDQRLRNDAKNLYA |
| | ANSIGAVLAHKGYFRKRQEL |
| | ESENKKLNNDLNELRKAVAD |
| | LQMTDIVQACHDSIKAALDI |
| | SIKSDSSLDAVLSQDLILAD |
| | GPAVEVGDSLVKELRRLEDQ |
| | LASLQQELAALALKQRRMNR |
| | VGNESLNPAVAKAAETIIDT |
| G3 | SKELVEKSEAVRKMSSSGKL |
| | WKPRQMASMGTLAFHEYGRP |
| | FLIIKDQDRKDTRIAKRMAM |
| | LIYTDFLCMAPISFFYLSLL |
| | RMYLSPPCIHCLGPIKLELL |
| | AVKLLIERATNQGWMLRVKW |
| | LYHLAVRASSDGEGTMSLPA |
| | SVDGSPVSPSCSPPSPSALI |
| | QEMEERLWEKEQEVAERYHF |
| | LEERLARLGLTEVYQWYLDL |
| | FFGMHVQEYGSDWPPPNTRR |
| | VYISYLFIYQSLDAIDGRQA |
| | RRTNSSSPLGNWVQQQAYGM |
| | LAGIPVTICTYPFVFMDARR |
| | VPPKDLRVKKNLKKFRYVKL |
| | VKKNRQNGMHTLYLLDIKVK |
| | EQSLEPNYADTLISQESFEK |
| | KGFLSAPQSLGDGGLVCPYM |
| | EFLKNENNELPKLQWSLSLA |
| | EKSILHEECIKSPVVETVPA |
| | PPDPLEPPSLPAQRPGPPTP |
| | AAAHSVKGGYQESSLLRVLV |
| | TLNDLEPKAAKQGKDLTVLD |
| | HTLPTALTSPELSSWHSSFV |
| | AVTEEGTKAAAATGIGFTVT |
| G4 | SYQPAVTAPEPDSSPRLAVD |
| | FALPKEDPYLEDSEASLVPL |
| | AYQPIPFSQSPLKIFAQDGE |
| | GQHIDIQMKNRMDGTTEPGD |
| | YNINILFTDTHIPGSPFKAH |
| | NSLATVTMEDLIQPWFPEFS |
| | EARAIKRYRVRSYEAVAVAA |
| | AASAYNYAEQANMCRCLVTL |
| | KVLMADKPGNTVNFRHLLQS |
| | KSGTQPHTATRLSLQQPRAE |
| | SLQHQLPQPGAQHFATRGYP |
| | MEDMKDVKKLITDEFVKHKY |
| | LDYARVPNSNSFVIPFIHLP |
| | EIINLYNLSEQNDVFRDSVK |
| | SCRVHVPGKTLTCGKGRRDF |
| | YKCLSCTKTFPNVPRAARHA |
| | ATHGPERAQGELFPDRASSS |
| | WGNAAAAAGDKIPASQHTQN |
| | WSTTWTKDSKRRDKRKVHLA |
| | KSSGSALMVWGTGNPRRQFI |
| | VYCDFSTGETCIQAQPENIP |
| | AKNWYAILSFGELIFCGMPA |
| | EMLDFFNDCGPKPFRPILGL |
| | HLNLGILYYVYMGLLVFYIE |
| | ACESGSMVNHLPDNINVYAT |
| G5 | STLYGRVLAPLPERAGGAAS |
| | GGGGNSKPDLTAALRDIWAQ |
| | YETIAAKNISGTGGSGPGLS |
| | RTRALSTGALPPLAPIEDLL |
| | PASYFSTILLGVQTDQRVLR |
| | RIEVMALPKPGGTHSLALVT |
| | VPSMGVEEDESAMPKKDSRT |
| | LLARFNEQIEPTMASVPALQ |
| | LTLPTHHPRRCPIPKLRKRH |
| | RKHTQQRVAALSLSTLASPK |
| | VDPEDQSQVTLASYGFAFRY |
| | CPSGKMNCREVTFVPGLCKI |
| | FDEILVNAADSLHEICSKIM |
| | AEWKNAKALACSSLQMSGRE |
| | GGKRKPLKQPKKQAKEMDEE |
| | EIAVLELAKSCPHVINLHEV |
| | YENTSGASAPGKSMEASMDV |
| | SAPKVEADVSSSDQTNDSEG |
| | ASRPAPFEMPSSVSESARIP |
| | EQLDVTAHGVYAPEDVYRFR |
| | KLGTRLVPAERRKKLKTSRD |
| | KLRKSLRQITVNDLPVGHSV |
| | DEALRLVQAFLSTMERSELA |
| | KKTGTTPDIILDDLLMPAQR |
| | LLLLLTLLLPGLGIFGSTST |
| G6 | LLSSRSPLAEHRPDAYQDVS |
| | MVLLPMAAADGALLEAAALE |
| | QPAELPASVRHYNYMCNSSC |
| | MGSMNRRPILTIITLFQYNY |
| | EYLGNSGQLVITPLTDRCYM |
| | FIQRDYSSGTRCLFQTKFPA |
| | ELENRAVRTPYTMGYGEPMY |
| | YNDAYGALDYLLLAAHRFLA |
| | TELSQFSPSLISEKIEQTEA |
| | AAREEILTNGGSLSHHHGVG |
| | STLATIQALASGCSLPITSL |
| | DATGNKQQPQDNFKNNVQKS |
| | QLPVQLDLGGMDKDGYFWFV |
| | ARSDDVILSSGYRIGDEMVN |
| | TICDVLQKPEQFPLVQGVAI |
| | MTEYKLVVVGAVGVGKSALT |
| | IQLIQIEMWISILELNELEY |
| | AAVELHQAKDNKQWDQHFRS |
| | MKHQYEQKITELRQKKPKED |
| | KLRLAQEVSDMVIYCKSVHF |
| | ELKHMVMSFRVSALQVLLGF |
| | AGRNKKVTQTELMRESFTQK |
| | QEATESLKCQEKKKPHIKKP |
| | LNVFMLYMKEMRAKVSGSKK |
| | KGPPDADSPLYLPYKTLVST |
| G7 | EALRSQAALGRVLLDCGASC |
| | AADPGTSSTSTPSSTPGMTW |
| | ILTELTTAATQDVCENFWMV |
| | WSDAMAHGCFLNAKRMTEYK |
| | LVVVGAGRVGKSALTIQLIQ |
| | QIGALILATDISCQNEYLSL |
| | FRSHLELYQRMLKGPPPPPP |
| | EPAASAAQGTIIAKLLADEL |
| | ISLKDIDPVQRYVPLASLLT |
| | SGLELYCKKGLSMTVEADPA |
| | NCPTKKYMPAVTLTPTVNQH |
| | ETSTSREKVDNVWTEESSNQ |
| | FLQELETGQRDSQFLAPDVT |
| | STPVNTVVSGALDRLHMSSM |
| | EHTEEGLQERLADAMAESPS |
| | SDNSGCDAPGNSYPSLSVPS |
| | SAESEVAASEKSTGKIFLYD |
| | GRGDNQPLHIELYQRMLKGP |
| | PPPAARARSQRRPRHIIGVT |
| | LFSSTKEDFQQYAAALQAGM |
| | CAMLRRLEKRILIDLPSREA |
| | RQAMIMFEVCFESKGTGPIP |
| | DQLVILDMKHLRDVDAKALV |
| | RSHFLLVYGDVISNIEHRKD |
| | LGLTTVWKDQLSYLLSPALA |
| G8 | APRLGSYSGTTILHLLNSTS |
| | NNLYLPPACSSSSSSLFLAW |
| | ARRLGRGSVSYQHKFQDDDQ |
| | THVKGSLKAGFFGTPPAVIS |
| | QMLLLDSPHKEPIRLRYKLP |
| | KVRERHFSESTYIDNALSRL |
| | TLGNEHKVLLTGTPLQKTVE |
| | ELFSLLHFLPYHRSQSSSSV |
| | LNNKSMDSINYPSDLSDQEN |
| | APPILPRRPSEGLGPSPHLE |
| | VLLLSMRDMNITKLTSVDAP |
| | LFNAPGHDKMLSPNFDVHHT |
| | AMLTRGEQHLHKSLKLYQVI |
| | FKGEIGKGNLGGIAAQPHVN |
| | GGPLYSKNSISIAPPPPPMA |
| | PAPPARTSRGQVSERANEAG |
| | GQVGAKMKWTNLLYKAGGVK |
| | IRDDERLLQNANFDLRTSQS |
| | VRAMLAYNMRDQHLMTEYKL |
| | VVVGADGVGKSALTIQLIQG |
| | IALVMRVDGAVSSCFRQLFL |
| | AQQRGSRKERQVYSKALNRL |
| | FGVEASGRRKDLAKRLLVGK |
| | SDSVDAEKSMLSKLEQSAER |
| | CVSTLLNLIQTKVNYVVQE |
| G9 | TNITTETSKILLRKNMQIAN |
| | HTLKYSVLSPPPEKAGATAA |
| | TLLPHEVAPLRCRQGATRLP |
| | LPLLLRDYLLLRVEGELDSL |
| | ITAITTNRAHPSKCVTIQRT |
| | YMCNSSCMGGMNQRPILTII |
| | TLEDSECTDGSSFVDEVEKV |
| | MKCGCTRCVSLLLNLAENPA |
| | MTSELLRAQVPSSLGTLVQN |
| | LVNNGYVWDETVRAAPYDWR |
| | KVAKPKKAAKSATKAVKPKA |
| | AKPKVEAHVAKGKSVFLHQM |
| | KKFVEWLQNAAKLLGLLAEL |
| | RSLNEAYGYQIQHIQFEHLK |
| | WNDMMEEVYQTLFLLQHLWS |
| | NLHGDGIALWYTLDRLVPGP |
| | VFGSKISLLKLTQGETLCKL |
| | YEQHHVVQDMLSTSRPPQHL |
| | GGLKKPTYDPVSEDQEESRE |
| | YTEDGQVTKETRYSYNTEWR |
| | PPARKKFVIPLDVDEVPPGV |
| | AKPLFEEHLSIEDFTQASGM |
| | TPAAFSALPRRQLFLSENRR |
| | KEILQRIIVELVEFIALKEK |
| | YDSAAAMGLNSRGKLVQVNS |
| G10 | QSLESSSLEGSHMGVYFSAH |
| | WCPPCGGFGLALGTHANCLD |
| | SPPMFAGAGLKESRPPRKFP |
| | SDIIFEAISSMFPDKVDQSP |
| | KPLIIGPVEDYDPGYFNNES |
| | TSGSQGQSTQSSGYSSSYGY |
| | PPSSLSTPCWIELHLNGLLQ |
| | WLDKVLTQMGLRGNHESRQI |
| | TQFYGFYDECQTKYGYCKFK |
| | NTEDITFSSVYVGLKDKLSG |
| | GEVLSRRCVNLLNTALRPDM |
| | WPKSEEALLHPFFAGLTLEE |
| | RSFHTSRNPSQTNHCYIAIL |
| | NISQGEVDPTQVHKSAGAQR |
| | VGLPGPPAPPGPPGKPGQDG |
| | VDSYEDEWGRLHESLWDSAG |
| | VLREGHLCIVDDWICEEVIT |
| | GTDALLRRMLLQFSQIYPVV |
| | CVLSSFFIGDSGIPLADTER |
| | FYTRESTQFLQQNPVTEYMK |
| | ISHLIEPLANAAQAEASQLG |
| | HKVSQPQTLLDQKVKVVNVA |
| | RNAKDVAVSYNEQACEDMDI |
| | LKLESYGTVVRISPQPRSAP |
| | SLRPKDYDVDATLKSLNNQI |

TABLE 48A
GRANITE Subject HLAs

| Patient | HLA-A | HLA-A | HLA-B | HLA-B | HLA-C | HLA-C |
|---|---|---|---|---|---|---|
| G1 | A*02:01 | A*32:01 | B*18:01 | B*44:05 | C*02:02 | C*07:04 |
| G2 | A*30:04 | A*02:01 | B*08:01 | B*57:01 | C*07:01 | C*06:02 |
| G3 | A*02:01 | A*11:01 | B*27:02 | B*35:01 | C*04:01 | C*02:02 |
| G4 | A*25:01 | A*03:01 | B*15:17 | B*18:01 | C*07:01 | C*07:01 |
| G5 | A*01:01 | A*03:01 | B*07:02 | B*08:01 | C*07:01 | C*07:02 |
| G6 | A*01:01 | A*31:01 | B*37:01 | B*40:01 | C*03:04 | C*06:02 |
| G7 | A*01:01 | A*02:01 | B*08:01 | B*40:01 | C*03:04 | C*07:01 |
| G8 | A*03:01 | A*33:01 | B*42:01 | B*53:01 | C*04:01 | C*17:01 |
| G9 | A*02:01 | A*03:01 | B*35:12 | B*45:01 | C*04:01 | C*06:02 |
| G10 | A*01:01 | A*02:01 | B*08:01 | B*15:01 | C*04:01 | C*07:01 |

A shared neoantigen cancer vaccine (“SLATE”) was administered in combination with immune checkpoint blockade in patients with advanced cancer. The SLATE heterologous prime/boost vaccine regimen included (1) a ChAdV that is used as a prime vaccination [GRT-C903] and (2) a SAM formulated in a LNP that is used for boost vaccinations [GRT-R904] following GRT-C903. Both GRT-C903 and GRT-R904 expressed the same 20 shared neoantigens derived from a specific list of oncogenic mutations (see Table 34) as well as two universal CD4 T-cell epitopes (PADRE and Tetanus Toxoid). For subject inclusion, tumors were used for whole-exome and transcriptome sequencing to detect somatic mutations, and blood was used for HLA typing. Enrolled SLATE subjects were determined to have HLA A02:01 and KRAS mutation G12C predicted to be presented by HLA A02:01 (Patients S1, S2, and S3), HLA A01:01 and KRAS mutation Q61H predicted to be presented by HLA A01:01 (Patients S4 and S7), or HLA A03:01 or A11:01 and KRAS mutation G12V predicted to be presented by HLA A03:01 or A11:01 (A03:01 for Patient S9; A11:01 for Patients S11 and S15).

Both treatment studies (ie, the GRANITE and SLATE vaccine regimens) administered the vaccine via IM injection bilaterally (eg, in each deltoid muscle) in combination with immune checkpoint blockade, specifically SC ipilimumab and IV nivolumab. The studies followed two sequential phases.

GRT-C901 and GRT-C903 are replication-defective, E1 and E3 deleted adenoviral vectors based on chimpanzee adenovirus 68. The vector contained an expression cassette encoding 20 neoantigens as well as two universal CD4 T-cell epitopes (PADRE and Tetanus Toxoid). GRT-C901 and GRT-C903 were formulated in solution at 5×10¹¹ vp/mL and 1.0 mL was injected IM at each of 2 bilateral vaccine injection sites in opposing deltoid muscles. The GRT-C901 and GRT-C903 vectors differ only by the encoded neoantigens within the cassette.

GRT-R902 and GRT-R904 are SAM vectors derived from an alphavirus. The GRT-R902 and GRT-R904 vectors encoded the viral proteins and the 5′ and 3′ RNA sequences required for RNA amplification but encoded no structural proteins. The SAM vectors were formulated in LNPs that included 4 lipids: an ionizable amino lipid, a phosphatidylcholine, cholesterol, and a PEG-based coat lipid to encapsulate the SAM and form LNPs. The GRT-R902 vector contained the same neoantigen expression cassette as used in GRT-C901 for each patient, respectively. The GRT-R904 vector contained the same neoantigen expression cassette as used in GRT-C903. GRT-R902 and GRT-R904 were formulated in solution at 1 mg/mL and was injected IM at each of 2 bilateral vaccine injection sites in opposing deltoid muscles (deltoid muscle preferred, gluteus [dorso or ventro] or rectus femoris on each side may be used). The boost vaccination sites were as close to the prime vaccination site as possible. The injection volume was based on the dose to be administered. The dose level amount refers explicitly to the amount of the SAM vector, i.e., it does not refer to other components, such as the LNP. The ratio of LNP:SAM was approximately 24:1. Accordingly, the dose of LNP was 720 μg, 2400 μg, and 7200 μg for each respective GRT-R902/GRT-R904 dose level (see below).

Ipilimumab is a human monoclonal IgG1 antibody (see SEQ ID NO: 29520 and 29521) that binds to the cytotoxic T-lymphocyte associated antigen 4 (CTLA-4). Ipilimumab was formulated in solution at 5 mg/mL and was injected SC proximally (within ˜2 cm) to each of the bilateral vaccination sites. Ipilimumab was administered at a dose of 30 mg of antibody in four 1.5 mL (7.5 mg) injections proximal to the vaccine draining LN at each of the bilateral vaccination sites (ie, 1.5 mL below the vaccination site and 1.5 mL above the vaccination site on each bilateral side in each deltoid, ventrogluteal, dorsogluteal, or rectus femoris [deltoid preferred, but dependent on clinical site and patient preference])

Nivolumab is a human monoclonal IgG4 antibody (see SEQ ID NO: 29522 and 29523) that blocks the interaction of PD-1 and its ligands, PD-L1 and PD-L2. Nivolumab was formulated in solution at 10 mg/mL and was administered as an IV infusion (480 mg) through a 0.2-micron to 1.2-micron pore size, low-protein binding in-line filter at the protocol-specified doses. It was not administered as an IV push or bolus injection. Nivolumab infusion was promptly followed by a flush of diluent to clear the line. Nivolumab was administered following each vaccination (i.e., each of GRT-C901, GRT-R902, GRT-C903, or GRT-R904) with or without ipilimumab on the same day. The dose and route of nivolumab was based on the Food and Drug Administration approved dose and route.

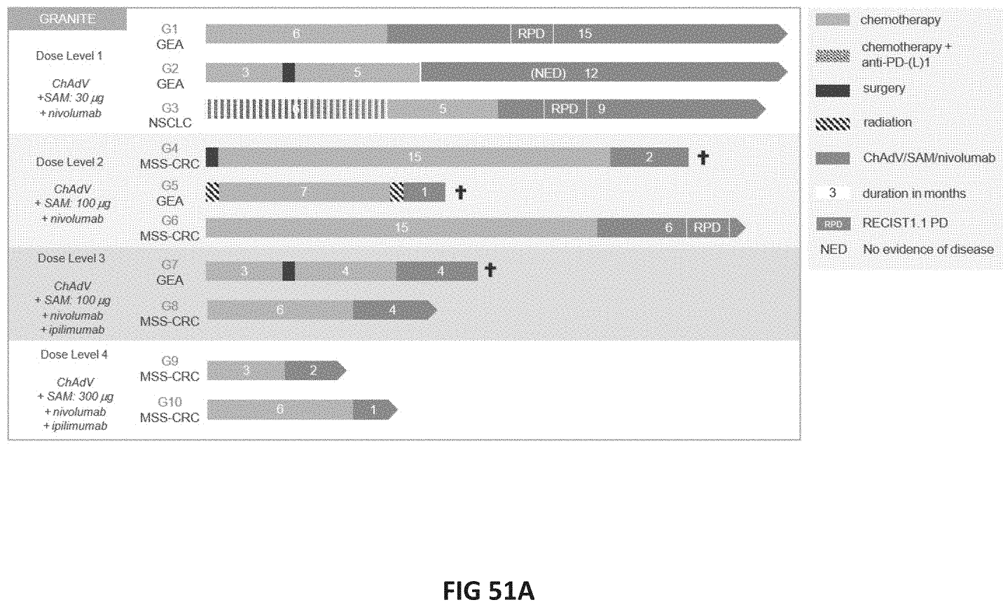

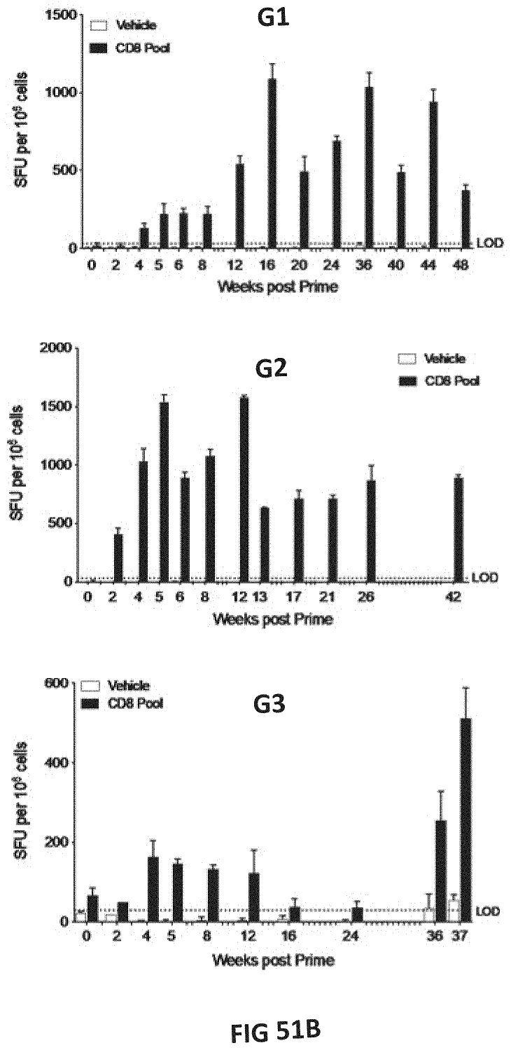

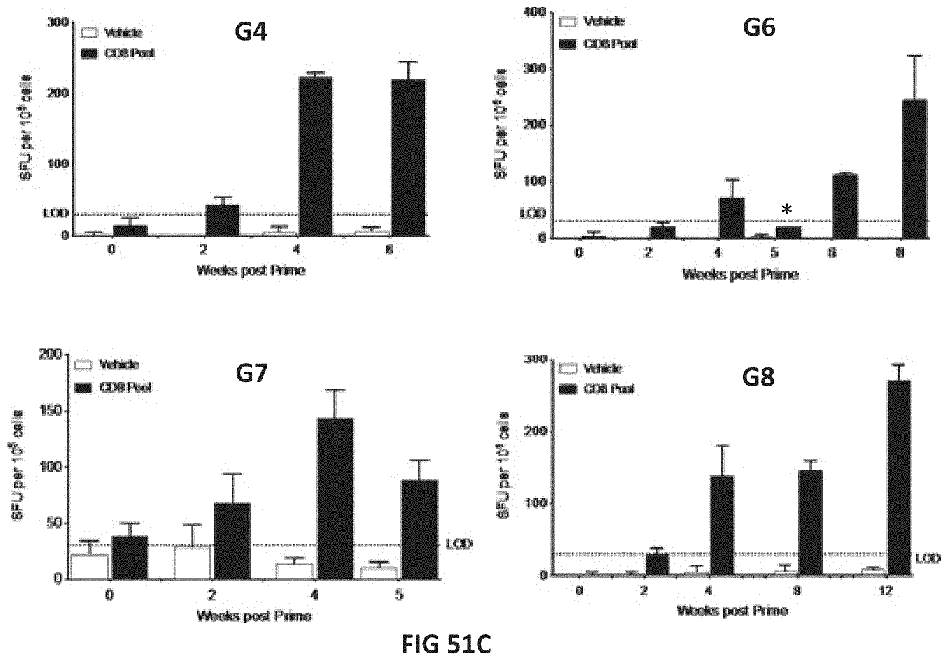

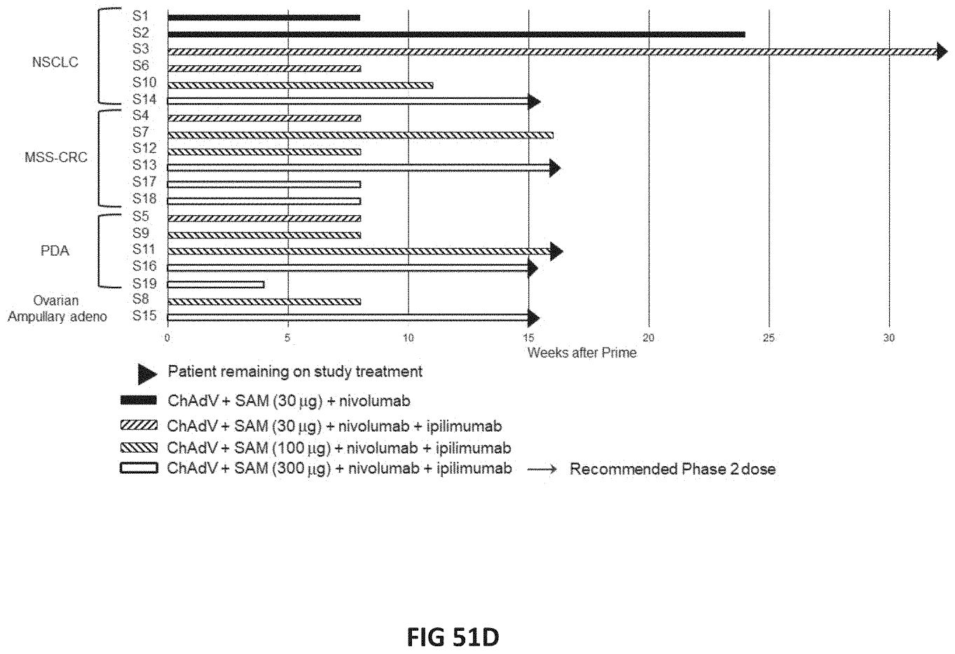

As shown in Table 49, subjects included those with advanced or metastatic NSCLC, MSS-CRC, gastroesophageal adenocarcinoma (GEA), ovarian adenocarcinoma, ampullary adenocarcinoma. As further shown in Table 49 and shown in FIG. 51A and FIG. 51D, all GRANITE and SLATE subjects were administered the priming GRT-C901 or GRT-C903 dose of 1×10¹² vp, respectively, and were administered the IV infusion of nivolumab at 480 mg Q4W, as described above. Boosting doses of GRT-R902 or GRT-R904 for GRANITE and SLATE subjects, respectively, where administered every four weeks following the initial priming dose for the first 6 months and then in 3 month intervals. As shown in FIG. 51A, Table 50B, and Table 50C, three GRANITE subjects (G1-G3) were administered 30 μg of GRT-R902, three subjects (G4-G6) were administered 100 μg of GRT-R902 boosting dose, two subjects (G7-G8) were administered 100 μg of GRT-R902 in combination with SC ipilimumab (30 mg) at every dose, and two subjects (G9-G10) were administered 300 μg of GRT-R902 in combination with SC ipilimumab (30 mg) at every dose. As shown in FIG. 51D, Table 50D, and Table 50E, two SLATE subjects (S1-S2) were administered 30 μg of GRT-R904, four subjects (S3-S6) were administered 30 μg of GRT-R904 in combination with SC ipilimumab (30 mg) at every dose, six subjects (S7-S12) were administered 100 μg of GRT-R904 in combination with SC ipilimumab (30 mg) at every dose, and six subjects (S13-S19) were administered 300 μg of GRT-R904 in combination with SC ipilimumab (30 mg) at every dose.

As shown in Tables 50A-C, adverse events were recorded for GRANITE but no dose limiting toxicities were observed. Fever was the only severe adverse event observed. Grade 3 hyperthyroidism and Grade 3 asymptomatic CK elevation were observed in one patient each. The data overall demonstrate the general safety and tolerability of administering a heterologous prime/boost of 1×10¹² vp (GRT-C901) and 30 μg, 100 μg, 100 μg+SC ipilimumab, and 300 μg+SC ipilimumab GRT-R902.

As shown in Tables 50A and D-E, adverse events were also recorded for SLATE. Two dose limiting toxicities were observed out of 19 subjects after administering GRT-C903 in combination with nivolumab and ipilimumab (1 each of CPI-induced autoimmune hepatitis and myositis). Severe adverse events observed were mostly attributable to checkpoint immunotherapy treatment. Grade 3 fever, reversible Grade 3 ALT together Grade 4 AST elevation (attributable to nivolumab), Grade 3 neutropenia together with thrombocytopenia, and Grade 3 rhabdomyolysis were observed in one patient each. Transient fever and injection site reactions were as expected, as well as customary CPI-induced immune-related adverse events. The data overall demonstrate the general safety and tolerability of a heterologous prime/boost of 1×10² vp of GRT-C903 and 30 μg, 30 μg+SC ipilimumab, 100 μg+SC ipilimumab, and 300 μg+SC ipilimumab GRT-R904.

Immune responses following vaccinations for GRANITE subjects was assessed. Blood draws were performed for subjects and PBMCs were collected. T-cell responses were assessed by IFN-gamma ELISpot. Peripheral blood mononuclear cells (PBMCs) were plated at 2×10⁵ cells/well (ex vivo) or 10⁵ cells/well (post-IVS) and stimulated overnight in the presence of minimal epitope peptides (8-11mers) or controls in ELISpot plates coated with anti-human Interferon-gamma antibody. Following 20 h incubation in a humidified, 5% CO₂, 37° C. incubator, cells were removed and ELISpot plates developed according to standard protocols. Data are reported as spot-forming cells (SFC) per 10⁶ splenocytes.

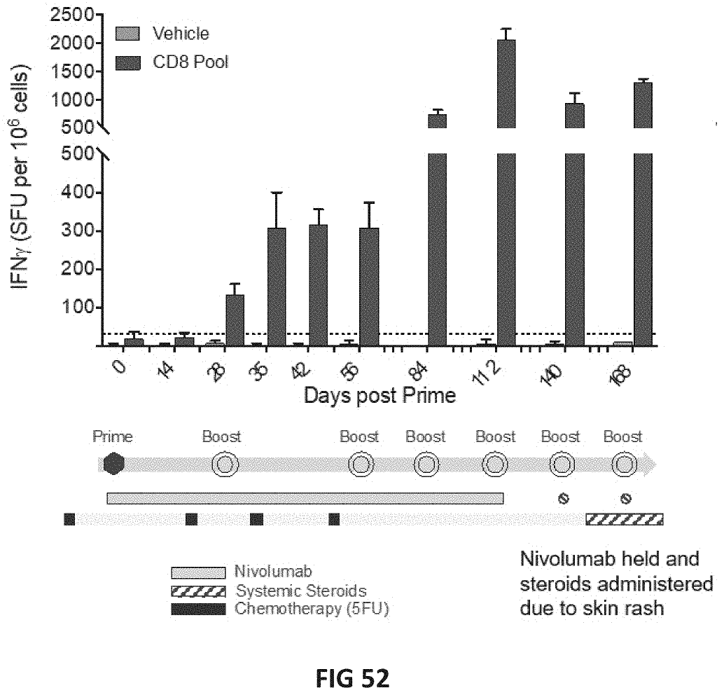

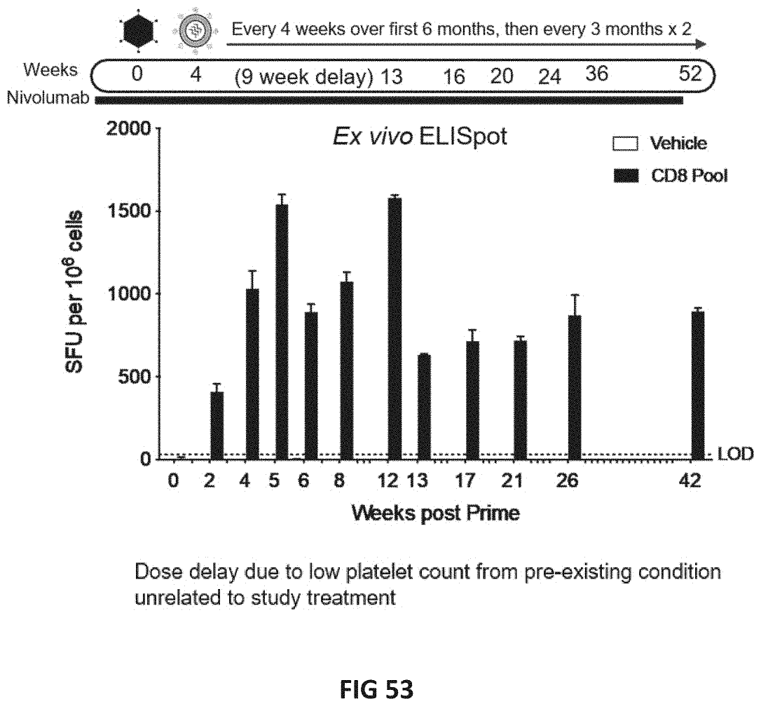

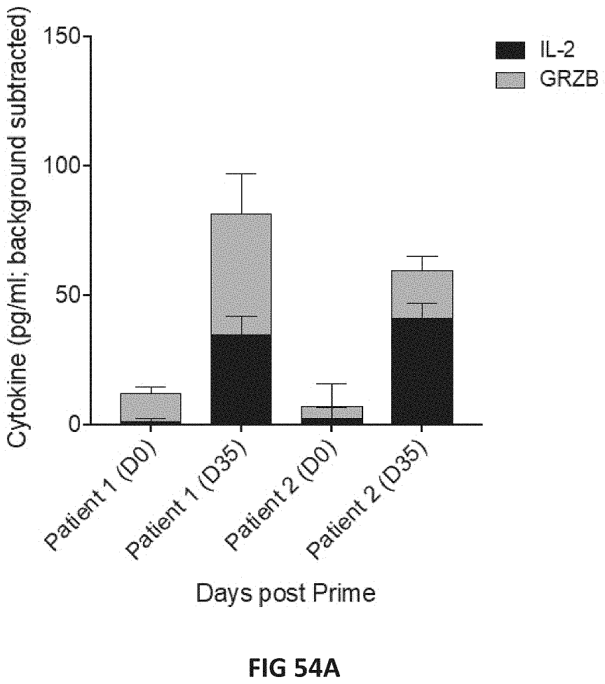

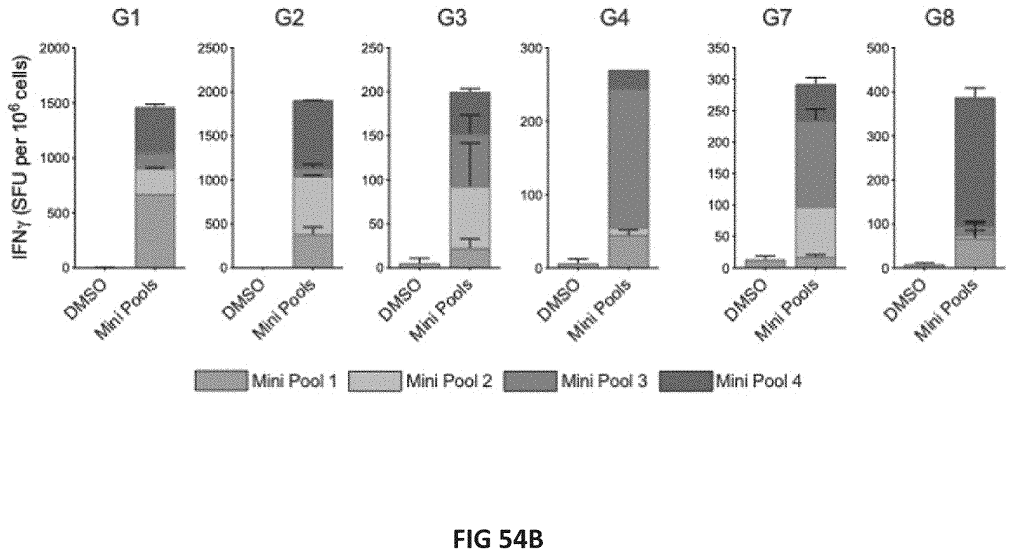

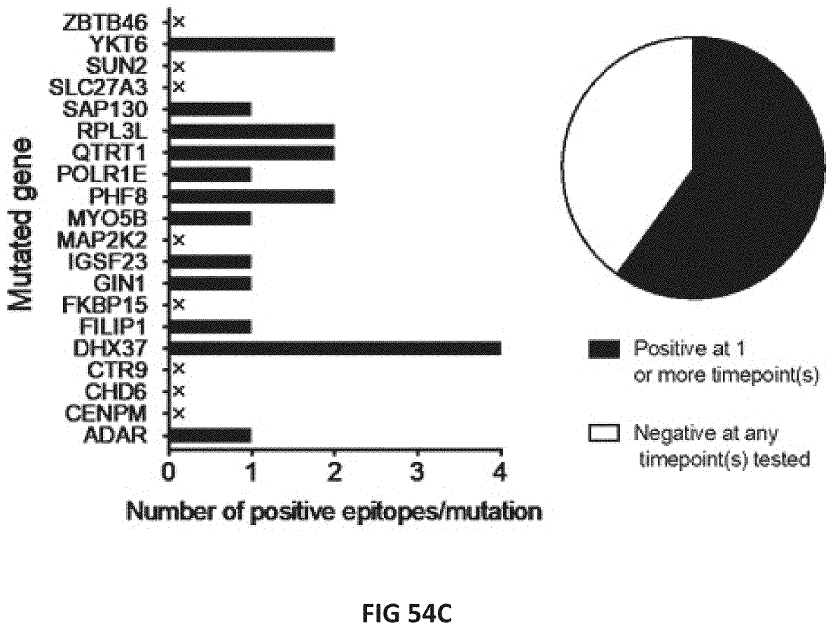

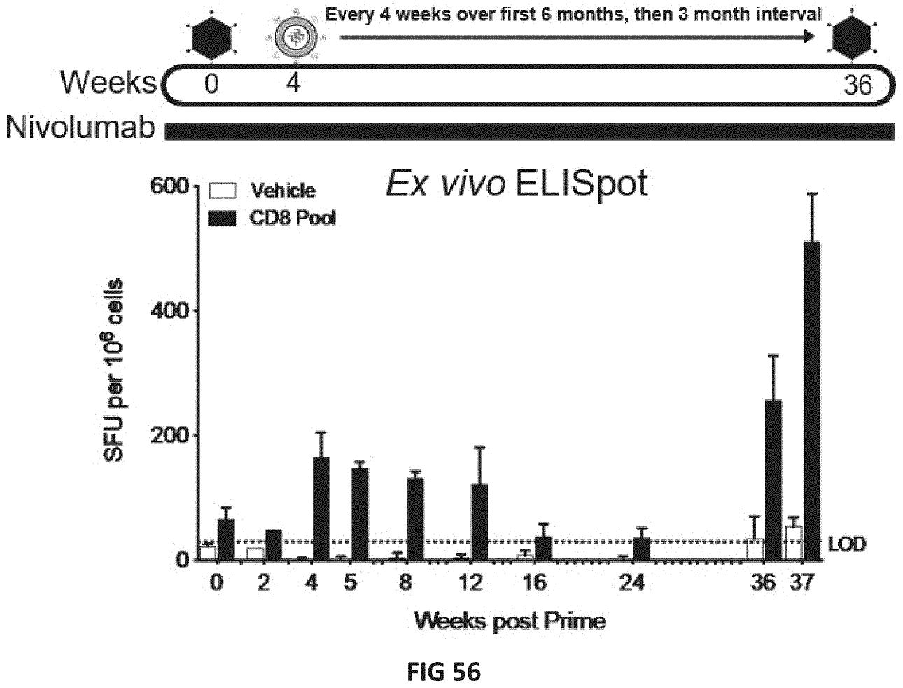

Shown in FIG. 51B (GRANITE patients G1-G3), FIG. 51B (GRANITE patients G4, G6, G7, and G8), FIG. 52 (GRANITE patient G1), FIG. 53 (GRANITE patient G2), and FIG. 56 (GRANITE patient G3) are CD8 T cell responses to peptide stimulation using epitopes encoded by the neoantigen cassette (peptides used for each GRANITE patient are presented in Tables 51A-51F). Quantification of ELISpot data for FIGS. 51-53 and 56 is presented in Tables 52A-52D, respectively for select GRANITE patients and timepoints. The priming GRT-C901 dose elicited a CD8 T cell immune response to vaccine specific peptides as early as Week 2 (Day 14) demonstrating effective priming of subjects. In addition, the GRT-R902 boosting doses resulted in further increased CD8 T cell responses. Notably, GRANITE patient G1 demonstrated an initial further increase following the first GRT-R902 boosting dose, as well as an even further increase following subsequent GRT-R902 boosting doses (FIG. 52). GRANITE patients G6 and G8 demonstrated similar additional increases following subsequent doses (FIGS. 51B and 51C). GRANITE patient G2 also demonstrated an increase following at least the second boost, which was delayed to 9 weeks post prime do to a pre-existing condition (FIG. 53). Notably, GRANITE patient G3 also demonstrated an increase following a boost with the adenovirus GRT-C901 dose at week 36 (FIG. 56). As shown in FIG. 54, CD8 T cells also demonstrated an increase in peptide-stimulated IL-2 and Granzyme B production for GRANITE patients G1 and G2 (FIG. 54A and quantified in Table 53A) and polyclonal responses to different subsets for GRANITE patients G1, G2, G3, G4, G7, and G8 (see pools referred to as “Mini” pools in Tables 51A-51F) of vaccine-encoded neoantigens (FIG. 54B, with G1 and G2 quantified in Table 53B) a week after the first GRT-R902 boosting dose. Notably, PMBCs were isolated for patient G2 using leukapheresis, further assessed for the polyclonal response to each of the 40 peptides individually (“full deconvolution”), and demonstrated an immune response to 12 of the 20 vaccine-encoded neoantigens (FIG. 54C). Accordingly, the results demonstrate that the heterologous prime/boost protocol resulted in a polyclonal T cell response to the vaccine-encoded neoantigens.

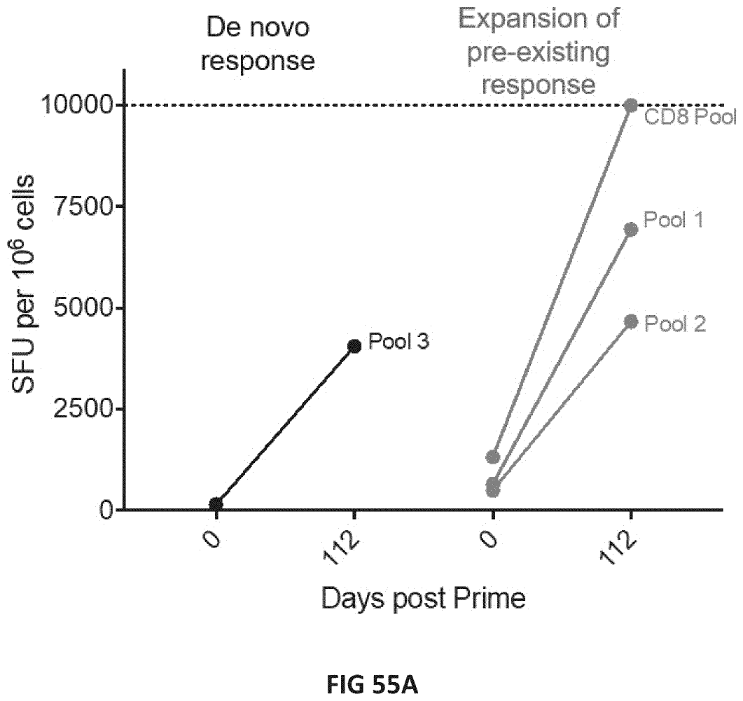

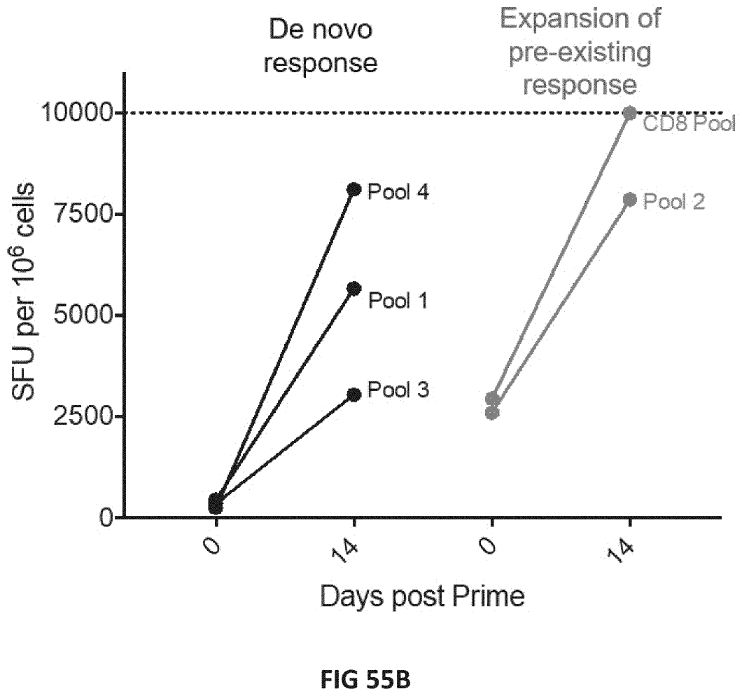

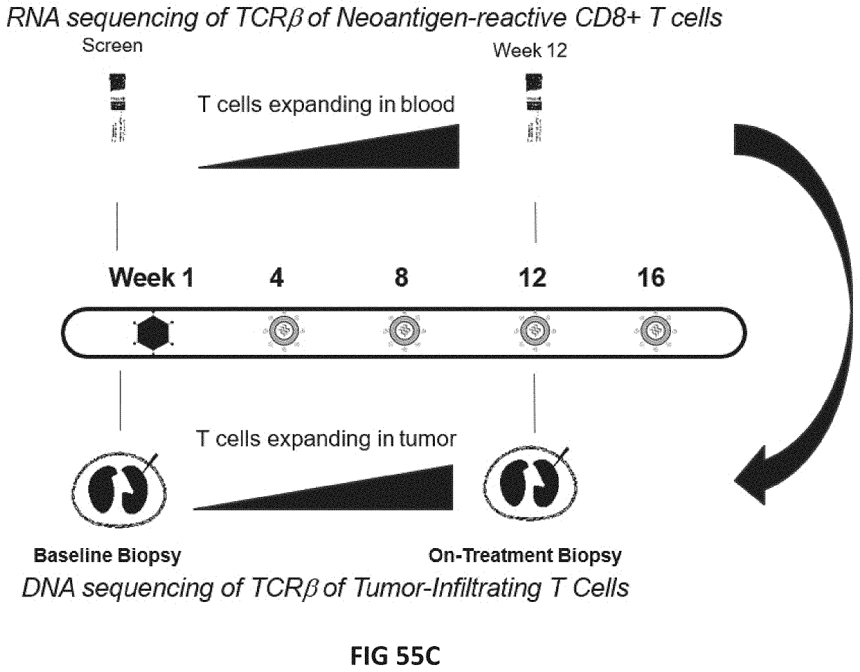

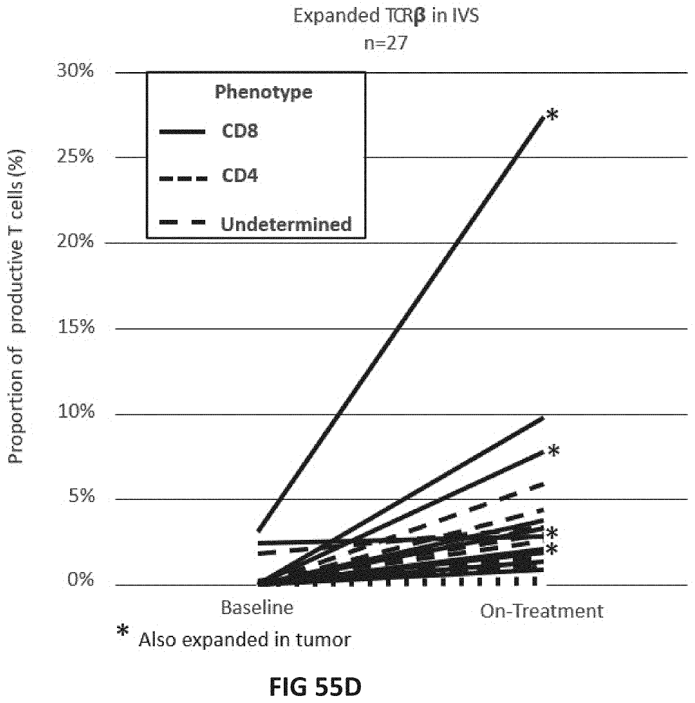

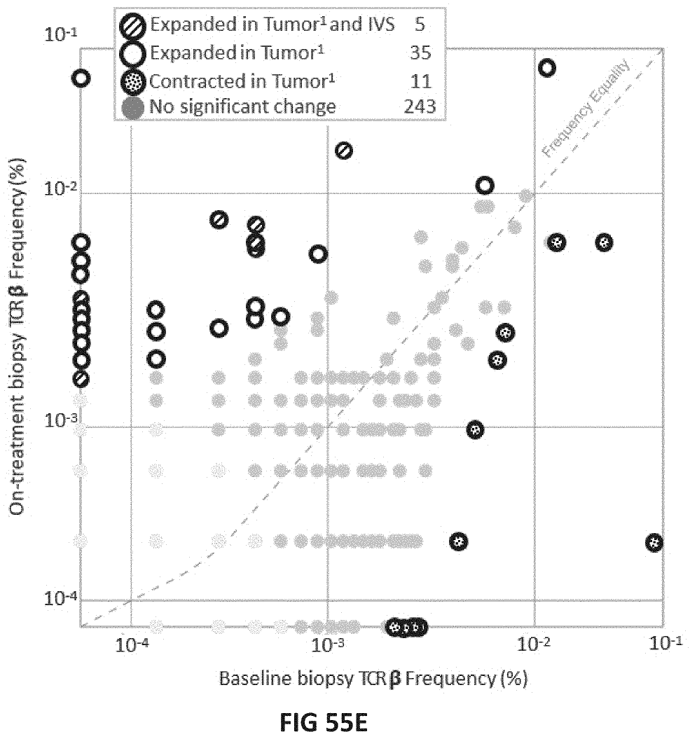

The CD8 T cell pools responsive to vaccination for GRANITE subjects were further assessed and characterized. PBMCs were collected and expanded for 2 weeks in the presence of peptide pools (In Vitro Stimulation (IVS) culture), and T-cell responses were assessed by IFN-gamma ELISpot for each possible vaccine-encoded peptide (40 total to cover different distinct epitopes for each of the neoantigens, i.e., two or more distinct epitopes from the same neoantigen that may both elicit and immune response to that neoantigen) and reported as spot-forming cells (SFC) per 10⁶ splenocytes. As shown in FIG. 55A (GRANITE patient G1) and FIG. 55B (GRANITE patient G2) and quantified in Table 54, subsets of the expanded CD8 T cells were below the limit of detection prior to the priming GRT-C901 dose protocol (see GRANITE patient G1 pool 3; GRANITE patient G2 pools 1, 3, 4). Accordingly, these T cells were considered to be de novo expanded naïve CD8 T cells that were effectively primed by the heterologous prime/boost protocol. In contrast, other subsets of the expanded CD8 T cells were detected prior to the priming GRT-C901 dose (GRANITE patient G1 pools 1 and 2; GRANITE patient G2 pool 2). Accordingly, these T cells were considered to be pre-existing antigen-experienced CD8 T cells that were effectively expanded by the heterologous prime/boost protocol. The results demonstrate that that the heterologous prime/boost protocol resulted in expansion of both naïve CD8 T cells and pre-existing antigen-experienced CD8 T cells.

The TCRs of the expanded CD8 T cells were further assessed by TCR β sequencing. The general sequencing workflow is shown in FIG. 55C. PBMCs from the blood were sequenced by RNA sequencing (10× Genomics) and tumor-infiltrating T cells were sequenced by DNA sequencing (Adaptive Biotechnoliges), with each population assessed at baseline and Week 12 of treatment. As shown in FIG. 55D, 27 TCR-βs from PBMCs stimulated by IVS for GRANITE patient G3 expanded following treatment as determined by the percent proportion of productive T cells, including 5 of which had TCR-βs associated with tumor-infiltrating T cells. As shown in FIG. 55E, 40 TCR-βs from tumor-infiltrating T cells for GRANITE patient G3 expanded following treatment, including the 5 associated with the stimulated PBMCs. Accordingly, the sequencing results demonstrate the vaccine therapy leads to expansion of neoantigen reactive CD8 T cells in both the blood and tumor indicating the treatment is driving tumor T cell infiltration.

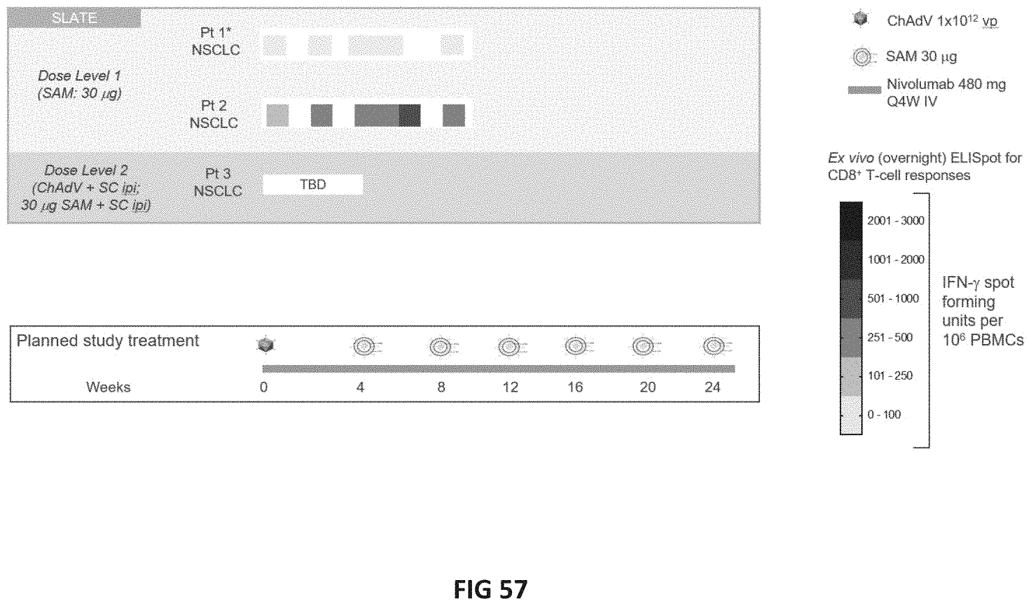

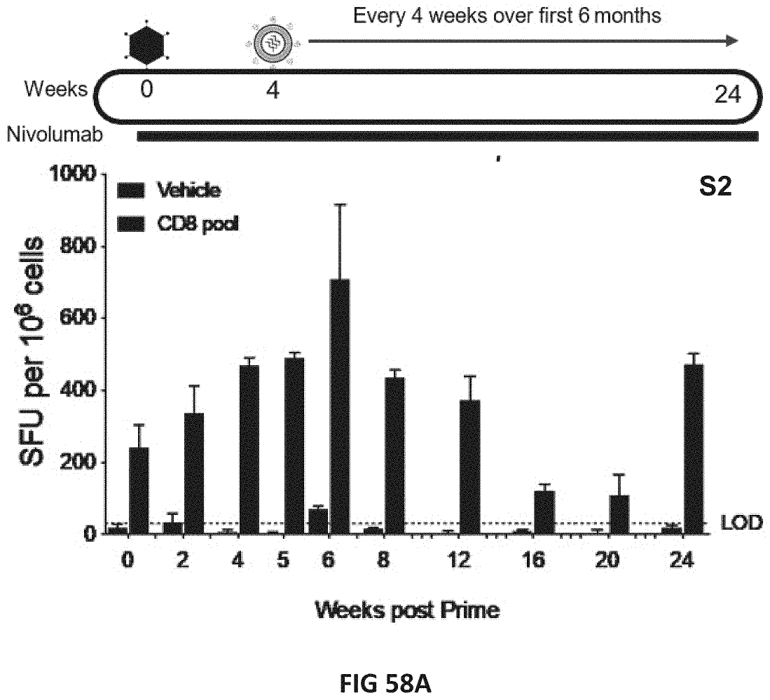

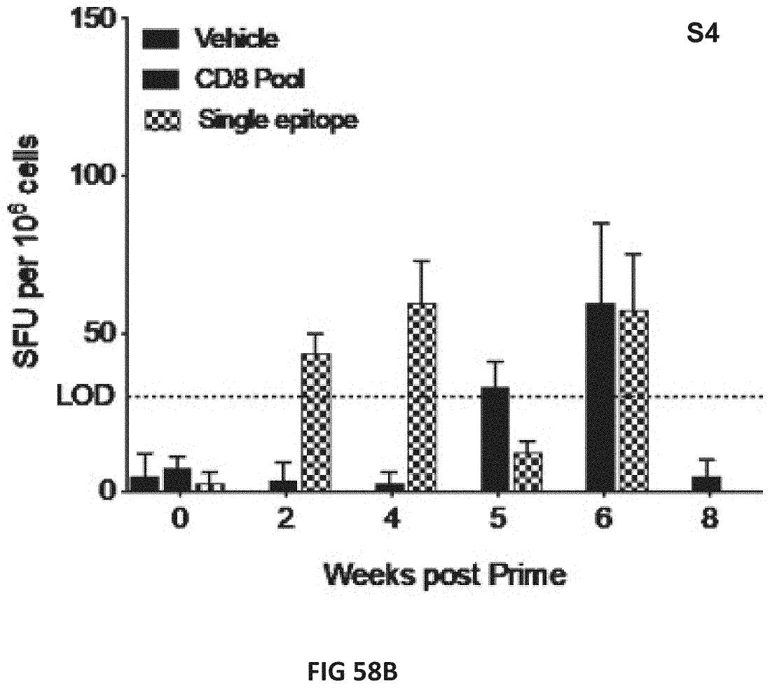

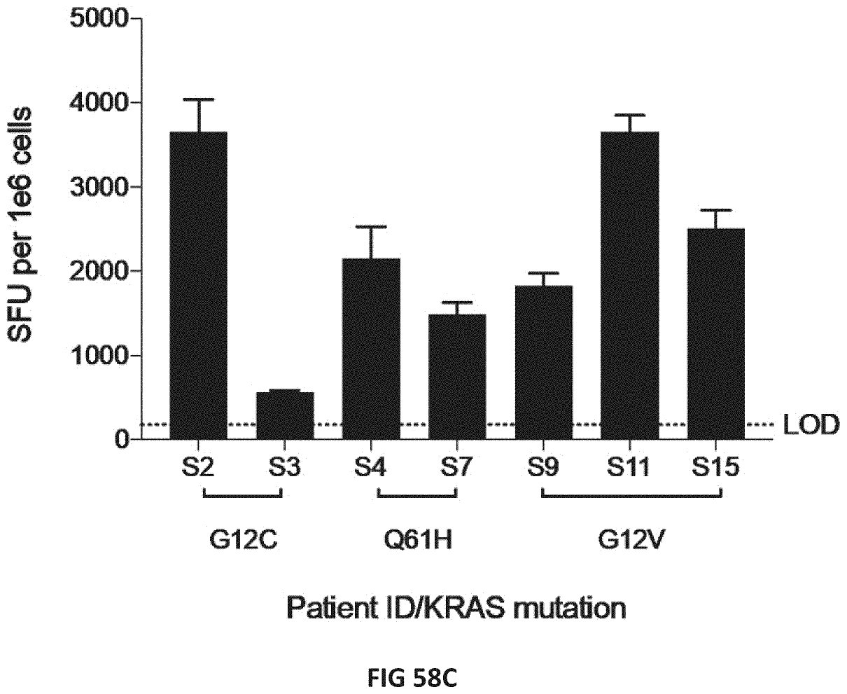

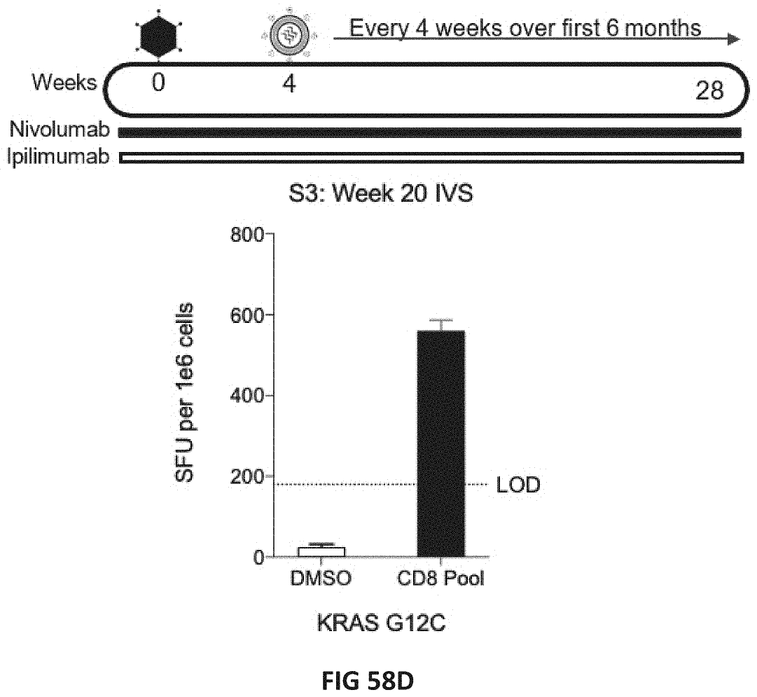

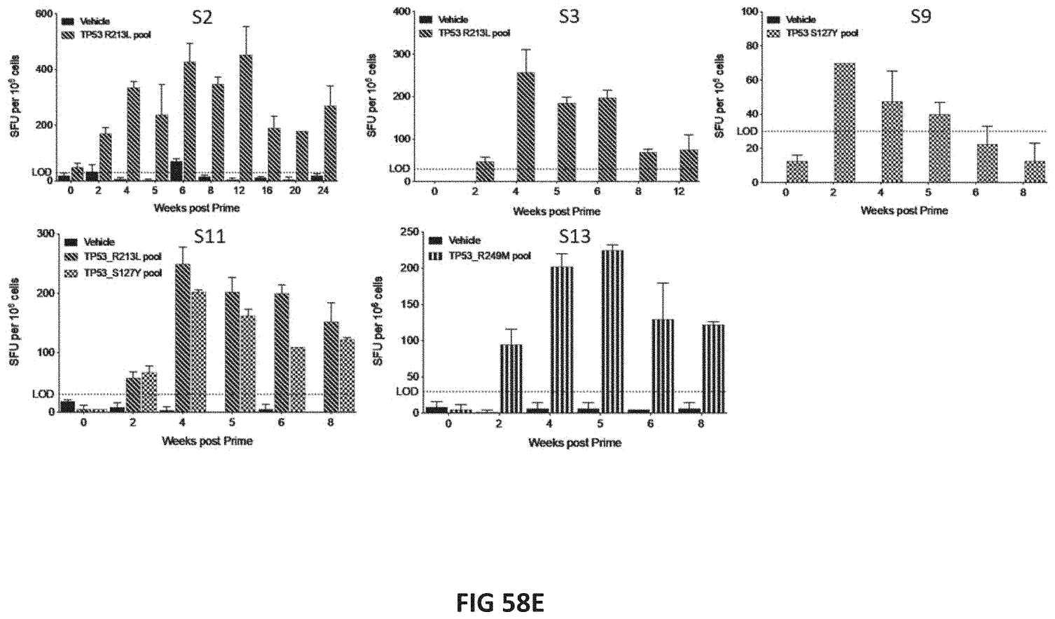

Shown in FIG. 57 (summary of 3 SLATE patients), FIG. 58A (SLATE patient S2), and FIG. 58D (SLATE patient S3) are CD8 T cell responses to peptide stimulation using a pool of KRAS G12C epitopes (KRAS G12C epitope encoded in SLATE vaccine cassette with peptides used for stimulation presented in Table 55A). Shown in FIG. 58B (SLATE patient S4) are CD8 T cell responses to peptide stimulation using a pool of KRAS Q61H epitopes (KRAS Q61H epitope encoded in SLATE vaccine cassette with peptides used for stimulation presented in Table 55A) and stimulation with the single peptide ILDTAGHEEY. For each figure, the left column at each time point represents vehicle and right column at each time point represents peptide stimulation. Shown in FIG. 58C are CD8 T cell responses to peptide stimulation using G12C, Q61H, or G12V peptide pools (see Table 55A), as indicated, with data shown for Week 4 for S4 and S11; Week 8 for S7, S9, and S15; Week 12 for S2; and Week 20 for S3. Quantification of ELISpot data for FIGS. 57-58 is presented in Tables 56A-56B, respectively for select SLATE patients and timepoints. SLATE patients S1-S3 were determined to have a KRAS G12C mutation. Vaccination of SLATE patient S1 did not result in a robust T cell response at the time points examined. In contrast, the priming GRT-C903 dose in SLATE patient S2 elicited a CD8 T cell immune response to a pool of KRAS G12C epitopes immediately (response seen Day 0, see FIG. 58A and Table 56B) demonstrating effective priming. In addition, the GRT-R904 boosting doses (30 μg) resulted in further increased CD8 T cell responses. For SLATE patient S3, the priming GRT-C903 dose elicited a CD8 T cell immune response to a pool of KRAS G12C epitopes as assessed at Week 20 (FIG. 58D). SLATE patient S4 was determined to have a KRAS Q61H mutation. The priming GRT-C903 dose in patient S4 elicited a CD8 T cell immune response by Week 2 when stimulated with the single Q61H peptide ILDTAGHEEY demonstrating priming, although responses when stimulated with a pool of KRAS Q61H epitopes was not observed (FIG. 58B). Following the GRT-R904 boosting dose in S4 (30 μg+SC ipilimumab), CD8 T cell responses were observed when stimulated with the Q61H pool. As shown in FIG. 58C, CD8 T cell immune responses were observed when stimulated the indicated G12C, Q61H, and G12V peptide pools. In addition, CD8 T cell responses to peptide stimulation using pools featuring TP53 mutations R213L, S127Y, and R249M (see Table 55B; each presented by different Class I HLA alleles) was also assessed demonstrating a robust, and potentially immunodominant, T cell response (FIG. 58E). Accordingly, the results demonstrate that the heterologous prime/boost protocol resulted in a T cell response to the vaccine-encoded subjects-specific neoantigens KRAS G12C, KRAS Q61H, and KRAS G12V, as well as additional encoded TP53 peptides R213L, S127Y, and R249M.

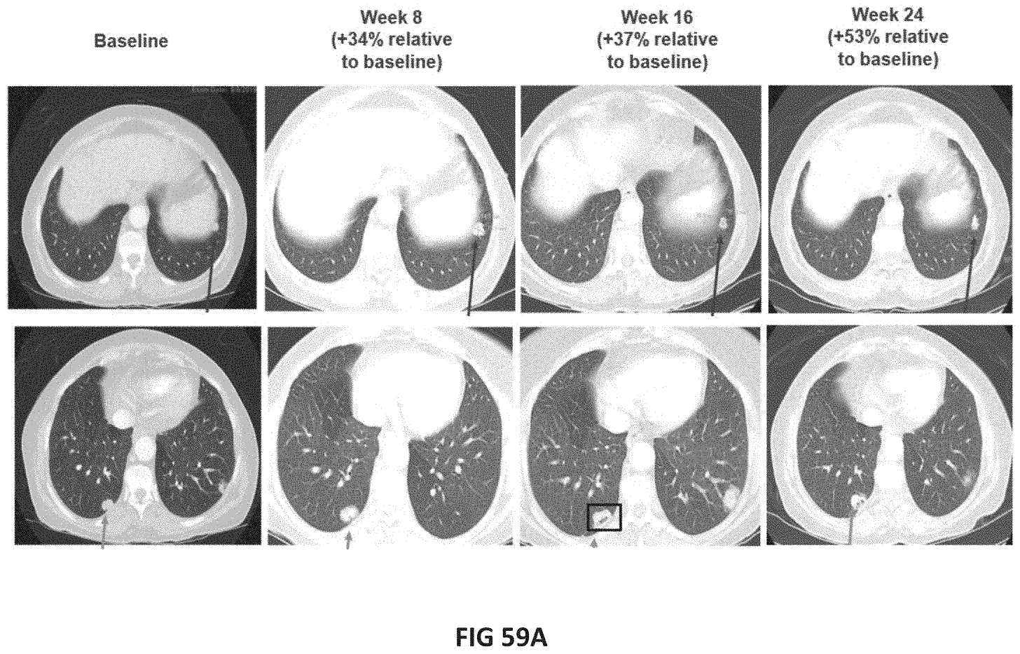

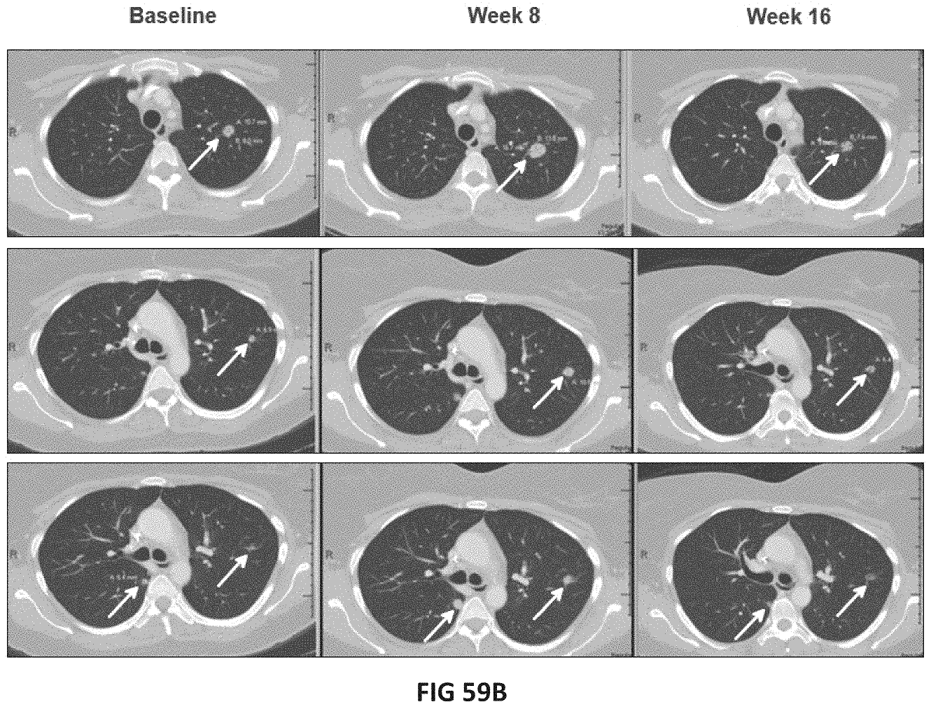

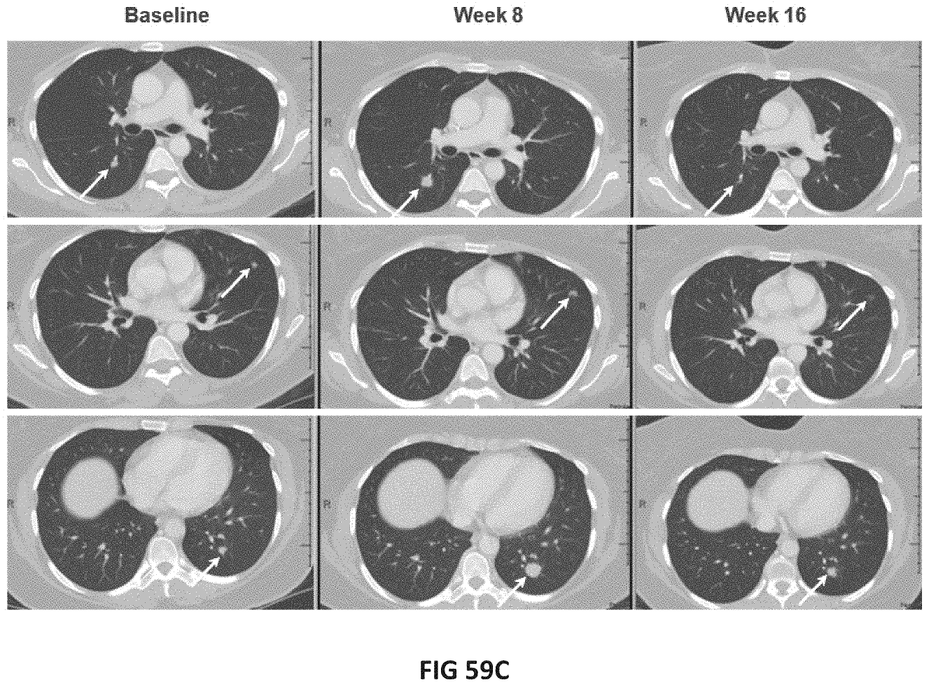

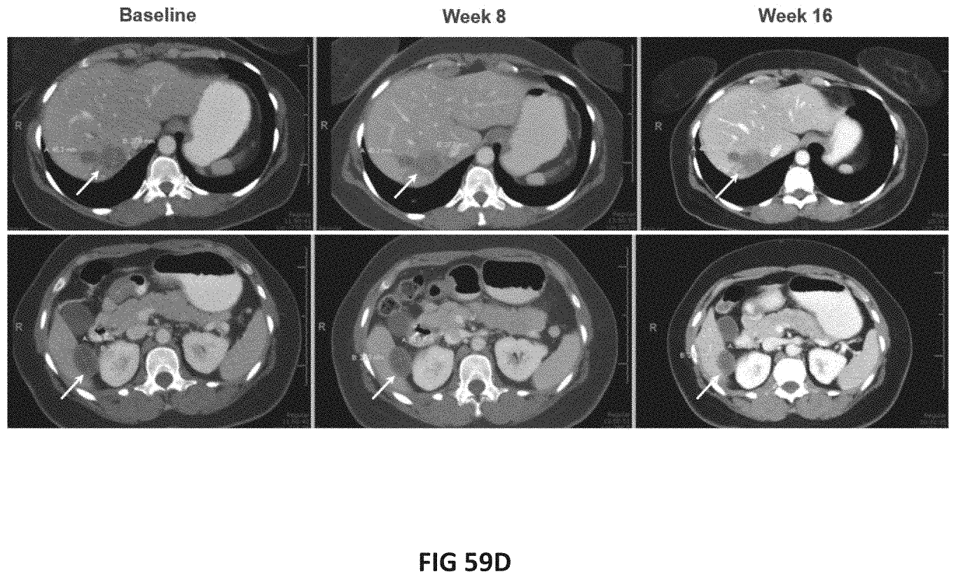

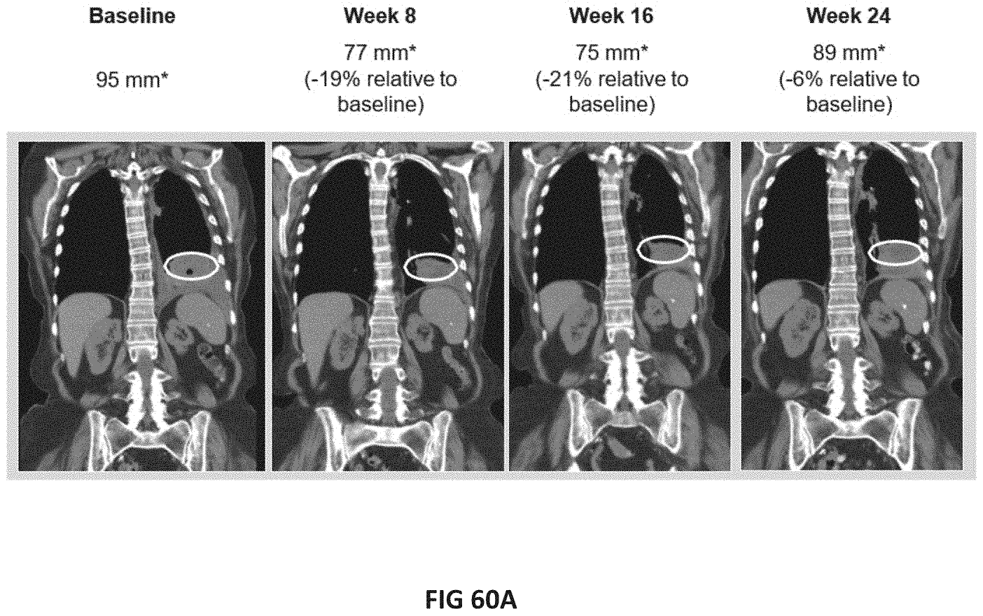

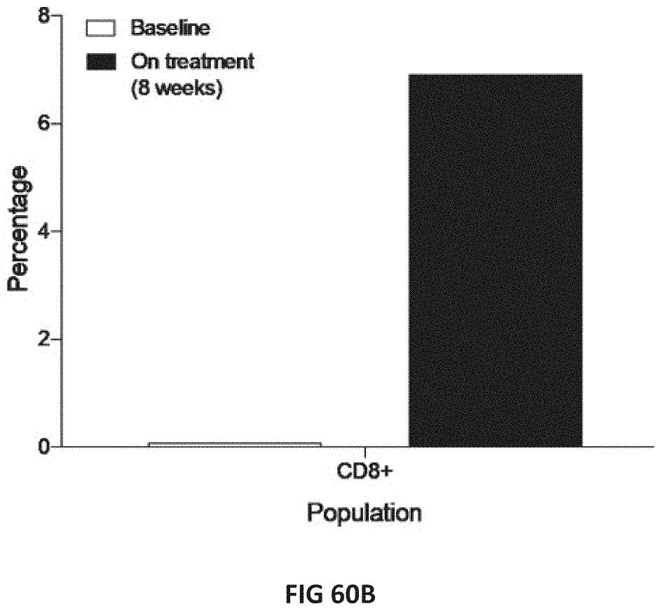

Disease progression and vaccine efficacy was further examined through radiological assessment of CT scans. As shown in FIG. 59A, compared to baseline (left panels), GRANITE patient G3's disease progressed at week 8 (second column of panels; +34% relative to baseline), stabilized by week 16 (third column of panels; +37% relative to baseline and +3% relative to week 8), and minimally progressed at week 24 (right panels; +53% relative to baseline), demonstrating a slow expansion of lung nodules with cavitation and no new lesions. As shown in FIGS. 59B and 59C, multiple lung lesions in patient G8 transiently expanded at week 8, potentially due to T cell infiltration (middle panels), then contracted at week 16 (right panels) relative to baseline (left panels). As shown in FIG. 59D, one liver lesion in patient G8 contracted (bottom panels), while another remained stable (top panels). As shown in FIG. 60A, SLATE patient S2 demonstrated a 19% reduction in tumor size at week 8 (second column panels) with a continued reduction at week 16 (third column of panels; −21% relative to baseline), then an increase that remained below baseline (right panels; −6% relative to baseline), as compared to baseline (left panels). In addition, SLATE patient S2 also demonstrated immune infiltration, particularly of CD8 T cells (FIG. 60B), in a post-treatment (week 8) biopsy as assessed by IHC (data not shown) for a panel of markers (CD4, CD8a, CD45RO, Granzyme B, FoxP3, PD-L1, CD68, Pan-Cytokeratin). Accordingly, the radiological assessment demonstrated efficacy of the vaccine therapy.

The clinical status of patients was also monitored. GRANITE subject G2 (who had received prior therapies of FOLFOXIRI for 2 months, followed by surgery, then FOLFOXIRI for another 2 months), was on the study for 365+ days then discontinued study treatment per patient request, during which no evidence of disease at any timepoint on study (post-surgery) was observed while CD8 T-cell expansion with 12 neoantigen-specific CD8 T-cell clones was observed. GRANITE subject G3 (who had received prior therapies of chemoradiation+durvalumab for 9 months but demonstrating progressive disease, followed by carboplatin+gemcitabine for 4 months leading to stable disease), was on the study for 180+ days symptomatically improving since study entry without any complaint compared to prior lines of treatment and treated beyond radiologic progression, during which tumor control was observed until T cell decline. GRANITE subject G8 (who had received prior therapies of FOLFOX/bev for 15 months but demonstrating progressive disease, followed by FOLFIRI/bev for 6 months), was on the study for 112+ days and clinically feeling well, during which stable disease was observed at week 16 (one liver lesion stable, all other lesions shrinking) as well as CD8 T-cell expansion with priming dose and a further expansion with the boosting dose. SLATE subject S2 (who had received prior therapies of Pembrolizumab for 4 months but demonstrating progressive disease, followed by Anti-TIGIT for 10 months leading to stable disease, then Carboplatin/pemetrexed/SBRT for 2 months), was on the study for 168 days then declined further treatment due to fatigue, during which stable disease with a 20% reduction from baseline was observed as well as an expansion of pre-existing CD8 T-cell against KRAS G12C. SLATE subject S3 (who had received prior therapies of Pembrolizumab+Carboplatin/pemetrexed for 8 months but demonstrating progressive disease), was on the study for 196+ and clinically doing well, during which stable disease with a 15% reduction from baseline was observed at weeks 24 and 32 as well as CD8 T cells against KRAS G12C following in vitro stimulation.

For GRANITE, the data overall demonstrate good tolerability at dose level 3 (100 μg+SC IPI) with evaluation of dose level 4 still in progress. Strong, consistent induction of killer CD8+ T cells, specific to multiple neoantigens, demonstrated which have been shown to accumulate in tumors of each of two patients studied. Dose level 1 (30 μg) efficacy data suggest induction of disease control that is also potentially more durable in an adjuvant-like context. Dose level 2 (100 μg) data suggest that development of T cell response may take multiple weeks (and thus, presumably, any consequent benefit), indicating patients about to progress and die within a few weeks may not benefit from this form of immunotherapy. Dose level 3 (100 μg+SC IPI) data demonstrated no observed disease progression as well as a clear clinical benefit ongoing in a metastatic colorectal cancer patient (MSS genotype).

For SLATE, the data overall demonstrate good tolerability at dose level 4 (300 μg+SC IPI). Induction of CD8+ T cells to KRAS neoantigens was observed, although inconsistent. Strong, consistent induction of CD8+ T cells to TP53 neoantigens was observed. Evidence of clinical benefit and minor but sustained tumor shrinkage in multiple NSCLC patients with KRAS G12C mutations who had progressed on prior CPI therapy was observed.

TABLE 49
Subjects Enrolled in Heterologous Prime/Boost Studies

| | GRANITE | SLATE |
|---|---|---|
| | (n = 10) | (n = 19) |
| Age (mean, raμge) | 59 (38-76) | 58 33-83) |
| Gender (Female/Male) | 4/6 | 12/7 |
| # of doses | | |
| ChAdV | 11 | 19 |
| SAM | 44 | 39 |
| Nivolumab (IV) | 61 | 58 |
| Ipilimumab (SC) | 14 | 47 |
| Tumor Types | | |
| NSCLC | 1 | 6 |
| Microsatellite stable (MSS)-CRC | 5 | 6 |
| Gastroesophageal adenocarcinoma (GEA) | 4 | 0 |
| Pancreatic ductal adenocarcinoma | N/A | 5 |
| Ovarian adenocarcinoma | N/A | 1 |
| Ampullary adenocarcinoma | N/A | 1 |
| Prior anti-PD(L)1 therapy | 1 | 6 |

TABLE 50A
Heterologous Prime/Boost Adverse Events Summary

| | GRANITE (n = 10) | | SLATE (n = 19) | |
|---|---|---|---|---|
| | Grade 1/2 | Grade 3/4 | Grade 1/2 | Grade 3/4 |
| Treatment-related | | | | |
| adverse events | | | | |
| Fever | 9 | 0 | 14 | 1 |
| Skin rash | 3 | 0 | 0 | 0 |
| Diarrhea | 2 | 0 | 4 | 0 |
| Fatigue | 4 | 0 | 9 | 0 |
| CK Elevation | 0 | 1^{a} | 0 | 0 |
| Injection-site | 7 | 0 | 1 | 0 |
| reactions | | | | |
| Myalgia | 2 | 0 | 2 | 0 |
| Pruritus | 1 | 0 | 4 | 0 |
| Anorexia | 2 | 0 | 1 | 0 |
| Chills | 1 | 0 | 3 | 0 |
| Congestion | 1 | 0 | 0 | 0 |
| Hot flashes | 1 | 0 | 0 | 0 |
| Hyperthyroidism | 0 | 1 | 0 | 0 |
| Hypotension | 1 | 0 | 0 | 0 |
| Infusion-related | 1 | 0 | 0 | 0 |
| reaction | | | | |
| Vomiting | 1 | 0 | 3 | 0 |
| Nausea | 0 | 0 | 4 | 0 |
| ALT increased | 0 | 0 | 0 | 1^{e} |
| AST increased | 0 | 0 | 0 | 1^{e} |
| Neutropenia | 0 | 0 | 0 | 1 |
| Thrombocytopenia | 0 | 0 | 0 | 1 |
| Rash | 0 | 0 | 2 | 0 |
| Arthralgia | 0 | 0 | 1 | 0 |
| Dizziness | 0 | 0 | 1 | 0 |
| Dry skin | 0 | 0 | 1 | 0 |
| Gait disturbance | 0 | 0 | 1 | 0 |
| Generalized | 0 | 0 | 1 | 0 |
| weakness | | | | |
| Headache | 0 | 0 | 1 | 0 |
| Insomnia | 0 | 0 | 1 | 0 |
| Myositis | 0 | 0 | 1 | 0 |
| Night Sweats | 0 | 0 | 1 | 0 |
| Rhabdomyolysis | 0 | 0 | 0 | 1 |
| Serious Adverse | | | | |
| Events (SAEs) | | | | |
| Fever | 2^{b} | 0 | 0 | 2^{b} |
| Cervical Fracture | 0 | 0 | 0 | 1^{c} |
| Heart Failure | 0 | 1^{c} | 0 | 0 |
| Abdominal pain | 1^{c} | 0 | 1^{c} | 0 |
| Bradycardia | 1^{c} | 0 | 0 | 0 |
| Hyperthyroidism | 0 | 1 | 0 | 0 |
| Respiratory Failure | 0 | 1^{d} | 0 | 0 |
| Acute kidney failure | 0 | 0 | 1^{c} | |
| Anemia | 0 | 0 | 1^{c} | |
| ALT elevation | 0 | 0 | 0 | 1^{e} |
| AST elevation | 0 | 0 | 0 | 1^{e} |
| Hypotension | 0 | 0 | 1^{c} | |
| Rhabdomyolysis | 0 | 0 | 0 | 1 |
| Myositis | 0 | 0 | 1 | 0 |
| Nausea | 0 | 0 | 2^{c} | 0 |
| Neutropenia | 0 | 0 | 0 | 1 |
| Right Frontal Brain | 0 | 0 | 1^{c} | 0 |
| Metastasis | | | | |
| Small bowel | 0 | 0 | 1^{c} | 0 |
| obstruction | | | | |
| Vomiting | 0 | 0 | 1^{c} | 0 |

^{a}Self-limiting, asymptomatic increase in creatine kinase
^{b}Both SAEs of fever occurring in the same patient
^{c}Not treatment-related
^{d}Grade 5 respiratory failure resulted from checkpoint inhibitor-induced acute thyrotoxicosis, with associated aspiration pneumonia, respiratory failure leading to patient death and is not related to vaccine treatment
^{e}attributed to nivolumab

TABLE 50B
GRANITE Treatment-related adverse events (TRAEs)

| | n = 10 (all patients treated with concurrent nivolumab) | | | | | |
|---|---|---|---|---|---|---|
| | | | SAM | | | |
| | ChAdV (1 × 10¹² vp) | | | | 100 ug + | 300 ug + |
| | No SC IPI | With SC IPI | 30 ug | 100 ug^{a} | SC IPI | SC IPI |
| Safety | (n = 5) | (n = 5) | (n = 3) | (n = 2) | (n = 2) | (n = 2) |

Treatment-related adverse events

| | | | | | | |
|---|---|---|---|---|---|---|
| Fever | 4 | 3 | 1 | — | 1 | — |
| Injection-site reactions | 1 | 4 | 1 | 1 | — | — |
| Fatigue | 1 | 1 | 1 | | 1 | — |
| Skin rash | 1 | — | 1 | | 1 | — |
| Anorexia | — | 1 | — | — | 1 | — |
| Chills | — | — | — | — | 1 | — |
| CK Elevation | — | — | 1 | — | — | — |
| Congestion | — | 1 | | — | — | — |
| Diarrhea | — | — | 1 | — | 1 | — |
| Hot flashes | — | 1 | — | — | | — |
| Hyperthyroidism | — | — | — | — | 1 | — |
| Hypotension | — | — | 1 | — | — | — |
| Infusion-related reaction | — | — | — | 1 | — | — |
| Myalgia | — | 1 | — | 1 | — | — |
| Pruritus | — | — | 1 | — | — | — |
| Vomiting | — | — | — | | 1 | — |

^{a}One patient did not receive SAM boosts

TABLE 50C
GRANITE serious adverse events (TRAEs)

| | n = 10 (all patients treated with concurrent nivolumab) | | | | | |
|---|---|---|---|---|---|---|
| | | | SAM | | | |
| | ChAdV (1 × 10¹² vp) | | | | | 300 ug + |
| | No SC IPI | With SC IPI | 30 ug | 100 ug^{a} | 100 ug + SC IPI | SC IPI |
| Safety | (n = 5) | (n = 5) | (n = 3) | (n = 2) | (n = 2) | (n = 2) |

SAEs

| | | | | | | |
|---|---|---|---|---|---|---|
| Abdominal paine | 1^{e} | — | | — | — | — |
| Fever | 1^{b} | — | 1^{b} | — | — | — |
| Bradycardia | — | — | 1^{c,2} | — | — | — |
| Heart Failure | — | — | 1^{c,2} | — | — | — |
| Hyperthyroidism | — | — | — | — | 1^{d} | — |
| Respiratory Failure | — | — | — | — | 1^{d,e} | — |

^{a}One patient did not receive SAM boosts
^{b}Occurring in the same patient
^{c}Occurring in the same patient
^{d}Occurring in the same patient
^{e}Not treatment-related

TABLE 50D

| SLATE Treatment-related adverse events (TRAEs) | | | | | | |
|---|---|---|---|---|---|---|
| | n = 19 (all patients treated with concurrent nivolumab) | | | | | |
| | | | SAM | | | |
| | ChAdV (1 × 10¹² vp) | | | 30 ug + | 100 ug + | 300 ug + |
| | No SC IPI | With SC IPI | 30 ug | SC IPI | SC IPI | SC IPI |
| Safety | (n = 2) | (n = 17) | (n = 2) | (n = 4) | (n = 6) | (n = 6^{a}) |

Treatment-related adverse events

| | | | | | | |
|---|---|---|---|---|---|---|
| Fever | — | 7 | — | 1 | 2 | 5 |
| Fatigue | — | 6 | — | — | 1 | 2 |
| Diarrhea | — | 2 | — | — | 1 | 1 |
| Nausea | — | 2 | — | — | 1 | 1 |
| Pruritus | 1 | 1 | — | 1 | 1 | — |
| Chills | — | 2 | — | — | — | 1 |
| Vomiting | — | 2 | — | — | — | 1 |
| LALT increased | — | 1 | — | — | — | 1 |
| AST increased | — | 1 | — | — | — | 1 |
| Myalgia | — | 2 | — | — | — | — |
| Neutropenia | — | 1 | — | — | — | 1 |
| Rash | — | — | 1 | — | 1 | — |
| Anorexia | — | — | — | 1 | — | — |
| Arthralgia | — | 1 | — | — | — | — |
| Dizziness | — | — | 1 | — | — | — |
| Dry skin | — | — | 1 | — | — | — |
| Gait disturbance | — | — | 1 | — | — | — |
| Generalized weakness | — | — | 1 | — | — | — |
| Headache | — | 1 | — | — | — | — |
| Injection site reaction | — | 1 | — | — | — | — |
| Insomnia | — | 1 | — | — | — | — |
| Myositis | — | 1 | — | — | — | — |
| Night Sweats | — | 1 | — | — | — | — |
| Rhabdomyolysis | — | 1 | — | — | — | — |
| Thrombocytopenia | — | — | — | — | — | 1 |

^{a}1 patient discontinued after ChAdV due to autoimmune myositis

TABLE 50E
SLATE serious adverse events (SAEs)

| | Safetyn = 19 (all patients treated with concurrent nivolumab) | | | | | |
|---|---|---|---|---|---|---|
| | | | SAM | | | |
| | ChAdV (1 × 10¹² vp) | | | 30 ug + | 100 ug + | 300 ug + |
| | No SC IPI | With SC IPI | 30 ug | SC IPI | SC IPI | SC IPI |
| | (n = 2) | (n = 17) | (n = 2) | (n = 4) | (n = 6) | (n = 6^{f}) |
| SAEs | | | | | | |
| Abdominal pain | — | — | — | — | § | 1^{a} |
| Acute kidney failure | — | 1^{a,b} | — | — | — | — |
| Anemia | — | — | — | 1^{a,f} | — | — |
| ALT elevation | — | 1^{c} | — | — | — | — |
| AST elevation\| | — | 1^{c} | — | — | — | — |
| Cervical fracture | — | — | 1^{a} | — | — | — |
| Fever | — | 1^{c} | — | — | — | 1^{c,g} |
| Hypotension | — | — | — | 1^{a,f} | — | — |
| Myositis | — | 1^{c} | — | — | — | — |
| Nausea | — | — | — | 1^{a,b} | 1^{a,d} | — |
| Neutropenia | — | — | — | — | — | 1^{c,g} |
| Right Frontal Brain Metastasis | — | 1^{a} | — | — | — | — |
| Rhabdomyolysis | — | 1^{c} | — | — | — | — |
| Small bowel obstruction | — | — | — | 1^{a,b} | — | — |
| Vomiting | — | — | — | — | 1^{a,d} | — |

^{a}Not treatment-related
^{b}Occurring in the same patient
^{c}Occurring in the same patient
^{d}Occurring in the same patient
^{e}Occurring in the same patient
^{f}Occurring in the same patient
^{g}One patient not treated with nivolumab or ipilimumab in combination with SAM

TABLE 51A
GRANITE Patient G1 Peptides in
Restimulation Assay

| Peptide sequence | | |
|---|---|---|
| (amino acids) | Peptide pools | |
| TVAEFSHQC | CD8 Pool | Mini 1 |
| VLTAILSSA | CD8 Pool | Mini 1 |
| FIMTLNTSL | CD8 Pool | Mini 1 |
| VDIDVPDVNF | CD8 Pool | Mini 1 |
| FEAPEGKL | CD8 Pool | Mini 1 |
| FEAPEGKLK | CD8 Pool | Mini 1 |
| LSISYLREY | CD8 Pool | Mini 1 |
| IEMVNRGQIQI | CD8 Pool | Mini 1 |
| IDVPDVNF | CD8 Pool | Mini 1 |
| IEMVNRGQI | CD8 Pool | Mini 1 |
| EHDEALKNTW | CD8 Pool | Mini 1 |
| EEHDEALKNTW | CD8 Pool | Mini 1 |
| NQIDTLQEV | CD8 Pool | Mini 2 |
| SLFHQSEVEL | CD8 Pool | Mini 2 |
| YIVGANTETYL | CD8 Pool | Mini 2 |
| IVGANTETY | CD8 Pool | Mini 2 |
| YIVGANTETY | CD8 Pool | Mini 2 |
| GEDCLTSQTRL | CD8 Pool | Mini 2 |
| NESPGNNVV | CD8 Pool | Mini 2 |
| TEILHSHVV | CD8 Pool | Mini 2 |
| AEVDVREREI | CD8 Pool | Mini 2 |
| SDELAQLY | CD8 Pool | Mini 2 |
| NESPGNNVVDI | CD8 Pool | Mini 2 |
| DELVTFSDEL | CD8 Pool | Mini 2 |
| DEFIQANA | CD8 Pool | Mini 2 |
| DELAQLYHH | CD8 Pool | Mini 2 |
| DEFIQANAT | CD8 Pool | Mini 2 |
| KLPEADVAVL | CD8 Pool | Mini 3 |
| PEADVAVL | CD8 Pool | Mini 3 |
| ADVAVLRTF | CD8 Pool | Mini 3 |
| PEADVAVLRTF | CD8 Pool | Mini 3 |
| DVAVLRTF | CD8 Pool | Mini 3 |
| EADVAVLRTF | CD8 Pool | Mini 3 |
| DEYATLMM | CD8 Pool | Mini 3 |
| GLTEVIQSL | CD8 Pool | Mini 4 |
| QVLHLMVGV | CD8 Pool | Mini 4 |
| VLHLMVGV | CD8 Pool | Mini 4 |
| HEMLQVLHL | CD8 Pool | Mini 4 |
| TEVIQSLKM | CD8 Pool | Mini 4 |
| AEQKGLTEVI | CD8 Pool | Mini 4 |

TABLE 51B
GRANITE Patient G2 Peptides
in Restimulation Assay

| Peptide sequence | | |
|---|---|---|
| (amino acids) | Peptide pools | |
| ALFGSALVPTG | CD8 Pool | Mini 1 |
| ALFGSALVPT | CD8 Pool | Mini 1 |
| GVSSFHVTW | CD8 Pool | Mini 1 |
| SRPPHEPKF | CD8 Pool | Mini 1 |
| TGVSSFHVTW | CD8 Pool | Mini 1 |
| VSSFHVTW | CD8 Pool | Mini 1 |
| QSRPPHEPKF | CD8 Pool | Mini 1 |
| SRPPHEPKFV | CD8 Pool | Mini 1 |
| VTGVSSFHVTW | CD8 Pool | Mini 1 |
| VFPTRTAL | CD8 Pool | Mini 1 |
| QLASLQQEL | CD8 Pool | Mini 2 |
| SLNPAVAKA | CD8 Pool | Mini 2 |
| SLQQELAAL | CD8 Pool | Mini 2 |
| VLYKGEARV | CD8 Pool | Mini 2 |
| KLNNDLNEL | CD8 Pool | Mini 2 |
| RLEDQLASL | CD8 Pool | Mini 2 |
| VLYKGEARVV | CD8 Pool | Mini 2 |
| YAANSIGAV | CD8 Pool | Mini 2 |
| RVGNESLNPAV | CD8 Pool | Mini 2 |
| NLYAANSIGA | CD8 Pool | Mini 2 |
| KPMERSSVL | CD8 Pool | Mini 2 |
| SGASVNFDY | CD8 Pool | Mini 2 |
| YKGEARVV | CD8 Pool | Mini 2 |
| LYKGEARVV | CD8 Pool | Mini 2 |
| HDSIKAAL | CD8 Pool | Mini 2 |
| RVVLLKAAY | CD8 Pool | Mini 2 |
| GSVSTIRQY | CD8 Pool | Mini 3 |
| KTFPAAIW | CD8 Pool | Mini 3 |
| ILADGPAVEV | CD8 Pool | Mini 4 |
| LILADGPAV | CD8 Pool | Mini 4 |
| LLMGTEDAL | CD8 Pool | Mini 4 |
| KLADFVKYY | CD8 Pool | Mini 4 |
| KLADFVKYYY | CD8 Pool | Mini 4 |
| FYGPPQKI | CD8 Pool | Mini 4 |
| STLLAGNLRI | CD8 Pool | Mini 4 |
| TQKAKVGKL | CD8 Pool | Mini 4 |
| LPLTTARYSTL | CD8 Pool | Mini 4 |
| KAIINIFFF | CD8 Pool | Mini 4 |
| GTVAKKVAW | CD8 Pool | Mini 4 |
| TVAKKVAW | CD8 Pool | Mini 4 |

TABLE 51C
GRANITE Patient G3 Peptides
in Restimulation Assay

| Peptide sequence | | |
|---|---|---|
| (amino acids) | Peptide pools | |
| LVEKSEAVRK | CD8 Pool | Mini 1 |
| ASMGTLAFHEY | CD8 Pool | Mini 1 |
| ASMGTLAFH | CD8 Pool | Mini 1 |
| MGTLAFHEY | CD8 Pool | Mini 1 |
| GTLAFHEY | CD8 Pool | Mini 1 |
| ATNQGWMLR | CD8 Pool | Mini 1 |
| GTMSLPASV | CD8 Pool | Mini 1 |
| RVPPKDLRVK | CD8 Pool | Mini 1 |
| DTLISQESFEK | CD8 Pool | Mini 1 |
| ISQESFEKK | CD8 Pool | Mini 1 |
| TLISQESFEK | CD8 Pool | Mini 1 |
| ISQESFEK | CD8 Pool | Mini 1 |
| FLKNENNEL | CD8 Pool | Mini 1 |
| SPVVETVPA | CD8 Pool | Mini 1 |
| LEPPSLPAQR | CD8 Pool | Mini 1 |
| MAMLIYTDF | CD8 Pool | Mini 2 |
| AMLIYTDFL | CD8 Pool | Mini 2 |
| SPSALIQEM | CD8 Pool | Mini 2 |
| SDWPPPNTR | CD8 Pool | Mini 2 |
| DAIDGRQAR | CD8 Pool | Mini 2 |
| MLAGIPVTI | CD8 Pool | Mini 2 |
| GMLAGIPVTI | CD8 Pool | Mini 2 |
| GMLAGIPVT | CD8 Pool | Mini 2 |
| TLYLLDIKV | CD8 Pool | Mini 2 |
| SFVAVTEEGTK | CD8 Pool | Mini 2 |
| FVAVTEEGTK | CD8 Pool | Mini 2 |
| YLSPPCIHC | CD8 Pool | Mini 3 |
| ARLGLTEVY | CD8 Pool | Mini 3 |
| FLEERLARL | CD8 Pool | Mini 3 |
| RLARLGLTEV | CD8 Pool | Mini 3 |
| LARLGLTEVY | CD8 Pool | Mini 3 |
| LARLGLTEV | CD8 Pool | Mini 3 |
| SLLRVLVTL | CD8 Pool | Mini 3 |
| VLVTLNDLEPK | CD8 Pool | Mini 3 |
| VLDHTLPTA | CD8 Pool | Mini 4 |
| TVLDHTLPTA | CD8 Pool | Mini 4 |
| LPTALTSPEL | CD8 Pool | Mini 4 |
| TVLDHTLPT | CD8 Pool | Mini 4 |
| TVLDHTLPTAL | CD8 Pool | Mini 4 |
| VLDHTLPTAL | CD8 Pool | Mini 4 |

TABLE 51D
GRANITE Patient G4 Peptides
in Restimulation Assay

| Peptide sequence | | |
|---|---|---|
| (amino acids) | Peptide pools | |
| YEAVAVAA | CD8 Pool | Mini 1 |
| AVAAAASAY | CD8 Pool | Mini 1 |
| YEAVAVAAA | CD8 Pool | Mini 1 |
| AVAVAAAASAY | CD8 Pool | Mini 1 |
| AVAAAASAYNY | CD8 Pool | Mini 1 |
| RVRSYEAVAV | CD8 Pool | Mini 1 |
| SYEAVAVAA | CD8 Pool | Mini 1 |
| VAAAASAY | CD8 Pool | Mini 1 |
| KTFPNVPRAAR | CD8 Pool | Mini 1 |
| VAVAAAASAY | CD8 Pool | Mini 1 |
| SYEAVAVA | CD8 Pool | Mini 1 |
| RVHVPGKTL | CD8 Pool | Mini 1 |
| SEASLVPLAY | CD8 Pool | Mini 2 |
| EASLVPLAY | CD8 Pool | Mini 2 |
| SEASLVPLA | CD8 Pool | Mini 2 |
| ASLVPLAY | CD8 Pool | Mini 2 |
| SEASLVPL | CD8 Pool | Mini 2 |
| IQAQPENIPAK | CD8 Pool | Mini 2 |
| DSEASLVPLAY | CD8 Pool | Mini 2 |
| ELFPDRASSSW | CD8 Pool | Mini 3 |
| HTQNWSTTW | CD8 Pool | Mini 3 |
| GELIFCGM | CD8 Pool | Mini 3 |
| GQHIDIQMK | CD8 Pool | Mini 3 |
| DEFVKHKY | CD8 Pool | Mini 4 |
| TDEFVKHKY | CD8 Pool | Mini 4 |
| GTQPHTATR | CD8 Pool | Mini 4 |
| ITDEFVKHK | CD8 Pool | Mini 4 |
| GTQPHTATRL | CD8 Pool | Mini 4 |
| HLNLGILYY | CD8 Pool | Mini 4 |
| VTLKVLMADK | CD8 Pool | Mini 4 |
| TDTHIPGSPF | CD8 Pool | Mini 4 |
| HLNLGILY | CD8 Pool | Mini 4 |
| QLPQPGAQH | CD8 Pool | Mini 4 |
| KLITDEFVKHK | CD8 Pool | Mini 4 |
| LPEIINLY | CD8 Pool | Mini 4 |
| VTMEDLIQPW | CD8 Pool | Mini 4 |
| TLKVLMADK | CD8 Pool | Mini 4 |
| MVWGTGNPR | CD8 Pool | Mini 4 |
| DSSPRLAVDF | CD8 Pool | Mini 4 |
| ESGSMVNHL | CD8 Pool | Mini 4 |

TABLE 51E
GRANITE Patient G7 Peptides
in Restimulation Assay

| Peptide sequence | | |
|---|---|---|
| (amino acids) | Peptide pools | |
| SSSSVLNNK | CD8 Pool | Mini 1 |
| SQSSSSVLNNK | CD8 Pool | Mini 1 |
| HVNGGPLYSK | CD8 Pool | Mini 1 |
| QSSSSVLNNK | CD8 Pool | Mini 1 |
| QTHVKGSLK | CD8 Pool | Mini 1 |
| ENAPPILPR | CD8 Pool | Mini 1 |
| VIFKGEIGK | CD8 Pool | Mini 1 |
| GSYSGTTILH | CD8 Pool | Mini 1 |
| VVVGADGVGK | CD8 Pool | Mini 1 |
| VNGGPLYSK | CD8 Pool | Mini 1 |
| SMRDMNITK | CD8 Pool | Mini 2 |
| TLLNLIQTK | CD8 Pool | Mini 2 |
| STLLNLIQTK | CD8 Pool | Mini 2 |
| YIDNALSRL | CD8 Pool | Mini 2 |
| TSRGQVSER | CD8 Pool | Mini 2 |
| SMRDMNITKL | CD8 Pool | Mini 2 |
| VGKSDSVDAEK | CD8 Pool | Mini 2 |
| TYIDNALSR | CD8 Pool | Mini 2 |
| KSDSVDAEK | CD8 Pool | Mini 2 |
| NLIQTKVNYV | CD8 Pool | Mini 2 |
| LLNLIQTK | CD8 Pool | Mini 2 |
| DSVDAEKSMLS | CD8 Pool | Mini 2 |
| DLRTSQSVR | CD8 Pool | Mini 3 |
| VLLTGTPLQK | CD8 Pool | Mini 3 |
| KVLLTGTPLQK | CD8 Pool | Mini 3 |
| LLTGTPLQK | CD8 Pool | Mini 3 |
| NFDLRTSQSVR | CD8 Pool | Mini 3 |
| QVYSKALNR | CD8 Pool | Mini 4 |
| LLYKAGGVK | CD8 Pool | Mini 4 |
| NFDVHHTAM | CD8 Pool | Mini 4 |
| DSPHKEPIRLR | CD8 Pool | Mini 4 |
| SLFLAVVAR | CD8 Pool | Mini 4 |
| AVSSCFRQL | CD8 Pool | Mini 4 |
| SSSSSLFL | CD8 Pool | Mini 4 |
| SSSSSSLFL | CD8 Pool | Mini 4 |
| DGAVSSCFR | CD8 Pool | Mini 4 |
| SSSSLFLAV | CD8 Pool | Mini 4 |
| LFLAVVAR | CD8 Pool | Mini 4 |
| QVYSKALNRL | CD8 Pool | Mini 4 |
| DVHHTAMLTR | CD8 Pool | Mini 4 |

TABLE 51F
GRANITE Patient G8 Peptides
in Restimulation Assay

| Peptide sequence | | |
|---|---|---|
| (amino acids) | Peptide pools | |
| SATKAVKPK | CD8 Pool | Mini 4 |
| FVDEVEKVM | CD8 Pool | Mini 2 |
| KSATKAVKPK | CD8 Pool | Mini 4 |
| ATAATLLPH | CD8 Pool | Mini 1 |
| TRLPLPLLL | CD8 Pool | Mini 3 |
| SVFLHQMKK | CD8 Pool | Mini 2 |
| GLNSRGKLVQV | CD8 Pool | Mini 2 |
| SLNEAYGYQI | CD8 Pool | Mini 3 |
| LLAELRSLNEA | CD8 Pool | Mini 3 |
| MMEEVYQTL | CD8 Pool | Mini 1 |
| ATKAVKPK | CD8 Pool | Mini 4 |
| KSATKAVKP | CD8 Pool | Mini 4 |
| NPAMTSELL | CD8 Pool | Mini 4 |
| ILQRIIVEL | CD8 Pool | Mini 1 |
| KLTQGETLCK | CD8 Pool | Mini 4 |
| FTQASGMTPA | CD8 Pool | Mini 1 |
| SLITAITTNR | CD8 Pool | Mini 2 |
| PAMTSELL | CD8 Pool | Mini 4 |
| REYTEDGQVTK | CD8 Pool | Mini 1 |
| ETSKILLRK | CD8 Pool | Mini 2 |
| FVIPLDVDEV | CD8 Pool | Mini 3 |
| YVWDETVRA | CD8 Pool | Mini 2 |
| GLLAELRSL | CD8 Pool | Mini 3 |
| LITAITTNR | CD8 Pool | Mini 2 |
| NPAMTSEL | CD8 Pool | Mini 4 |
| YVWDETVRAA | CD8 Pool | Mini 2 |
| ATRLPLPLLL | CD8 Pool | Mini 3 |
| ITAITTNR | CD8 Pool | Mini 2 |
| AMTSELLRA | CD8 Pool | Mini 4 |
| ALWYTLDRL | CD8 Pool | Mini 3 |
| NQRPILTII | CD8 Pool | Mini 1 |
| RPPQHLGGLK | CD8 Pool | Mini 3 |
| KSATKAVK | CD8 Pool | Mini 4 |
| DVDEVPPGV | CD8 Pool | Mini 3 |
| GMNQRPILT | CD8 Pool | Mini 1 |
| GGMNQRPIL | CD8 Pool | Mini 1 |
| TTETSKILLRK | CD8 Pool | Mini 2 |
| TQASGMTPA | CD8 Pool | Mini 1 |
| YTEDGQVTK | CD8 Pool | Mini 1 |
| TSRPPQHLGGL | CD8 Pool | Mini 3 |

TABLE 52A
GRANITE Patient G1 ELISpot

| Time- | Dose | | | | | | |
|---|---|---|---|---|---|---|---|
| point | administered | Vehicle | | | CD8 Pool | | |
| Day 0 | ChAdV Prime | 5 | 5 | 0 | 20 | 0 | 35 |
| Day 14 | | 5 | 0 | 0 | 15 | 35 | 15 |
| Day 28 | SAM Boost 1 | 10 | 10 | 0 | 105 | 160 | 135 |
| Day 35 | | 0 | 0 | 6.9 | 409.7 | 229.2 | 284.7 |
| Day 42 | | 6.9 | 0 | 0 | 298.6 | 284.7 | 361.1 |
| Day 56 | SAM Boost 2 | 13.9 | 0 | 0 | 256.9 | 284.7 | 381.9 |
| Day 84 | SAM Boost 3 | 0 | 0 | 0 | 812.5 | 770.8 | 680.6 |
| Day 91 | | | | | | | |
| Day 112 | SAM Boost 4 | 18.9 | 0 | 0 | 2232.5 | 2081.2 | 1873.1 |
| Day 120 | | | | | | | |
| Day 140 | SAM Boost 5 | 9.5 | 0 | | 813.6 | 1059.5 | |
| | (no | | | | | | |
| | Nivolumab) | | | | | | |
| Day 148 | | | | | | | |
| Day 168 | SAM Boost 6 | 9.5 | 9.5 | 9.5 | 1267.6 | 1381.1 | 1277.1 |
| | (no | | | | | | |
| | Nivolumab) | | | | | | |

TABLE 52B
GRANITE Patient G2 ELISpot

| Timepoint | Dose administered | Vehicle | | | CD8 Pool | | |
|---|---|---|---|---|---|---|---|
| Day 0 | ChAdV Prime | 0 | 0 | 0 | 15 | 5 | 0 |
| Day 14 | | 0 | 0 | 0 | 355 | 445 | 430 |
| Day 28 | SAM Boost 1 | 0 | 0 | 0 | 1065 | 1120 | 905 |
| Day 35 | | 0 | 0 | 0 | 2579.5 | 2706 | 2512.1 |
| Day 42 | | 8.4 | 0 | 0 | 1424.6 | 1584.8 | 1500.5 |
| Day 56 | | 0 | 0 | 0 | 1837.7 | 1896.7 | 1711.3 |
| Day 84 | | 0 | 0 | 0 | 2689.1 | 2630.1 | 2672.3 |
| Day 91 | SAM Boost 2 | 0 | 0 | 0 | 1079 | 1062.2 | 1062.2 |
| Day 112 | | | | | | | |
| Day 120 | SAM Boost 3 | 0 | 0 | 0 | 1331.9 | 1087.5 | 1180.2 |
| Day 140 | | | | | | | |
| Day 148 | SAM Boost 4 | 0 | 0 | 0 | 1083.1 | 1039.5 \| | 1003.1 |
| Day 168 | | | | | | | |
| Day 183 | SAM Boost 5 | 0 | 0 | 0 | 1112.2 | 1461.1 | 1221.2 |

TABLE 52C
GRANITE Patient G3 ELISpot

| Timepoint | Dose administered | Vehicle | | | CD8 Pool | | |
|---|---|---|---|---|---|---|---|
| Day 0 | ChAdV Prime | 25 | 20 | | 55 | 80 | |
| Day 14 | | 20 | | | 50 | | |
| Day 28 | SAM Boost 1 | 0 | 0 | 5 | 165 | 205 | 125 |
| Day 35 | | 0 | 5.5 | | 170.2 | 153.8 | |
| Day 42 | | | | | | | |
| Day 56 | SAM Boost 2 | 0 | 11 | | 153.8 | 137.3 | |
| Day 84 | SAM Boost 3 | 5.5 | | 11 | 71.4 | 137.3 | 197.7 |

TABLE 52D
GRANITE Patient G4 ELISpot

| Timepoint | Dose administered | Vehicle | | | CD8 Pool | | |
|---|---|---|---|---|---|---|---|
| Day 0 | ChAdV Prime | 0 | 5 | 0 | 25 | 5 | 15 |
| Day 14 | | 0 | 0 | 0 | 40 | 55 | 35 |
| Day 28 | SAM Boost 1 | 15 | 0 | 0 | 230 | 220 | 220 |

TABLE 53A
IL-2 and Granzyme B Production by Peptide Stimulated T Cells

| Sample | IL-2 | | | Granzyme B | | |
|---|---|---|---|---|---|---|
| Patient G1— | 0.97 | 0.46 | 2.37 | 8.77 | 11.54 | 13.23 |
| Day 0 | | | | | | |
| Patient G1— | 42.96 | 31.58 | 30.67 | 28.82 | 55.32 | 55.15 |
| Day 35 | | | | | | |
| Patient G2— | 0.00 | 7.24 | 0.90 | 0.00 | 14.19 | 0.00 |
| Day 0 | | | | | | |
| Patient G2— | 42.30 | 46.17 | 35.35 | 20.94 | 21.99 | 12.38 |
| Day 35 | | | | | | |

Replicate values for each cytokine and each timepoint are shown. Data are background subtracted based on vehicle control. Cytokine levels in supernatants (pg/ml)

TABLE 53B
IFN-gamma Production by Peptide Stimulated T Cells

| Sample | Vehicle | | | Mini 1 | | Mini 2 | | Mini 3 | | Mini 4 | |
|---|---|---|---|---|---|---|---|---|---|---|---|
| Pat G1 - Day 0 | 5 | 5 | 0 | 0 | | 20 | 10 | 5 | 10 | 5 | 10 |
| Pat Gl - Day 84 | 0 | 0 | 0 | 930.6 | 930.6 | 319.4 | 333.3 | 208.3 | 208.3 | 534.7 | 590.3 |
| Pat G2 - Day 0 | 0 | 0 | 0 | 5 | 0 | 0 | 0 | 5 | 5 | 0 | 5 |
| Pat G2 - Day 84 | 0 | 0 | 0 | 767.1 | 767.1 | 1180.2 | 1374.1 | 143.3 | 160.2 | 1593.2 | 1483.7 |

Replicate values for each stimulation condition and each timepoint are shown as Interferon-gamma spot forming units per million peripheral blood mononuclear cells as measured by ELISpot assay. Values from different assay batches are normalized to batch 1 based on bridging samples stimulated with CMV-EBV-Flu peptide pools.

TABLE 54
Expansion of de novo and pre-existing T cells subsets

| Patient | Stimulus | Day 0 | | | Day 112 | | |
|---|---|---|---|---|---|---|---|
| | CD8 Pool | 1350 | 1780 | | 10000 | 10000 | |
| G1 | Mini 1 | 910 | 970 | | 7280 | 6600 | |
| | Mini 2 | 650 | 660 | | 4980 | 4350 | |
| | Mini 3 | 170 | 180 | | 4060 | | |
| Patient | Stimulus | Day 0 | | | Day 14 | | |
| | CD8 Pool | 2830 | 3220 | 2780 | 10000 | 10000 | 10000 |
| | Mini 1 | 320 | 570 | | 5530 | 5790 | |
| G2 | Mini 2 | 2960 | 2240 | | 8130 | 7590 | |
| | Mini 3 | 370 | 340 | | 2400 | 3680 | |
| | Mini 4 | 210 | 290 | | 8730 | 7490 | |

Replicate values for each stimulation condition and each timepoint are shown as Interferon-gamma spot forming units per million peripheral blood mononuclear cells as measured by post-IVS ELISpot assay. Pool 4 for patient 1 not tested due to insufficient PBMC numbers. IFNgamma spot forming units per million PBMCs

TABLE 55A
SLATE KRAS Peptide Pools in Restimulation Assay

| KRAS G12C | KRAS Q61H | KRAS G12V |
|---|---|---|
| Peptides | Peptides | Peptides |
| KLVWGAC | DILDTAGH | KLVVVGAV |
| LVVVGACG | ILDTAGHE | LVVVGAVG |
| VVVGACGV | LDTAGHEE | VVVGAVGV |
| VVGACGVG | DTAGHEEY | VVGAVGVG |
| VGACGVGK | TAGHEEYS | VGAVGVGK |
| GACGVGKS | AGHEEYSA | GAVGVGKS |
| ACGVGKSA | GHEEYSAM | AVGVGKSA |
| CGVGKSAL | HEEYSAMR | VGVGKSAL |
| YKLVVVGAC | LDILDTAGH | YKLVVVGAV |
| KLVVVGACG | DILDTAGHE | KLVVVGAVG |
| LVVVGACGV | ILDTAGHEE | LVVVGAVGV |
| VVVGACGVG | LDTAGHEEY | VVVGAVGVG |
| VVGACGVGK | DTAGHEEYS | VVGAVGVGK |
| VGACGVGKS | TAGHEEYSA | VGAVGVGKS |
| GACGVGKSA | AGHEEYSAM | GAVGVGKSA |
| ACGVGKSAL | GHEEYSAMR | AVGVGKSAL |
| CGVGKSALT | HEEYSAMRD | VGVGKSALT |
| EYKLVVVGAC | LLDILDTAGH | EYKLVVVGAV |
| YKLVVVGACG | LDILDTAGHE | YKLVVVGAVG |
| KLVVVGACGV | DILDTAGHEE | KLVVVGAVGV |
| LVVVGACGVG | ILDTAGHEEY | LVVVGAVGVG |
| VVVGACGVGK | LDTAGHEEYS | VVVGAVGVGK |
| VVGACGVGKS | DTAGHEEYSA | VVGAVGVGKS |
| VGACGVGKSA | TAGHEEYSAM | VGAVGVGKSA |
| GACGVGKSAL | AGHEEYSAMR | GAVGVGKSAL |
| ACGVGKSALT | GHEEYSAMRD | AVGVGKSALT |
| CGVGKSALTI | HEEYSAMRDQ | VGVGKSALTI |
| TEYKLVVVGAC | CLLDILDTAGH | TEYKLVVVGAV |
| EYKLVVVGACG | LLDILDTAGHE | EYKLVVVGAVG |
| YKLVVVGACGV | LDILDTAGHEE | YKLVVVGAVGV |
| KLVVVGACGVG | DILDTAGHEEY | KLVVVGAVGVG |
| LVVVGACGVGK | ILDTAGHEEYS | LVVVGAVGVGK |
| VVVGACGVGKS | LDTAGHEEYSA | VVVGAVGVGKS |
| VVGACGVGKSA | DTAGHEEYSAM | VVGAVGVGKSA |
| VGACGVGKSAL | TAGHEEYSAMR | VGAVGVGKSAL |
| GACGVGKSALT | AGHEEYSAMRD | GAVGVGKSALT |
| ACGVGKSALTI | GHEEYSAMRDQ | AVGVGKSALTI |
| CGVGKSALTIQ | HEEYSAMRDQY | VGVGKSALTIQ |

TABLE 55B
SLATE TP53 Peptide Pools in Restimulation Assay

| TP53 R213L | TP53 S217Y | TP53 R249M |
|---|---|---|
| Peptides | Peptides | Peptides |
| LDDRNTFL | KSVTCTYY | CMGGMNRM |
| DDRNTFLH | SVTCTYYP | MGGMNRMP |
| DRNTFLHS | VTCTYYPA | GGMNRMPI |
| RNTFLHSV | TCTYYPAL | GMNRMPIL |
| NTFLHSVV | CTYYPALN | MNRMPILT |
| TFLHSVVV | TYYPALNK | NRMPILTI |
| FLHSVVVP | YYPALNKM | RMPILTII |
| LHSVVVPY | YPALNKMF | MPILTIIT |
| YLDDRNTFL | AKSVTCTYY | SCMGGMNRM |
| LDDRNTFLH | KSVTCTYYP | CMGGMNRMP |
| DDRNTFLHS | SVTCTYYPA | MGGMNRMPI |
| DRNTFLHSV | VTCTYYPAL | GGMNRMPIL |
| RNTFLHSVV | TCTYYPALN | GMNRMPILT |
| NTFLHSVVV | CTYYPALNK | MNRMPILTI |
| TFLHSVVVP | TYYPALNKM | NRMPILTII |
| FLHSVVVPY | YYPALNKMF | RMPILTIIT |
| LHSVVVPYE | YPALNKMFC | MPILTIITL |
| EYLDDRNTFL | TAKSVTCTYY | SSCMGGMNRM |
| YLDDRNTFLH | AKSVTCTYYP | SCMGGMNRMP |
| LDDRNTFLHS | KSVTCTYYPA | CMGGMNRMPI |
| DDRNTFLHSV | SVTCTYYPAL | MGGMNRMPIL |
| DRNTFLHSVV | VTCTYYPALN | GGMNRMPILT |
| RNTFLHSVVV | TCTYYPALNK | GMNRMPILTI |
| NTFLHSVVVP | CTYYPALNKM | MNRMPILTII |
| TFLHSVVVPY | TYYPALNKMF | NRMPILTIIT |
| FLHSVVVPYE | YYPALNKMFC | RMPILTIITL |
| LHSVVVPYEP | YPALNKMFCQ | MPILTIITLE |
| VEYLDDRNTFL | GTAKSVTCTYY | NSSCMGGMNRM |
| EYLDDRNTFLH | TAKSVTCTYYP | SSCMGGMNRMP |
| YLDDRNTFLHS | AKSVTCTYYPA | SCMGGMNRMPI |
| LDDRNTFLHSV | KSVTCTYYPAL | CMGGMNRMPIL |
| DDRNTFLHSVV | SVTCTYYPALN | MGGMNRMPILT |
| DRNTFLHSVVV | VTCTYYPALNK | GGMNRMPILTI |
| RNTFLHSVVVP | TCTYYPALNKM | GMNRMPILTII |
| NTFLHSVVVPY | CTYYPALNKMF | MNRMPILTIIT |
| TFLHSVVVPYE | TYYPALNKMFC | NRMPILTIITL |
| FLHSVVVPYEP | YYPALNKMFCQ | RMPILTIITLE |
| LHSVVVPYEPP | YPALNKMFCQL | MPILTIITLED |

TABLE 56A
SLATE Patient S1 ELISpot

| Timepoint | Dose administered | Vehicle | | CD8 Pool | |
|---|---|---|---|---|---|
| Day 0 | ChAdV Prime | 5 | 5 | | |
| Day 14 | | 0 | 10 | | |
| Day 28 | SAM Boost 1 | 0 | 10 | 35 | 40 |
| Day 35 | | 0 | 0 | | |
| Day 42 | | 15 | 0 | 50 | 55 |
| Day 56 | EOT | 15 | 25 | 30 | 25 |

TABLE 56B
SLATE Patient S2 ELISpot

| Timepoint | Dose administered | Vehicle | | | CD8 Pool | |
|---|---|---|---|---|---|---|
| Day 0 | ChAdV Prime | 10 | 30 | 15 | 195 | 285 |
| Day 14 | | 30 | 60 | 10 | 390 | 285 |
| Day 28 | SAM Boost 1 | 10 | 0 | 10 | 485 | 455 |
| Day 35 | | 5 | 0 | 5 | 480 | 500 |
| Day 42 | | 60 | 80 | 70 | 565 | 855 |
| Day 56 | SAM Boost 2 | 10 | 15 | 20 | 420 | 450 |

### XXVI. Clinical Assessment of a Neoantigen Vaccine

An open-label, multi-center, multi-dose Phase 1/2 study is performed to assess the dose, safety and tolerability, immunogenicity, and early clinical activity of a heterologous prime/boost vaccination strategy. Two vaccine programs, GRANITE and SLATE, are assessed.

A personalized neoantigen cancer vaccine (“GRANITE”) is administered in combination with immune checkpoint blockade in patients with advanced cancer strategies. The GRANITE heterologous prime/boost vaccine regimen involves (1) a ChAdV that is used as a prime vaccination [GRT-C901] and (2) a SAM formulated in a LNP that is used for boost vaccinations [GRT-R902] following GRT-C901. Both GRT-C901 and GRT-R902 express the same 20 personalized neoantigens as well as two universal CD4 T-cell epitopes (PADRE and Tetanus Toxoid). Tumors are used for whole-exome and transcriptome sequencing to detect somatic mutations, and blood is used for HLA typing and detection/subtraction of germline exome variants to generate the personalized neoantigen cassette using the EDGE algorithm.

A shared neoantigen cancer vaccine (“SLATE”) is administered in combination with immune checkpoint blockade in patients with advanced cancer. The SLATE heterologous prime/boost vaccine regimen involves (1) a ChAdV that is used as a prime vaccination [GRT-C903] and (2) a SAM formulated in a LNP that is used for boost vaccinations [GRT-R904]following GRT-C903. Both GRT-C903 and GRT-R904 express the same 20 shared neoantigens derived from a specific list of oncogenic mutations (see Table 34) as well as two universal CD4 T-cell epitopes (PADRE and Tetanus Toxoid). For subject inclusion, tumors are used for whole-exome and transcriptome sequencing to detect somatic mutations, and blood is used for HLA typing.

GRT-C901 and GRT-C903 are replication-defective, E1 and E3 deleted adenoviral vectors based on chimpanzee adenovirus 68. The vectors contain an expression cassette encoding 20 neoantigens as well as two universal CD4 T-cell epitopes (PADRE and Tetanus Toxoid). GRT-C901 and GRT-C903 are formulated in solution at 5×10¹¹ vp/mL and 1.0 mL is injected IM at each of 2 bilateral vaccine injection sites in opposing deltoid muscles (deltoid muscle preferred, gluteus [dorso or ventro] or rectus femoris on each side may be used). The GRT-C901 and GRT-C903 vectors differ only by the encoded neoantigens.

GRT-R902 and GRT-R904 are SAM vectors derived from an alphavirus. The GRT-R902 and GRT-R904 vectors encode the viral proteins and the 5′ and 3′ RNA sequences required for RNA amplification but encoded no structural proteins. The SAM vectors are formulated in LNPs composed of 4 lipids: an ionizable amino lipid, a phosphatidylcholine, cholesterol, and a PEG-based coat lipid to encapsulate the SAM and form LNPs. The GRT-R902 vector contains the same neoantigen expression cassette as used in GRT-C901 for each patient, respectively. The GRT-R904 vector contains the same neoantigen expression cassette as used in GRT-C903. GRT-R902 and GRT-R904 are formulated in solution at 1 mg/mL and was injected IM at each of 2 bilateral vaccine injection sites in opposing deltoid muscles (deltoid muscle preferred, gluteus [dorso or ventro] or rectus femoris on each side may be used). The boost vaccination sites is as close to the prime vaccination site as possible. The injection volume is based on the dose to be administered. The dose level amount refers explicitly to the amount of the SAM vector, i.e., it does not refer to other components, such as the LNP. The ratio of LNP:SAM is approximately 24:1. Accordingly, the dose of LNP is 720 μg, 2400 μg, and 7200 μg for each respective GRT-R902/GRT-R904 dose level (see below).

Ipilimumab is a human monoclonal IgG1 antibody that binds to the cytotoxic T-lymphocyte associated antigen 4 (CTLA-4). Ipilimumab is formulated in solution at 5 mg/mL and is injected SC proximally (within ˜2 cm) to each of the bilateral vaccination sites. The SC route of ipilimumab is distinct from the approved IV route of administration. Ipilimumab is administered at a does of 30 mg in one of two methods listed below: 1. Four 1.5 mL (7.5 mg) injections proximal to the vaccine draining LN at each of the bilateral vaccination sites (ie, 1.5 mL below the vaccination site and 1.5 mL above the vaccination site on each bilateral side in each deltoid, ventrogluteal, dorsogluteal, or rectus femoris [deltoid preferred, but dependent on clinical site and patient preference]). 2. Six 1 mL (5 mg) injections proximal to the vaccine draining LN at each of the bilateral vaccination sites (ie, 1 mL below the vaccination site, 1 mL to the side of the vaccination site, and 1 mL above the vaccination site on each bilateral side in each deltoid, ventrogluteal, dorsogluteal, or rectus femoris [deltoid preferred, but dependent on clinical site and patient preference]).

Nivolumab is a human monoclonal IgG4 antibody that blocks the interaction of PD-1 and its ligands, PD-L1 and PD-L2. Nivolumab is formulated in solution at 10 mg/mL and is administered as an IV infusion through a 0.2-micron to 1.2-micron pore size, low-protein binding in-line filter at the protocol-specified doses. It is not administered as an IV push or bolus injection. When the dose is fixed (eg, 240 mg flat dose), nivolumab injection may be infused undiluted or diluted so as not to exceed a total infusion volume of 160 mL. Nivolumab infusion is promptly followed by a flush of diluent to clear the line. Nivolumab is administered following each vaccination (i.e., each of GRT-C901, GRT-R902, GRT-C903, or GRT-R904) with or without ipilimumab on the same day. The dose and route of nivolumab is based on the Food and Drug Administration approved dose and route. Doses of nivolumab may be interrupted, delayed, or discontinued depending on how well the participant tolerates the treatment. Dosing visits are not skipped, only delayed. Vaccination does not occur in the absence of nivolumab unless the Investigator and Sponsor believe treatment with GRT-R902/GRT-R904 in the absence of nivolumab is in the best interest of the patient.

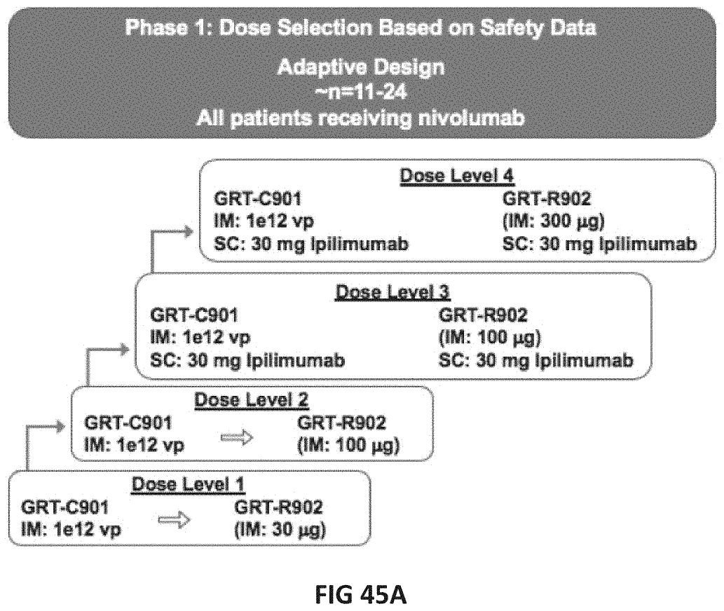

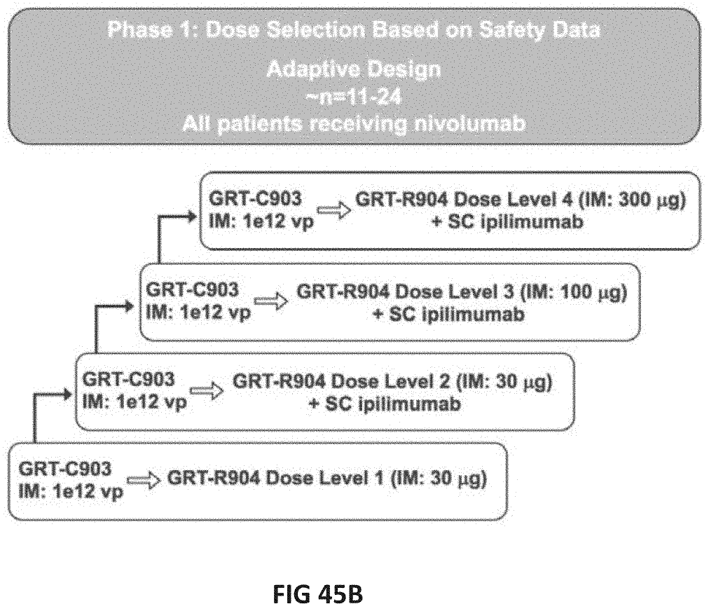

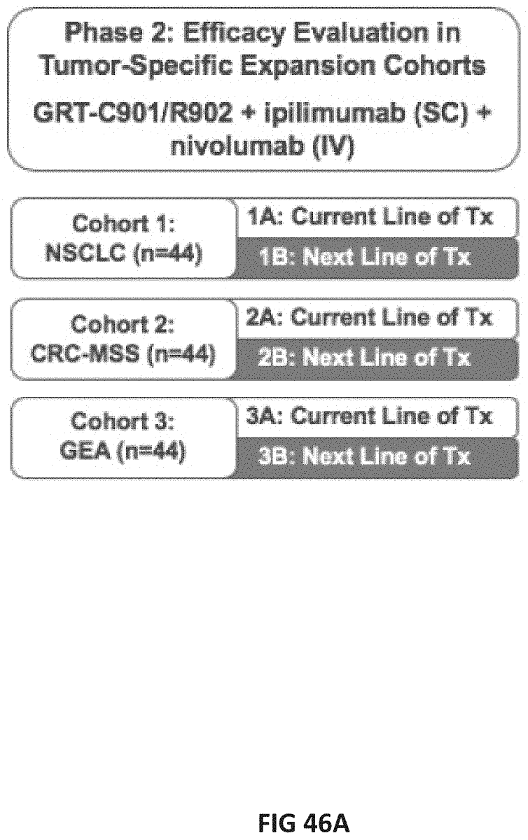

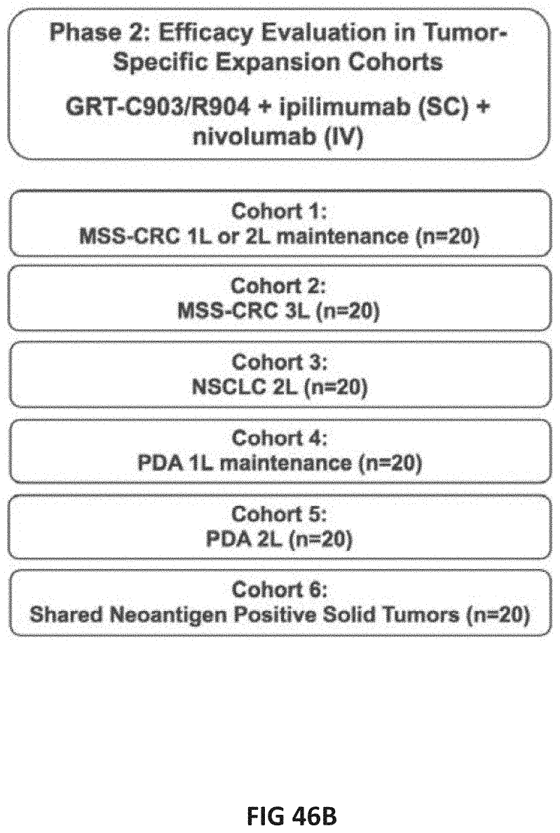

As illustrated in FIG. 45A, Phase 1 of GRANITE examines prime vaccination with GRT-C901 followed by multiple dose levels of GRT-R902 with all patients receiving IV nivolumab. Eligible patients include those with advanced or metastatic NSCLC, GEA, mUC or CRC-MSS. Following the initial demonstration of the safety and tolerability of Dose Level 1 (30 μg), the dose of GRT-R902 is escalated to Dose Level 2 (100 μg). If this combination is safe and well-tolerated, patients are treated at Dose Level 3 which incorporates SC ipilimumab (30 mg) at the same dose of GRT-R902 as in Dose Level 2 (100 μg). If this combination is safe and well-tolerated, then the dose of GRT-R902 is escalated to Dose Level 4 (300 μg) with all patients continuing to receive SC ipilimumab (30 mg). As illustrated in FIG. 46B, Phase 2 involves tumor-specific expansion cohorts.

As illustrated in FIG. 45B, Phase 1 of SLATE examines prime vaccination with GRT-C903 followed by multiple dose levels of GRT-R904 with all patients receiving IV nivolumab. Eligible patients include those with advanced or metastatic MSS-CRC, NSCLC, PDA, and other mutation-positive solid tumors. Following the initial demonstration of the safety and tolerability of Dose Level 1 (30 μg), patients are treated at Dose Level 2 which incorporates SC ipilimumab (30 mg) at the same dose of GRT-R904 as in Dose Level 1 (30 μg). If this combination is safe and well-tolerated, then the dose of GRT-R904 is escalated to Dose Level 3 (100 μg) followed by Dose Level 4 (300 μg) with all patients continuing to receive SC ipilimumab (30 mg). As illustrated in FIG. 46B, Phase 2 involves tumor-specific expansion cohorts and a cohort of mutation-positive tumors outside of tumor types already represented by other expansion cohorts. Cohorts 1 and 4 enroll patients who have not experienced disease progression to routine therapy and Cohorts 2, 3, 5, and 6 enroll patients who experienced disease progression on, or after, routine therapy. Patients in Phase 2 receive SC ipilimumab at the dose determined to be well tolerated in Phase 1. Patients receive IV nivolumab at 480 mg Q4W throughout Phase 1 and 2.

GRANITE Protocol Stages

For GRANITE, the personalized nature of the vaccine requires a specialized manufacturing process and thus, the patient's participation in the study is conducted in 2 stages:
1) Vaccine Production Stage
   a. Neoantigen prediction: NGS of a patient's tumor and blood followed by prediction of a patient's neoantigens using the EDGE machine learning model.
   b. Vaccine manufacturing: generating a patient-specific heterologous prime/boost vaccine that incorporates the identified neoantigens.
2) Study Treatment Stage: delivering the vaccine regimen to the patient consisting of the heterologous prime/boost vaccine in combination with immune checkpoint blockade.

Neoantigen prediction and vaccine manufacturing occur while the patient is receiving routine therapy for metastatic disease. The Study Treatment Stage will begin when the vaccine is ready and the patient meets eligibility criteria for the Study Treatment Stage.

The neoantigen vaccine strategy is outlined in the following steps:
Obtain formalin-fixed paraffin-embedded (FFPE) tumor specimen and blood from patient
Sequence the tumor and normal DNA and tumor RNA to identify non-synonymous exome mutations
Perform transcriptome analysis to determine expression level of mutated proteins/peptides
Determine the patient's HLA type from normal DNA
Predict which of the mutant peptides are likely to be presented by the patient's class I HLA alleles using the EDGE model
Assemble a prioritized list of candidate neoantigens based on predicted HLA presentation (including removal of epitopes identical to self-proteins to prevent potential autoimmune reactions)
Insert the top 20 patient-specific predicted neoantigens into an expression cassette
Generate two plasmids encoding the identical neoantigen expression cassette; one plasmid for GRT-C901 and one plasmid for GRT-R902
Utilize plasmids to generate the ChAdV68 and SAM vectors
Formulate the SAM in LNP
Confirm the potency, identity, and sterility of ChAdV and SAM-LNP
Immunize the patient with the patient-specific, selected neoantigen vaccine, in combination with checkpoint blockade

GRANITE Vaccine Production Stage

The first step in manufacturing GRT-C901 and GRT-R902 is to determine the presence of neoantigens in a patient's tumor. Patients have available and sufficient tumor tissue and blood for genomic sequencing and HLA typing. For inclusion in the study, each patient has an available FFPE tumor specimen (FFPE block/slides/scrolls sufficient to yield approximately 40 microns tissue thickness and preferably from the most recent available biopsy) and blood. When equally recent FFPE tumor specimens are available from both primary tumor and distant metastases, the primary tumor specimen is preferred. The tumor is used for whole-exome and transcriptome sequencing to detect somatic mutations, and the blood for HLA typing and detection/subtraction of germline exome variants.

GRANITE Study Treatment Stage

When a patient's vaccine is ready for administration, patients receive study treatment according to the study Phase/cohort in which they are enrolled, as described above. Patients who experience disease progression on or after, or intolerance to, routine therapy while vaccine is being manufactured receive study treatment as their next line of therapy. For patients who have SD or better response to their routine therapy while the vaccine is being manufactured, study treatment is either administered concurrent with their routine therapy or these patients interrupt or stop routine therapy and receive only study treatment if deemed clinically appropriate (eg, in the setting of cumulative toxicity, patient preference/refusal to continue routine therapy).

The study uses a sequential approach in Phase 1 to assess the safety of the intended GRANITE vaccine regimen consisting of a heterologous prime/boost vaccine in combination with nivolumab and ipilimumab. Patients receive a prime vaccination with GRT-C901 followed by boost vaccinations with escalating doses of GRT-R902. All patients receive IV nivolumab. As part of the dose escalation of GRT-R902, SC ipilimumab is combined with the GRT-C901 prime vaccination and GRT-R902 boost vaccinations for patients treated in Dose Levels 3 and 4. Given the immunogenicity data observed in NHPs (data not shown), the addition of SC ipilimumab is expected to boost T-cell responses providing the best opportunity for a patient to generate the most robust T-cell response.

Phase 2 consists of tumor-specific expansion cohorts that each comprise two treatment settings for each tumor type; an “A” arm that enrolls patients who have SD or better response to routine therapy and a “B” arm that enrolls patients who experience disease progression on, or after, routine therapy (ie, post-progression). Patients in Phase 2 receive SC ipilimumab at the dose determined to be well tolerated in Phase 1. Patients receive IV nivolumab at 480 mg Q4W throughout Phase 1 and 2. In one GRANITE phase 2 arm, MSS-CRC cohorts, including following FOLFOX/FOLFIRI treatment, are assessed using the RP2D determined. In another GRANITE phase 2 arm, Gastro-esophageal cancer (GEA) cohorts, including following 2^{nd} line chemotherapy, are assessed using the RP2D determined.

If patients progress prior to vaccine availability, the patient can begin treatment with nivolumab until the vaccine is available (ie, nivolumab bridging). Once the vaccine is available, the vaccine is administered upon the next treatment with nivolumab to align vaccine administration with nivolumab administration.

SLATE Protocol Stages

For SLATE, as shared tumor neoantigens are presented via specific HLA proteins on the surface of the tumor cells for recognition by CD8 T cells, the patient's HLA Class I alleles are identified to confirm the patient's tumor-specific neoantigen can be presented. Therefore, patient's participation in the study is conducted in 2 stages:

1) HLA Screening Stage

2) Study Treatment Stage

Given that ˜33% of patients with a relevant mutation are expected to have the matching HLA allele, patients are HLA typed prior to being eligible for study treatment. Patients eligible for the HLA Screening Stage are briefly described below; these patients include those with advanced or metastatic:
MSS-CRC who are currently receiving systemic chemotherapy OR who have experienced disease progression following systemic chemotherapy, but have not initiated a new line of therapy
NSCLC who are currently receiving an anti-PD-(L)1 antibody in combination with platinum-based chemotherapy OR who have experienced disease progression following treatment with an anti-PD-(L)1 antibody in combination with platinum-based chemotherapy, but have not initiated a new line of therapy
PDA who are currently receiving first-line systemic chemotherapy for metastatic disease OR who have experienced disease progression on 1 L systemic cytotoxic chemotherapy, but have not initiated a new line of therapy
Other solid tumor histology where the patient's tumor has the specified mutation and has experienced disease progression on available therapies known to confer clinical benefit

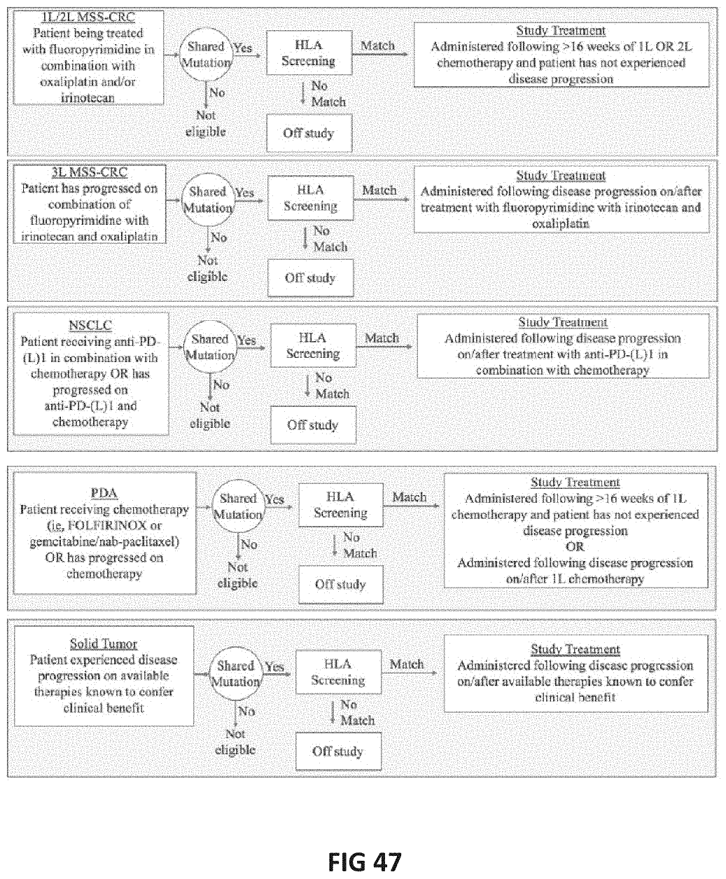

Patients with a matching specified mutation and HLA allele are eligible for study treatment. FIG. 47 illustrates the flow of patients through the study for each tumor type eligible for treatment with GRT-C903/GRT-R904.

SLATE HLA Screening Stage

The HLA Screening Stage of the study identifies patients who may be eligible for study treatment. A blood sample is submitted to the Sponsor to perform HLA typing to determine whether a patient possesses an HLA allele that matches the specified oncogenic mutation in that patient's tumor. Patients who do not possess the appropriate HLA alleles for the tumor-specific mutation are not be eligible to participate in the Study Treatment Stage.

Patients are assessed in the HLA Screening Stage if their tumors are known to have at least one mutation included in the expression cassette of GRT-C903/GRT-R904 based on a suitable molecular profiling assay performed according to institutional standards in a Clinical Laboratory Improvement Amendments (CLIA)-certified laboratory by a validated assay (eg, institutional or commercially available gene panel) (see Table 34). The mutation is clearly identified in the report and subclonal mutations are not considered eligible. HLA typing is performed by a centralized third-party CLIA-certified laboratory designated by the Sponsor. The Sponsor confirms the mutation result based on the mutation test report provided by the site and determines whether the patient's HLA Class I proteins are adequate for antigen presentation of the patient's tumor shared neoantigen(s).

SLATE Study Treatment Stage

Patients that meet eligibility criteria are administered the vaccine prime and boosts in combination with ipilimumab and nivolumab to augment T-cell responses. Patients receive study treatment according to the study Phase/cohort in which they are enrolled once their eligibility for the Study Treatment Stage has been confirmed.

Study treatment are administered either as maintenance therapy or as a new line of therapy as briefly described below:
MSS-CRC 1L/2L maintenance: patients who have not experienced PD with at least 16 weeks of routine 1L or 2L chemotherapy
MSS-CRC 3L: patients who have experienced PD on 2L chemotherapy
NSCLC 2L: patients who have experienced PD following 1L treatment with anti-PD-(L)1 in combination with platinum-based chemotherapy (or PD following 1L treatment with anti-PD-(L)1 alone if patient refused platinum-based chemotherapy)
PDA 1L maintenance: patients who have not experienced PD with at least 16 weeks of routine 1L chemotherapy
PDA 2L: patients who have experienced PD on 1L chemotherapy
Mutation positive solid tumor: patients who have experienced PD with all available therapies known to confer benefit

Some patients may be eligible to receive the shared neoantigen vaccine in the maintenance setting. For patients with MSS-CRC who have received >16 weeks of 1L or 2L chemotherapy or with PDA who have received >16 weeks of 1L chemotherapy, but have not experienced disease progression, study treatment may be administered concurrent with their routine therapy or these patients may interrupt or stop routine therapy and receive only study treatment if deemed clinically appropriate by the Investigator (eg, in the setting of cumulative toxicity, patient preference/refusal to continue routine therapy).

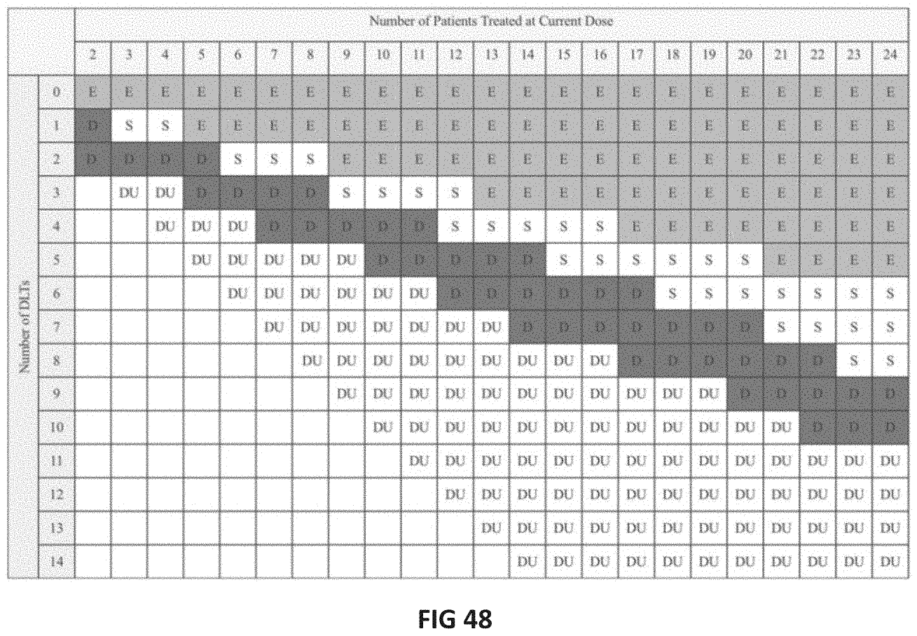

Phase 1 utilizes adaptive statistical methods to guide dose selection with the goal of determining the highest vaccine dose with a dose-limiting toxicity rate below 30%. This target toxicity rate takes into account the known safety profile of nivolumab and ipilimumab while optimizing the number of patients needed to establish a preliminary toxicity profile of the vaccine in combination with nivolumab and ipilimumab compared to other statistical approaches (eg, a 3+3 design). The dosing decisions in Phase 1 is based on a safety review of the treatment by the Study Committee (eg, to determine whether a DLT has occurred). An established adaptive method termed the modified toxicity probability interval (mTPI) incorporates the currently available safety information and indicates what dose subsequent patients should receive (ie, dose escalation, de-escalation, continue at the same dose, or close that dose level). FIG. 48 illustrates an improved version of the dose selection design, referred to as mTPI-2. This design allows for real-time adjustment of the dose based on observed toxicities and has a lower risk of treating patients at a dose higher than the target toxicity rate compared to a traditional 3+3 design and is more likely to select the most tolerable dose. The mTPI-2 model incorporates available information for all patients treated at a particular dose level to guide dosing of subsequent patients. The mTPI-2 model may be regarded as providing guidance and information to be integrated with a clinical assessment of the toxicity profiles observed at the time of analysis in determining the next dose to be investigated.

Phase 1 enrolls a maximum sample size of 24 patients. The full sample size may not be reached if either of the following criteria are met:
There are no DLTs observed in >5 patients at the highest available dose level
At least 8 subjects have been treated at the highest available dose level and the mTPI-2 model continues to recommend dose escalation

If either of these criteria are met, then the study may proceed to the next Phase (ie, from Phase 1 to Phase 2) prior to reaching the maximum sample size provided that higher dose levels are not available (eg, after ≥5 patients have been treated at Dose Level 4 in Phase 1 with no DLTs, the study can proceed to Phase 2).

A dose is excluded from further consideration due to unacceptable toxicity (DU) if at least 3 patients have been evaluated at that dose and the posterior probability that the true DLT rate is >30% exceeds 95%. Patients who are receiving study treatment concurrent with chemotherapy may be considered separately if there is evidence that toxicity may be due to the concurrent treatment with chemotherapy.

For all new dose levels, there is >24 hours between the first and second patients receiving GRT-C903 or the first dose of GRT-R904 to allow for observation of any severe acute toxicities as an initial assessment of the safety of this therapeutic approach and to inform subsequent treatment of patients.

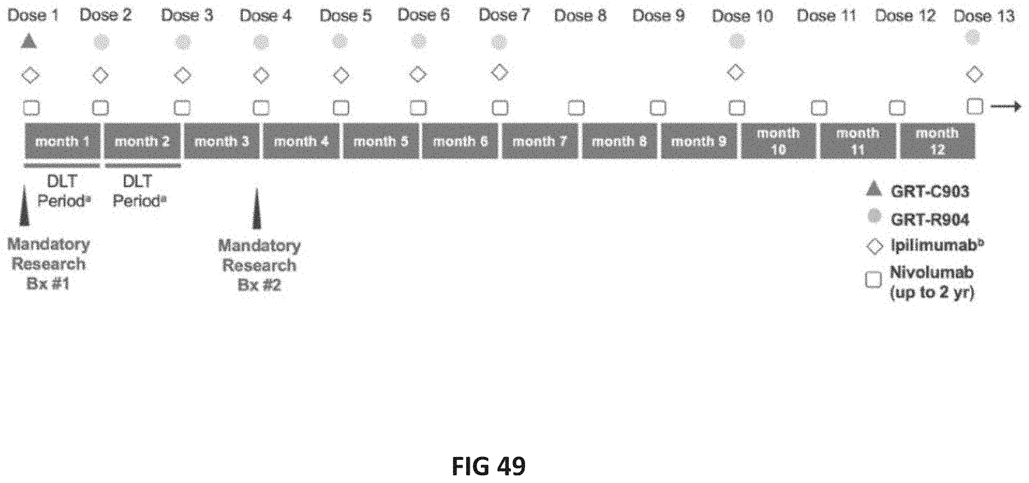

FIG. 49 illustrates the Phase 1 dosing schedule GRT-C903, GRT-R904, Nivolumab, and Ipilimumab. Phase 1 begins with patients receiving GRT-C903 at a fixed dose of 1×10¹² viral particles (vp) with no planned dose escalation in combination with IV nivolumab. Based on the existing safety profile of adenovirus-based vaccines in multiple clinical studies in thousands of patients, GRT-C903 is anticipated to be well-tolerated at this dose. However, if DLTs are observed and the mTPI model calls for de-escalation, the dose of adenovirus can be de-escalated to 3×10¹¹ vp with a further de-escalation to 1×10¹¹ vp if indicated by the mTPI model. Once patients treated with GRT-C903 have completed the first 28-day DLT observation period, patients receive GRT-R904 as vaccine boosts administered IM bilaterally every 4 weeks (Q4W). The first 28 days after the first treatment with GRT-R904 as a boost vaccination (ie, following priming with GRT-C903) are treated as a second DLT window for the assessment of GRT-R904-related toxicity. Patients continue to receive GRT-R904 as vaccine boosts.

The mTPI-2 model determines whether doses of GRT-R904 can be increased from 30 to 100 to 300 μg in subsequent patients. Patients beginning study treatment at Dose Level 1 receive their first dose (Dose 1) of 30 μg of GRT-R904 in combination with IV nivolumab followed by the DLT observation period of 28 days per patient. Dose escalation decisions are made based on the mTPI-2 model once at least two patients have completed the DLT observation period at any dose level. For example, in the absence of any observed DLTs in the first two patients treated at Dose Level 1, dose escalation is permitted as indicated by the mTPI-2 model.

If the mTPI-2 model dictates dose escalation, then subsequent patients are treated at Dose Level 2 (ie, 30 μg of GRT-R904 in combination with 30 mg of SC ipilimumab). Once at least two patients have completed the DLT observation period, the mTPI model determines the subsequent dose of GRT-R904 to treat additional patients in combination with 30 mg of SC ipilimumab. For example, if no DLTs are observed in at least two patients in Dose Level 2, the mTPI model dictates dose escalation resulting in subsequent patients being administered GRT-R904 at the next dose level of 100 μg in combination with 30 mg of SC ipilimumab (ie, Dose Level 3) followed by the DLT observation period of 28 days. Similarly, if no DLTs are observed in at least two patients receiving 100 μg, subsequent patients receive GRT-R904 at the next dose level of 300 μg in combination with 30 mg of SC ipilimumab (ie, Dose Level 4) followed by a DLT observation period of 28 days. If DLTs are observed at the initial 30 μg dose and the mTPI model indicates de-escalation, a lower dose (Dose Level −1) of 10 μg of GRT-R904 may be evaluated in these patients.

If a DLT is observed at a particular dose level and the mTPI-2 model dictates dose de-escalation, then subsequent patients are treated at the next lower dose level below the dose level at which the DLT was observed. For example, if a DLT was observed within the 28-day DLT observation period in Dose Level 4 (ie, 300 μg dose of GRT-R904 in combination with 30 mg of SC ipilimumab) and the mTPI-2 model dictates dose de-escalation, subsequent patients start Dose 1 at Dose Level 3 (ie, 100 μg of GRT-R904 in combination with 30 mg of SC ipilimumab).

Each patient could receive a total of 9 vaccine doses including one dose of GRT-C903 (Dose 1) and 8 doses of GRT-R904 (Doses 2 to 7, 10, and 13). For doses 8, 9, 11, 12, 14, and onward patients only receive nivolumab.

The RP2D for GRT-R904 is declared once the criteria for moving from Phase 1 to Phase 2 have been met as described above (no DLTs in at least 5 patients, observed DLT rate <30% in at least 8 patients treated at the highest dose and mTPI-2 continues to recommend escalation, or maximum number of patients have been treated).

Once the study has moved to Phase 2, patients treated at Dose Levels 1 and 2 may receive the RP2D for GRT-R904 in combination with SC ipilimumab to minimize patient exposure to potentially sub-therapeutic doses and increase the amount of safety data obtained with the RP2D dose.

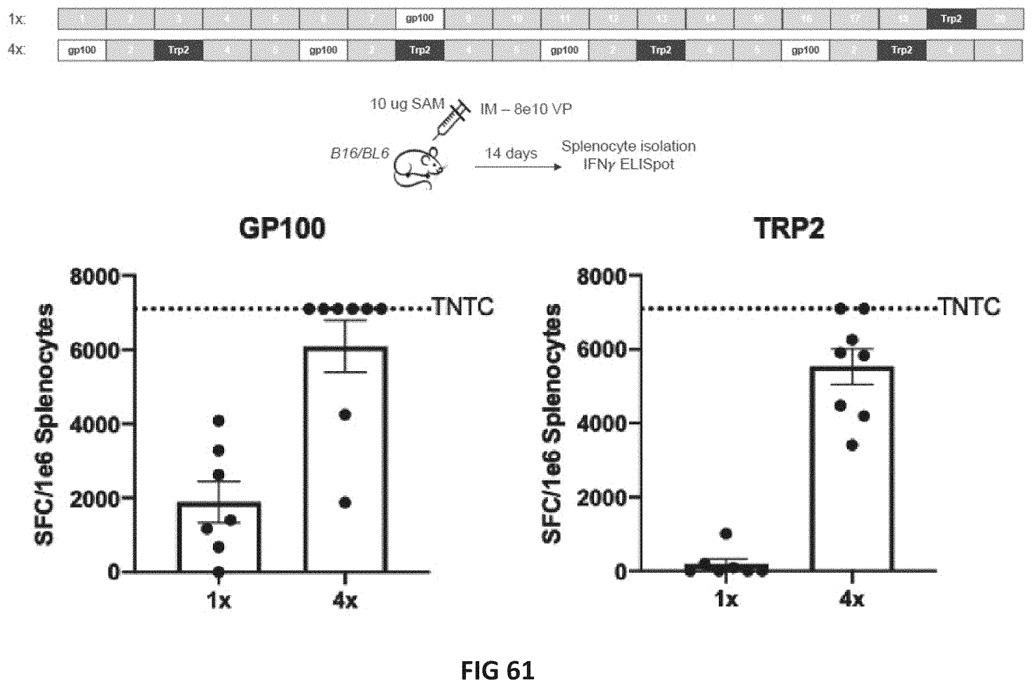

Following evaluation of the safety and tolerability of the vaccine regimen in Phase 1, Phase 2 enrolls tumor-specific cohorts in Phase 2 to assess early signs of clinical activity. Patients are treated in Phase 2 as in Phase 1. The vaccine regimen includes patients receiving GRT-C903/GRT-R904 IM bilaterally in combination with IV nivolumab with SC ipilimumab based on the tolerability in Phase 1. The dose of GRT-C903/GRT-R904 and SC ipilimumab is the RP2D based on data from Phase 1. Each expansion cohort enrolls approximately 20 patients. In one SLATE Phase 2 arm, patients with TP53 mutations, including ovarian cancer cohorts and others, are assessed using dose level 4 (300 μg+SC IPI). In another SLATE Phase 2 arm, NSCLC cohorts, including patients following immunotherapy and/or chemotherapy, are assessed using dose level 4 (300 μg+SC IPI) with either the original SLATE cassette or a new SLATE cassette design (such as one that features repeated epitopes, including KRAS epitopes). A representative preclinical cassette featuring multiple iterations of epitopes and corresponding preclinical data is shown in FIG. 61.

### XXVII. Clinical Assessment of a Neoantigen Vaccine

Appendix A is a document 35 pages in length (including title slip sheet) describing embodiments of the invention. Appendix A is hereby incorporated by reference, in its entirety, for all purposes.

It should be noted that the language used in Appendix A has been principally selected for readability and instructional purposes, and it may not have been selected to delineate or circumscribe the inventive subject matter. Accordingly, the disclosure of Appendix A is intended to be illustrative, but not limiting, of the scope of the invention.

In Appendix A, clinical evaluation results are presented for the open-label, multi-center, multi-dose Phase 1/2 studies that were performed to assess the dose, safety and tolerability, immunogenicity, and early clinical activity of a heterologous prime/boost vaccination strategy for the two vaccine programs, GRANITE and SLATE, described herein. The results demonstrated clear signals of efficacy.

Also presented in Appendix A are further studies that are performed to continue to assess clinical efficacy and safety.

While the invention has been particularly shown and described with reference to a preferred embodiment and various alternate embodiments, it will be understood by persons skilled in the relevant art that various changes in form and details can be made therein without departing from the spirit and scope of the invention.

### Sequences

Table A

Refer to Sequence Listing, SEQ ID NOS. 10,755-21,015. For clarity, each peptide that was predicted to associate with a given HLA allele peptide with an HLA allele with an EDGE score >0.001 is assigned a unique SEQ ID. NO. Each of the above sequence identifiers is associated with the amino acid sequence of the peptide, HLA subtype, the gene name corresponding to the peptide, the mutation associated with the peptide, and whether the prevalence of the peptide:HLA pair was greater than 0.1% (noted as “1”) or less than 0.1% (noted as “0”).

Table A is disclosed in its entirety in U.S. Provisional Application No. 62/675,559, filed December May 23, 2018, which is hereby incorporated by reference in its entirety.

AACR GENIE Results

Refer to Sequence Listing, SEQ ID NOS. 21,016-29,357. For clarity, each peptide that was predicted to associate with a given HLA allele peptide with an HLA allele with an EDGE score >0.001 and prevalence >0.1% is assigned a unique SEQ ID. NO. Each of the above sequence identifiers is associated with the gene name and mutations corresponding to the peptide, HLA subtype, and amino acid sequence of the peptide.

Additional MS Validated Neoantigens
(SEQ ID NOs 29513-29519)

| | | | | Restricted |
|---|---|---|---|---|
| SEQ | | | HLA | Peptide |
| ID | | | Class 1 | amino acid |
| NO: | Gene | Mutation | subtype | sequence |
| 29513 | CTNNB1 | S37Y point | HLA-A* | YLDSGIHYGA |
| | | mutation | 02:01 | |
| 29514 | CHD4 | CHD4_K73fs | HLA-B* | TVRAATIL |
| | | | 08:01 | |
| 29515 | CTNNB1 | CTNNB1S45P | A*11:01 | TTAPPLSGK |
| 29516 | CTNNB1 | CTNNB1T41A | A*11:01 | ATAPSLSGK |
| 29517 | KRAS | KRASG12V | A*03:01 | VVGAVGVGK |
| 29518 | KRAS | KRASQ61R | A*01:01 | ILDTAGREEY |
| 29519 | TP53 | TP53R213L | A*02:01 | YLDDRNTFL |

Table 1.2

Refer to Sequence Listing, SEQ ID NOS. 57-10,754. Predicted shared antigens associated with gene expressed at a level of at least 10 TPM in at least 0.98% of cancer cases. Each of the above sequence identifiers is associated with the gene name, amino acid sequence of the peptide, Ensembl ID, and corresponding HLA allele(s).

### Certain Sequences

Vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT NoSFL nucleotide (SEQ ID NO: 36)
Ub7625merPDTT NoSFL polypeptide (SEQ ID NO: 37)
ChAdV68.5WTnt.MAG25mer (SEQ ID NO: 2); AC_000011.1 with E1 (nt 577 to 3403)
and E3 (nt 27,125-31,825) sequences deleted; corresponding ATCC VR-594
nucleotides substituted at five positions; model neoantigen cassette under
the control of the CMV promoter/enhancer inserted in place of deleted E1;
SV40 polyA 3′ of cassette
Venezuelan equine encephalitis virus [VEE] (SEQ ID NO: 3) GenBank: L01442.2
VEE-MAG25mer (SEQ ID NO: 4); contains MAG-25merPDTT nucleotide (bases 30-
1755)
Venezuelan equine encephalitis virus strain TC-83 [TC-83](SEQ ID NO: 5)
GenBank: L01443.1
VEE Delivery Vector (SEQ ID NO: 6); VEE genome with nucleotides 7544-11175
deleted [alphavirus structural proteins removed]
TC-83 Delivery Vector(SEQ ID NO: 7); TC-83 genome with nucleotides 7544-
11175 deleted [alphavirus structural proteins removed]
VEE Production Vector (SEQ ID NO: 8); VEE genome with nucleotides 7544-
11175 deleted, plus 5′ T7-promoter, plus 3′ restriction sites
TC-83 Production Vector(SEQ ID NO: 9); TC-83 genome with nucleotides 7544-
11175 deleted, plus 5′ T7-promoter, plus 3′ restriction sites
VEE-UbAAY (SEQ ID NO: 14); VEE delivery vector with MHC class 1 mouse tumor
epitopes SIINFEKL (SEQ ID NO: 29362) and AH1-A5 inserted
VEE-Luciferase (SEQ ID NO: 15); VEE delivery vector with luciferase gene
inserted at 7545
ubiquitin (SEQ ID NO: 38)>UbG76 0-228
Ubiquitin A76 (SEQ ID NO: 39)>UbA76 0-228
HLA-A2 (MHC class 1) signal peptide (SEQ ID NO: 40)>MHC SignalPep 0-78
HLA-A2 (MHC class 1) Trans Membrane domain (SEQ ID NO: 41)>HLA A2 TM Domain
0-201
IgK Leader Seq (SEQ ID NO: 42)>IgK Leader Seq 0-60
Human DC-Lamp (SEQ ID NO: 43)>HumanDCLAMP 0-3178
Mouse LAMPI (SEQ ID NO: 44)>MouseLampl 0-1858
Human Lampl cDNA (SEQ ID NO: 45)>Human Lampl 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50)>WPRE 0-593
IRES (SEQ ID NO: 51)>eGFP IRES SEAP Insert 1746-2335
GFP (SEQ ID NO: 52)
SEAP (SEQ ID NO: 53)
Firefly Luciferase (SEQ ID NO: 54)
FMDV 2A (SEQ ID NO: 55)
Ipilimumab Heavy Chain (SEQ ID NO: 29520)
QVQLVESGGGVVQPGRSLRLSCAASGFTFSSYTMHWVRQAPGKGLEWVTFISYDGNNKYYADSVKGRFTISRDN
SKNTLYLQMNSLRAEDTAIYYCARTGWLGPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLSL
SPGK
Ipilimumab Light Chain (SEQ ID NO: 29521)
EIVLTQSPGTLSLSPGERATLSCRASQSVGSSYLAWYQQKPGQAPRLLIYGAFSRATGIPDRFSGSGSGTDFTL
TISRLEPEDFAVYYCQQYGSSPWTFGQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
Nivolumab Heavy Chain (SEQ ID NO: 29522)
QVQLVESGGGVVQPGRSLRLDCKASGITFSNSGMHWVRQAPGKGLEWVAVIWYDGSKRYYADSVKGRFTISRDN
SKNTLFLQMNSLRAEDTAVYYCATNDDYWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPC
PAPEFLGGPSVFLFPPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLSLSLGK
Nivolumab Light Chain (SEQ ID NO: 29523)
EIVLTQSPATLSLSPGERATLSCRASQSVSSYLAWYQQKPGQAPRLLIYDASNRATGIPARFSGSGSGTDFTLT
ISSLEPEDFAVYYCQQSSNWPRTFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC

### REFERENCES

1. Desrichard, A., Snyder, A. & Chan, T. A. Cancer Neoantigens and Applications for Immunotherapy. Clin. Cancer Res. Off J. Am. Assoc. Cancer Res. (2015). doi: 10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637-COMPOSITIONS AND METHODS OF IDENTIFYING TUMOR SPECIFIC NEOANTIGENS. (A1). at <http://appft1.uspto.gov/netacgi/nph-Parser?Sect1=PTO1&Sect2=HITOFF&d=PG01&p=1&u=/netahtml/PTO/srchnum.html&r=1& f=G&1=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Stemberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. Patent Application: 0120208706-OPTIMIZATION OF MULTIGENE ANALYSIS OF TUMOR SAMPLES. (A1). at <http://appft1.uspto.gov/netacgi/nph-Parser?Sect1=PTO1&Sect2=HITOFF&d=PG01&p=1&u=/netahtml/PTO/srchnum.html&r=1& f=G&1=50&s1=20120208706.PGNR.>
17. Target Capture for NextGen Sequencing-IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf. Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf. Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf. Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf. Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf. Engl. (2015). doi:10.1093/bioinformatics/btv639
46. Jorgensen, K. W., Rasmussen, M., Buus, S. & Nielsen, M. NetMHCstab-predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Keşmir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M11.011429 (2012).
50. Duan, F. et al. Genomic and bioinformatic profiling of mutational neoepitopes reveals new rules to predict anticancer immunogenicity. J Exp. Med. 211, 2231-2248 (2014).
51. Janeway's Immunobiology: 9780815345312: Medicine & Health Science Books @ Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3;103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March;14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September;10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng KH. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January;13(1):22-4. doi: 10.1002/pmic.201200439. Epub 2012 Dec. 4.
62. Eng J K, Hoopmann M R, Jahan T A, Egertson J D, Noble W S, MacCoss MJ. A deeper look into Comet-implementation and features. J Am Soc Mass Spectrom. 2015 November;26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):i42-i48, August 2008
66. Kinney R M, BJ Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frederique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pastemack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Kruse, M., Konopitzky, R., Brouwenstijn, N., and Schrier, P.I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H.M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G.M., Bertoletti, A., and Gehring, A. J. (2014). Building and optimizing a virus-specific T cell receptor library for targeted immunotherapy in viral infections. Scientific Reports 4, 4166.
73. Comet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M.F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M.I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R.W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22;239(2):389-401.
83. Strauss, J H and E G Strauss. The alphaviruses: gene expression, replication, and evolution. Microbiol Rev. 1994 September; 58(3): 491-562.
84. Rheme C, Ehrengruber M U, Grandgirard D. Alphaviral cytotoxicity and its implication in vector development. Exp Physiol. 2005 January;90(1):45-52. Epub 2004 Nov. 12.
85. Riley, Michael K. II, and Wilfred Vermerris. Recent Advances in Nanomaterials for Gene Delivery-A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September;4(7):837-56.
88. Bo Li and C. olin N. Dewey. RSEM: accurate transcript quantification from RNA-Seq data with or without a referenfe genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sebastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrors, Fulvia Vascotto, John C. Castle, Arbel D. Tadmor, Stephen P. Schoenberger, Christoph Huber, Ozlem Tureci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M.E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. NN-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS DB: de novo sequencing assisted database search for sensitive and accurate peptide identification. Molecular & Cellular Proteomics. 11(4):1-8. 1/2/2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.

1. A composition for delivery of a self-amplifying alphavirus-based expression system,
wherein the composition for delivery of the self-amplifying alphavirus-based expression system comprises:
(A) the self-amplifying alphavirus-based expression system, wherein the self-amplifying alphavirus-based expression system comprises one or more vectors, wherein the one or more vectors comprises:
(a) an RNA alphavirus backbone, wherein the RNA alphavirus backbone comprises:
(i) at least one promoter nucleotide sequence, and
(ii) at least one polyadenylation (poly(A)) sequence; and
(b) a cassette, wherein the cassette comprises:
(i) at least one antigen-encoding nucleic acid sequence comprising:
a. an epitope-encoding nucleic acid sequence, optionally comprising at least one alteration that makes the encoded epitope sequence distinct from the corresponding peptide sequence encoded by a wild-type nucleic acid sequence,
b. optionally a 5′ linker sequence, and
c. optionally a 3′ linker sequence;
(ii) optionally, a second promoter nucleotide sequence operably linked to the at least one antigen-encoding nucleic acid sequence; and
(iii) optionally, at least one second poly(A) sequence, wherein the second poly(A) sequence is a native poly(A) sequence or an exogenous poly(A) sequence to the alphavirus, and
(B) a lipid-nanoparticle (LNP), wherein the LNP encapsulates the self-amplifying alphavirus-based expression system, and
wherein the composition comprises at least 10 μg of each of the one or more vectors.

2. (canceled)

3. The composition of claim 1, wherein the composition for delivery of the self-amplifying alphavirus-based expression system comprises at least 30 μg of each of the one or more vectors.

4. The composition of claim 1, wherein the composition for delivery of the self-amplifying alphavirus-based expression system comprises at least 100 μg of each of the one or more vectors.

5. (canceled)

6. (canceled)

7. The composition of claim 1, wherein the composition for delivery of the self-amplifying alphavirus-based expression system comprises between 10-30 μg, 10-100 μg, or 10-300 μg of each of the one or more vectors.

8-15. (canceled)

16. The composition claim 1, wherein the one or more vectors is at a concentration of 1 mg/mL.

17-89. (canceled)

90. A composition for delivery of a chimpanzee adenovirus (ChAdV)-based expression system,
wherein the composition for delivery of the ChAdV-based expression system comprises:
the ChAdV-based expression system, wherein the ChAdV-based expression system comprises a viral particle comprising a ChAdV vector, wherein the ChAdV vector comprises:
(a) a ChAdV backbone, wherein the ChAdV backbone comprises:
(i) at least one promoter nucleotide sequence, and
(ii) at least one polyadenylation (poly(A)) sequence; and
(b) a cassette, wherein the cassette comprises:
(i) at least one antigen-encoding nucleic acid sequence comprising:
a. an epitope-encoding nucleic acid sequence, optionally comprising at least one alteration that makes the encoded epitope sequence distinct from the corresponding peptide sequence encoded by a wild-type nucleic acid sequence,
b. optionally a 5′ linker sequence, and
c. optionally a 3′ linker sequence; and
wherein the cassette is operably linked to the at least one promoter nucleotide sequence and the at least one poly(A) sequence, and wherein the composition comprises 1×10¹² or less of the viral particles.

91. (canceled)

92. (canceled)

93. The composition of claim 90, wherein the composition for delivery of the ChAdV-based expression system comprises at least 1×10¹¹ of the viral particles.

94. The composition of claim 90, wherein the composition for delivery of the ChAdV-based expression system comprises between 1×10¹².

95. The composition of claim 90, wherein the composition for delivery of the ChAdV-based expression system comprises 1×10¹¹, 3×10¹¹, or 1×10¹² of the viral particles.

96. The composition of claim 90, wherein the viral particles are at a concentration of at 5×10¹¹ vp/mL.

97. The composition of claim 1, wherein the epitope-encoding nucleic acid sequence encodes an epitope known or suspected to be presented by MHC class I on a surface of a cell, wherein the surface of the cell is a tumor cell surface or an infected cell surface, and optionally wherein the cell is a subject's cell.

98. The composition of claim 97, wherein the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer, or wherein the cell is an infected cell selected from the group consisting of: a pathogen infected cell, a virally infected cell, a bacterially infected cell, an fungally infected cell, and a parasitically infected cell.

99-112. (canceled)

113. The composition of claim 90, wherein the ChAdV backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) nucleotides 34,916 to 35,642 of the sequence shown in SEQ ID NO:1 corresponding to a partial E4 deletion; optionally wherein the antigen cassette is inserted within the E1 deletion.

114-132. (canceled)

133. The composition of claim 1, wherein the epitope-encoding nucleic acid sequence encodes an epitope known or suspected to be expressed in a subject known or suspected to have cancer selected from the group consisting of: MSS-CRC, NSCLC, and PDA.

134-178. (canceled)

179. The composition of claim 1, wherein the composition for delivery of the expression system is formulated in a pharmaceutical composition comprising a pharmaceutically acceptable carrier.

180. A kit comprising the composition for delivery of the expression system of claim 1, and instructions for use.

181. (canceled)

182. (canceled)

183. (canceled)

184. (canceled)

185. The composition of claim 1, wherein the epitope-encoding nucleic acid sequence comprises an epitope selected from the group consisting of SEQ ID NO: 57-29,357 and SEQ ID NO: 29,512-29,519.

186. (canceled)

187. (canceled)

188. (canceled)

189. The composition of claim 1, wherein the at least one antigen-encoding nucleic acid sequence comprises at least each of:
(A) a KRAS_G12C MHC class I epitope encoding nucleic acid sequence,
(B) a KRAS_G12D MHC class I epitope encoding nucleic acid sequence, and
(C) a KRAS_G12V MHC class I epitope encoding nucleic acid sequence.

190. (canceled)

191. (canceled)

192. (canceled)

193. (canceled)

194. (canceled)

195. (canceled)

196. A method for stimulating an immune response in a subject, the method comprising administering to the subject a composition for delivery of a self-amplifying alphavirus-based expression system and administering to the subject a composition for delivery of a chimpanzee adenovirus (ChAdV)-based expression system, and
wherein either:
a. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles,
b. wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors, or
c. the composition for delivery of the ChAdV-based expression system comprises the ChAdV-based expression system, wherein the ChAdV-based expression system comprises a viral particle comprising a ChAdV vector, and wherein the composition comprises 1×10¹² or less of the viral particles and wherein the composition for delivery of the self-amplifying alphavirus-based expression system comprises the self-amplifying alphavirus-based expression system, wherein the self-amplifying alphavirus-based expression system comprises one or more vectors, and wherein the composition comprises at least 10 μg of each of the one or more vectors.

197. The method of claim 196, wherein the composition for delivery of the ChAdV-based expression system is administered as a priming dose and the composition for delivery of the self-amplifying alphavirus-based expression system is administered as one or more boosting doses.

198-434. (canceled)
